(19) United States
(12) Patent Application Publication
(10) Pub. No.: US 2026/0327232 A1
(43) Pub. Date: Oct. 22, 2026

(54) Heteroaryl Compounds as Ligand Directed Degraders of IRAK4

(71) Applicant: Celgene Corporation, Princeton, NJ (US)

(72) Inventors: Geraint Davies, Arlington, MA (US); Paul E. Gormisky, Belmont, MA (US); Timothy Gordon Rasmusson, Sudbury, MA (US)

(73) Assignee: Celgene Corporation, Princeton, NJ (US)

(21) Appl. No.: 19/481,290

(22) PCT Filed: May 9, 2024
(86) PCT No.: PCT/US2024/028517

§ 371 Date: Nov. 4, 2025

Related U.S. Application Data
(60) Provisional application No. 63/465,603, filed on May 11, 2023.

(51) Int. Cl. C07D 487/04; A61K 31/519

(52) U.S. Cl. CPC C07D 487/04; A61K 31/519

(57) ABSTRACT

Provided herein are compounds and compositions thereof for modulating IRAK4. In some embodiments, the compounds and compositions are provided for treatment of inflammatory or autoimmune diseases.

### CROSS-REFERENCE TO RELATED APPLICATIONS

This application claims priority to U.S. Provisional Application No. 63/465,603, filed on May 11, 2023, which is incorporated herein by reference in its entirety for any purpose.

### FIELD

The present disclosure relates generally to compounds, compositions, and methods for their preparation and use of the compounds and compositions for treating inflammatory or autoimmune diseases.

### BACKGROUND

The recruitment of immune cells to sites of injury involves the concerted interactions of a large number of soluble mediators. Several cytokines appear to play key roles in these processes, including interleukin-1 (IL-1). IL-1 produces proinflammatory responses and contributes to the tissue degeneration observed in chronic inflammatory conditions. IL-1 has also been implicated in the process of bone resorption and adipose tissue regulation. Thus, IL-1 plays a key role in a large number of pathological conditions including rheumatoid arthritis, inflammatory bowel disease, multiple sclerosis, diabetes, obesity, cancer, and sepsis.

IL-1 treatment of cells induces the formation of a complex consisting of the two IL-1 receptor chains, IL-1R1 and IL-1RAcP, and the resulting heterodimer recruits an adaptor molecule designated as MyD88, which binds to IL-1 receptor associated kinase (IRAK) (Wesche et al., J. Biol. Chem. 1999, 274, 19403-19410; O'Neill et al., J. Leukoc. Biol. 1998, 63, 650-657; Auron, Cytokine Growth Factor Rev. 1998, 9:221-237; and O'Neill, Biochem. Soc. Trans. 2000, 28, 557-563). Four members of the IRAK family have been identified: IRAK1, IRAK2, IRAK3, and IRAK4. These proteins are characterized by a typical N-terminal death domain that mediates interaction with MyD88-family adaptor proteins and a centrally located kinase domain. Of the four members in the mammalian TRAK family, IRAK-4 is considered to be the “master IRAK.” IRAK-4 is a serine/threonine kinase that plays an essential role in signal transduction by Toll/IL-1 receptors (TIRs). Under overexpression conditions, all TRAKs can mediate the activation of nuclear factor-kappa B and stress-induced mitogen activated protein kinase (MAPK)-signaling cascades. Studies have shown that IRAK4 kinase activity is essential for cytokine production, activation of MAPKs, and induction of NF-kappa B regulated genes in response to TLR ligands (Koziczak-Holbro M. et al., J Biol. Chem. 2007, 282, 13552-13560). Given the central role of IRAK4 in Toll-like/IL-1R signaling and immunological protection, compounds that modulate the function of IRAK4 may be useful in treating inflammatory, cell proliferative, and immune-related conditions and diseases associated with IRAK-mediated signal transduction such as rheumatoid arthritis, inflammatory bowel disease, multiple sclerosis, diabetes, obesity, allergic disease, psoriasis, asthma, graft rejection, cancer and sepsis.

Protein degradation is a highly regulated and essential process that maintains cellular homeostasis. Selective identification and removal of damaged, misfolded, or excess proteins is achieved through the ubiquitin-proteasome pathway (UPP). The UPP is central to the regulation of almost all cellular processes. Ubiquitination of the protein is accomplished by an E3 ubiquitin ligase that binds to a protein and adds ubiquitin molecules to the protein, thus marking the protein for proteasome degradation.

Harnessing the UPP for therapeutic use has received significant interest (Zhou et al., Mol. Cell 2000, 6, 751-756). One promising therapy uses proteolysis targeting chimeras, commonly referred to as PROTACs, to effect removal of unwanted proteins by protein degradation (Scheepstra et al., Comp. Struct. Biotech. J. 2019, 17, 160-176). PROTACS are ligand directed degraders that bring together an E3 ligase and a target protein that is to be degraded. These bivalent molecules usually consist of an E3 ligase ligand connected through a linker moiety to small molecule that binds to the target protein. A PROTAC positions the E3 ligase at the appropriate distance and orientation to the target protein, allowing the latter to be ubiquitinated. The ubiquitinated target protein is subsequently recognized by the proteasome, where it is degraded.

Accordingly, in one aspect, provided herein are compounds that target IRAK4 for degradation.

### SUMMARY

Described herein, in certain embodiments, are compounds and compositions thereof for degrading IRAK4. In various embodiments, the compounds and compositions thereof may be used in treatment of inflammatory or autoimmune diseases.

The present embodiments can be understood more fully by reference to the detailed description and examples, which are intended to exemplify non-limiting embodiments.

Embodiment 1 is a compound of Formula (I):

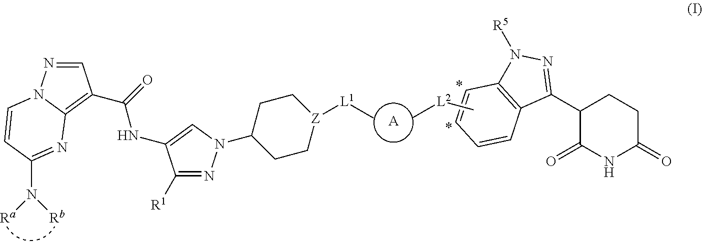

or a pharmaceutically acceptable salt thereof, wherein:
R¹ is C₁-C₆ haloalkyl;
R^{a} is H or C₁-C₆ alkyl;
R^{b} is C₁-C₆ alkyl, 5- to 6-membered heteroaryl, —(C₁-C₆ alkylene)(5- to 6-membered heteroaryl), or —(C₁-C₆ alkylene)NH₂, wherein the heteroaryl contains 1-2 nitrogen atoms and is optionally substituted by 1-5 R² groups;
or the dashed line between R^{a} and R^{b} represents a ring structure where R^{a} and R^{b} are taken together with the nitrogen atom to which they are attached to form a 5- to 10-membered monocyclic or bicyclic heterocyclyl optionally containing 1-2 additional heteroatoms selected from N and O, and is optionally substituted by 1-5 R² groups;
each R² is independently —NH₂, halo, C₁-C₆ alkyl, C₁-C₆ haloalkyl, —CN, or 5- to 6-membered heteroaryl, wherein the heteroaryl contains 1-2 nitrogen atoms and is optionally substituted by 1-5 groups selected from C₁-C₆ alkyl, halo, and C₁-C₆ haloalkyl;
L¹ is —C(O)N(H)—, —C(O)—, —(C₁-C₆ alkylene)N(R³)—, or C₁-C₆ alkylene;
L² is a bond, —C(O)—, —N(R³)—, or O;
each R³ is independently H or C₁-C₆ alkyl;
Ring A is a monocyclic 4- to 6-membered heterocyclylene or a bicyclic 6- to 9-membered spiro heterocyclylene, wherein the heterocyclylene contains 1-2 nitrogen atoms, and wherein the heterocyclylene is substituted by m R⁴ groups;
each R⁴ is independently halo, C₁-C₆ alkyl, or C₁-C₆ haloalkyl;
m is 0-5;
R⁵ is H or C₁-C₆ alkyl;
Z is CH or N; and
* indicates an attachment point for the L²-containing moiety.

Embodiment 2 is the compound of embodiment 1, or a pharmaceutically acceptable salt thereof, wherein:
R¹ is C₁-C₃ haloalkyl.

Embodiment 3 is the compound of embodiment 2, or a pharmaceutically acceptable salt thereof, wherein:
R¹ is —CHF₂.

Embodiment 4 is the compound of any one of embodiments 1-3, or a pharmaceutically acceptable salt thereof, wherein:
R^{a} is H or C₁-C₃ alkyl;
R^{b} is C₁-C₃ alkyl, 6-membered heteroaryl, —(C₁-C₃ alkylene)(6-membered heteroaryl), or —(C₁-C₃ alkylene)NH₂, wherein the heteroaryl contains 1-2 nitrogen atoms and is optionally substituted by 1-2 R² groups; and
each R² is independently —NH₂, halo, C₁-C₃ alkyl, C₁-C₃ haloalkyl, or —CN.

Embodiment 5 is the compound of embodiment 4, or a pharmaceutically acceptable salt thereof, wherein:

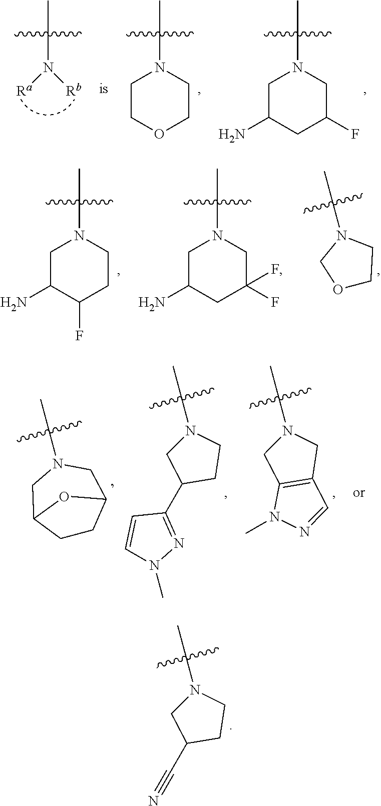

Embodiment 6 is the compound of any one of embodiments 1-3, or a pharmaceutically acceptable salt thereof, wherein:
R^{a} and R^{b} are taken together with the nitrogen atom to which they are attached to form a 5- to 8-membered monocyclic or bicyclic heterocyclyl optionally containing 1-2 additional heteroatoms selected from N and O, and optionally substituted by 1-3 R² groups; and
each R² is independently —NH₂, halo, C₁-C₃ alkyl, C₁-C₃ haloalkyl, —CN, or 5-membered heteroaryl, wherein the heteroaryl contains 1-2 nitrogen atoms and is optionally substituted by 1-3 groups selected from C₁-C₃ alkyl, halo, and C₁-C₃ haloalkyl.

Embodiment 7 is the compound of embodiment 6, or a pharmaceutically acceptable salt thereof, wherein:

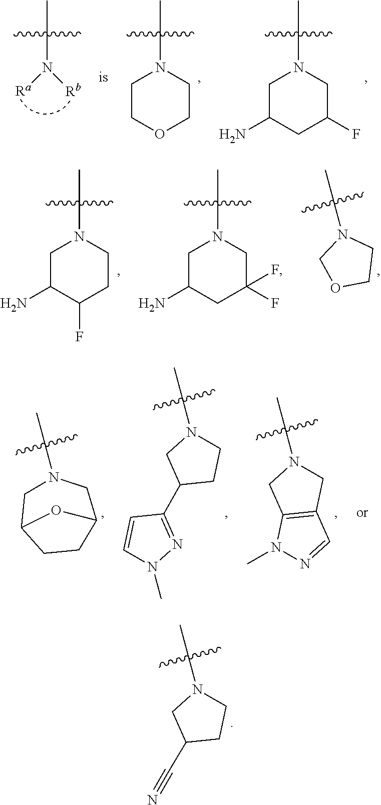

Embodiment 8 is the compound of any one of embodiments 1-7, or a pharmaceutically acceptable salt thereof, wherein:
L¹ is —C(O)N(H)—, —C(O)—, —(C₁-C₃ alkylene)N(R³)—, or C₁-C₃ alkylene;
L² is a bond, —C(O)—, —N(R³)—, or O; and
each R³ is independently H or C₁-C₃ alkyl.

Embodiment 9 is the compound of any one of embodiments 1-8, or a pharmaceutically acceptable salt thereof, wherein:
Ring A is:

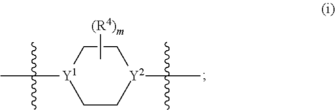

and
Y¹ and Y² are independently CH or N, provided that at least one of Y¹ and Y² is N;

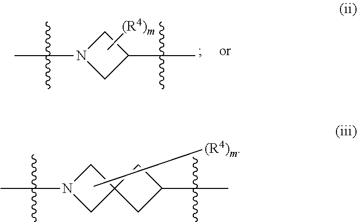

Embodiment 10 is the compound of any one of embodiments 1-9, or a pharmaceutically acceptable salt thereof, wherein:
each R⁴ is independently halo, C₁-C₃ alkyl, or C₁-C₃ haloalkyl.

Embodiment 11 is the compound of any one of embodiments 1-10, or a pharmaceutically acceptable salt thereof, wherein:
m is 0, 1, or 2.

Embodiment 12 is the compound of any one of embodiments 1-11, or a pharmaceutically acceptable salt thereof, wherein:

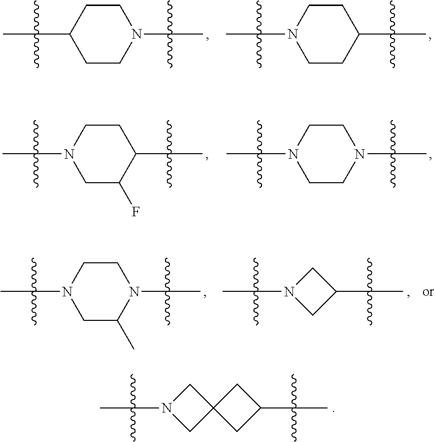

Embodiment 13 is the compound of any one of embodiments 1-12, or a pharmaceutically acceptable salt thereof, wherein:
R⁵ is H or C₁-C₃ alkyl.

Embodiment 14 is the compound of embodiment 13, or a pharmaceutically acceptable salt thereof, wherein:
R⁵ is —CH₃.

Embodiment 15 is the compound of any one of embodiments 1-14, or a pharmaceutically acceptable salt thereof, wherein:

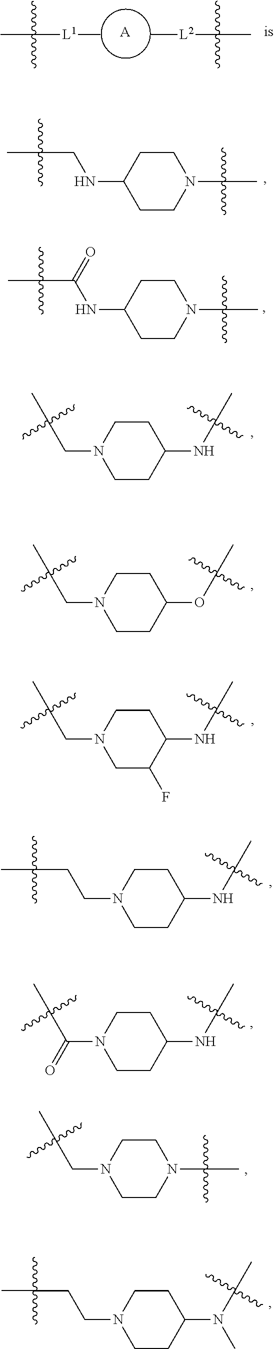

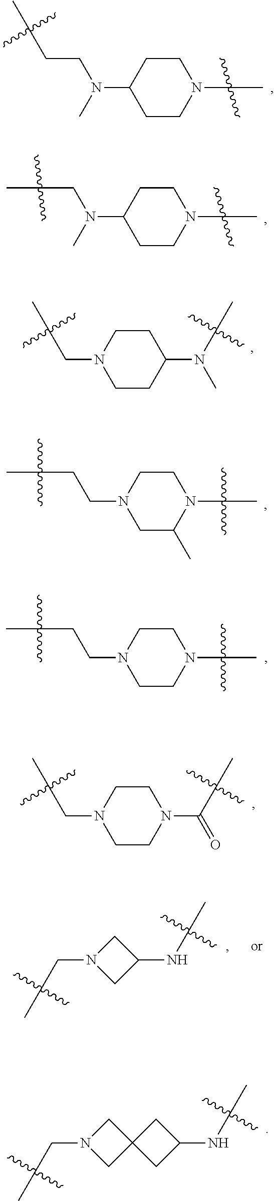

Embodiment 16 is the compound of any one of embodiments 1-15, or a pharmaceutically acceptable salt thereof, wherein the compound is of Formula (IA), (IB), (IIa), (IIb), (IIIa), or (IVa):

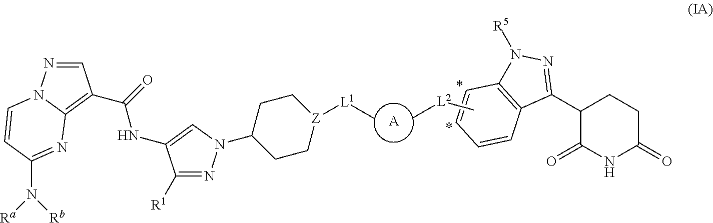

wherein:
R^{a} is H or C₁-C₆ alkyl; and
R^{b} is C₁-C₆ alkyl, 5- to 6-membered heteroaryl, —(C₁-C₆ alkylene)(5- to 6-membered heteroaryl), or —(C₁-C₆ alkylene)NH₂, wherein the heteroaryl contains 1-2 nitrogen atoms and is optionally substituted by 1-5 R² groups;

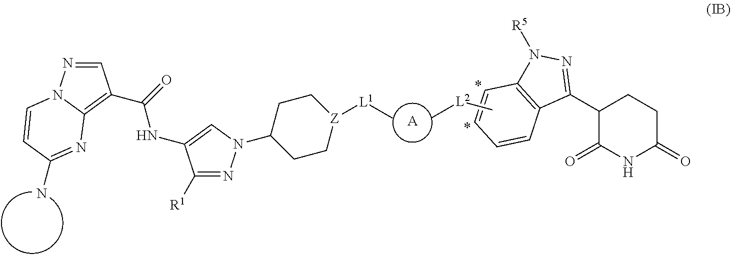

wherein:

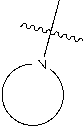 [SMILES: *N]

is a 5- to 10-membered monocyclic or bicyclic heterocyclyl optionally containing 1-2 additional heteroatoms selected from N and O, and is optionally substituted by 1-5 R² groups;

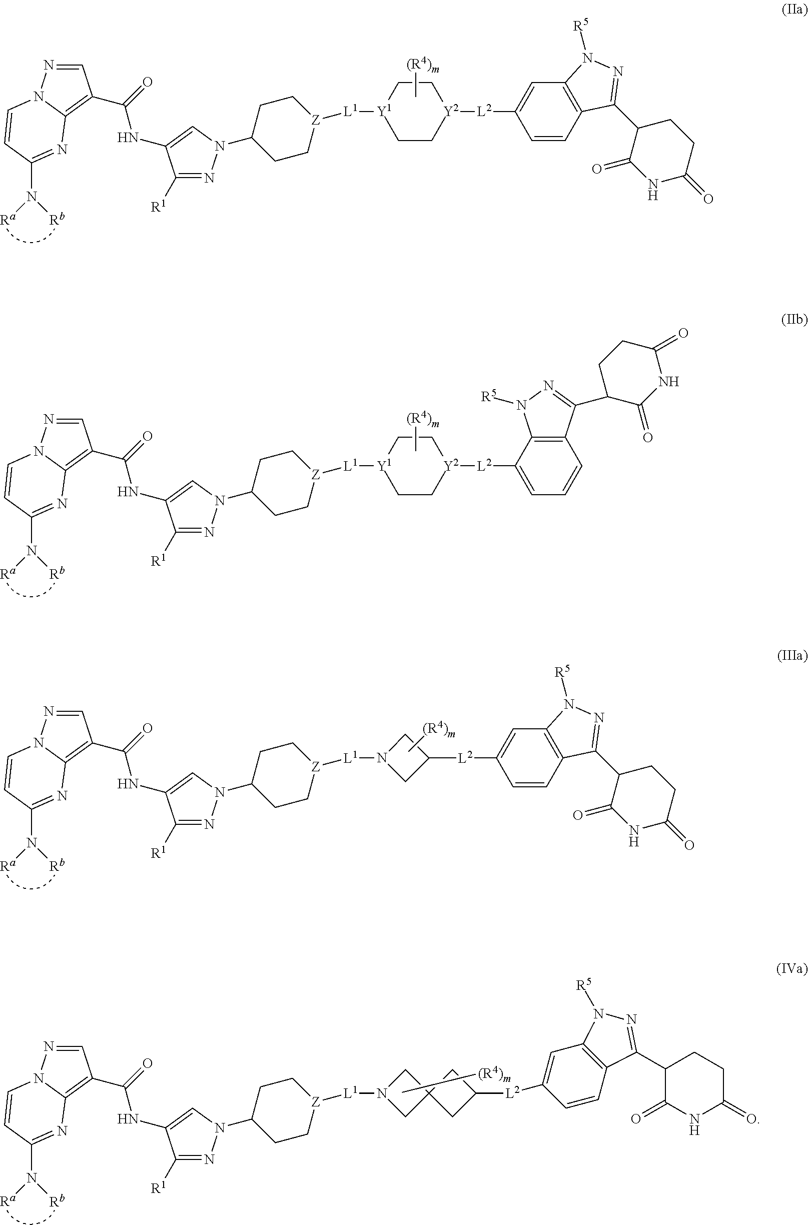

Embodiment 17 is a compound selected from the compounds of Table 1 and pharmaceutically acceptable salts thereof.

Embodiment 18 is a pharmaceutical composition comprising the compound of any one of embodiments 1-17, or a pharmaceutically acceptable salt thereof, and a pharmaceutically acceptable excipient.

Embodiment 19 is a method of modulating interleukin-1 (IL1) receptor-associated kinase 4 (IRAK4) comprising contacting IRAK4 with an effective amount of the compound of any one of embodiments 1-17, or a pharmaceutically acceptable salt thereof, or the pharmaceutical composition of embodiment 18.

Embodiment 20 is a method of treating an inflammatory or autoimmune disease in a subject in need thereof, comprising administering to the subject an effective amount of the compound of any one of embodiments 1-17, or a pharmaceutically acceptable salt thereof, or the pharmaceutical composition of embodiment 18, optionally wherein the inflammatory or autoimmune disease is atopic dermatitis, asthma, lupus, rheumatoid arthritis, familial mediterranean fever, psoriasis, generalized pustular psoriasis, cryoprin-associated periodic syndrome, hidradenitis suppurativa, Bechet's syndrome, or familial cold autoinflammatory syndrome.

### DETAILED DESCRIPTION

### Definitions

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the terms “about” and “approximately” mean±20%, ±10%, ±5%, or ±1% of the indicated range, value, or structure, unless otherwise indicated.

An “alkyl” group is a saturated, partially saturated, or unsaturated straight chain or branched non-cyclic hydrocarbon having from 1 to 10 carbon atoms (C₁-C₁₀ alkyl), typically from 1 to 8 carbons (C₁-C₈ alkyl) or, in some embodiments, from 1 to 6 (C₁-C₆ alkyl), 1 to 3 (C₁-C₃ alkyl), or 2 to 6 (C₂-C₆ alkyl) carbon atoms. In some embodiments, the alkyl group is a saturated alkyl group. Representative saturated alkyl groups include -methyl, -ethyl, -n-propyl, -n-butyl, -n-pentyl and -n-hexyl; while saturated branched alkyls include -isopropyl, -sec-butyl, -isobutyl, tert-butyl, -isopentyl, -neopentyl, tertpentyl, -2-methylpentyl, -3-methylpentyl, -4-methylpentyl, -2,3-dimethylbutyl and the like. In some embodiments, an alkyl group is an unsaturated alkyl group, also termed an alkenyl or alkynyl group. An “alkenyl” group is an alkyl group that contains one or more carbon-carbon double bonds. An “alkynyl” group is an alkyl group that contains one or more carbon-carbon triple bonds. Examples of unsaturated alkyl groups include, but are not limited to, vinyl, allyl, CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, CH₂C≡C(CH₃) and CH₂C≡C(CH₂CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen; hydroxy; alkoxy; cycloalkyloxy, aryloxy, heterocyclyloxy, heteroaryloxy, heterocycloalkyloxy, cycloalkylalkyloxy, aralkyloxy, heterocyclylalkyloxy, heteroarylalkyloxy, heterocycloalkylalkyloxy; oxo (═O); amino, alkylamino, cycloalkylamino, arylamino, heterocyclylamino, heteroarylamino, heterocycloalkylamino, cycloalkylalkylamino, aralkylamino, heterocyclylalkylamino, heteroaralkylamino, heterocycloalkylalkylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)₂. In certain embodiments, when the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

A “cycloalkyl” group is a saturated, or partially saturated cyclic alkyl group of from 3 to 10 carbon atoms (C₃-C₁₀ cycloalkyl) having a single cyclic ring or multiple condensed or bridged rings that can be optionally substituted. In some embodiments, the cycloalkyl group has 3 to 8 ring carbon atoms (C₃-C₈ cycloalkyl), whereas in other embodiments the number of ring carbon atoms ranges from 3 to 5 (C₃-C₅ cycloalkyl), 3 to 6 (C₃-C₆ cycloalkyl), or 3 to 7 (C₃-C₇ cycloalkyl). In some embodiments, the cycloalkyl groups are saturated cycloalkyl groups. Such saturated cycloalkyl groups include, by way of example, single ring structures such as cyclopropyl, cyclobutyl, cyclopentyl, cyclohexyl, cycloheptyl, cyclooctyl, 1methylcyclopropyl, 2methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. In other embodiments, the cycloalkyl groups are unsaturated cycloalkyl groups. Examples of unsaturared cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

An “aryl” group is an aromatic carbocyclic group of from 6 to 14 carbon atoms (C₆-C₁₄ aryl) having a single ring (e.g., phenyl) or multiple condensed rings (e.g., naphthyl or anthryl). In some embodiments, aryl groups contain 6-14 carbons (C₆-C₁₄ aryl), and in others from 6 to 12 (C₆-C₁₂ aryl) or even 6 to 10 carbon atoms (C₆-C₁₀ aryl) in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “halogen” or “halo” is fluorine, chlorine, bromine or iodine.

“Haloalkyl” refers to an alkyl radical, as defined above, that is substituted by one or more halo radicals, as defined above, e.g., trifluoromethyl, difluoromethyl, trichloromethyl, 2,2,2trifluoroethyl, 1,2difluoroethyl, 3bromo2fluoropropyl, 1,2dibromoethyl, and the like. In some embodiments, the haloalkyl group has one to six carbon atoms and is substituted by one or more halo radicals (C₁-C₆ haloalkyl), or the haloalkyl group has one to three carbon atoms and is substituted by one or more halo radicals (C₁-C₃ haloalkyl). The halo radicals may be all the same or the halo radicals may be different. Unless specifically stated otherwise, a haloalkyl group is optionally substituted.

A “heteroaryl” group is an aromatic ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1Hpyrrolo[2,3b]pyridyl), indazolyl, benzimidazolyl (e.g., 1Hbenzo[d]imidazolyl), imidazopyridyl (e.g., azabenzimidazolyl or 1Himidazo[4,5b]pyridyl), pyrazolopyridyl, triazolopyridyl, benzotriazolyl (e.g., 1Hbenzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl (e.g., 3,4dihydroisoquinolin-1(2H)-onyl), tetrahydroquinolinyl, quinoxalinyl, and quinazolinyl groups. A heteroaryl group can be substituted or unsubstituted.

A “heterocyclyl” is a non-aromatic cycloalkyl in which one to four of the ring carbon atoms are independently replaced with a heteroatom selected from O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocycloalkyl group can be substituted or unsubstituted. Heterocyclyl groups encompass saturated and partially saturated ring systems. Further, the term heterocyclyl is intended to encompass any non-aromatic ring containing at least one heteroatom, which ring may be fused to an aryl or heteroaryl ring, regardless of the attachment to the remainder of the molecule. The phrase also includes bridged polycyclic ring systems containing a heteroatom. Representative examples of a heterocyclyl group include, but are not limited to, aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4dioxaspiro[4.5]decanyl, homopiperazinyl, quinuclidyl, or tetrahydropyrimidin-2(1H)-one. Representative substituted heterocyclyl groups may be monosubstituted or substituted more than once, such as, but not limited to, pyridyl or morpholinyl groups, which are 2-, 3-, 4-, 5-, or 6 substituted, or disubstituted with various substituents such as those listed below.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl) aryloxy; aralkyloxy; heterocyclyloxy; and heterocyclyl alkoxy.

Certain commonly used alternative chemical names may be used. For example, a divalent group such as a divalent “alkyl” group, a divalent “phenyl” group, a divalent “heteroaryl” group, a divalent “heterocyclyl” group etc., may also be referred to as an “alkylene” group, a “phenylene” group, a “heteroarylene” group, or a “heterocyclylene” group, respectively.

Embodiments of the disclosure are meant to encompass pharmaceutically acceptable salts, tautomers, isotopologues, and stereoisomers of the compounds provided herein, such as the compounds of Formula (I).

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds of formula (I) include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (Nmethyl-glucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and ptoluenesulfonic acid. Specific non-toxic acids include hydrochloric, hydrobromic, maleic, phosphoric, sulfuric, and methanesulfonic acids. Examples of specific salts thus include hydrochloride, formic, and mesylate salts. Others are well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereoisomerically pure” means one stereoisomer of a particular compound that is substantially free of other stereoisomers of that compound. For example, a stereoisomerically pure compound having one chiral center will be substantially free of the opposite enantiomer of the compound. A stereoisomerically pure compound having two chiral centers will be substantially free of other diastereomers of the compound. A typical stereoisomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds disclosed herein can have chiral centers and can occur as racemates, individual enantiomers or diastereomers, and mixtures thereof. All such isomeric forms are included within the embodiments disclosed herein, including mixtures thereof.

The use of stereoisomerically pure forms of the compounds disclosed herein, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (WileyInterscience, New York, 1981); Wilen, S. H., et al., Tetrahedron 33:2725 (1977); Eliel, E. L., Stereochemistry of Carbon Compounds (McGrawHill, NY, 1962); Wilen, S. H., Tables of Resolving Agents and Optical Resolutions p. 268 (E. L. Eliel, Ed., Univ. of Notre Dame Press, Notre Dame, IN, 1972); Todd, M., Separation Of Enantiomers: Synthetic Methods (Wiley-VCH Verlag GmbH & Co. KGaA, Weinheim, Germany, 2014); Toda, F., Enantiomer Separation: Fundamentals and Practical Methods (Springer Science & Business Media, 2007); Subramanian, G. Chiral Separation Techniques: A Practical Approach (John Wiley & Sons, 2008); Ahuja, S., Chiral Separation Methods for Pharmaceutical and Biotechnological Products (John Wiley & Sons, 2011).

It should also be noted the compounds disclosed herein can include E and Z isomers, or a mixture thereof, and cis and trans isomers or a mixture thereof. In certain embodiments, the compounds are isolated as either the E or Z isomer. In other embodiments, the compounds are a mixture of the E and Z isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

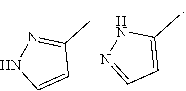

As readily understood by one skilled in the art, a wide variety of functional groups and other structures may exhibit tautomerism and all tautomers of compounds of Formula (I) are within the scope of the present disclosure.

It should also be noted the compounds disclosed herein can contain unnatural proportions of atomic isotopes at one or more of the atoms. For example, the compounds may be radiolabeled with radioactive isotopes, such as for example tritium (³H), iodine-125 (¹²⁵), sulfur35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically encriched compounds are useful as therapeutic agents, e.g., cancer therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds disclosed herein, for example, the isotopologues are deuterium, carbon-13, and/or nitrogen-15 enriched compounds. As used herein, “deuterated”, means a compound wherein at least one hydrogen (H) has been replaced by deuterium (indicated by D or ²H), that is, the compound is enriched in deuterium in at least one position.

It is understood that, independently of stereoisomerical or isotopic composition, each compound disclosed herein can be provided in the form of any of the pharmaceutically acceptable salts discussed herein. Equally, it is understood that the isotopic composition may vary independently from the stereoisomerical composition of each compound referred to herein. Further, the isotopic composition, while being restricted to those elements present in the respective compound or salt thereof disclosed herein, may otherwise vary independently from the selection of the pharmaceutically acceptable salt of the respective compound.

It should be noted that if there is a discrepancy between a depicted structure and a name for that structure, the depicted structure is to be accorded more weight.

“Treating” as used herein, means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself. In one embodiment, the disorder is a neurodegenerative disease, as described herein, or a symptom thereof.

“Preventing” as used herein, means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition. In one embodiment, the disorder is a neurodegenerative disease, as described herein, or symptoms thereof.

The term “effective amount” in connection with a compound disclosed herein means an amount capable of treating or preventing a disorder, disease or condition, or symptoms thereof, disclosed herein.

The term “subject” or “patient” as used herein include an animal, including, but not limited to, an animal such a cow, monkey, horse, sheep, pig, chicken, turkey, quail, cat, dog, mouse, rat, rabbit or guinea pig, in one embodiment a mammal, in another embodiment a human. In one embodiment, a subject is a human having or at risk for having an S1P5 mediated disease, or a symptom thereof.

Although various features of the invention may be described in the context of a single embodiment, the features may also be provided separately or in any suitable combination. Conversely, although the invention may be described herein in the context of separate embodiments for clarity, the invention may also be implemented in a single embodiment.

### Compounds

In one aspect, provided herein is a compound of Formula (I):

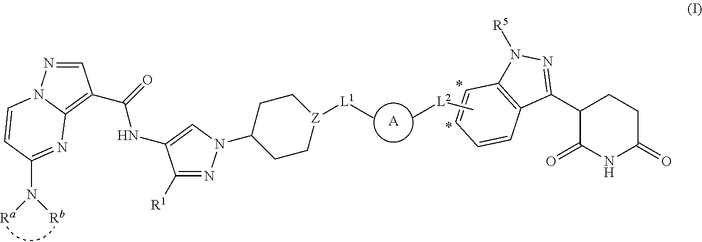

or a pharmaceutically acceptable salt thereof, wherein:
R¹ is C₁-C₆ haloalkyl;
R^{a} is H or C₁-C₆ alkyl;
R^{b} is C₁-C₆ alkyl, 5- to 6-membered heteroaryl, —(C₁-C₆ alkylene)(5- to 6-membered heteroaryl), or —(C₁-C₆ alkylene)NH₂, wherein the heteroaryl contains 1-2 nitrogen atoms and is optionally substituted by 1-5 R² groups;
or the dashed line between R^{a} and R^{b} represents a ring structure where R^{a} and R^{b} are taken together with the nitrogen atom to which they are attached to form a 5- to 10-membered monocyclic or bicyclic heterocyclyl optionally containing 1-2 additional heteroatoms selected from N and O, and is optionally substituted by 1-5 R² groups;
each R² is independently —NH₂, halo, C₁-C₆ alkyl, C₁-C₆ haloalkyl, —CN, or 5- to 6-membered heteroaryl, wherein the heteroaryl contains 1-2 nitrogen atoms and is optionally substituted by 1-5 groups selected from C₁-C₆ alkyl, halo, and C₁-C₆ haloalkyl;
L¹ is —C(O)N(H)—, —C(O)—, —(C₁-C₆ alkylene)N(R³)—, or C₁-C₆ alkylene;
L² is a bond, —C(O)—, —N(R³)—, or O;
each R³ is independently H or C₁-C₆ alkyl;
Ring A is a monocyclic 4- to 6-membered heterocyclylene or a bicyclic 6- to 9-membered spiro heterocyclylene, wherein the heterocyclylene contains 1-2 nitrogen atoms, and wherein the heterocyclylene is substituted by m R⁴ groups;
each R⁴ is independently halo, C₁-C₆ alkyl, or C₁-C₆ haloalkyl;
m is 0-5;
R⁵ is H or C₁-C₆ alkyl;
Z is CH or N; and
* indicates an attachment point for the L²-containing moiety.

In some embodiments, R¹ is C₁-C₆ haloalkyl. In some embodiments, R¹ is C₁-C₃ haloalkyl. In some embodiments, R¹ is —CHF₂ or —CF₃. In some embodiments, R¹ is —CHF₂.

In some embodiments, R¹ is C₁-C₆ haloalkyl. In some embodiments, R¹ is C₁-C₆ haloalkyl containing 1-13 halogen atoms. In some embodiments, R¹ is C₁-C₃ haloalkyl. In some embodiments, R¹ is C₁-C₃ haloalkyl containing 1-7 halogen atoms. In some embodiments, R¹ is —CF₃, —CHF₂, —CH₂F, —CCl₃, —CHCl₂, —CH₂Cl, —CF₂Cl, —CFCl₂, —CH₂CF₃, —CH₂CHF₂, or —CH₂CCl₃. In some embodiments, R¹ is —CF₃. In some embodiments, R¹ is —CHF₂.

In some embodiments, R^{a} is H or C₁-C₃ alkyl; R^{b} is C₁-C₃ alkyl, 6-membered heteroaryl, —(C₁-C₃ alkylene)(6-membered heteroaryl), or —(C₁-C₃ alkylene)NH₂, wherein the heteroaryl contains 1-2 nitrogen atoms and is optionally substituted by 1-2 R² groups; and each R² is independently —NH₂, halo, C₁-C₃ alkyl, C₁-C₃ haloalkyl, or —CN. In some embodiments, R^{a} is H or —CH₃; R^{b} is —CH₃, —CH₂(pyridyl), —CH₂CH₂NH₂, or pyridyl, wherein the pyridyl is optionally substituted by 1 R² group; and R² is —CH₃.

In some embodiments, R^{a} is H or C₁-C₆ alkyl. In some embodiments, R^{a} is H or C₁-C₃ alkyl. In some embodiments, R^{a} is H or —CH₃.

In some embodiments, R^{a} is H.

In some embodiments, R^{a} is C₁-C₆ alkyl. In some embodiments, R^{a} is C₁-C₃ alkyl. In some embodiments, R^{a} is methyl, ethyl, n-propyl, or isopropyl. In some embodiments, R^{a} is methyl. In some embodiments, R^{a} is ethyl. In some embodiments, R^{a} is n-propyl. In some embodiments, R^{a} is isopropyl.

In some embodiments, R^{b} is C₁-C₆ alkyl, 5- to 6-membered heteroaryl, —(C₁-C₆ alkylene)(5- to 6-membered heteroaryl), or —(C₁-C₆ alkylene)NH₂, wherein the heteroaryl contains 1-2 nitrogen atoms and is optionally substituted by 1-5 R² groups. In some embodiments, R^{b} is C₁-C₃ alkyl, 6-membered heteroaryl, —(C₁-C₃ alkylene)(6-membered heteroaryl), or —(C₁-C₃ alkylene)NH₂, wherein the heteroaryl contains 1-2 nitrogen atoms and is optionally substituted by 1-2 R² groups. In some embodiments, R^{b} is —CH₃, —CH₂(pyridyl), —CH₂CH₂NH₂, or pyridyl, wherein the pyridyl is optionally substituted by 1 R² group.

In some embodiments, R^{b} is C₁-C₆ alkyl. In some embodiments, R^{b} is C₁-C₃ alkyl. In some embodiments, R^{b} is methyl, ethyl, n-propyl, or isopropyl. In some embodiments, R^{b} is methyl. In some embodiments, R^{b} is ethyl. In some embodiments, R^{b} is n-propyl. In some embodiments, R^{b} is isopropyl.

In some embodiments, R^{b} is 5- to 6-membered heteroaryl, wherein the heteroaryl contains 1-2 nitrogen atoms and is optionally substituted by 1-5 R² groups. In some embodiments, R^{b} is 5- to 6-membered heteroaryl, wherein the heteroaryl contains 1-2 nitrogen atoms and is optionally substituted by 1-3 R² groups. In some embodiments, R^{b} is 5- to 6-membered heteroaryl, wherein the heteroaryl contains 1-2 nitrogen atoms and is optionally substituted by 1 or 2 R² groups. In some embodiments, R^{b} is 5- to 6-membered heteroaryl, wherein the heteroaryl contains 1-2 nitrogen atoms and is optionally substituted by 1 R² group. In some embodiments, R^{b} is 5- to 6-membered heteroaryl, wherein the heteroaryl contains 1-2 nitrogen atoms and is unsubstituted. In some embodiments, R^{b} is a 5-membered heteroaryl containing 1-2 nitrogen atoms. In some embodiments, R^{b} is a 5-membered heteroaryl containing 1 nitrogen atom. In some embodiments, R^{b} is a 5-membered heteroaryl containing 2 nitrogen atoms. In some embodiments, R^{b} is pyrrolyl, imidazolyl, or pyrazolyl. In some embodiments, R^{b} is a 6-membered heteroaryl containing 1-2 nitrogen atoms. In some embodiments, R^{b} is a 6-membered heteroaryl containing 1 nitrogen atom. In some embodiments, R^{b} is a 6-membered heteroaryl containing 2 nitrogen atoms. In some embodiments, R^{b} is pyridinyl, pyrazinyl, pyrimidinyl, or pyridazinyl. In some embodiments, R^{b} is pyridinyl.

In some embodiments, R^{b} is —(C₁-C₆ alkylene)(5- to 6-membered heteroaryl), wherein the heteroaryl contains 1-2 nitrogen atoms and is optionally substituted by 1-5 R² groups. In some embodiments, R^{b} is —(C₁-C₃ alkylene)(5- to 6-membered heteroaryl), wherein the heteroaryl contains 1-2 nitrogen atoms and is optionally substituted by 1-5 R² groups. In some embodiments, R^{b} is —(C₁-C₃ alkylene)(5- to 6-membered heteroaryl), wherein the heteroaryl contains 1-2 nitrogen atoms and is optionally substituted by 1-3 R² groups. In some embodiments, R^{b} is —(C₁-C₃ alkylene)(5- to 6-membered heteroaryl), wherein the heteroaryl contains 1-2 nitrogen atoms and is optionally substituted by 1 or 2 R² groups. In some embodiments, R^{b} is —(C₁-C₃ alkylene)(5- to 6-membered heteroaryl), wherein the heteroaryl contains 1-2 nitrogen atoms and is optionally substituted by 1 R² group. In some embodiments, R^{b} is —(C₁-C₃ alkylene)(5- to 6-membered heteroaryl), wherein the heteroaryl contains 1-2 nitrogen atoms and is unsubstituted.

In some embodiments, R^{b} is —(C₁-C₆ alkylene)(5-membered heteroaryl), wherein the heteroaryl contains 1-2 nitrogen atoms and is optionally substituted by 1-5 R² groups. In some embodiments, R^{b} is —(C₁-C₃ alkylene)(5-membered heteroaryl), wherein the heteroaryl contains 1-2 nitrogen atoms and is optionally substituted by 1-5 R² groups. In some embodiments, R^{b} is —(C₁-C₃ alkylene)(5-membered heteroaryl), wherein the heteroaryl contains 1-2 nitrogen atoms and is optionally substituted by 1-3 R² groups. In some embodiments, R^{b} is —(C₁-C₃ alkylene)(5-membered heteroaryl), wherein the heteroaryl contains 1-2 nitrogen atoms and is optionally substituted by 1 or 2 R² groups. In some embodiments, R^{b} is —(C₁-C₃ alkylene)(5-membered heteroaryl), wherein the heteroaryl contains 1-2 nitrogen atoms and is optionally substituted by 1 R² group. In some embodiments, R^{b} is —(C₁-C₃ alkylene)(5-membered heteroaryl), wherein the heteroaryl contains 1 nitrogen atom and is optionally substituted by 1-5 R² groups. In some embodiments, R^{b} is —(C₁-C₃ alkylene)(5-membered heteroaryl), wherein the heteroaryl contains 2 nitrogen atoms and is optionally substituted by 1-5 R² groups. In some embodiments, R^{b} is —(C₁-C₃ alkylene)pyrrolyl, —(C₁-C₃ alkylene)imidazolyl, or —(C₁-C₃ alkylene)pyrazolyl. In some embodiments, R^{b} is —CH₂(pyrrolyl), —CH₂(imidazolyl), or —CH₂(pyrazolyl). In some embodiments, R^{b} is —CH₂CH₂(pyrrolyl), —CH₂CH₂(imidazolyl), or —CH₂CH₂(pyrazolyl).

In some embodiments, R^{b} is —(C₁-C₆ alkylene)NH₂. In some embodiments, R^{b} is —(C₁-C₃ alkylene)NH₂. In some embodiments, R^{b} is —CH₂NH₂, —CH₂CH₂NH₂, or —CH₂CH₂CH₂NH₂. In some embodiments, R^{b} is —CH₂CH₂NH₂.

In some embodiments,

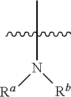

is:

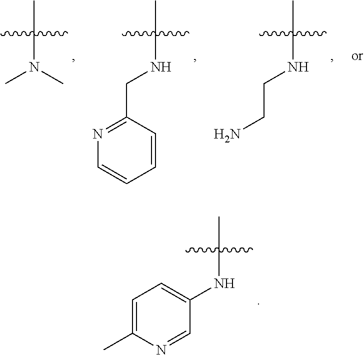

In some embodiments, R^{a} and R^{b} are taken together with the nitrogen atom to which they are attached to form a 5- to 10-membered monocyclic or bicyclic heterocyclyl optionally containing 1-2 additional heteroatoms selected from N and O, and is optionally substituted by 1-5 R² groups. In some embodiments, R^{a} and R^{b} are taken together with the nitrogen atom to which they are attached to form a 5- to 8-membered monocyclic or bicyclic heterocyclyl optionally containing 1-2 additional heteroatoms selected from N and O, and optionally substituted by 1-3 R² groups. In some embodiments, R^{a} and R^{b} are taken together with the nitrogen atom to which they are attached to form a 5- to 6-membered monocyclic heterocyclyl or 8-membered fused or bridged bicyclic heterocyclyl, wherein the heterocyclyl optionally contains 1-2 additional heteroatoms selected from N and O, and is optionally substituted by 1-3 R² groups.

In some embodiments, R^{a} and R^{b} are taken together with the nitrogen atom to which they are attached to form a 5- to 10-membered monocyclic heterocyclyl optionally containing 1-2 additional heteroatoms selected from N and O and optionally substituted by 1-5 R² groups. In some embodiments, R^{a} and R^{b} are taken together with the nitrogen atom to which they are attached to form a 5- to 6-membered monocyclic heterocyclyl optionally containing 1-2 additional heteroatoms selected from N and O and optionally substituted by 1-5 R² groups. In some embodiments, R^{a} and R^{b} are taken together with the nitrogen atom to which they are attached to form a 5- to 6-membered monocyclic heterocyclyl optionally containing 1 additional heteroatom selected from N and O and optionally substituted by 1-5 R² groups. In some embodiments, R^{a} and R^{b} are taken together with the nitrogen atom to which they are attached to form a 5- to 6-membered monocyclic heterocyclyl containing 1 additional nitrogen atom and optionally substituted by 1-5 R² groups. In some embodiments, R^{a} and R^{b} are taken together with the nitrogen atom to which they are attached to form a 5- to 6-membered monocyclic heterocyclyl containing 1 oxygen atom and optionally substituted by 1-5 R² groups. In some embodiments, R^{a} and R^{b} are taken together with the nitrogen atom to which they are attached to form a 5- to 6-membered monocyclic heterocyclyl containing no additional heteroatoms and optionally substituted by 1-5 R² groups. In some embodiments, the 5- to 6-membered monocyclic heterocyclyl is optionally substituted by 1-3 R² groups. In some embodiments, the 5- to 6-membered monocyclic heterocyclyl is optionally substituted by 1 or 2 R² groups. In some embodiments, the 5- to 6-membered monocyclic heterocyclyl is optionally substituted by 1 R² group. In some embodiments, the 5- to 6-membered monocyclic heterocyclyl is unsubstituted. In some embodiments, the 5- to 6-membered monocyclic heterocyclyl is a 5-membered heterocyclyl. In some embodiments, the 5-membered heterocyclyl is pyrrolidinyl or oxazolidinyl. In some embodiments, the 5- to 6-membered monocyclic heterocyclyl is a 6-membered heterocyclyl. In some embodiments, the 6-membered heterocyclyl is morpholinyl, piperazinyl, or piperidinyl.

In some embodiments, R^{a} and R^{b} are taken together with the nitrogen atom to which they are attached to form a 5- to 10-membered bicyclic heterocyclyl optionally containing 1-2 additional heteroatoms selected from N and O and optionally substituted by 1-5 R² groups. In some embodiments, R^{a} and R^{b} are taken together with the nitrogen atom to which they are attached to form a 5- to 8-membered bicyclic heterocyclyl optionally containing 1-2 additional heteroatoms selected from N and O and optionally substituted by 1-5 R² groups. In some embodiments, R^{a} and R^{b} are taken together with the nitrogen atom to which they are attached to form an 8-membered fused or bridged bicyclic heterocyclyl optionally containing 1-2 additional heteroatoms selected from N and O and optionally substituted by 1-5 R² groups. In some embodiments, the bicyclic heterocyclyl is substituted by 1-3 R² groups. In some embodiments, the bicyclic heterocyclyl is substituted by 1 or 2 R² groups. In some embodiments, the bicyclic heterocyclyl is substituted by one R² group. In some embodiments, the bicyclic heterocyclyl is unsubstituted.

In some embodiments, R^{a} and R^{b} are taken together with the nitrogen atom to which they are attached to form an 8-membered fused bicyclic heterocyclyl optionally containing 1-2 additional heteroatoms selected from N and O and optionally substituted by 1-5 R² groups. In some embodiments, R^{a} and R^{b} are taken together with the nitrogen atom to which they are attached to form an 8-membered bridged bicyclic heterocyclyl optionally containing 1-2 additional heteroatoms selected from N and O and optionally substituted by 1-5 R² groups. In some embodiments, the bicyclic heterocyclyl contains one oxygen atom. In some embodiments, the bicyclic heterocyclyl contains one additional nitrogen atom. In some embodiments, the bicyclic heterocyclyl contains two additional nitrogen atoms. In some embodiments, the bicyclic heterocyclyl contains one additional nitrogen atom and one oxygen atom. In some embodiments, the bicyclic heterocyclyl is partially unsaturated. In some embodiments, the bicyclic heterocyclyl is fully saturated. In some embodiments, the bicyclic heterocyclyl is a saturated heterocyclic ring fused to a partially unsaturated ring. In some embodiments, the bicyclic heterocyclyl is a saturated heterocyclic ring fused to an aromatic ring. In some embodiments, the bicyclic heterocyclyl is a saturated heterocyclic ring fused to a heteroaryl ring.

In some embodiments, each R² is independently —NH₂, halo, C₁-C₆ alkyl, C₁-C₆ haloalkyl, —CN, or 5- to 6-membered heteroaryl, wherein the heteroaryl contains 1-2 nitrogen atoms and is optionally substituted by 1-5 groups selected from C₁-C₆ alkyl, halo, and C₁-C₆ haloalkyl. In some embodiments, each R² is independently —NH₂, halo, C₁-C₃ alkyl, C₁-C₃ haloalkyl, —CN, or 5-membered heteroaryl, wherein the heteroaryl contains 1-2 nitrogen atoms and is optionally substituted by 1-3 groups selected from C₁-C₃ alkyl, halo, and C₁-C₃ haloalkyl. In some embodiments, each R² is independently —NH₂, F, Cl, —CH₃, or —CF₃, —CN, or pyrazolyl optionally substituted by 1-2 groups selected from —CH₃ and Cl.

In some embodiments, R² is —NH₂.

In some embodiments, R² is halo. In some embodiments, R² is Cl, F, or Br. In some embodiments, R² is Cl. In some embodiments, R² is F. In some embodiments, R² is Br.

In some embodiments, R² is C₁-C₆ alkyl. In some embodiments, R² is C₁-C₃ alkyl. In some embodiments, R² is methyl, ethyl, n-propyl, or isopropyl. In some embodiments, R² is methyl. In some embodiments, R² is ethyl. In some embodiments, R² is n-propyl. In some embodiments, R² is isopropyl.

In some embodiments, R² is C₁-C₆ haloalkyl. In some embodiments, R² is C₁-C₆ haloalkyl containing 1-13 halogen atoms. In some embodiments, R² is C₁-C₃ haloalkyl. In some embodiments, R² is C₁-C₃ haloalkyl containing 1-7 halogen atoms. In some embodiments, R² is —CF₃, —CHF₂, —CH₂F, —CCl₃, —CHCl₂, —CH₂Cl, —CF₂Cl, —CFCl₂, —CH₂CF₃, —CH₂CHF₂, or —CH₂CCl₃. In some embodiments, R² is —CF₃. In some embodiments, R² is —CHF₂.

In some embodiments, R² is —CN.

In some embodiments, R² is 5- to 6-membered heteroaryl containing 1-2 nitrogen atoms and optionally substituted by 1-5 groups selected from C₁-C₆ alkyl, halo, and C₁-C₆ haloalkyl. In some embodiments, R² is 5- to 6-membered heteroaryl containing 1 nitrogen atom and optionally substituted by 1-5 groups selected from C₁-C₆ alkyl, halo, and C₁-C₆ haloalkyl. In some embodiments, R² is 5- to 6-membered heteroaryl containing 2 nitrogen atoms and optionally substituted by 1-5 groups selected from C₁-C₆ alkyl, halo, and C₁-C₆ haloalkyl.

In some embodiments, R² is a 5-membered heteroaryl containing 1-2 nitrogen atoms and optionally substituted by 1-5 groups selected from C₁-C₆ alkyl, halo, and C₁-C₆ haloalkyl. In some embodiments, R² is a 6-membered heteroaryl containing 1-2 nitrogen atoms and optionally substituted by 1-5 groups selected from C₁-C₆ alkyl, halo, and C₁-C₆ haloalkyl. In any of these variations, the heteroaryl is optionally substituted by 1-5 groups selected from C₁-C₃ alkyl (such as methyl, ethyl, or propyl), halo (such as F or Cl), and C₁-C₃ haloalkyl (such as —CF₃ or —CHF₂). In some embodiments, the heteroaryl is pyrrolyl, pyrazolyl, imidazolyl, pyridinyl, pyrazinyl, pyrimidinyl, or pyridazinyl. In some embodiments, the heteroaryl is pyrazolyl.

In some embodiments,

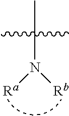

is:

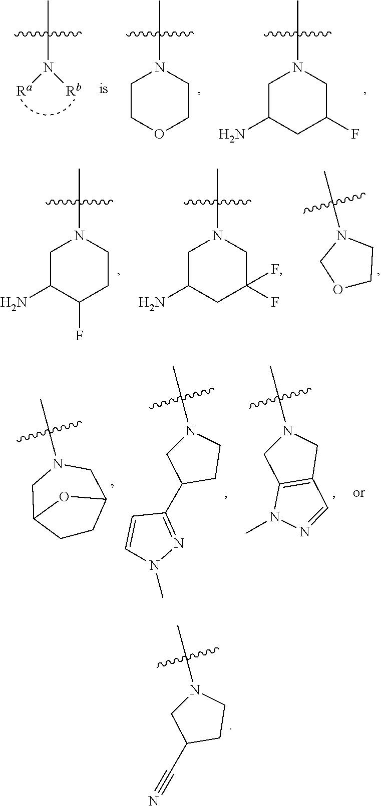

In some embodiments, L¹ is —C(O)N(H)—, —C(O)—, —(C₁-C₆ alkylene)N(R³)—, or C₁-C₆ alkylene. In some embodiments, L¹ is —C(O)N(H)—, —C(O)—, —(C₁-C₃ alkylene)N(R³)—, or C₁-C₃ alkylene; and R³ is H or C₁-C₃ alkyl. In some embodiments, L¹ is —C(O)N(H)—, —C(O)—, —CH₂N(H)—, —CH₂N(CH₃)—, —CH₂CH₂N(CH₃)—, —CH₂—, or —CH₂CH₂—.

In some embodiments, L¹ is —C(O)N(H)—.

In some embodiments, L¹ is —C(O)—.

In some embodiments, L¹ is —(C₁-C₆ alkylene)N(R³)—. In some embodiments, L¹ is —(C₁-C₃ alkylene)N(R³)—. In some embodiments, L¹ is —CH₂N(R³)—. In some embodiments, L¹ is —CH₂CH₂N(R³)—. In some embodiments, L¹ is —CH₂CH₂CH₂N(R³)—. In any of these variations, R³ is H or C₁-C₆ alkyl. In some embodiments, R³ is H. In some embodiments, R³ is C₁-C₆ alkyl. In some embodiments, R³ is C₁-C₃ alkyl. In some embodiments, R³ is methyl, ethyl, n-propyl, or isopropyl. In some embodiments, R³ is methyl. In some embodiments, R³ is ethyl. In some embodiments, R³ is n-propyl. In some embodiments, R³ is isopropyl. In some embodiments, L¹ is —(C₁-C₆ alkylene)N(H)—. In some embodiments, L¹ is —(C₁-C₆ alkylene)N(1-C₆ alkyl)-. In some embodiments, L¹ is —CH₂N(H)—, —CH₂CH₂N(H)—, or —CH₂CH₂CH₂N(H)—. In some embodiments, L¹ is —CH₂N(H)—. In some embodiments, L¹ is —CH₂CH₂N(H)—. In some embodiments, L¹ is —CH₂N(CH₃)—, —CH₂CH₂N(CH₃)—, or —CH₂CH₂CH₂N(CH₃)—. In some embodiments, L¹ is —CH₂N(CH₃)—. In some embodiments, L¹ is —CH₂CH₂N(CH₃)—.

In some embodiments, L¹ is C₁-C₆ alkylene. In some embodiments, L¹ is C₁-C₃ alkylene. In some embodiments, L¹ is —CH₂—, —CH₂CH₂—, or —CH₂CH₂CH₂—. In some embodiments, L¹ is —CH₂—. In some embodiments, L¹ is —CH₂CH₂—.

In some embodiments, L² is a bond, —C(O)—, —N(R³)—, or 0.

In some embodiments, L² is a bond. In some embodiments, L² is —C(O)—. In some embodiments, L² is O.

In some embodiments, L² is —N(R³)—. In some variations, R³ is H or C₁-C₆ alkyl. In some embodiments, R³ is H. In some embodiments, R³ is C₁-C₆ alkyl. In some embodiments, R³ is C₁-C₃ alkyl. In some embodiments, R³ is methyl, ethyl, n-propyl, or isopropyl. In some embodiments, R³ is methyl. In some embodiments, R³ is ethyl. In some embodiments, R³ is n-propyl. In some embodiments, R³ is isopropyl. In some embodiments, L² is —N(H)—. In some embodiments, L² is —N(CH₃)—. In some embodiments, L² is —N(CH₂CH₃)—.

In some embodiments, Ring A is a monocyclic 4- to 6-membered heterocyclylene or a bicyclic 6- to 9-membered spiro heterocyclylene, wherein the heterocyclylene contains 1-2 nitrogen atoms, and wherein the heterocyclylene is substituted by m R⁴ groups. In some embodiments, Ring A is a monocyclic 4- to 6-membered heterocyclylene containing 1-2 nitrogen atoms and substituted by m R⁴ groups. In some embodiments, Ring A is a bicyclic 6- to 9-membered spiro heterocyclylene containing 1-2 nitrogen atoms and substituted by m R⁴ groups.

In some embodiments, Ring A is a monocyclic 4- to 6-membered heterocyclylene containing 1-2 nitrogen atoms and substituted by m R⁴ groups. In some embodiments, Ring A is a monocyclic 4-membered heterocyclylene containing 1-2 nitrogen atoms and substituted by m R⁴ groups. In some embodiments, Ring A is a monocyclic 5-membered heterocyclylene containing 1-2 nitrogen atoms and substituted by m R⁴ groups. In some embodiments, Ring A is a monocyclic 6-membered heterocyclylene containing 1-2 nitrogen atoms and substituted by m R⁴ groups. In some embodiments, the monocyclic heterocyclylene contains 1 nitrogen atom. In some embodiments, the monocyclic heterocyclylene contains 2 nitrogen atoms. In some embodiments, the monocyclic heterocyclylene is azetidinylene, pyrrolidinylene, piperidinylene, or piperazinylene.

In some embodiments, Ring A is

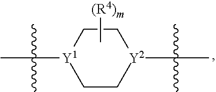

wherein Y¹ and Y² are independently CH or N, provided that at least one of Y¹ and Y² is N. In some embodiments, Y¹ is CH and Y² is N. In some embodiments, Y² is CH and Y¹ is N. In some embodiments, Y¹ and Y² are each N.

In some embodiments, Ring A is

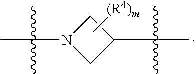

In some embodiments, Ring A is a bicyclic 6-membered spiro heterocyclylene containing 1-2 nitrogen atoms and substituted by m R⁴ groups. In some embodiments, Ring A is a bicyclic 7-membered spiro heterocyclylene containing 1-2 nitrogen atoms and substituted by m R⁴ groups. In some embodiments, Ring A is a bicyclic 8-membered spiro heterocyclylene containing 1-2 nitrogen atoms and substituted by m R⁴ groups. In some embodiments, Ring A is a bicyclic 9-membered spiro heterocyclylene containing 1-2 nitrogen atoms and substituted by m R⁴ groups. In some embodiments, the bicyclic spiro heterocyclylene contains 1 nitrogen atom. In some embodiments, the bicyclic spiro heterocyclylene contains 2 nitrogen atoms.

In some embodiments, Ring A is

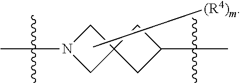

It is understood that either one of the ring systems or both ring systems of the spiro heterocyclylene can be substituted by a total of m R⁴ groups.

In some embodiments, each R⁴ is independently halo, C₁-C₆ alkyl, or C₁-C₆ haloalkyl. In some embodiments, each R⁴ is independently halo, C₁-C₃ alkyl, or C₁-C₃ haloalkyl. In some embodiments, each R⁴ is independently F, Cl, —CH₃, or —CF₃. In some embodiments, each R⁴ is independently F or —CH₃.

In some embodiments, R⁴ is halo. In some embodiments, R⁴ is Cl, F, or Br. In some embodiments, R⁴ is Cl. In some embodiments, R⁴ is F. In some embodiments, R⁴ is Br.

In some embodiments, R⁴ is C₁-C₆ alkyl. In some embodiments, R⁴ is C₁-C₃ alkyl. In some embodiments, R⁴ is methyl, ethyl, n-propyl, or isopropyl. In some embodiments, R⁴ is methyl. In some embodiments, R⁴ is ethyl. In some embodiments, R⁴ is n-propyl. In some embodiments, R⁴ is isopropyl.

In some embodiments, R⁴ is C₁-C₆ haloalkyl. In some embodiments, R⁴ is C₁-C₆ haloalkyl containing 1-13 halogen atoms. In some embodiments, R⁴ is C₁-C₃ haloalkyl. In some embodiments, R⁴ is C₁-C₃ haloalkyl containing 1-7 halogen atoms. In some embodiments, R⁴ is —CF₃, —CHF₂, —CH₂F, —CCl₃, —CHCl₂, —CH₂Cl, —CF₂Cl, —CFCl₂, —CH₂CF₃, —CH₂CHF₂, or —CH₂CCl₃. In some embodiments, R⁴ is —CF₃. In some embodiments, R⁴ is —CHF₂.

In some embodiments, m is 0-5. In some embodiments, m is 0. In some embodiments, m is 1 or 2. In some embodiments, m is 1. In some embodiments, m is 2. In some embodiments, m is 3. In some embodiments, m is 4. In some embodiments, m is 5.

In some embodiments, Ring A is:

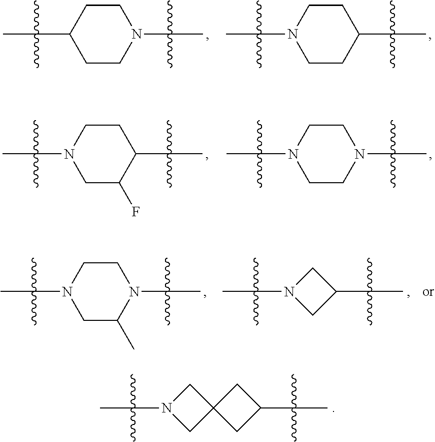

In some embodiments, R⁵ is H or C₁-C₆ alkyl. In some embodiments, R⁵ is H or C₁-C₃ alkyl. In some embodiments, R⁵ is H or —CH₃. In some embodiments, R⁵ is —CH₃.

In some embodiments, R⁵ is H.

In some embodiments, R⁵ is C₁-C₆ alkyl. In some embodiments, R⁵ is C₁-C₃ alkyl. In some embodiments, R⁵ is methyl, ethyl, n-propyl, or isopropyl. In some embodiments, R⁵ is methyl. In some embodiments, R⁵ is ethyl. In some embodiments, R⁵ is n-propyl. In some embodiments, R⁵ is isopropyl.

In some embodiments, Z is CH or N. In some embodiments, Z is CH. In some embodiments, Z is N.

In some embodiments,

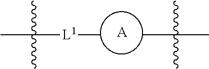

is:

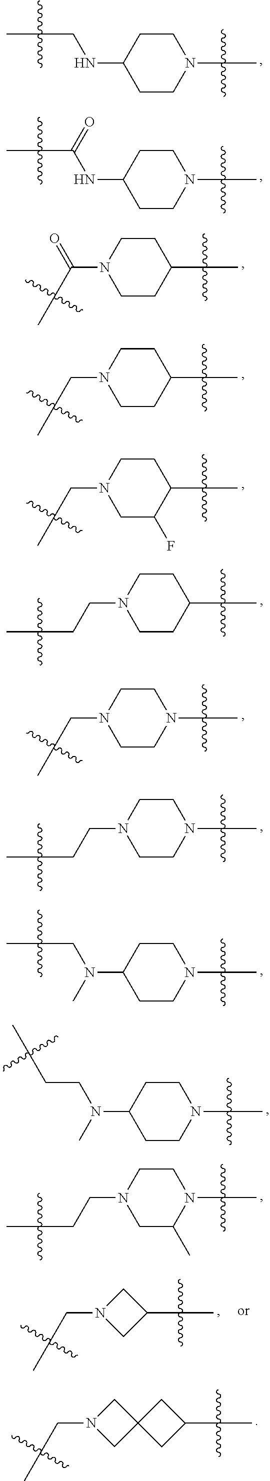

In some embodiments,

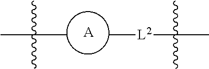

is:

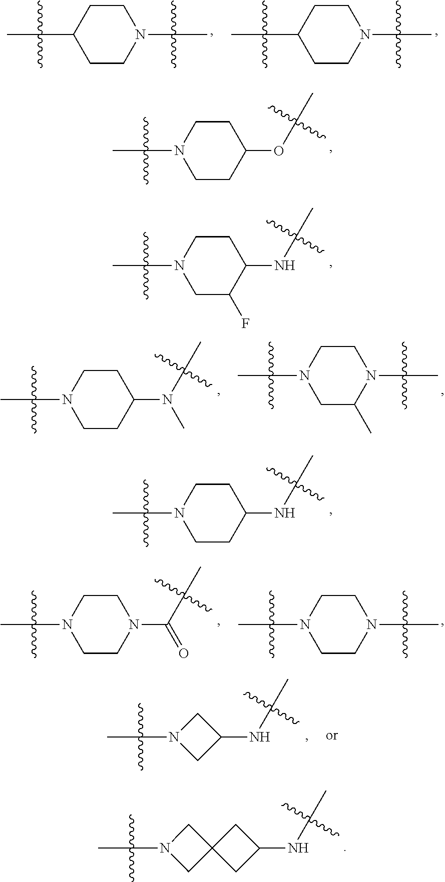

In some embodiments,

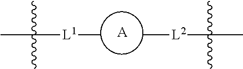

is:

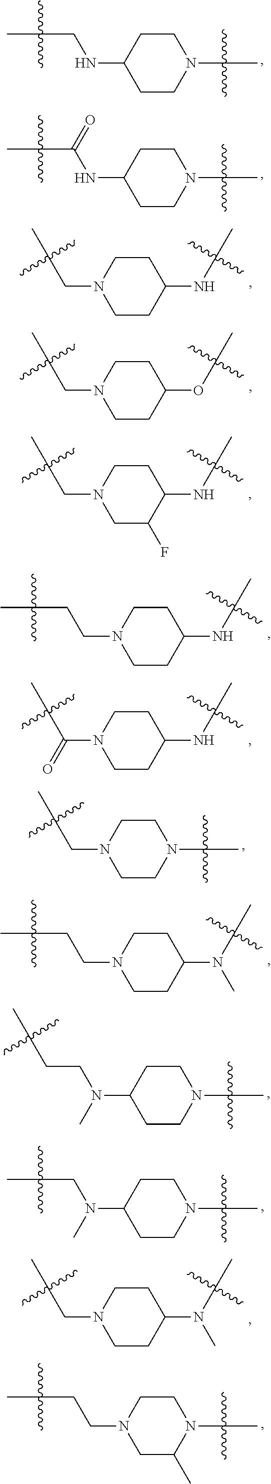

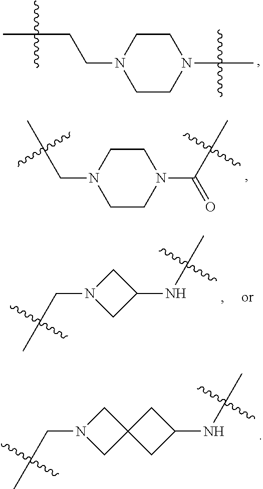 [SMILES: *CCN1CCN(*)CC1 ; *CN1CCN(C(*)=O)CC1 ; *CN1CC(N*)C1 ; *CN1CC2(CC(N*)C2)C1]

In some embodiments, the compound of Formula (I) is a compound of Formula (IA):

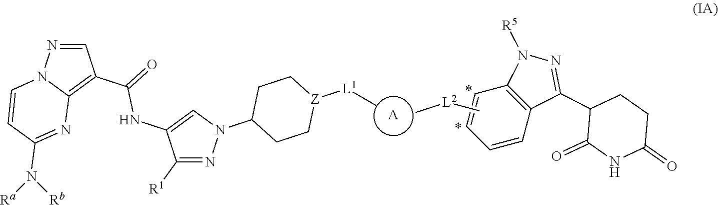

wherein:
R^{a} is H or C₁-C₆ alkyl; R^{b} is C₁-C₆ alkyl, 5- to 6-membered heteroaryl, —(C₁-C₆ alkylene)(5- to 6-membered heteroaryl), or —(C₁-C₆ alkylene)NH₂, wherein the heteroaryl contains 1-2 nitrogen atoms and is optionally substituted by 1-5 R² groups; and R¹, R⁵, Z, L¹, L², Ring A, and * are as described for Formula (I).

In some embodiments, the compound of Formula (I) is a compound of Formula (IB):

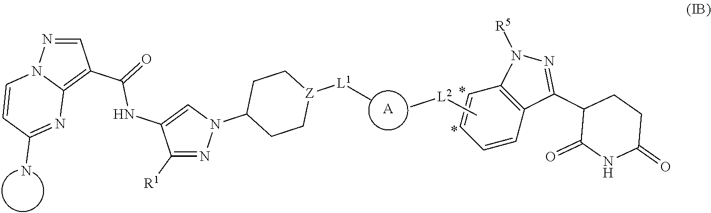

wherein

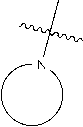 [SMILES: *N]

is a 5- to 10-membered monocyclic or bicyclic heterocyclyl optionally containing 1-2 additional heteroatoms selected from N and O, and is optionally substituted by 1-5 R² groups; and R¹, R⁵, Z, L¹, L², Ring A, and * are as described for Formula (I).

In some embodiments, the compound of Formula (I) is a compound of Formula (IIa) or (IIb):

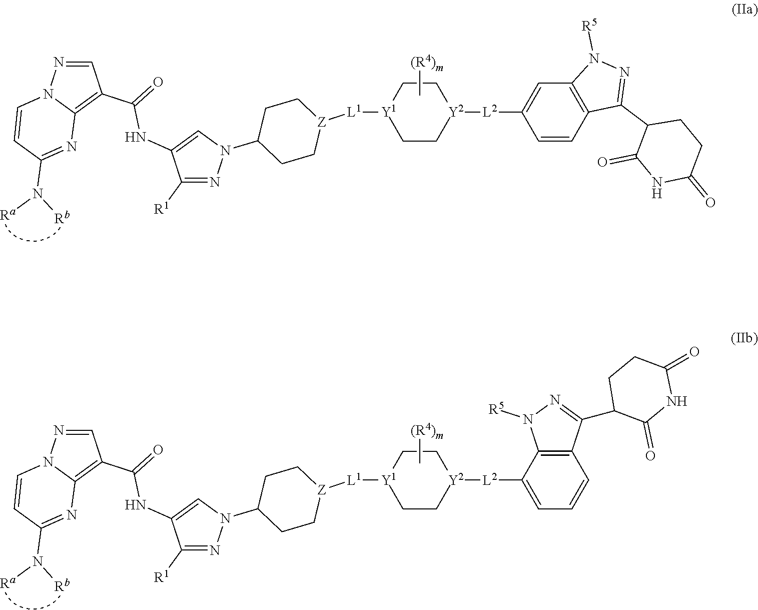

wherein R^{a}, R^{b}, R¹, R⁴, R⁵, m, Z, L¹, L², Y¹, and Y² are as described for Formula (I).

In some embodiments, the compound of Formula (I) is a compound of Formula (IIIa) or (IVa).

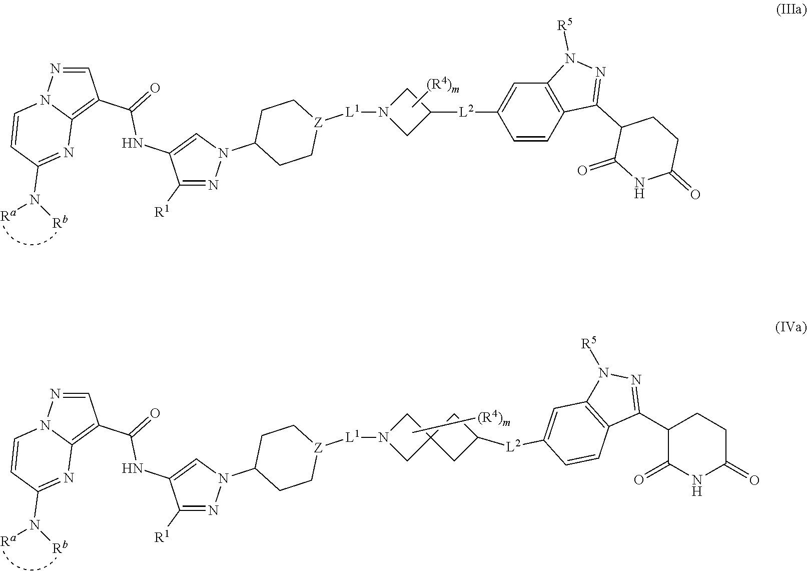

wherein R^{a}, R^{b}, R¹, R⁴, R⁵, m, Z, L¹, and L² are as described for Formula (I).

In some embodiments, the compound of Formula (I) is a compound of Formula (Va), (Vb), (Vc), or (Vd):

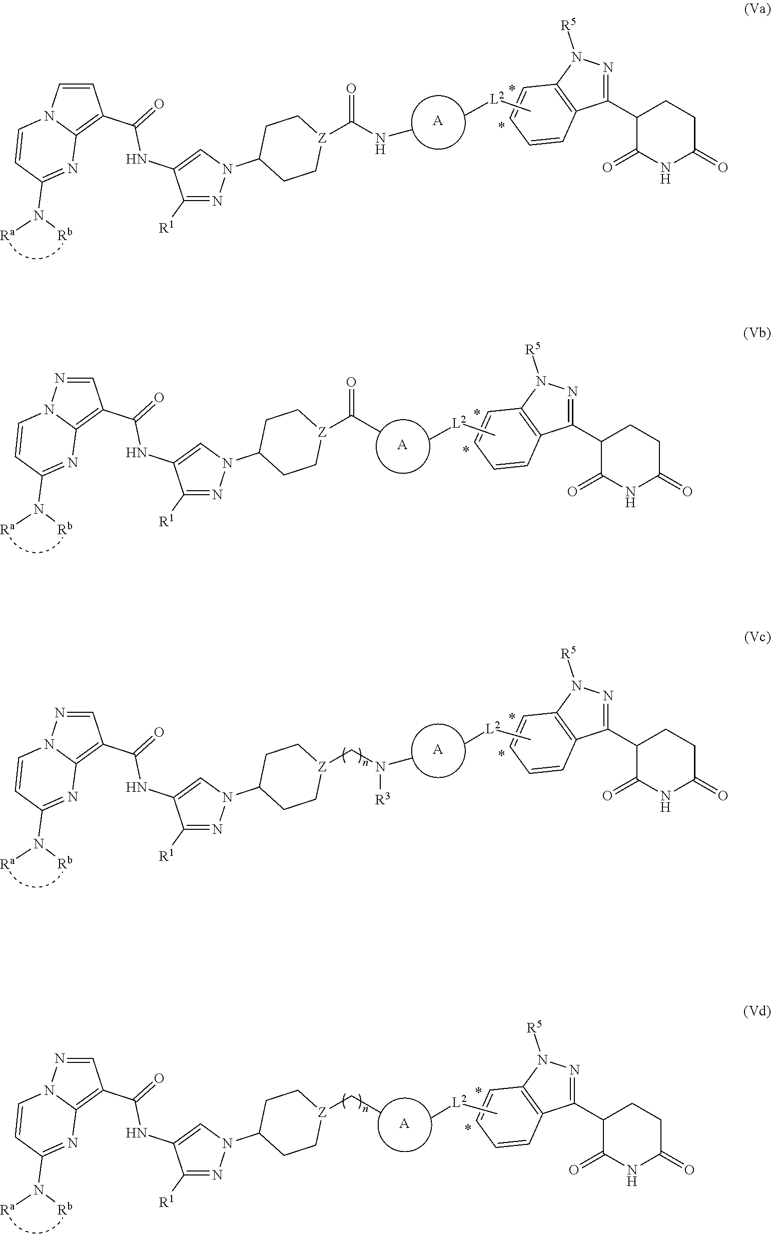

wherein Ring A, R^{a}, R^{b}, R¹, R³, R⁵, Z, L², and * are as described for Formula (I); and wherein n is an integer 1-6.

In some embodiments, the compound of Formula (I) is a compound of Formula (VIa), (VIb), (VIc), or (VId):

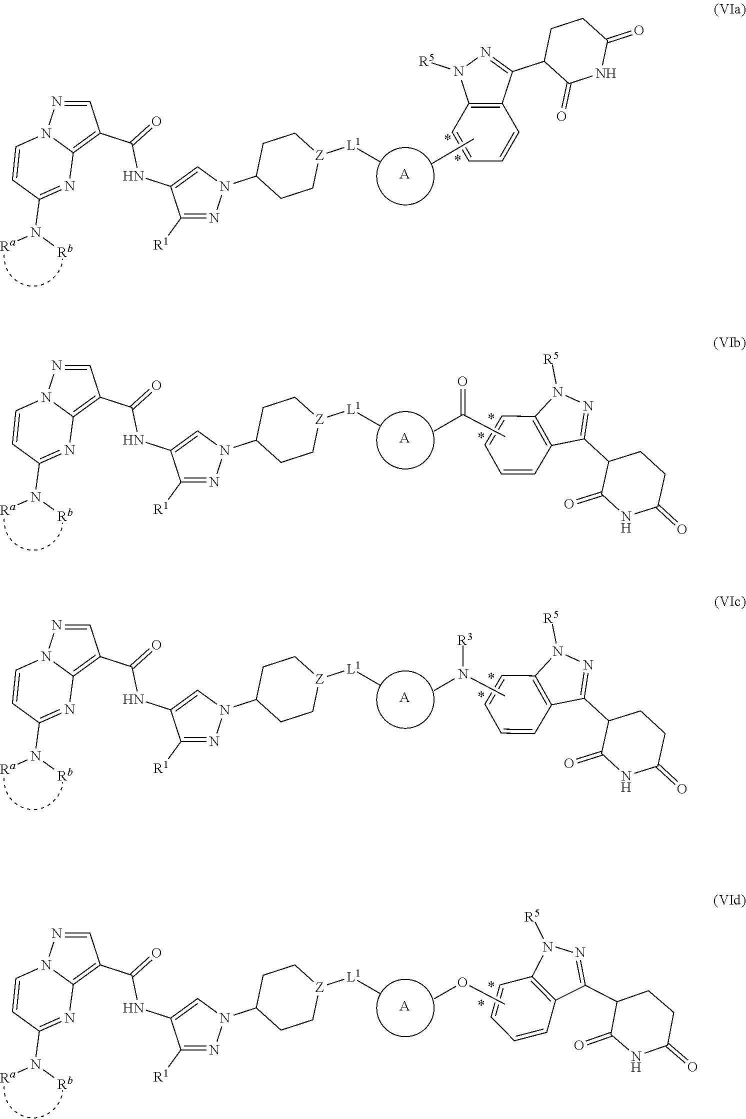

wherein Ring A, R^{a}, R^{b}, R¹, R³, R⁵, Z, L¹, and * are as described for Formula (I).

In some embodiments, the compound of Formula (I) is a compound of Formula (VIa), (VIb), (VIc), or (VId):

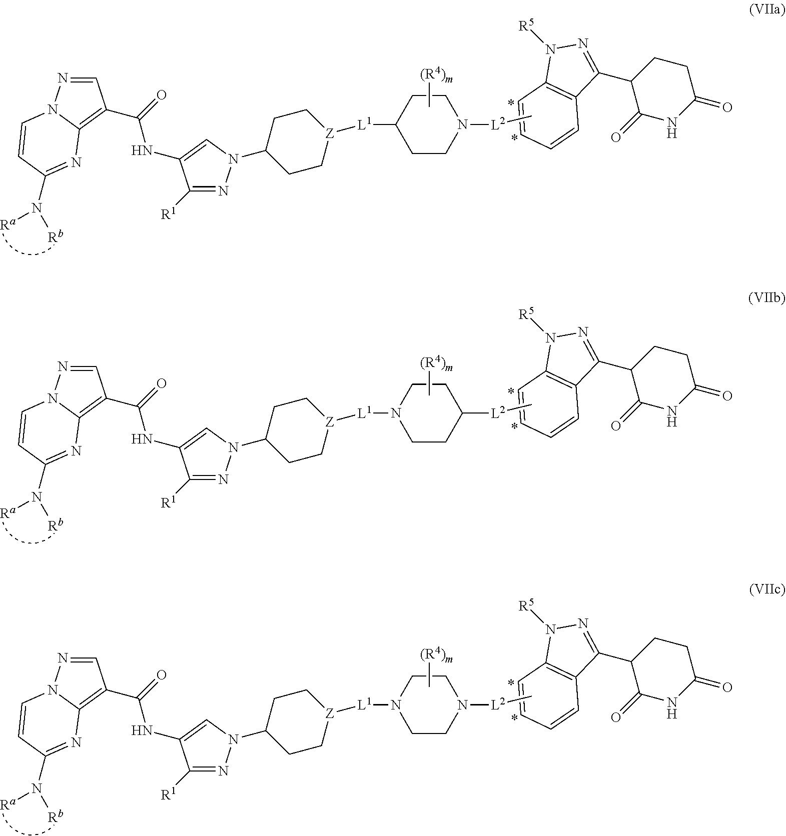

wherein R^{a}, R^{b}, R¹, R⁴, R⁵, Z, L, L², and * are as described for Formula (I).

In the descriptions herein, it is understood that every description, variation, embodiment, or aspect of a moiety may be combined with every description, variation, embodiment, or aspect of other moieties the same as if each and every combination of descriptions is specifically and individually listed. For example, every description, variation, embodiment, or aspect provided herein with respect to Ring A of Formula (I) may be combined with every description, variation, embodiment, or aspect of R¹, R^{a}, R^{b}, R², L¹, L², R³, R⁴, m, R⁵, and Z the same as if each and every combination were specifically and individually listed. It is also understood that all descriptions, variations, embodiments, or aspects of Formula (I), where applicable, apply equally to other formulae detailed herein, and are equally described, the same as if each and every description, variation, embodiment, or aspect were separately and individually listed for all formulae. For example, all descriptions, variations, embodiments, or aspects of Formula (I), where applicable, apply equally to any of the formulae as detailed herein, such as Formulae (IA), (IB), (IIa), (IIb), (IIIa), (IVa), (Va), (Vb), (Vc), (Vd), (VIa), (VIb), (VIc), (VId), (VIIa), (VIIb), and (VIIc) are equally described, the same as if each and every description, variation, embodiment, or aspect were separately and individually listed for all formulae.

In some embodiments, provided is a compound selected from the compounds in Table 1 or a pharmaceutically acceptable salt thereof. Although certain compounds described in the present disclosure, including in Table 1, are presented as specific stereoisomers and/or in a non-stereochemical form, it is understood that any or all stereochemical forms, including any enantiomeric or diastereomeric forms, and any tautomers or other forms of any of the compounds of the present disclosure, including in Table 1, are herein described.

TABLE 1

| Compound | |
|---|---|
| No. | Structure |
| P-1 | 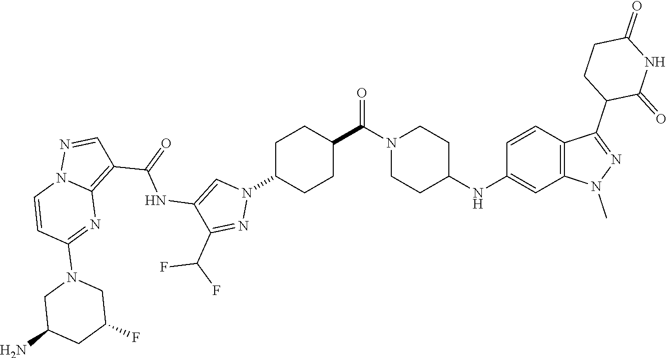 |
| P-2 | 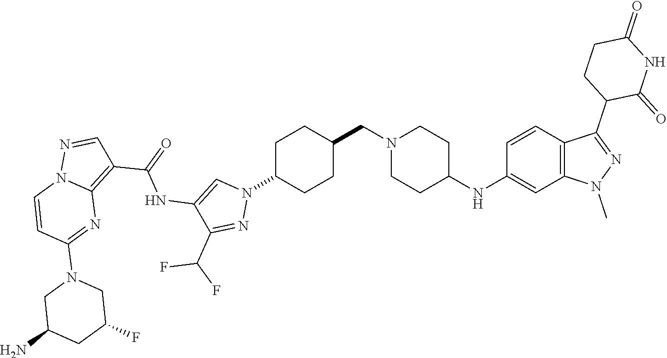 |
| P-3 | 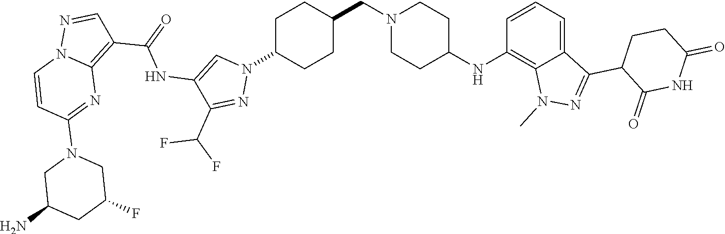 |
| P-4 | 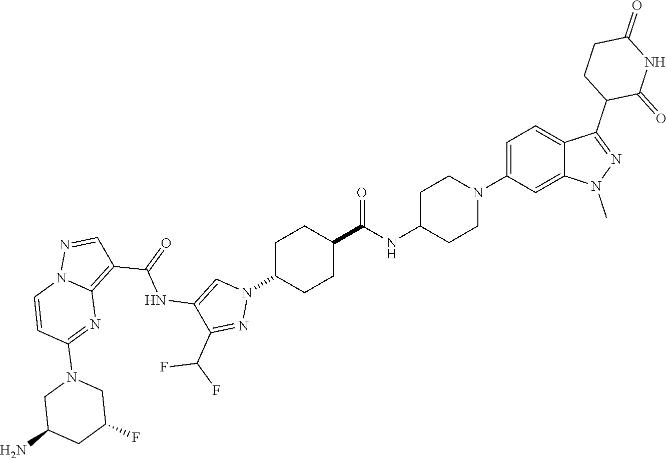 |
| P-5 | 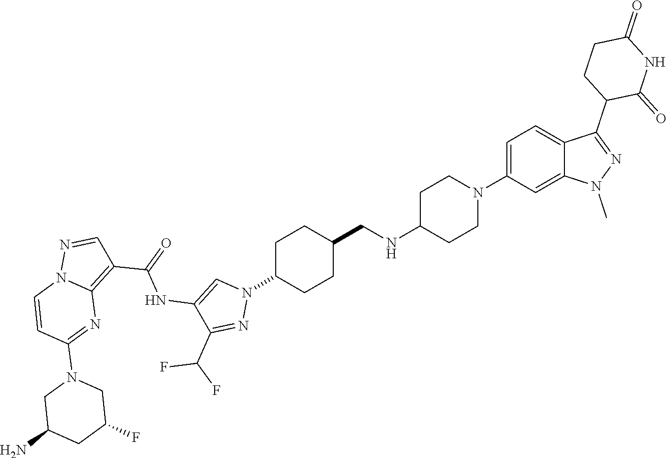 |
| P-6 | 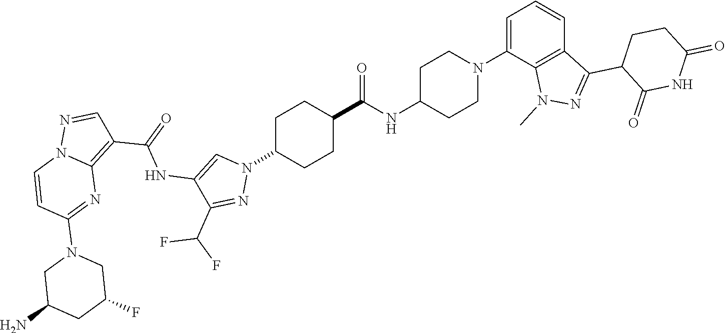 |
| P-7 | 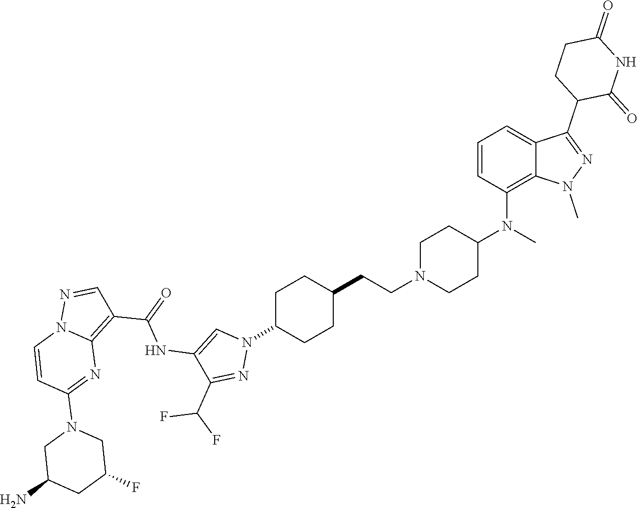 |
| P-8 | 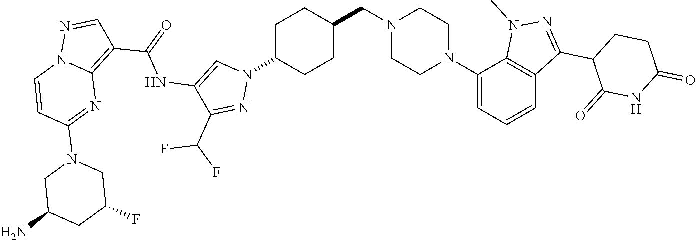 |
| P-9 | 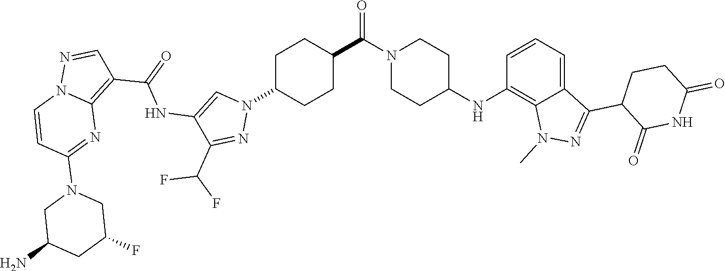 |
| P-10 | 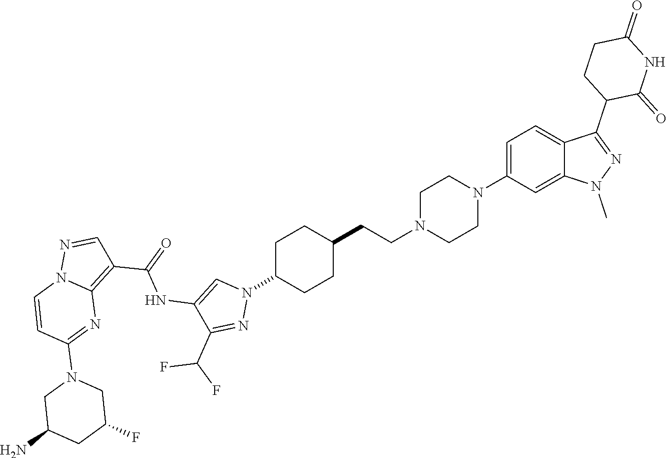 |
| P-11 | 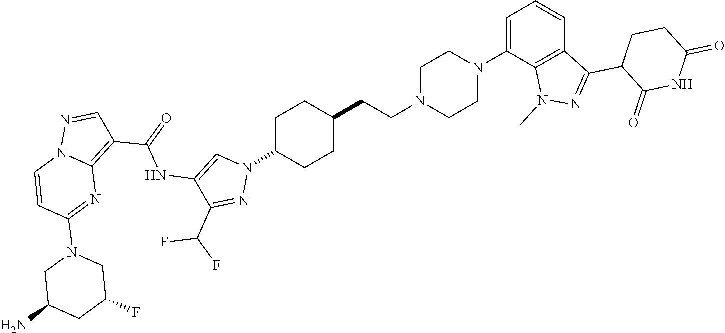 |
| P-12 | 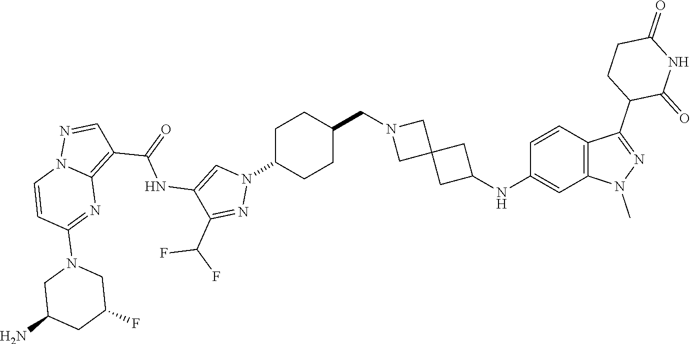 |
| P-13 | 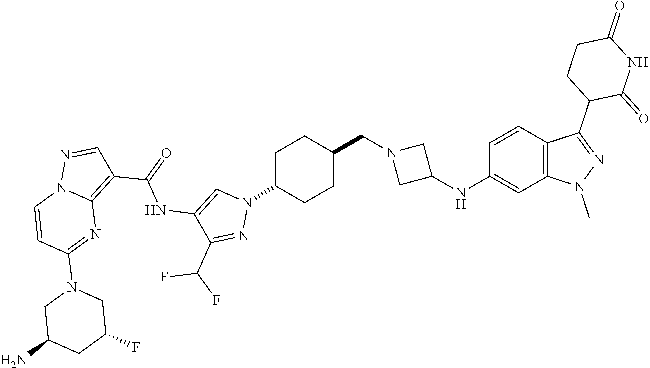 |
| P-14 | 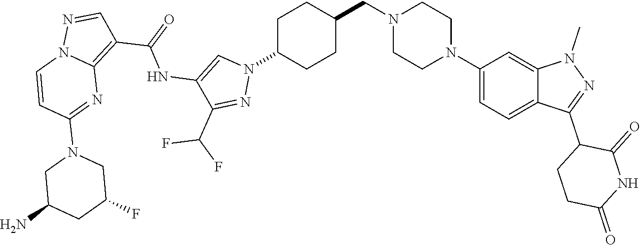 [SMILES: Cn1nc(C2CCC(=O)NC2=O)c2ccc(N3CCN(C[C@H]4CC[C@H](n5cc(NC(=O)c6cnn7ccc(N8C[C@H](N)C[C@@H](F)C8)nc67)c(C(F)F)n5)CC4)CC3)cc21] |
| P-15 | 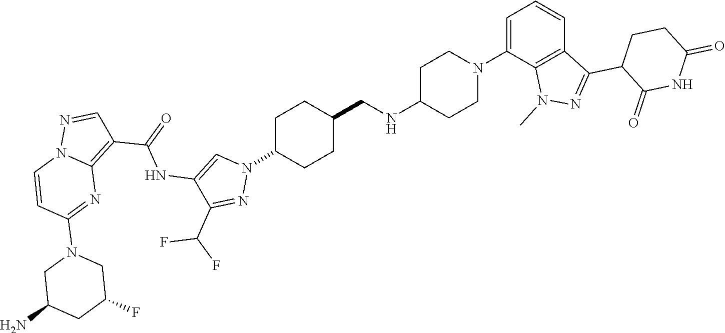 |
| P-16 | 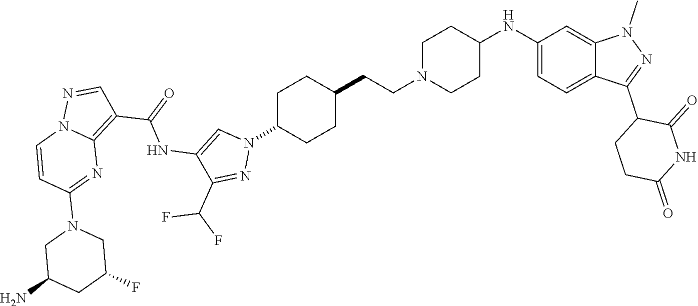 [SMILES: Cn1nc(C2CCC(=O)NC2=O)c2ccc(NC3CCN(CC[C@H]4CC[C@H](n5cc(NC(=O)c6cnn7ccc(N8C[C@H](N)C[C@@H](F)C8)nc67)c(C(F)F)n5)CC4)CC3)cc21] |
| P-17 | 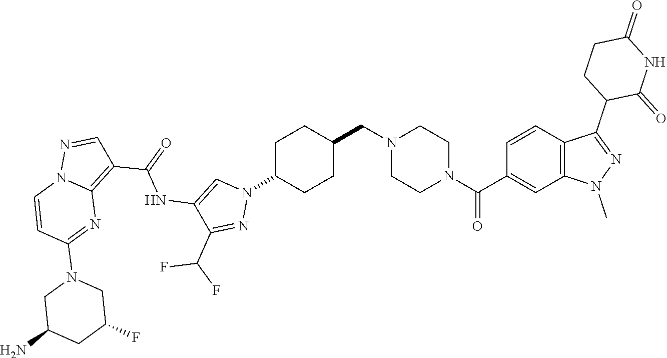 |
| P-18 | 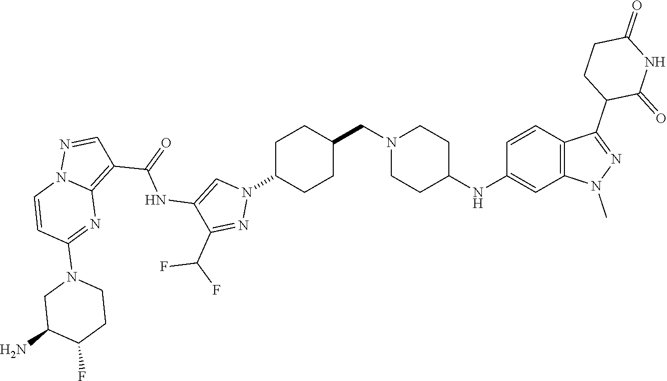 |
| P-19 | 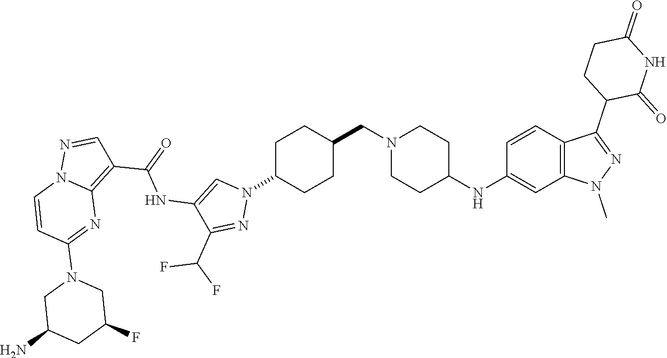 |
| P-20 | 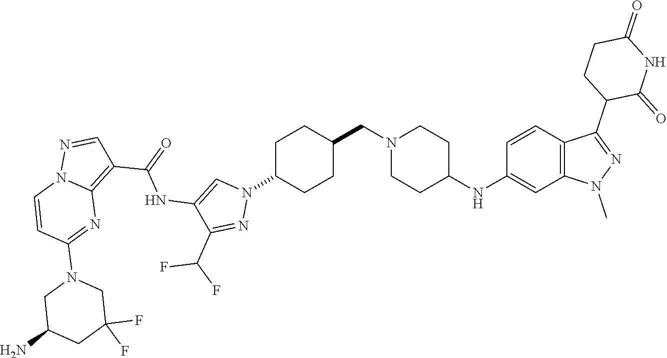 |
| P-21 | 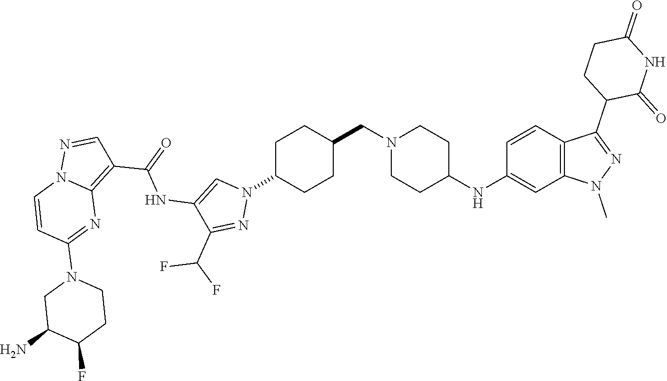 |
| P-22 | 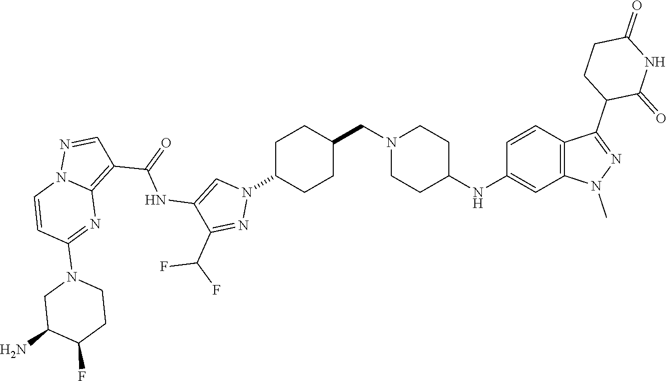 |
| P-23 | 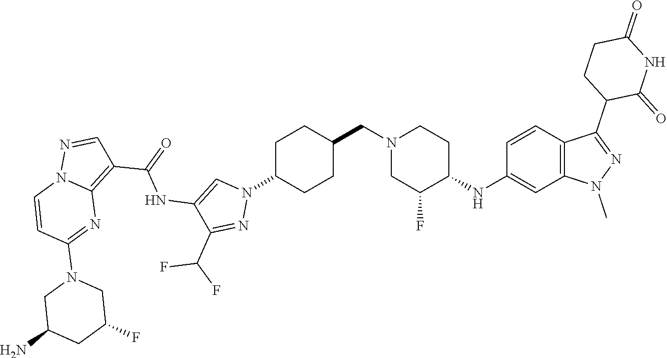 |
| P-24 | 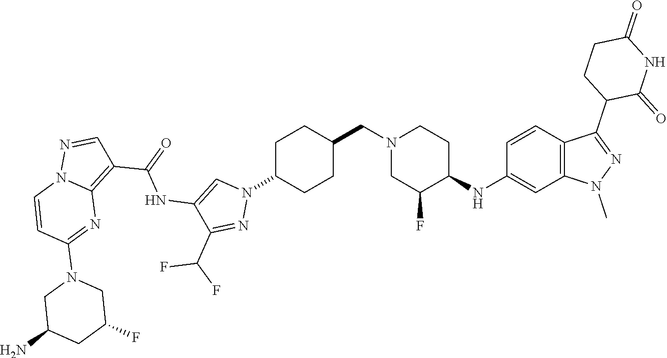 |
| P-25 | 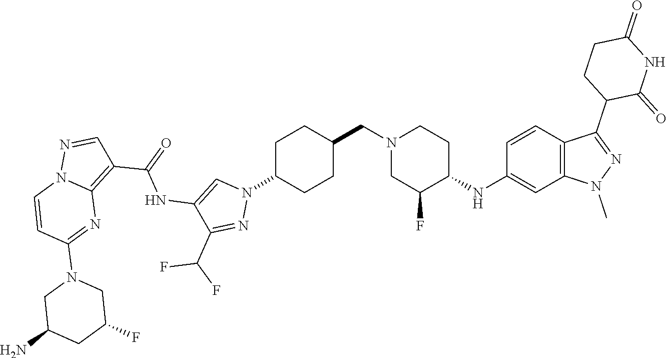 |
| P-26 | 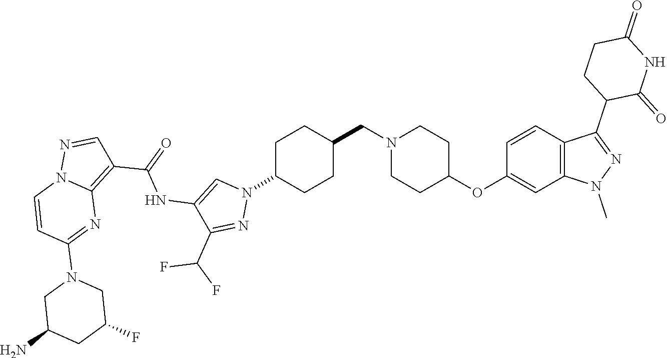 |
| P-27 | 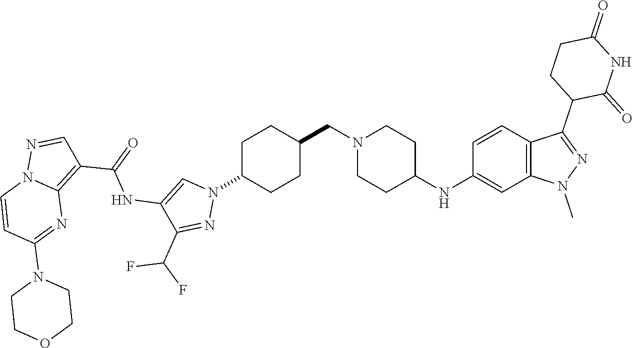 |
| P-28 | 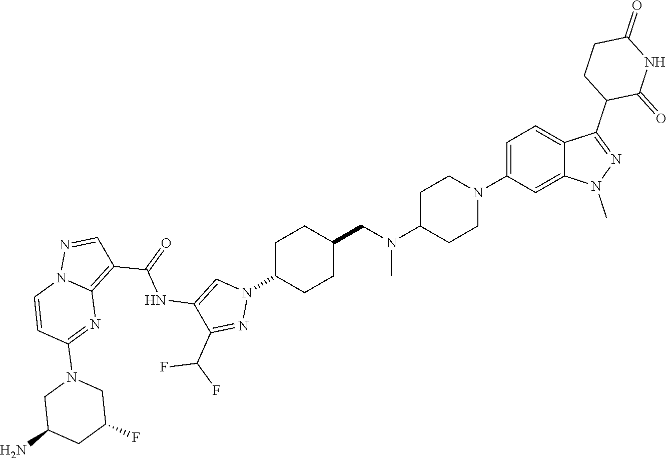 |
| P-29 | 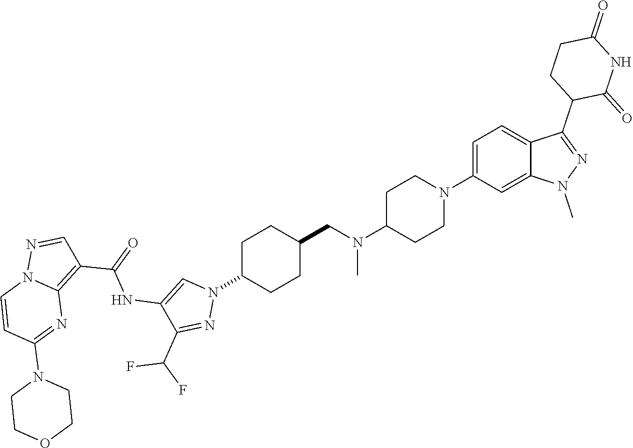 |
| P-30 | 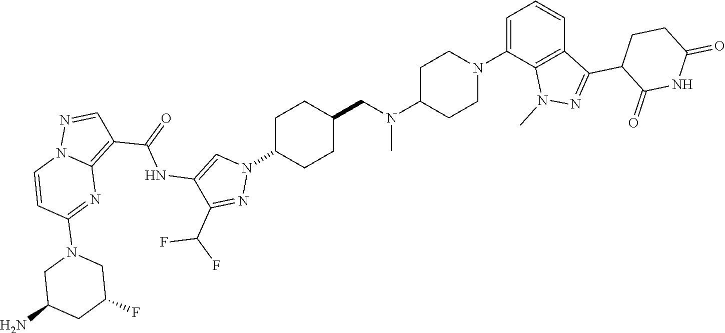 |
| P-31 | 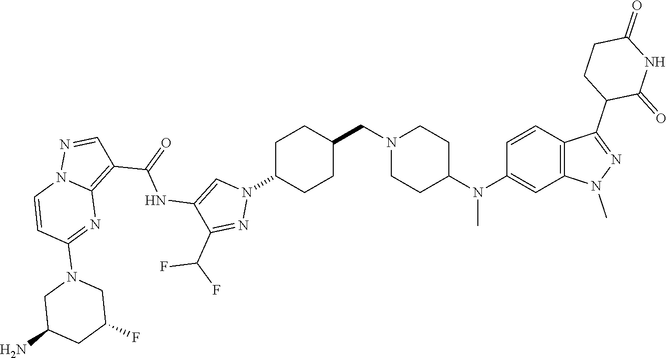 |
| P-32 | 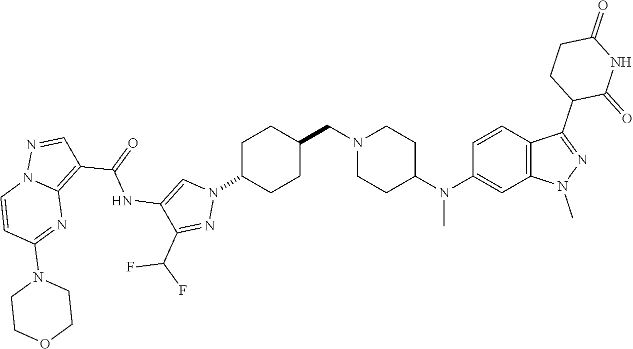 |
| P-33 | 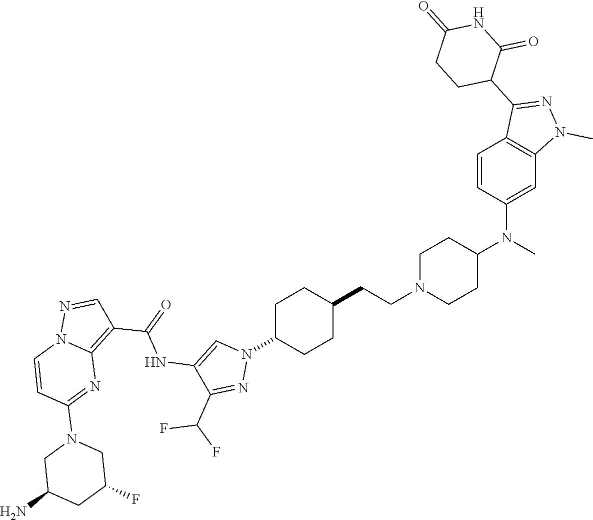 |
| P-34 | 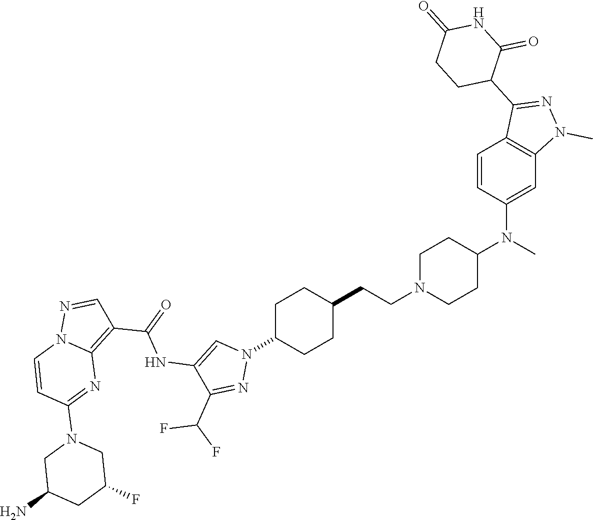 |
| P-35 | 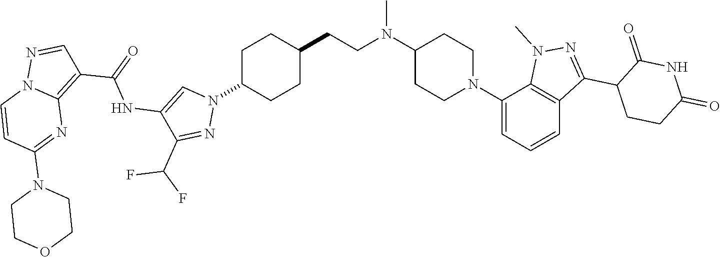 |
| P-36 | 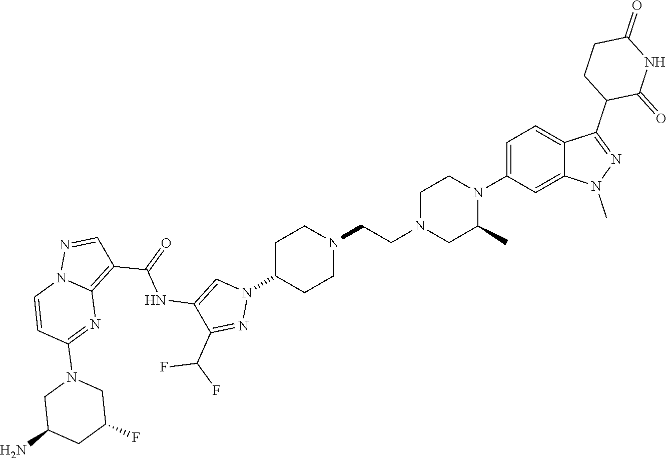 |
| P-37 | 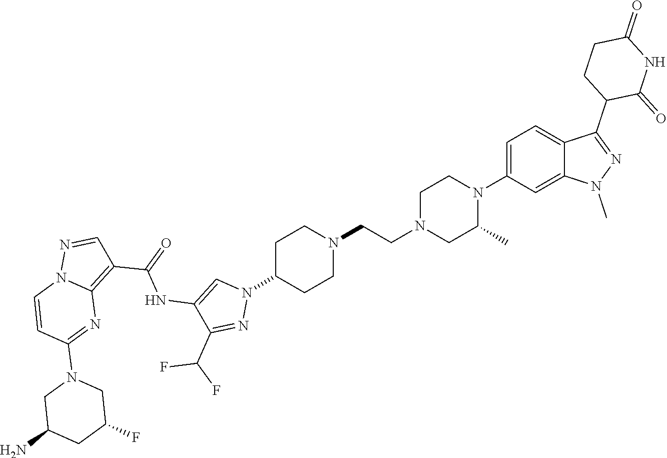 |
| P-38 | 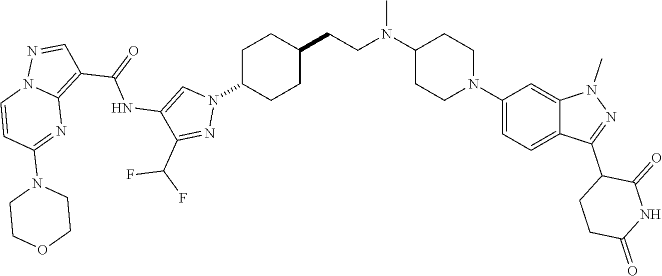 [SMILES: CN(CC[C@H]1CC[C@H](n2cc(NC(=O)c3cnn4ccc(N5CCOCC5)nc34)c(C(F)F)n2)CC1)C1CCN(c2ccc3c(C4CCC(=O)NC4=O)nn(C)c3c2)CC1] |
| P-39 | 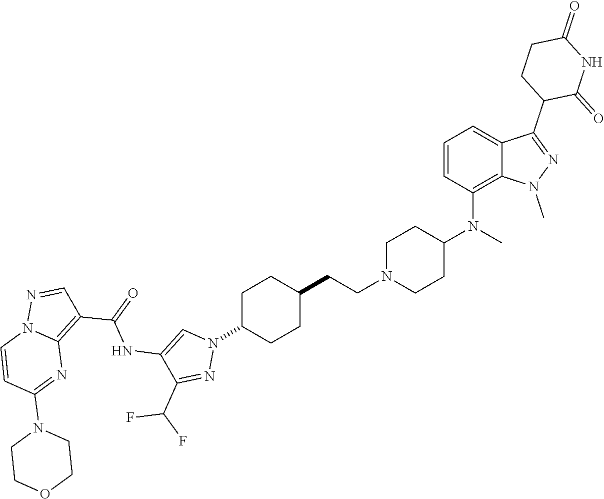 |
| P-40 | 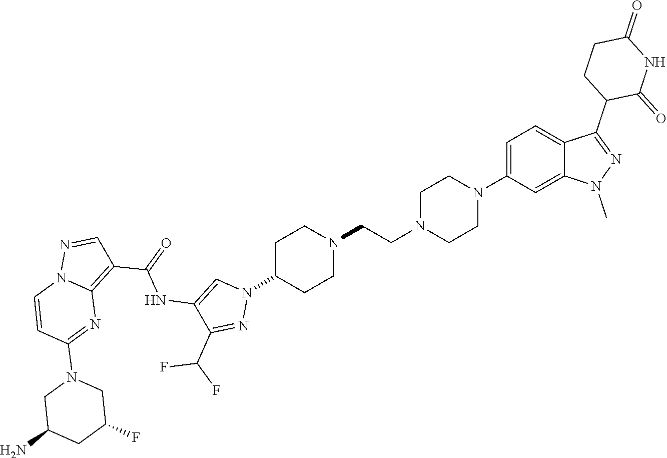 |
| P-41 | 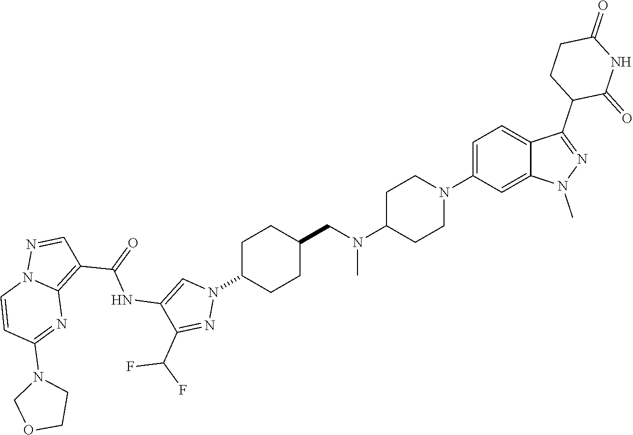 |
| P-42 | 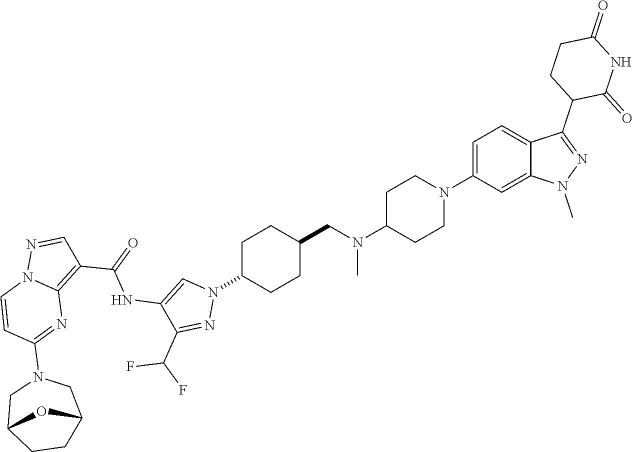 |
| P-43 | 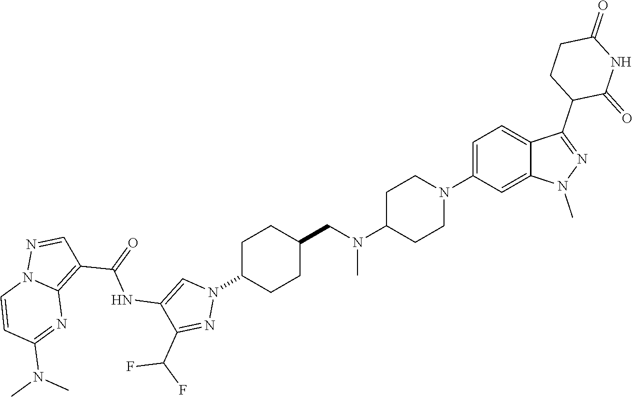 |
| P-44 | 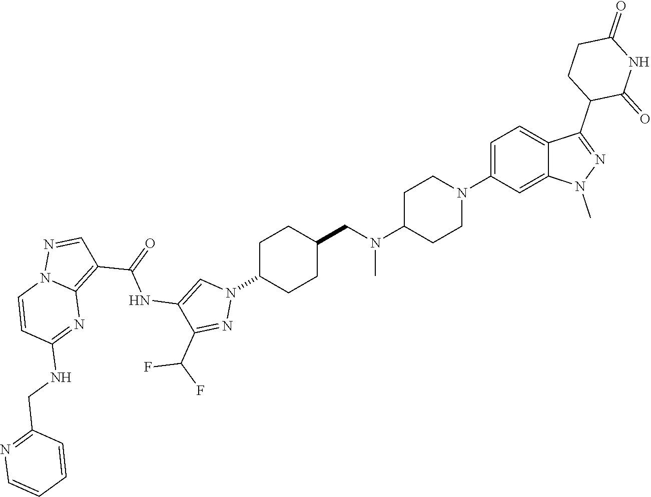 |
| P-45 | 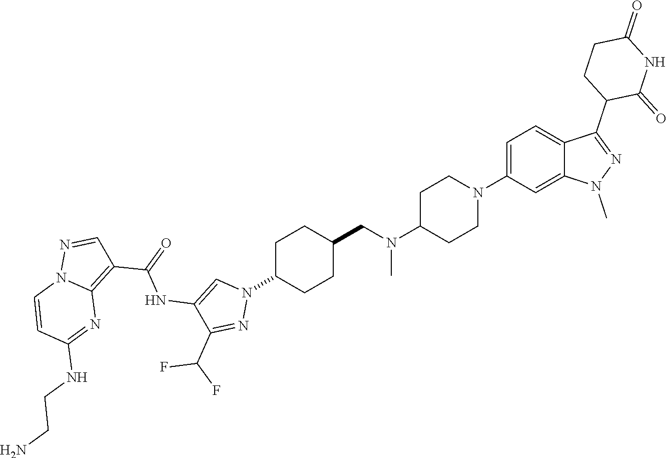 |
| P-46 | 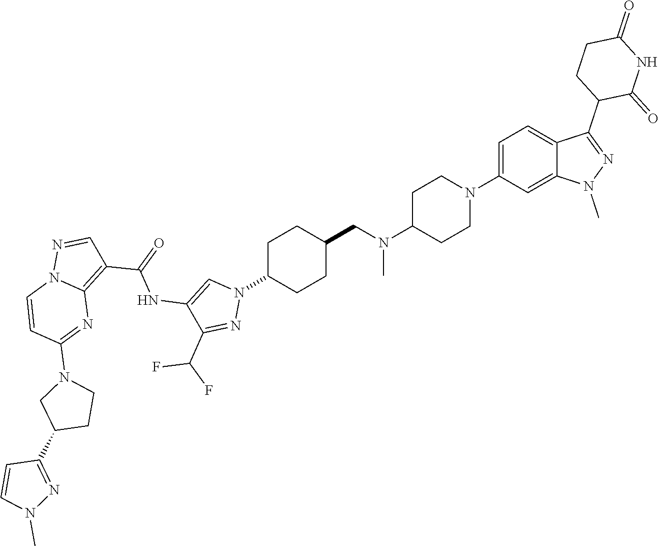 |
| P-47 | 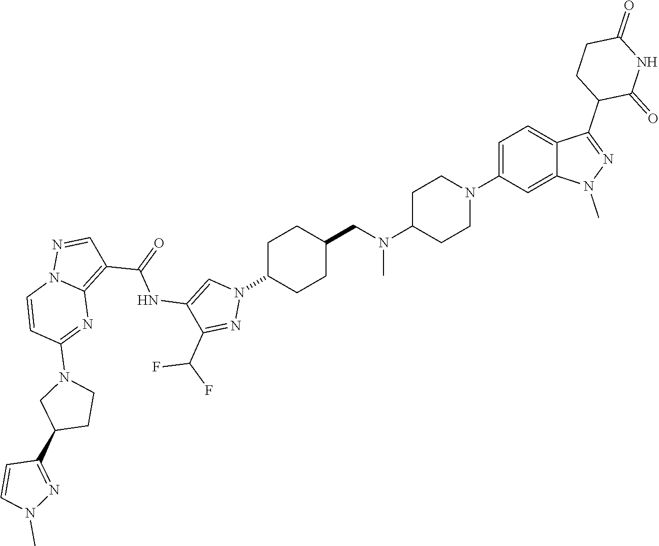 |
| P-48 | 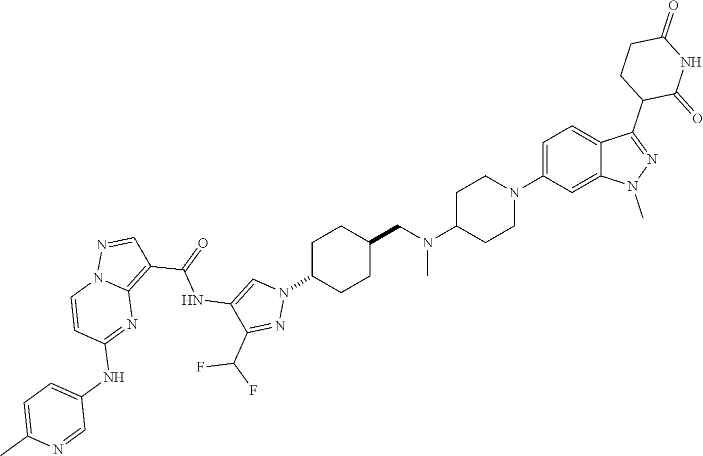 |
| P-49 | 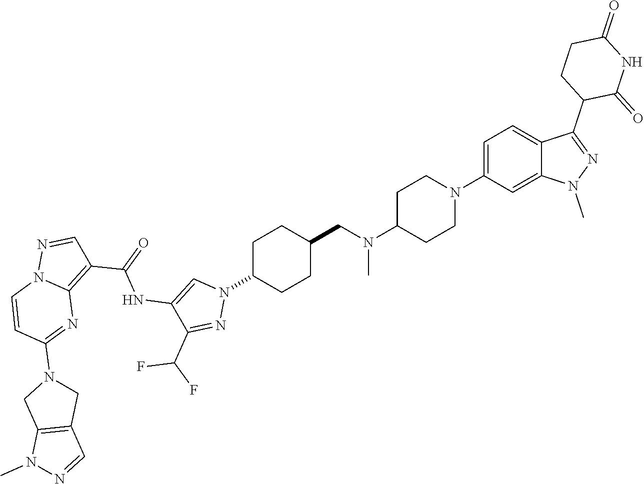 |
| P-50 | 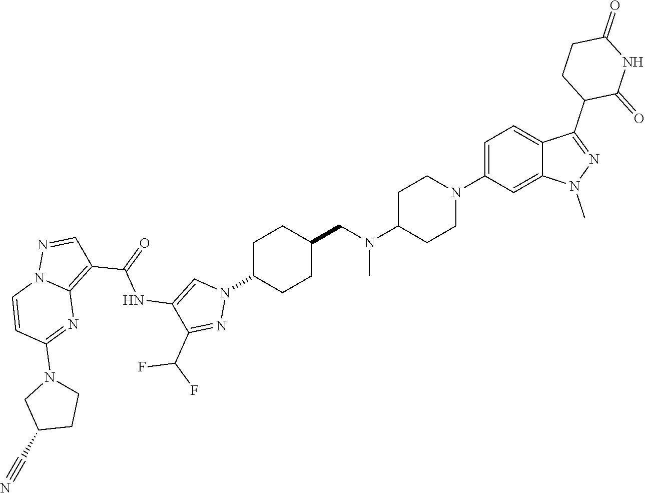 |

or a pharmaceutically acceptable salt thereof, wherein:

It is understood that in the present description, combinations of substituents and/or variables of the depicted formulae are permissible only if such contributions result in stable compounds.

Furthermore, all compounds of Formula (I) that exist in free base or acid form can be converted to their pharmaceutically acceptable salts by treatment with the appropriate inorganic or organic base or acid by methods known to one skilled in the art. Salts of the compounds of Formula (I) can be converted to their free base or acid form by standard techniques.

### Methods of Synthesis

The compounds described herein can be made using conventional organic syntheses and commercially available starting materials, or the methods provided herein. By way of example and not limitation, compounds of Formula (I) can be prepared as outlined in Schemes 1-5, as well as in the examples set forth herein. It should be noted that one skilled in the art would know how to modify the procedures set forth in the illustrative schemes and examples to arrive at the desired products.

Compounds of Formula A can be prepared as outlined in Scheme 1. Coupling of intermediate a-1 with intermediate a-2 under basic conditions, such as in the presence of DIPEA, forms intermediate a-3, which is then deprotected under basic conditions to form intermediate a-4. Subsequent coupling of intermediate a-4 with intermediate a-5 using TCFH/NMI affords intermediate a-6, which is then deprotected under basic conditions to yield intermediate a-7, followed by coupling with intermediate a-8 (for example, using HATU) to generate intermediate a-9. Deprotection of a-9 under acidic conditions affords compounds of Formula A.

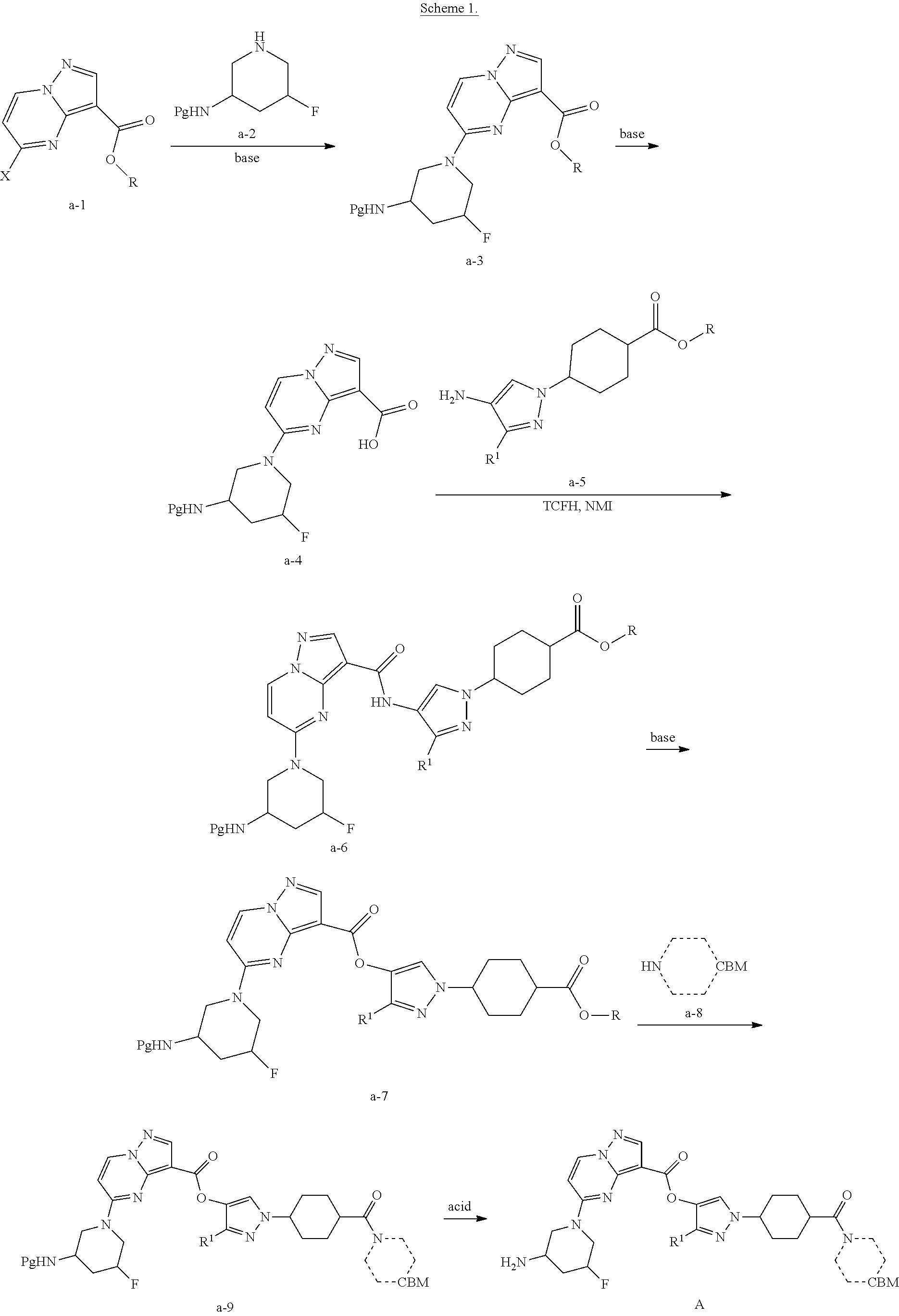

wherein X is halo such as chloro; Pg is a protecting group such as Boc; R is alkyl such as methyl or ethyl; R¹ is as described for Formula (I); and

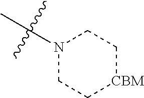

represents the portion of Formula (I) corresponding to

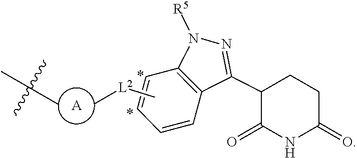

Compounds of Formula B can be prepared as outlined in Scheme 2. Coupling of intermediate a-1 with intermediate b-1 under basic conditions, such as in the presence of DIPEA, forms intermediate b-2, which is then deprotected under basic conditions to form intermediate b-3. Subsequent coupling of intermediate b-3 with intermediate b-5 (which can be prepared by reducing intermediate b-4, for example with NaBH₄) using TCFH/NMI affords intermediate b-6. Subsequent oxidation of intermediate b-6 followed by reductive amination with intermediate a-8 (for example, using IBX and NaBH(OAc)₃) affords compounds of Formula B.

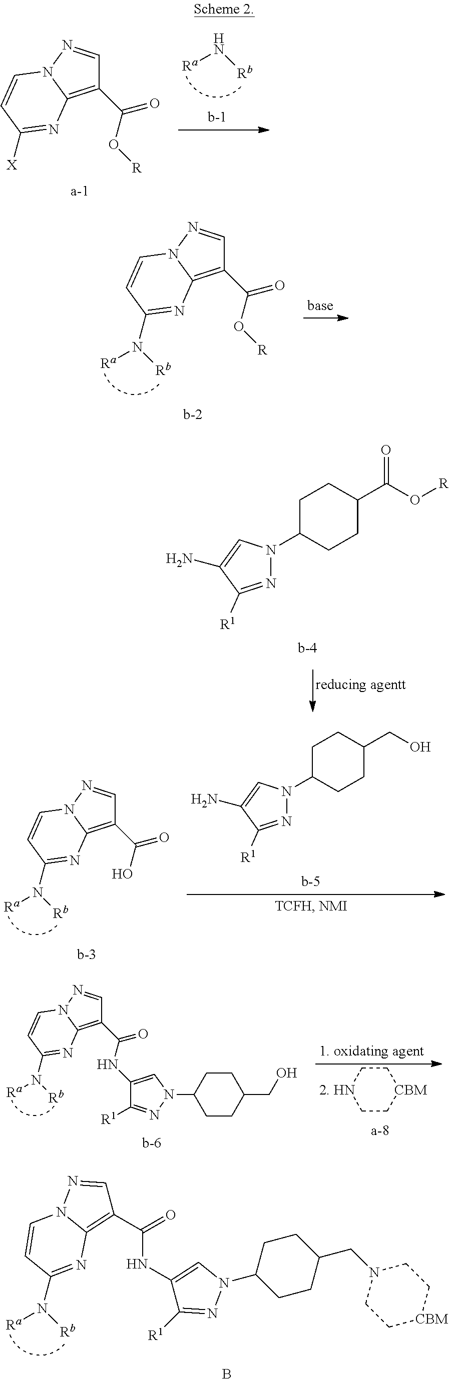

wherein X is halo such as chloro; each R is independently alkyl such as methyl or ethyl; R¹, R^{a}, and R^{b} are as described for Formula (I); and

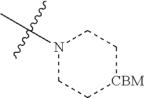

represents the portion of Formula (I) corresponding to

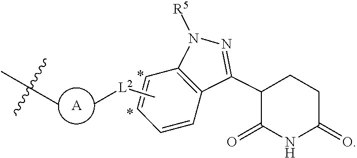

Compounds of Formula C can be prepared as outlined in Scheme 3. Reduction of intermediate c-1 (for example, with NaBH₄) affords intermediate c-2, which is then converted to intermediate c-3. Next, cyanlation of c-3 affords intermediate c-4, which is then reduced (for example, using DIBAL-H) to yield intermediate c-5. Next, c-5 is reduced (for example, using NaBH₄) to intermediate c-6, which is then hydrogenated under catalytic conditions to generate intermediate c-7. Subsequent coupling of intermediate c-7 with intermediate c-8 using TCFH/NMI affords intermediate c-9. A further oxidation of intermediate c-9 followed by reductive amination with intermediate a-8 (for example, using IBX and NaBH(OAc)₃) affords compounds of Formula C.

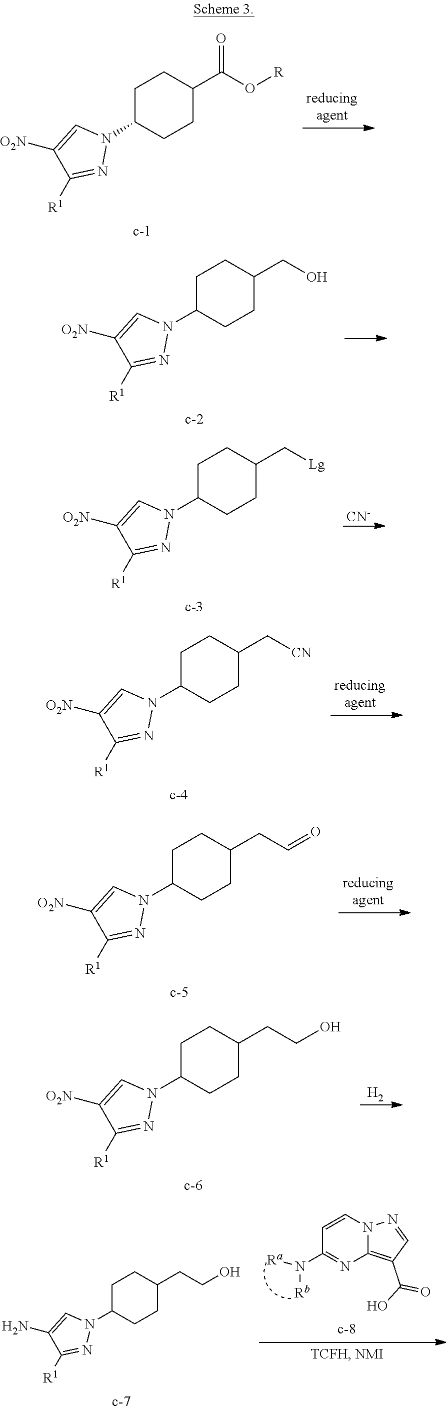

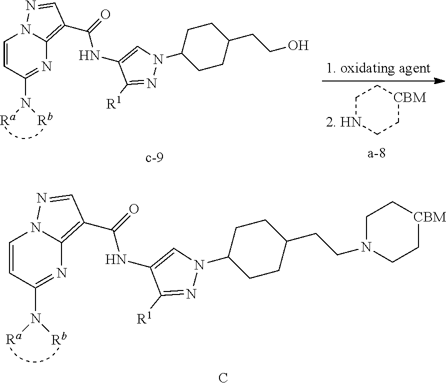

wherein Lg is a leaving group such as mesylate; R is alkyl such as methyl or ethyl; R¹, R^{a}, and R^{b} are as described for Formula (I); and

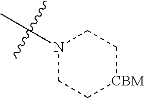

represents the portion of Formula (I) corresponding to

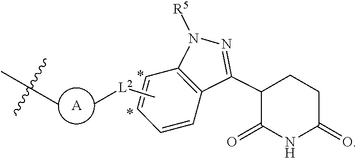

Compounds of Formula D can be prepared as outlined in Scheme 4. Intermediate b-6 can be prepared as described above with respect to the synthesis of compounds of Formula B and Scheme 2. Subsequent oxidation of intermediate b-6 followed by reductive amination with intermediate d-1 using, for example, IBX and NaBH(OAc)₃, affords compounds of Formula D.

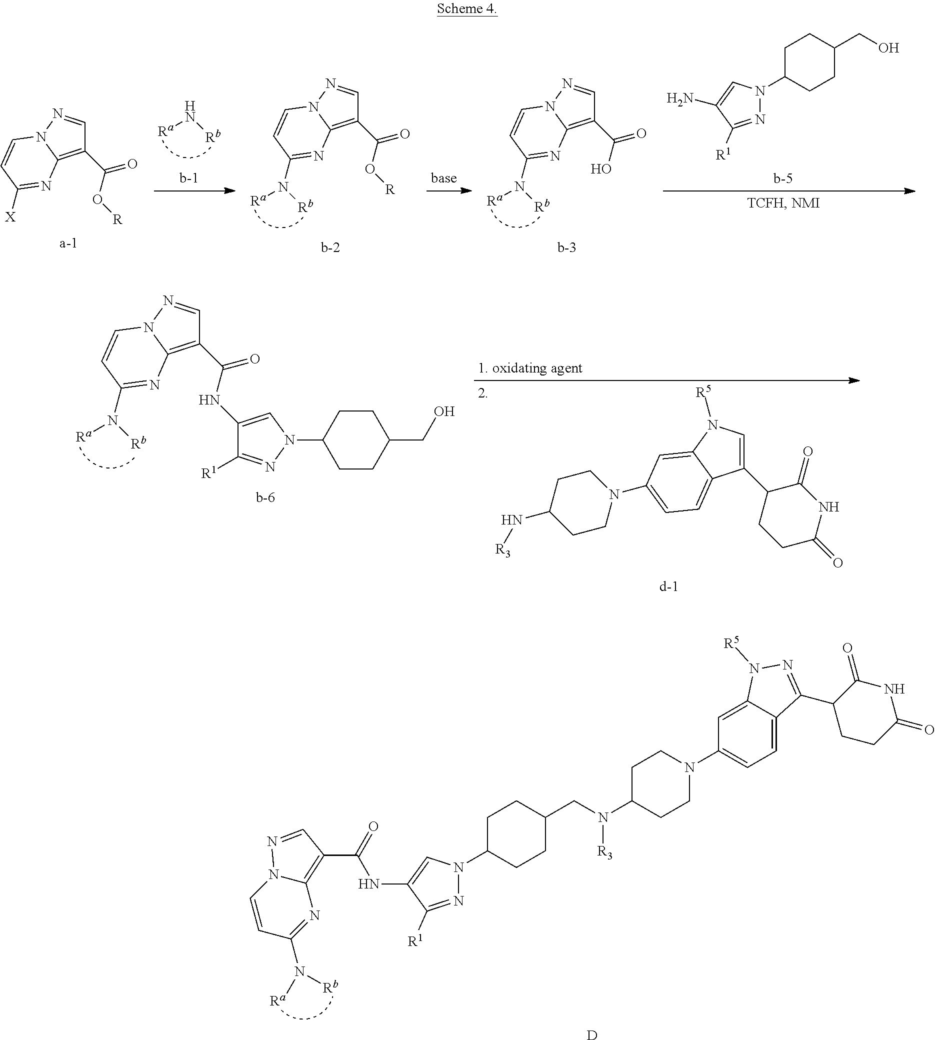

wherein X is a halo such as chloro; R is alkyl such as methyl or ethyl; and R¹, R^{a}, R^{b}, R³, and R⁵ are as described for Formula (I).

Compounds of Formula E can be prepared as outlined in Scheme 5. In a first series of reaction, intermediate e-1 is converted to intermediate e-2, followed by coupling with intermediate e-3 to form intermediate e-4. Reduction of e-4 to intermediate e-5 (for example, using Zn under acid conditions), followed by coupling using TCFH/NMI with intermediate a-4 affords intermediate e-6, which is then hydrogenated to yield intermediate e-7. In a second series of reactions, intermediate a-8 is coupled with intermediate e-8 under reducing conditions (for example, using NaBH(OAc)₃) to yield intermediate e-9, which is then deprotected under acidic conditions to generate intermediate e-10. Next, coupling of e-10 with e-7 under reducing conditions (for example, using using NaBH(OAc)₃) affords intermediate e-11, which is then deprotected under acid conditions to afford compounds of Formula E.

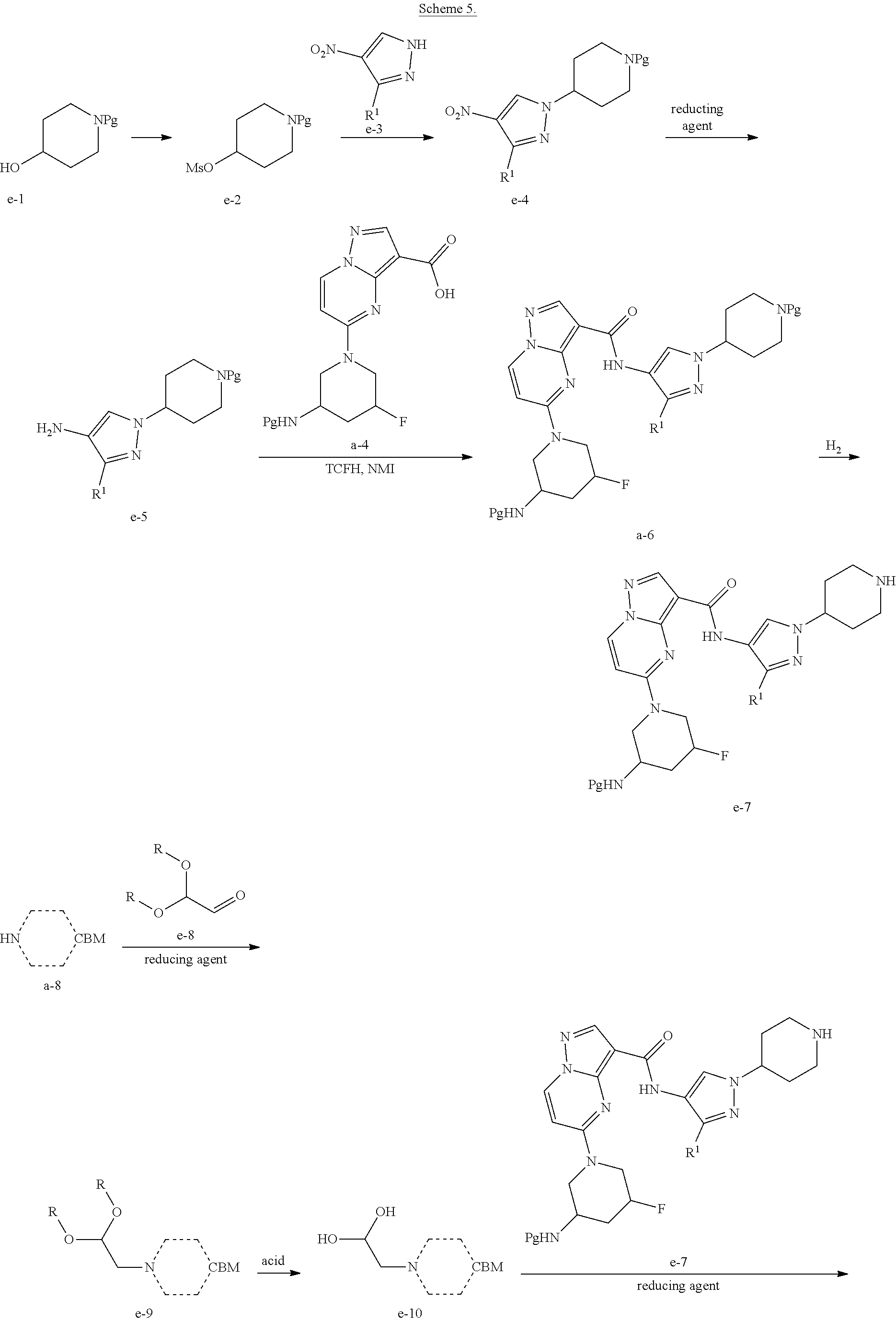

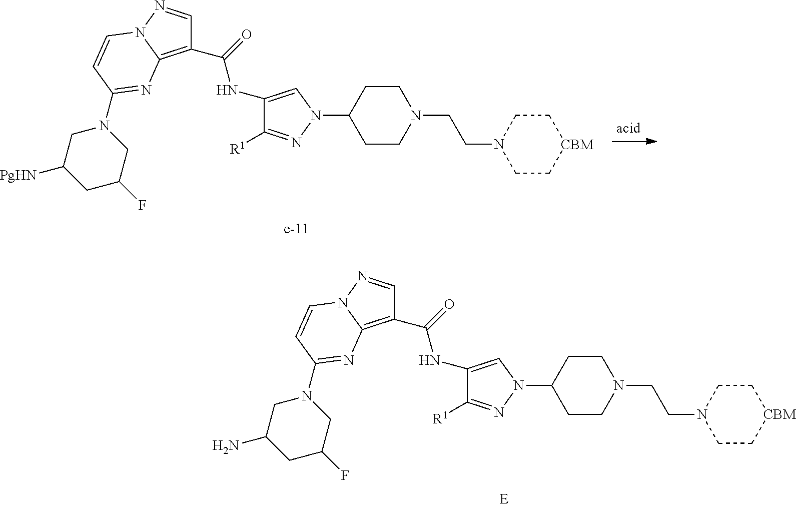

wherein each Pg is independently a protecting group such as Cbz or Boc; each R is independently alkyl such as methyl or ethyl; R¹ is as described for Formula (I); and

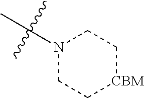

represents the portion of Formula (I) corresponding to

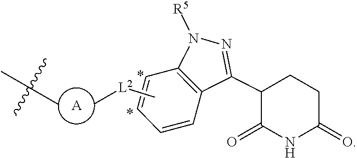

### Methods of Use

Embodiments of the present disclosure provide a method for modulating IRAK4 in a subject in need thereof, the method comprising administering to the subject an effective amount of a compound of Formula (I). Modulation (e.g., inhibition or activation) of IRAK4 can be assessed and demonstrated by a wide variety of ways known in the art. Kits and commercially available assays can be utilized for determining whether and to what degree IRAK4 has been modulated (e.g., inhibited or activated).

In one aspect, provided herein is a method of modulating IRAK4 comprising contacting IRAK4 with an effective amount of a compound of Formula (I) or any embodiment or variation thereof. In some embodiments, the compound of Formula (I) inhibits IRAK4. In other embodiments, the compound of Formula (I) activates IRAK4. In some embodiments, the compound of Formula (I) is an agonist of IRAK4. In some embodiments, the compound of Formula (I) is an antagonist of IRAK4.

In some embodiments, provided herein is a method for targeting IRAK4 for degradation comprising contacting IRAK4 with an effective amount of a compound of Formula (I) or any embodiment or variation thereof.

In some embodiments, a compound of Formula (I) modulates the activity of IRAK4 by about 1%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, a compound of Formula (I) modulates the activity of IRAK4 by about 1-100%, 5-100%, 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, 95-100%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-90%, 20-80%, 30-70%, or 40-60%.

Also provided in certain embodiments of the present disclosure is a method for degrading IRAK4 in a subject in need thereof, the method comprising administering to the subject an effective amount of a compound of Formula (I). Degradation of IRAK4 can be assessed and demonstrated by a wide variety of ways known in the art. Kits and commercially available assays, including cell-based assays, can be utilized for determining whether and to what degree IRAK4 has been degraded.

In one aspect, provided herein is a method of degrading IRAK4 comprising contacting IRAK4 with an effective amount of a compound of Formula (I) or any embodiment or variation thereof. In some embodiments, the compound of Formula (I) partially degrades IRAK3. In some embodiments, the compound of Formula (I) fully degrades IRAK4.

In some embodiments, a compound of Formula (I) degrades IRAK4 by about 1%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, a compound of Formula (I) degrades IRAK4 by about 1-100%, 5-100%, 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, 95-100%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-90%, 20-80%, 30-70%, or 40-60%.

In another aspect, provided herein is a method for treating an inflammatory or autoimmune disease in a subject in need thereof, comprising administering to the subject an effective amount of a compound of Formula (I). In some embodiments, provided herein is a method for treating an inflammatory disease in a subject in need thereof, comprising administering to the subject an effective amount of a compound of Formula (I). In some embodiments, provided herein is a method for treating an autoimmune disease in a subject in need thereof, comprising administering to the subject an effective amount of a compound of Formula (I). In some embodiments, provided herein is a method for preventing an inflammatory or autoimmune disease in a subject in need thereof, comprising administering to the subject an effective amount of a compound of Formula (I). In some embodiments, provided herein is a method for preventing an inflammatory disease in a subject in need thereof, comprising administering to the subject an effective amount of a compound of Formula (I). In some embodiments, provided herein is a method for preventing an autoimmune disease in a subject in need thereof, comprising administering to the subject an effective amount of a compound of Formula (I). Non-limiting examples of an inflammatory or autoimmune disease include atopic dermatitis, asthma, lupus, rheumatoid arthritis, familial mediterranean fever, psoriasis, generalized pustular psoriasis, cryoprin-associated periodic syndrome, hidradenitis suppurativa, Bechet's syndrome, or familial cold autoinflammatory syndrome.

In some embodiments, administering a compound of Formula (I) to a subject that is predisposed to an inflammatory or autoimmune disease prevents the subject from developing any symptoms of the inflammatory or autoimmune disease. In some embodiments, administering a compound of Formula (I) to a subject that does not yet display symptoms of an inflammatory or autoimmune disease prevents the subject from developing any symptoms of the inflammatory or autoimmune disease. In some embodiments, administering a compound of Formula (I) to a subject in need thereof diminishes the extent of the inflammatory or autoimmune disease in the subject. In some embodiments, administering a compound of Formula (I) to a subject in need thereof stabilizes the inflammatory or autoimmune disease (prevents or delays the worsening of the inflammatory or autoimmune disease). In some embodiments, administering a compound of Formula (I) to a subject in need thereof delays the occurrence or recurrence of the inflammatory or autoimmune disease. In some embodiments, administering a compound of Formula (I) to a subject in need thereof slows the progression of the inflammatory or autoimmune disease. In some embodiments, administering a compound of Formula (I) to a subject in need thereof provides a partial remission of the inflammatory or autoimmune disease. In some embodiments, administering a compound of Formula (I) to a subject in need thereof provides a total remission of the inflammatory or autoimmune disease. In some embodiments, administering a compound of Formula (I) to a subject in need thereof decreases the dose of one or more other medications required to treat the inflammatory or autoimmune disease. In some embodiments, administering a compound of Formula (I) to a subject in need thereof enhances the effect of another medication used to treat the inflammatory or autoimmune disease. In some embodiments, administering a compound of Formula (I) to a subject in need thereof delays the progression of the inflammatory or autoimmune disease. In some embodiments, administering a compound of Formula (I) to a subject in need thereof increases the quality of life of the subject having an inflammatory or autoimmune disease. In some embodiments, administering a compound of Formula (I) to a subject in need thereof prolongs survival of a subject having an inflammatory or autoimmune disease.

In one aspect, provided herein is method of preventing a subject that is predisposed to an inflammatory or autoimmune disease from developing any symptoms of the inflammatory or autoimmune disease, the method comprising administering a compound of Formula (I) to the subject. In some embodiments, provided herein is a method of preventing a subject that does not yet display symptoms of an inflammatory or autoimmune disease from developing any symptoms of the inflammatory or autoimmune disease, the method comprising administering a compound of Formula (I) to the subject.

In some aspects, provided herein is a method of diminishing the extent of an inflammatory or autoimmune disease in a subject, the method comprising administering a compound of Formula (I) to the subject. In some embodiments, provided herein is a method of stabilizing an inflammatory or autoimmune disease in a subject, the method comprising administering a compound of Formula (I) to the subject. In some embodiments, the method prevents the worsening of the inflammatory or autoimmune disease. In some embodiments, the method delays the worsening of the inflammatory or autoimmune disease.

In another aspect, provided herein is a method of delaying the occurrence or recurrence of an inflammatory or autoimmune disease in a subject, the method comprising administering a compound of Formula (I) to the subject.

In some embodiments, provided herein is a method of slowing the progression of an inflammatory or autoimmune disease in a subject, the method comprising administering a compound of Formula (I) to the subject. In some embodiments, the method provides a partial remission of the inflammatory or autoimmune disease. In some embodiments, the method provides a total remission of the inflammatory or autoimmune disease.

In further aspects, provided herein is a method of decreasing the dose of one or more other medications required to treat an inflammatory or autoimmune disease in a subject, the method comprising administering a compound of Formula (I) to the subject. In some embodiments, provided herein is a method of enhancing the effect of another medication used to treat an inflammatory or autoimmune disease in a subject, the method comprising administering a compound of Formula (I) to the subject.

Also provided here is a method of delaying the progression of an inflammatory or autoimmune disease in a subject, the method comprising administering a compound of Formula (I) to the subject. In some embodiments, the method increases the quality of life of the subject having an inflammatory or autoimmune disease. In some embodiments, the method prolongs survival of the subject having an inflammatory or autoimmune disease.

In another aspect, provided herein is a method for treating inflammatory or autoimmune symptoms caused by a disease in a subject in need thereof, comprising administering to the subject an effective amount of a compound of Formula (I). In some embodiments, provided herein is a method for preventing inflammatory or autoimmune symptoms caused by a disease in a subject in need thereof, comprising administering to the subject an effective amount of a compound of Formula (I). In some embodiments, administering a compound of Formula (I) to a subject that is predisposed to a disease which causes inflammatory or autoimmune symptoms prevents the subject from developing any inflammatory or autoimmune symptoms. In some embodiments, administering a compound of Formula (I) to a subject that does not yet display inflammatory or autoimmune symptoms of a disease which causes inflammatory or autoimmune symptoms prevents the subject from developing any inflammatory or autoimmune symptoms. In some embodiments, administering a compound of Formula (I) to a subject in need thereof diminishes the extent of the inflammatory or autoimmune symptoms caused by the disease in the subject. In some embodiments, administering a compound of Formula (I) to a subject in need thereof stabilizes the inflammatory or autoimmune symptoms of the disease (prevents or delays the worsening of the inflammatory or autoimmune symptoms). In some embodiments, administering a compound of Formula (I) to a subject in need thereof delays the occurrence or recurrence of the inflammatory or autoimmune symptoms caused by the disease. In some embodiments, administering a compound of Formula (I) to a subject in need thereof slows the progression of the inflammatory or autoimmune symptoms caused by the disease. In some embodiments, administering a compound of Formula (I) to a subject in need thereof provides a partial remission of the disease which causes inflammatory or autoimmune symptoms. In some embodiments, administering a compound of Formula (I) to a subject in need thereof provides a total remission of the disease which causes inflammatory or autoimmune symptoms. In some embodiments, administering a compound of Formula (I) to a subject in need thereof decreases the dose of one or more other medications required to treat the disease which causes inflammatory or autoimmune symptoms. In some embodiments, administering a compound of Formula (I) to a subject in need thereof enhances the effect of another medication used to treat the inflammatory or autoimmune symptoms of the disease. In some embodiments, administering a compound of Formula (I) to a subject in need thereof delays the progression of the disease which causes inflammatory or autoimmune symptoms. In some embodiments, administering a compound of Formula (I) to a subject in need thereof increases the quality of life of the subject having a disease which causes inflammatory or autoimmune symptoms. In some embodiments, administering a compound of Formula (I) to a subject in need thereof prolongs survival of a subject having a disease which causes inflammatory or autoimmune symptoms. In some embodiments, the disease is atopic dermatitis, asthma, lupus, rheumatoid arthritis, familial mediterranean fever, psoriasis, generalized pustular psoriasis, cryoprin-associated periodic syndrome, hidradenitis suppurativa, Bechet's syndrome, or familial cold autoinflammatory syndrome.

In some embodiments, compounds of Formula (I) are useful for treating a disorder selected from atopic dermatitis, asthma, lupus, rheumatoid arthritis, familial mediterranean fever, psoriasis, generalized pustular psoriasis, cryoprin-associated periodic syndrome, hidradenitis suppurativa, Bechet's syndrome, and familial cold autoinflammatory syndrome.

### Pharmaceutical Compositions and Routes of Administration

The compounds provided herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions.

The compounds disclosed herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the compounds of Formula (I) in the pharmaceutical composition may be at a level that will exercise the desired effect; for example, about 0.005 mg/kg of a subject's body weight to about 10 mg/kg of a subject's body weight in unit dosage for both oral and parenteral administration.

The dose of a compound of Formula (I) to be administered to a subject is rather widely variable and can be subject to the judgment of a health-care practitioner. In general, the compounds disclosed herein can be administered one to four times a day in a dose of about 0.001 mg/kg of a subject's body weight to about 10 mg/kg of a subject's body weight, but the above dosage may be properly varied depending on the age, body weight and medical condition of the subject and the type of administration. In any given case, the amount of the compound of Formula (I) administered may depend on such factors as the solubility of the active component, the formulation used and the route of administration.

A compound of Formula (I) can be administered orally for reasons of convenience. In one embodiment, when administered orally, a compound of Formula (I) is administered with a meal and water. In another embodiment, the compound of Formula (I) is dispersed in water or juice (e.g., apple juice or orange juice) or any other liquid and administered orally as a solution or a suspension.

The compounds disclosed herein can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend inpart upon the site of the medical condition.

In one embodiment, provided herein are capsules containing a compound of Formula (I) without an additional carrier, excipient or vehicle.

In another embodiment, provided herein are compositions comprising an effective amount of a compound of Formula (I) and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

The compositions can be in the form of tablets, chewable tablets, capsules, solutions, parenteral solutions, troches, suppositories and suspensions and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts, such as the hydrochloride salt. In general, all of the compositions are prepared according to known methods in pharmaceutical chemistry. Capsules can be prepared by mixing a compound of Formula (I) with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the dye. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a compound of Formula (I) as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the compound of Formula (I) can be delayed or prolonged by proper formulation. For example, a slowly soluble pellet of the compound of Formula (I) can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the compound of Formula (I) in oily or emulsified vehicles that allow it to disperse slowly in the serum.

### EXEMPLARY EMBODIMENTS

The present disclosure is further described by the following embodiments.

Embodiment P1. A compound of Formula (I):

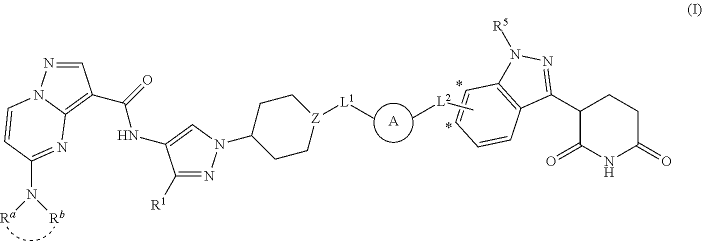

or a pharmaceutically acceptable salt thereof, wherein:
R¹ is C₁-C₆ haloalkyl;
R^{a} is H or C₁-C₆ alkyl;
R^{b} is C₁-C₆ alkyl, 5- to 6-membered heteroaryl, —(C₁-C₆ alkylene)(5- to 6-membered heteroaryl), or —(C₁-C₆ alkylene)NH₂, wherein the heteroaryl contains 1-2 nitrogen atoms and is optionally substituted by 1-5 R² groups;
or the dashed line between R^{a} and R^{b} represents a ring structure where R^{a} and R^{b} are taken together with the nitrogen atom to which they are attached to form a 5- to 10-membered monocyclic or bicyclic heterocyclyl optionally containing 1-2 additional heteroatoms selected from N and O, and is optionally substituted by 1-5 R² groups;
each R² is independently —NH₂, halo, C₁-C₆ alkyl, C₁-C₆ haloalkyl, —CN, or 5- to 6-membered heteroaryl, wherein the heteroaryl contains 1-2 nitrogen atoms and is optionally substituted by 1-5 groups selected from C₁-C₆ alkyl, halo, and C₁-C₆ haloalkyl;
L¹ is —C(O)N(H)—, —C(O)—, —(C₁-C₆ alkylene)N(R³)—, or C₁-C₆ alkylene;
L² is a bond, —C(O)—, —N(R³)—, or O;
each R³ is independently H or C₁-C₆ alkyl;
Ring A is a monocyclic 4- to 6-membered heterocyclylene or a bicyclic 6- to 9-membered spiro heterocyclylene, wherein the heterocyclylene contains 1-2 nitrogen atoms, and wherein the heterocyclylene is substituted by m R⁴ groups;
each R⁴ is independently halo, C₁-C₆ alkyl, or C₁-C₆ haloalkyl;
m is 0-5;
R⁵ is H or C₁-C₆ alkyl;
Z is CH or N; and
* indicates an attachment point for the L²-containing moiety.

Embodiment P2. The compound of embodiment P1, or a pharmaceutically acceptable salt thereof, wherein:
R¹ is C₁-C₃ haloalkyl.

Embodiment P3. The compound of embodiment P2, or a pharmaceutically acceptable salt thereof, wherein:
R¹ is —CHF₂ or —CF₃.

Embodiment P4. The compound of embodiment P3, or a pharmaceutically acceptable salt thereof, wherein:
R¹ is —CHF₂.

Embodiment P5. The compound of any one of embodiments P1-P4, or a pharmaceutically acceptable salt thereof, wherein:
R^{a} is H or C₁-C₃ alkyl;
R^{b} is C₁-C₃ alkyl, 6-membered heteroaryl, —(C₁-C₃ alkylene)(6-membered heteroaryl), or —(C₁-C₃ alkylene)NH₂, wherein the heteroaryl contains 1-2 nitrogen atoms and is optionally substituted by 1-2 R² groups; and
each R² is independently —NH₂, halo, C₁-C₃ alkyl, C₁-C₃ haloalkyl, or —CN.

Embodiment P6. The compound of embodiment P5, or a pharmaceutically acceptable salt thereof, wherein:
R^{a} is H or —CH₃;
R^{b} is —CH₃, —CH₂(pyridyl), —CH₂CH₂NH₂, or pyridyl, wherein the pyridyl is optionally substituted by 1 R² group; and
R² is —CH₃.

Embodiment P7. The compound of embodiment P6, or a pharmaceutically acceptable salt thereof, wherein:

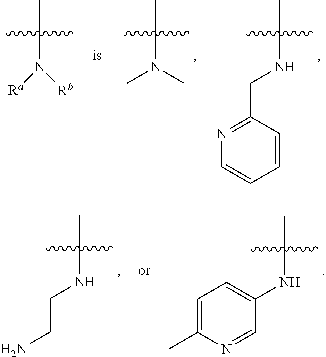

Embodiment P8. The compound of any one of embodiments P1-P4, or a pharmaceutically acceptable salt thereof, wherein:
R^{a} and R^{b} are taken together with the nitrogen atom to which they are attached to form a 5- to 8-membered monocyclic or bicyclic heterocyclyl optionally containing 1-2 additional heteroatoms selected from N and O, and optionally substituted by 1-3 R² groups; and
each R² is independently —NH₂, halo, C₁-C₃ alkyl, C₁-C₃ haloalkyl, —CN, or 5-membered heteroaryl, wherein the heteroaryl contains 1-2 nitrogen atoms and is optionally substituted by 1-3 groups selected from C₁-C₃ alkyl, halo, and C₁-C₃ haloalkyl.

Embodiment P9. The compound of embodiment P8, or a pharmaceutically acceptable salt thereof, wherein:
R^{a} and R^{b} are taken together with the nitrogen atom to which they are attached to form a 5- to 6-membered monocyclic heterocyclyl or 8-membered fused or bridged bicyclic heterocyclyl, wherein the heterocyclyl optionally contains 1-2 additional heteroatoms selected from N and O, and is optionally substituted by 1-3 R² groups; and
each R² is independently —NH₂, F, Cl, —CH₃, or —CF₃, —CN, or pyrazolyl optionally substituted by 1-2 groups selected from —CH₃ and Cl.

Embodiment P10. The compound of embodiment P9, or a pharmaceutically acceptable salt thereof, wherein:

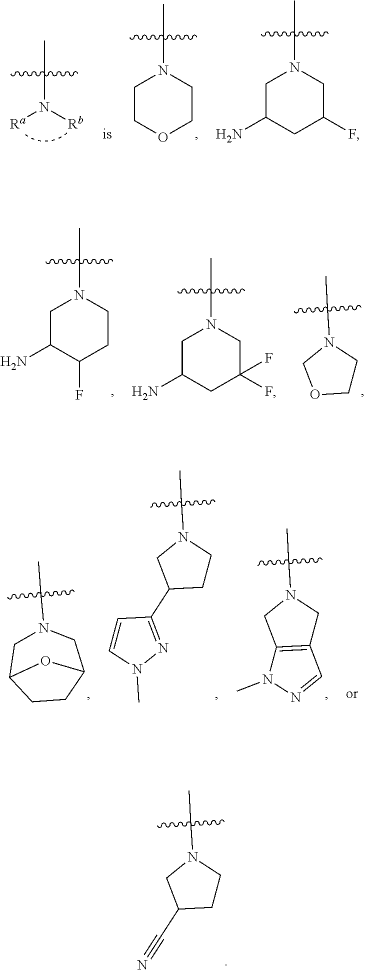

Embodiment P11. The compound of any one of embodiments P1-P10, or a pharmaceutically acceptable salt thereof, wherein:
L¹ is —C(O)N(H)—, —C(O)—, —(C₁-C₃ alkylene)N(R³)—, or C₁-C₃ alkylene; and
R³ is H or C₁-C₃ alkyl.

Embodiment P12. The compound of embodiment P11, or a pharmaceutically acceptable salt thereof, wherein:
L¹ is —C(O)N(H)—, —C(O)—, —CH₂N(H)—, —CH₂N(CH₃)—, —CH₂CH₂N(CH₃)—, —CH₂—, or —CH₂CH₂—.

Embodiment P13. The compound of any one of embodiments P1-P12, or a pharmaceutically acceptable salt thereof, wherein:
L² is a bond, —C(O)—, —N(R³)—, or O; and
R³ is H or C₁-C₃ alkyl.

Embodiment P14. The compound of embodiment P13, or a pharmaceutically acceptable salt thereof, wherein:
L² is a bond, —C(O)—, —N(H)—, —N(CH₃)—, or O.

Embodiment P15. The compound of any one of embodiments P1-P14, or a pharmaceutically acceptable salt thereof, wherein:
Ring A is a monocyclic 4- to 6-membered heterocyclylene containing 1-2 nitrogen atoms and substituted by m R⁴ groups.

Embodiment P16. The compound of embodiment P15, or a pharmaceutically acceptable salt thereof, wherein:
Ring A is

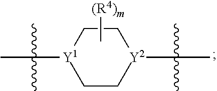

and
Y¹ and Y² are independently CH or N, provided that at least one of Y¹ and Y² is N.

Embodiment P17. The compound of embodiment P16, or a pharmaceutically acceptable salt thereof, wherein:
Y¹ is CH; and
Y² is N.

Embodiment P18. The compound of embodiment P16, or a pharmaceutically
acceptable salt thereof, wherein:
Y¹ is N; and
Y² is CH.

Embodiment P19. The compound of embodiment P16, or a pharmaceutically acceptable salt thereof, wherein:
Y¹ and Y² are each N.

Embodiment P20. The compound of embodiment P15, or a pharmaceutically acceptable salt thereof, wherein:
Ring A is

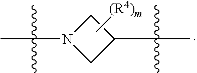

Embodiment P21. The compound of embodiment P15, or a pharmaceutically acceptable salt thereof, wherein:
Ring A is a bicyclic 6- to 9-membered spiro heterocyclylene containing 1-2 nitrogen atoms and substituted by m R⁴ groups.

Embodiment P22. The compound of embodiment P21, or a pharmaceutically acceptable salt thereof, wherein:
Ring A is

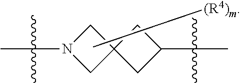

Embodiment P23. The compound of any one of embodiments P1-P22, or a pharmaceutically acceptable salt thereof, wherein:
each R⁴ is independently halo, C₁-C₃ alkyl, or C₁-C₃ haloalkyl.

Embodiment P24. The compound of embodiment P23, or a pharmaceutically acceptable salt thereof, wherein:
each R⁴ is independently F, Cl, —CH₃, or —CF₃.

Embodiment P25. The compound of embodiment P24, or a pharmaceutically acceptable salt thereof, wherein:
each R⁴ is independently F or —CH₃.

Embodiment P26. The compound of any one of embodiments P1-P22, or a pharmaceutically acceptable salt thereof, wherein:
m is 0.

Embodiment P27. The compound of any one of embodiments P1-P25, or a pharmaceutically acceptable salt thereof, wherein:
m is 1 or 2.

Embodiment P28. The compound of any one of embodiments P1-P27, or a pharmaceutically acceptable salt thereof, wherein:
Ring A is

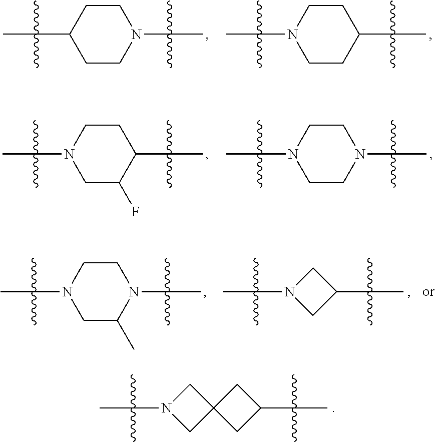

Embodiment P29. The compound of any one of embodiments P1-P28, or a pharmaceutically acceptable salt thereof, wherein:
R⁵ is H or C₁-C₃ alkyl.

Embodiment P30. The compound of embodiment P29, or a pharmaceutically acceptable salt thereof, wherein:
R⁵ is H or —CH₃.

Embodiment P31. The compound of embodiment P30, or a pharmaceutically acceptable salt thereof, wherein:
R⁵ is —CH₃.

Embodiment P32. The compound of any one of embodiments P1-P31, or a pharmaceutically acceptable salt thereof, wherein:
Z is CH.

Embodiment P33. The compound of any one of embodiments P1-P31, or a pharmaceutically acceptable salt thereof, wherein:
Z is N.

Embodiment P34. The compound of any one of embodiments P1-P33, or a pharmaceutically acceptable salt thereof, wherein:

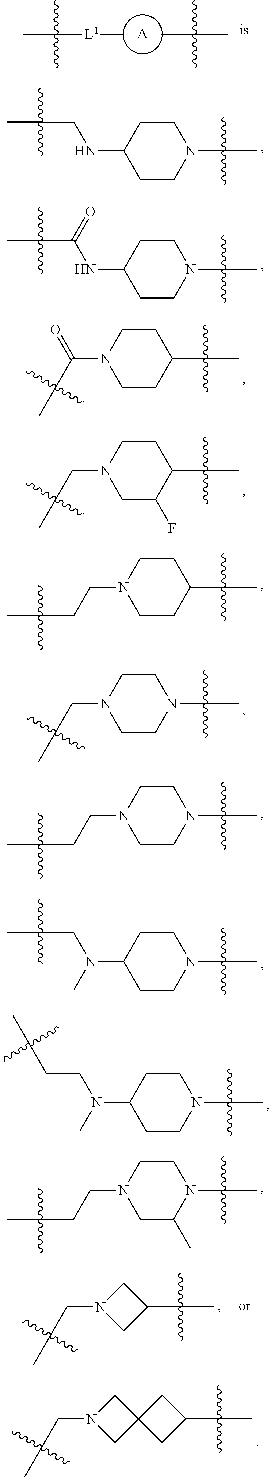

Embodiment P35. The compound of any one of embodiments P1-P34, or a pharmaceutically acceptable salt thereof, wherein:

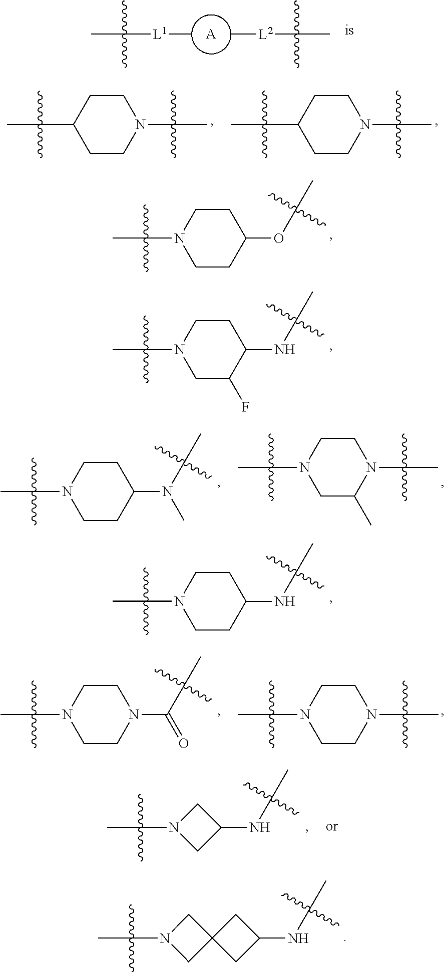

Embodiment P36. The compound of any one of embodiments P1-P35, or a pharmaceutically acceptable salt thereof, wherein:

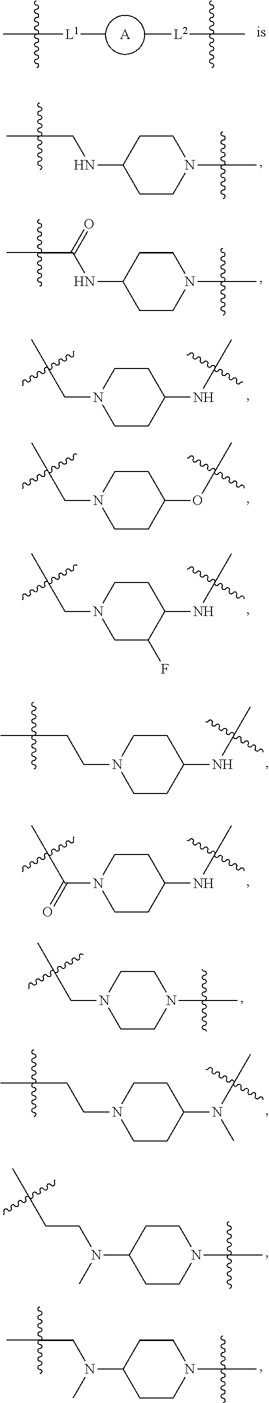

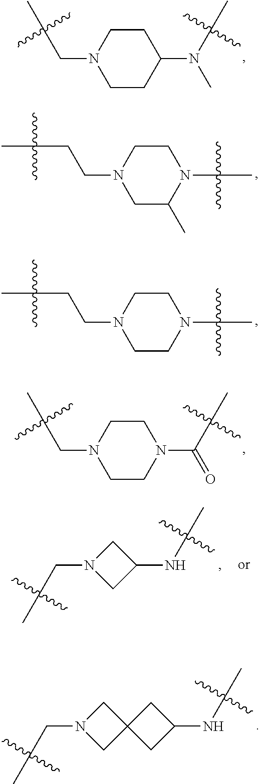 [SMILES: *CN1CCC(N(*)C)CC1 ; *CCN1CCN(*)C(C)C1 ; *CCN1CCN(*)CC1 ; *CN1CCN(C(*)=O)CC1 ; *CN1CC(N*)C1 ; *CN1CC2(CC(N*)C2)C1]

Embodiment P37. The compound of any one of embodiments P1-P7 and P11-P36, or a pharmaceutically acceptable salt thereof, wherein the compound is of Formula (IA):

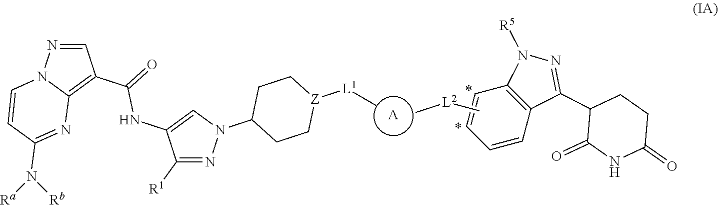

wherein:
R^{a} is H or C₁-C₆ alkyl; and
R^{b} is C₁-C₆ alkyl, 5- to 6-membered heteroaryl, —(C₁-C₆ alkylene)(5- to 6-membered heteroaryl), or —(C₁-C₆ alkylene)NH₂, wherein the heteroaryl contains 1-2 nitrogen atoms and is optionally substituted by 1-5 R² groups.

Embodiment P38. The compound of any one of embodiments 1-4 and 8-36, or a pharmaceutically acceptable salt thereof, wherein the compound is of Formula (IB):

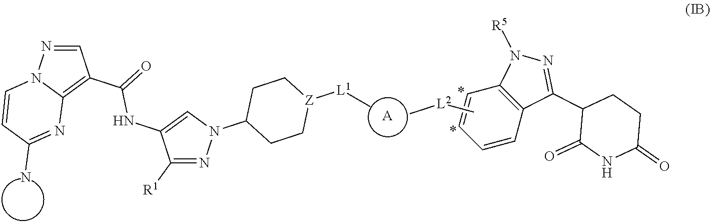

wherein:

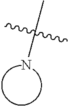 [SMILES: *N]

is a 5- to 10-membered monocyclic or bicyclic heterocyclyl optionally containing 1-2 additional heteroatoms selected from N and O, and is optionally substituted by 1-5 R² groups.

Embodiment P39. The compound of any one of embodiments P1-P19 and P23-P37, or a pharmaceutically acceptable salt thereof, wherein the compound is of Formula (IIa) or (IIb):

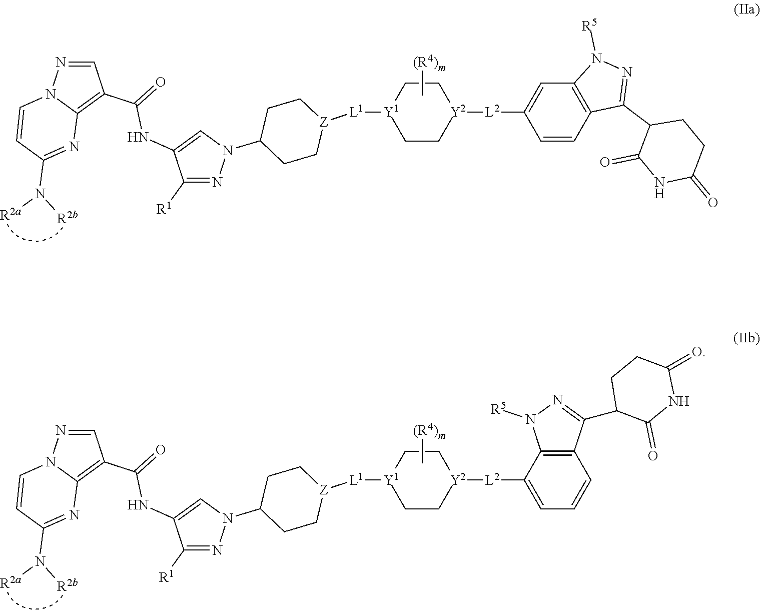

Embodiment P40. The compound of any one of embodiments P1-P15, P20, P21, and P23-P38, or a pharmaceutically acceptable salt thereof, wherein the compound is of Formula (IIIa) or (IVa):

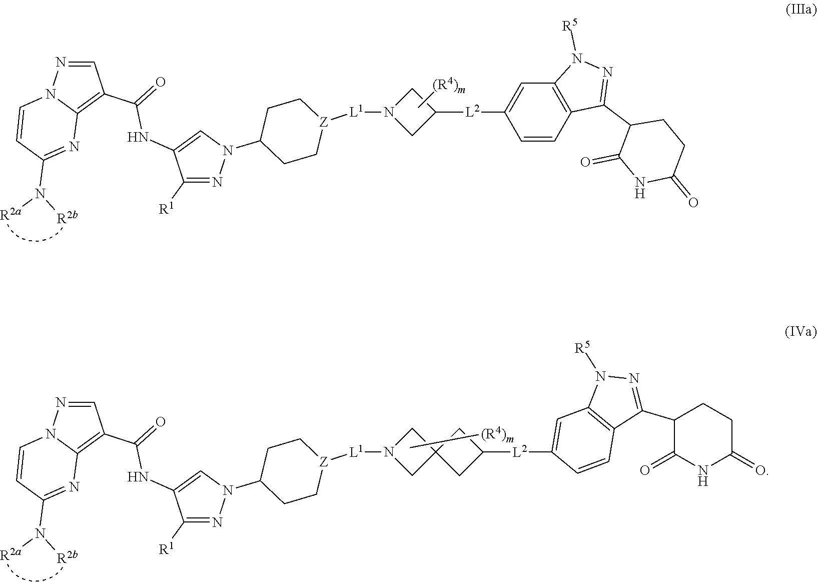

Embodiment P41. A compound selected from the compounds of Table 1 and pharmaceutically acceptable salts thereof.

Embodiment P42. A pharmaceutical composition comprising the compound of any one of embodiments P1-P41, or a pharmaceutically acceptable salt thereof, and a pharmaceutically acceptable excipient.

Embodiment P43. A method of modulating interleukin-1 (IL1) receptor-associated kinase 4 (IRAK4) comprising contacting IRAK4 with an effective amount of the compound of any one of embodiments P1-P41, or a pharmaceutically acceptable salt thereof, or the pharmaceutical composition of embodiment P42.

Embodiment P44. A method of treating an inflammatory or autoimmune disease in a subject in need thereof, comprising administering to the subject an effective amount of the compound of any one of embodiments P1-P41, or a pharmaceutically acceptable salt thereof, or the pharmaceutical composition of embodiment P42.

Embodiment P45. The method of embodiment P44, wherein the inflammatory or autoimmune disease is atopic dermatitis, asthma, lupus, rheumatoid arthritis, familial mediterranean fever, psoriasis, generalized pustular psoriasis, cryoprin-associated periodic syndrome, hidradenitis suppurativa, Bechet's syndrome, or familial cold autoinflammatory syndrome.

### EXAMPLES

The following Examples are presented by way of illustration, not limitation. Compounds are named using the automatic name generating tool provided in ChemBiodraw Ultra (Cambridgesoft), which generates systematic names for chemical structures, with support for the Cahn-Ingold-Prelog rules for stereochemistry. One skilled in the art can modify the procedures set forth in the illustrative examples to arrive at the desired products.

Salts of the compounds described herein can be prepared by standard methods, such as inclusion of an acid (for example TFA, formic acid, or HCl) in the mobile phases during chromatography purification, or stirring of the products after chromatography purification, with a solution of an acid (for example, aqueous HCl).

As used in certain of the chemical structures provided in the following Examples, designation of a particular atom with “*” or “orn” indicates that the absolute stereochemistry of the indicated atom was not determined.

The following abbreviations may be relevant for the application.

### Abbreviations

AcOH: acetic acid
aq or aq.: aqueous
BCA assay: Bicinchoninic acid assay
BuOH: butanol
CBM: Cereblon Binding Moiety
CV: column volume
DCE: 1,2-dichloroethane
DCM: dichloromethane
DIPEA: N, N-diisopropylethylamine
DMF: dimethylformamide
DMSO: dimethylsulfoxide
eq.: equivalents
ESI: electrospray ionization
Et₃N: triethylamine
EtOAc: ethyl acetate
EtOH: ethanol
FA: formic acid
FBS: fetal bovine serum
FC: flash chromatography
h: hour(s)
HATU: 1-[bis(dimethylamino)methylene]-1H-1,2,3-triazolo[4,5-b]pyridinium 3-oxide
hexafluorophosphate
HPLC: high-performance liquid chromatography
IBX: 2-iodoxybenzoic acid
LCMS: liquid chromatography mass spectrometry
MeCN: acetonitrile
MeOH: methanol
min: minute(s)
MsCl: mesyl chloride
MSD: mass selective detector
MTBE: methyl tert-butyl ether
NMI: 1-methylimidazole
NMO: N-methylmorpholine N-oxide
Pd(dppf)Cl₂: bis(diphenylphosphino)ferrocene)palladium(II) dichloride
prep: preparative
quant.: quantitative
RP: reverse phase
rt or r.t.: room temperature
RT: retention time
tBuOH: tert-butanol
TCFH: chloro-N,N,N′,N′-tetramethylformamidinium hexafluorophosphate
TFA: trifluoroacetic acid
THF: tetrahydrofuran
TLC: thin layer chromatography

### SYNTHETIC EXAMPLES

### LCMS Methods

### LCMS Method 1

Column: Luna C18 (2) 50×3 mm, 3 um. Temperature: 45° C., Flow: 1.5 mL/min, run time: 2.5 min. Mobile phase conditions: Initial 95% H₂O 0.1% FA/5% MeCN 0.1% FA, linear gradient to 95% MeCN 0.1% FA over 1.3 min then hold for 1.2 minute at 95% MeCN 0.1% FA. MSD: ESI Positive

### LCMS Method 2

Column: SunFire C18 75×4.6 mm, 3.5 um. Temperature: 45° C., Flow: 1.5 mL/min, run time: 6 min. Mobile phase conditions: Initial 95% H₂O+0.1% FA/5% MeCN+0.1% FA then linear gradient to 95% MeCN+0.1% FA for 4 min then hold for 2 min at 95% MeCN+0.1% FA. MSD: Positive

### LCMS Method 3

Column: Kinetex Polar C18 2.6 um, 50×3.0 mm. Temperature: 45° C., Flow: 1.2 mL/min, Run time: 3 min. Mobile phase conditions: Initial 95% H₂O+0.1% FA/5% CH₃CN+0.1% FA then linear gradient to 95% MeCN for 1.5 min then hold for 1.5 min at 95% MeCN. MSD: Positive

### LCMS Method 4

Column: Kinetex Polar C18 2.6 um, 50×3.0 mm. Temperature: 40° C., Flow: 1.2 mL/min, Run time: 6 min. Mobile phase conditions: Initial 95% H₂O+0.1% FA/5% CH₃CN+0.1% FA then linear gradient to 95% MeCN for 3.5 min then hold for 2.5 min at 95% MeCN. MSD: Positive

### LCMS Method 5

Column: C18 4.6×75 mm, Initial Gradient at 95% H₂O+0.1% FA/5% MeCN+0.1% FA 10 min run with 1.5 min equilibration Gradient 0 to 8 min at 95% H₂O to 0% and hold for 2 minute.

### LCMS Method 6

Column: Kinetex XB—C18, 75×3.0 mm, 2.6 m, Temperature: rt, Flow: 1.0 mL/min, Run Time: 5 minutes, Mobile Phase Conditions: Mobile Phase-A: 5.0 mm Ammonium formate pH 3.3: CH₃CN (98:02), Mobile Phase-B: CH₃CN: 5.0 mm Ammonium formate pH 3.3 (98:02), Gradient: Initial 70% Mobile Phase A and 30% Mobile Phase B linear gradient to 100% Mobile Phase B for 4.0 min. MSD positive.

### LCMS Method 7

Column: XBridge—C8, 50×4.6 mm, 3.5 m, Temperature: rt, Flow: 1.5 mL/min, Run Time: 6 minutes, Mobile Phase Conditions: Mobile Phase-A: 0.1% TFA in H₂O, Mobile Phase-B: 0.1% TFA in acetonitrile, Gradient: Initial 95% Mobile Phase A and 5% Mobile Phase B linear gradient to 5% Mobile phase A and 95% Mobile Phase B for 2.5 min. MSD positive.

### Synthesis of Intermediates

Reference to a particular intermediate compound by number, such as 1 or 2, is specific to the example in which it is described. As such, multiple examples may refer to the same intermediate compound number, such as 1 or 2, but the chemical structure of the compound will be different across the different examples.

### Example I-1. Synthesis of Common Intermediate T-1

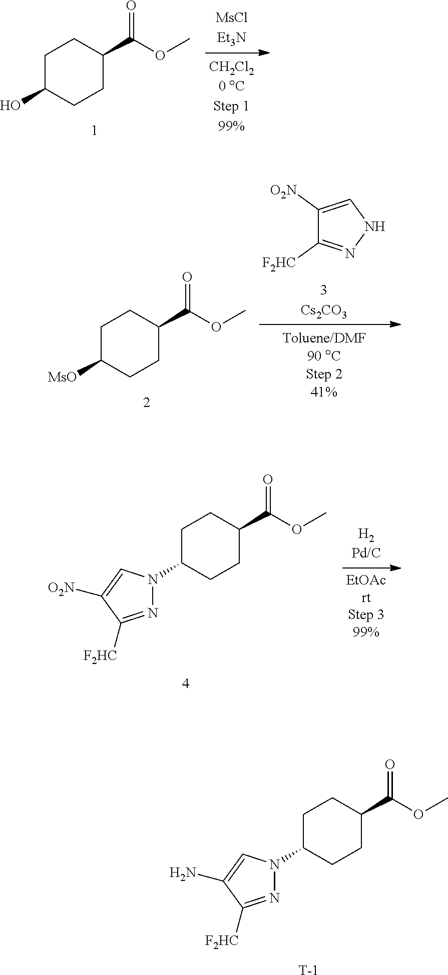

Step 1. Preparation of Methyl 4-methylsulfonyloxycyclohexanecarboxylate (2). Under nitrogen, a solution of methyl 4-hydroxycyclohexanecarboxylate 1 (5.0 g, 31.61 mmol, 1.0 eq.) in CH₂Cl₂ (105 mL, 0.3 M) was cooled to 0° C., then methanesulfonyl chloride (2.69 mL, 34.77 mmol, 1.1 eq.) was added, then triethylamine (5.28 mL, 37.93 mmol, 1.2 eq.) was added dropwise. After stirring 2 h at 0° C., TLC (CH₂Cl₂/MeOH 5.5:0.5, KMnO₄ stain) showed full conversion. The reaction was quenched by addition of water, then phases were separated and the aqueous phase was extracted 3 times with CH₂Cl₂. The combined organic phases were washed twice with brine, dried over magnesium sulfate, filtered and concentrated to give 2 (7.45 g, 99% yield) as a yellow oil.

¹H NMR (400 MHz, CDCl₃) δ ppm 1.67-1.84 (m, 4H), 1.87-1.99 (m, 2H), 2.00-2.09 (m, 2H), 2.36-2.46 (m, 1H), 3.02 (s, 3H), 3.69 (s, 3H), 4.87-4.96 (m, 1H).

Step 2. Preparation of Methyl 4-[3-(difluoromethyl)-4-nitro-pyrazol-1-yl]cyclohexanecarboxylate (4). Under nitrogen, in a flame-dried round-bottom flask, a solution of 2 (1.16 g, 4.91 mmol, 1.0 eq.), 3 (0.80 g, 4.91 mmol, 1.0 eq.) and DMF (12.3 mL, 0.4 M) was stirred at room temperature for 5 minutes before Cs₂CO₃ (3.20 g, 9.81 mmol, 2.0 eq.) was added. The resulting mixture was stirred at 90° C. for 16 h. Incomplete conversion of 3 was observed by LCMS (method 1), then a second portion of 2 (1.16 g, 4.91 mmol, 1.0 eq.) was added and stirring at 90° C. was resumed for 48 h. A 90% conversion of 3 was observed by LCMS. The reaction was quenched by addition of water. Ethyl acetate was added and phases were separated. The aqueous phase was extracted 3 times with ethyl acetate, then the combined organic phases were washed once with water and once with brine, dried over magnesium sulfate and concentrated. The residue was taken up in MTBE and water, then phases were separated. The organic phase was washed 5 times with water, then once with brine, dried over magnesium sulfate, filtered and concentrated to give an orange oil. The residue was then purified by normal phase flash chromatography (80 g silica column, elution: 0 to 30% MTBE/heptanes over 10 CV). Fractions were combined and concentrated to give impure 4 (883 mg). The residue was then purified by reverse phase flash chromatography (100 g C18 RediSep Rf Gold column, liquid deposit (DMSO), elution: 5% MeOH/0.1% HCOOH over 4 CV, then 5% to 100% MeOH/0.1% HCOOH over 15 CV). Fractions were combined and concentrated to give 4 (618 mg, 41% yield) as a white solid.

LCMS method 1: retention time: 1.745 min, 99.9% purity at 215 nm, [M+H]⁺=304.2.

¹H NMR (400 MHz, DMSO-d6) δ ppm 1.45-1.60 (m, 2H), 1.76-1.89 (m, 2H), 2.00-2.15 (m, 4H), 2.36-2.47 (m, 1H), 3.61 (s, 3H), 4.28-4.40 (m, 1H), 7.14-7.47 (m, 1H), 9.05 (s, 1H).

¹⁹F NMR (377 MHz, DMSO-d6) δ ppm −117.40 (s, 2F).

Step 3. Preparation of Methyl 4-[4-amino-3-(difluoromethyl)pyrazol-1-yl]cyclohexanecarboxylate (T-1). Under nitrogen, a solution of 4 (625 mg, 2.06 mmol, 1.0 eq.), and ethyl acetate (20.6 mL, 0.1 M) was degassed at room temperature by sparging with nitrogen for 15 minutes. Pd/C (438 mg, 10% w/w, 0.41 mmol, 0.2 eq.) was added and sparging was resumed for 15 minutes. The mixture was sparged with H₂ for 15 minutes, then the needle was kept just over the surface of the solvent and the mixture was stirred at room temperature for 16 h. Complete conversion of 4 was observed by LCMS (method 1). The mixture was filtered over celite, then the filter cake was washed thoroughly with ethyl acetate and the resulting solution was concentrated to give T-1 (563 mg, 99% yield) as a light orange solid.

LCMS method 1: retention time: 1.297 min, 99.9% purity at 215 nm, [M+H]+=274.2.

¹H NMR (400 MHz, CDCl₃) δ ppm 1.53-1.80 (m, 5H), 2.12-2.42 (m, 9H), 3.93-4.03 (m, 1H), 6.54-6.84 (m, 1H), 7.07 (s, 1H).

¹⁹F NMR (377 MHz, CDCl₃) δ ppm −112.22 (s, 2F).

### Synthesis of Common Intermediate C-1, C-7, and C-8

### Example I-2. Synthesis of Common Intermediate C-1

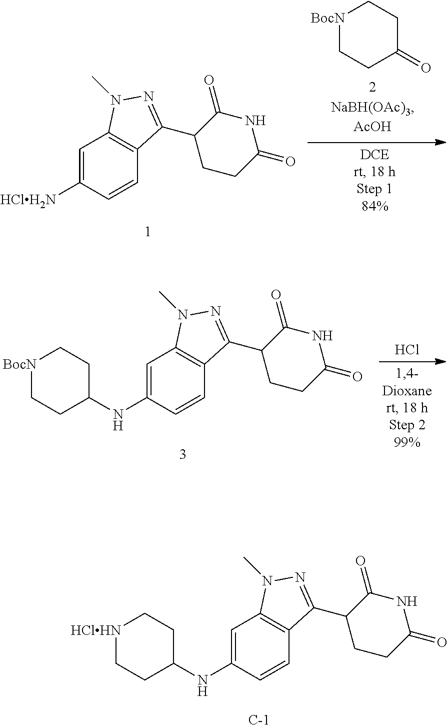

Step 1. Preparation of tert-butyl 4-[[3-(2,6-dioxo-3-piperidyl)-1-methyl-indazol-6-yl]amino]piperidine-1-carboxylate (3). 3-(6-amino-1-methyl-indazol-3-yl)piperidine-2,6-dione hydrochloride (1) (250 mg, 0.848 mmol, 1.0 equiv) and tert-butyl 4-oxopiperidine-1-carboxylate (2) (304 mg, 1.53 mmol, 1.8 equiv) were suspended in DCE (4.2 mL) under nitrogen atmosphere, then acetic acid (146 μL, 2.54 mmol, 3.0 equiv) was added. Sodium triacetoxyborohydride (431 mg, 2.04 mmol, 2.4 equiv) was added in one portion and the reaction mixture was stirred overnight at room temperature under nitrogen atmosphere. The volatiles were evaporated under reduced pressure, then the crude was dissolved in the minimum amount of DMSO and purified by reversed-phase flash chromatography (MeOH in 0.1% HCOOH(aq), 5% (3 CV)→100%, 50 g RediSep Rf Gold® C18, 20 CV, λ=214-254 nm, product with 75% MeOH) to afford, after evaporation of the fractions and lyophilisation, 3 (313.3 mg, 0.710 mmol, 84% yield) as a tan solid.

LCMS method 1: 99.9% purity at 215 nm, [M+H]+=442.2.

¹H NMR (400 MHz, DMSO-d6) δ ppm 1.19-1.32 (m, 2H), 1.41 (s, 9H), 1.94 (br d, J=11.4 Hz, 2H), 2.10-2.19 (m, 1H), 2.20-2.31 (m, 1H), 2.56-2.65 (m, 2H), 2.87-3.05 (m, 2H), 3.47-3.62 (m, 1H), 3.81 (s, 3H), 3.89 (br d, J=13.3 Hz, 2H), 4.18 (dd, J=8.7, 5.1 Hz, 1H), 5.79 (d, J=8.2 Hz, 1H), 6.43 (s, 1H), 6.52 (d, J=9.3 Hz, 1H), 7.33 (d, J=8.7 Hz, 1H), 10.82 (s, 1H).

Step 2. Preparation of 3-[1-methyl-6-(piperidin-1-ium-4-ylamino)indazol-3-yl]piperidine-2,6-dione chloride (C-1). HCl 4.0 M in 1,4-dioxane (2.7 mL, 10.6 mmol, 15.0 equiv) was added to tert-butyl 4-[[3-(2,6-dioxo-3-piperidyl)-1-methyl-indazol-6-yl]amino]piperidine-1-carboxylate (3) (313.3 mg, 0.710 mmol, 1.0 equiv) and the mixture was stirred overnight at room temperature. The solvent was evaporated under reduced pressure and the residue was then dried under high vacuum to remove all the volatiles. The crude product C-1 (265 mg, 0.702 mmol, 99% yield) was obtained as an off-white solid that was used without purification in next step.

LCMS method 1: 99.9% purity at 215 nm, [M−HCl+H]⁺=342.2.

¹H NMR (400 MHz, DMSO-d6) δ ppm 1.61-1.74 (m, 2H), 2.09-2.20 (m, 3H), 2.22-2.36 (m, 1H), 2.55-2.66 (m, 2H), 2.93-3.08 (m, 2H), 3.32 (br d, J=12.5 Hz, 2H), 3.60-3.72 (m, 1H), 3.85 (s, 3H), 4.21 (dd, J=9.0, 5.3 Hz, 1H), 6.63 (br d, J=8.3 Hz, 2H), 7.42 (br d, J=8.7 Hz, 1H), 8.77 (br d, J=7.7 Hz, 1H), 8.88 (br d, J=9.7 Hz, 1H), 10.84 (s, 1H).

The following compounds were synthesized via the same general routes with modifications to ketone 2 in step 1 (Table 2).

TABLE 2

| | Ketone | Structure |
|---|---|---|
| C-1 | 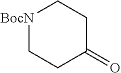 [SMILES: CC(C)(C)OC(=O)N1CCC(=O)CC1] | 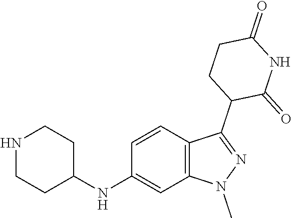 [SMILES: Cn1nc(C2CCC(=O)NC2=O)c2ccc(NC3CCNCC3)cc21] |
| C-7 | 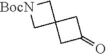 [SMILES: CC(C)(C)OC(=O)N1CC2(CC(=O)C2)C1] | 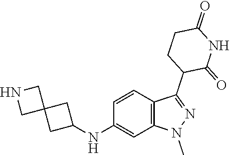 [SMILES: Cn1nc(C2CCC(=O)NC2=O)c2ccc(NC3CC4(CNC4)C3)cc21] |
| C-8 | 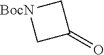 [SMILES: CC(C)(C)OC(=O)N1CC(=O)C1] | 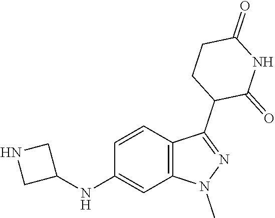 [SMILES: Cn1nc(C2CCC(=O)NC2=O)c2ccc(NC3CNC3)cc21] |

### Example 1-3. Synthesis of Common Intermediate C-2

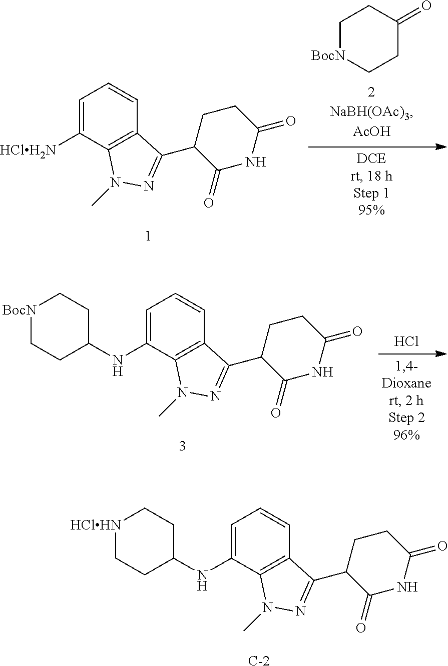

Step 1. Preparation of tert-butyl 4-[[3-(2,6-dioxo-3-piperidyl)-1-methyl-indazol-7-yl]amino]piperidine-1-carboxylate (3). 3-(7-amino-1-methyl-indazol-3-yl)piperidine-2,6-dione hydrochloride (1) (500 mg, 1.70 mmol, 1.0 equiv) and tert-butyl 4-oxopiperidine-1-carboxylate (2) (608 mg, 3.05 mmol, 1.8 equiv) were suspended in DCE (17 mL) under nitrogen atmosphere, then acetic acid (291 μL, 5.09 mmol, 3.0 equiv) was added. Sodium triacetoxyborohydride (863 mg, 4.07 mmol, 2.4 equiv) was added in one portion and the reaction mixture was stirred overnight at room temperature under nitrogen atmosphere. The volatiles were evaporated under reduced pressure, then the crude was dissolved in the minimum amount of DMSO and purified by reversed-phase flash chromatography (MeCN in 0.1% HCOOH(aq), 5% (3 CV)→60%, 100 g RediSep Rf Gold® C18, 20 CV, λ=214-254 nm, product with 55% MeCN) to afford, after evaporation of the fractions and lyophilisation, 3 (702 mg, 1.59 mmol, 95% yield) as a tan solid.

LCMS method 1: 98.5% purity at 215 nm, [M-t-Bu+H]⁺=386.4.

¹H NMR (400 MHz, DMSO-d6) δ ppm 1.41 (s, 9H), 1.42-1.51 (m, 2H), 1.95 (br d, J=12.6 Hz, 2H), 2.10-2.20 (m, 1H), 2.29 (dtd, J=13.7, 9.0, 4.8 Hz, 1H), 2.56-2.73 (m, 2H), 2.80-3.16 (m, 2H), 3.42-3.58 (m, 1H), 3.87 (br d, J=12.6 Hz, 2H), 4.23 (s, 3H), 4.25-4.32 (m, 1H), 4.99 (br d, J=6.6 Hz, 1H), 6.58 (d, J=7.3 Hz, 1H), 6.89 (t, J=7.7 Hz, 1H), 6.99 (d, J=7.9 Hz, 1H), 10.85 (s, 1H).

Step 2. Preparation of 3-[1-methyl-7-(4-piperidylamino)indazol-3-yl]piperidine-2,6-dione hydrochloride (C-2). HCl 4.0 M in 1,4-dioxane (9.7 mL, 38.7 mmol, 15.0 equiv) was added to tert-butyl 4-[[3-(2,6-dioxo-3-piperidyl)-1-methyl-indazol-7-yl]amino]piperidine-1-carboxylate 3 (1.16 g, 2.58 mmol, 1.0 equiv) and the mixture was stirred for 2 hours. The volatiles were evaporated under reduced pressure to yield the crude product as an off-white solid. The crude was dissolved in the minimum amount of DMSO and purified by reversed-phase flash chromatography (MeCN in 0.02 M HCl(aq), 5% (3 CV)→30%, 415 g RediSep Rf Gold® C18, 15 CV, λ=214-254 nm, product with 5-20% MeCN) to afford, after evaporation of the fractions to dryness, co-evaporation of the residue with water (3×25 mL) to remove all the residual HCl, and lyophilisation, C-2 (937 mg, 2.48 mmol, 96% yield) as a tan solid.

LCMS method 1: 99.9% purity at 215 nm, [M+H]⁺=342.4.

¹H NMR (400 MHz, DMSO-d6) δ ppm 1.71-1.89 (m, 2H), 2.03-2.21 (m, 3H), 2.21-2.40 (m, 1H), 2.53-2.73 (m, 2H), 2.92-3.10 (m, 2H), 3.29 (br d, J=11.9 Hz, 2H), 3.63 (br t, J=9.4 Hz, 1H), 4.27 (s, 3H), 4.28-4.37 (m, 1H), 6.76 (br d, J=6.6 Hz, 1H), 6.95 (t, J=7.7 Hz, 1H), 7.15 (br d, J=8.3 Hz, 1H), 8.73-8.97 (m, 1H), 9.10 (br d, J=9.4 Hz, 1H), 10.87 (s, 1H).

### Example I-4. Synthesis of Common Intermediate C-3

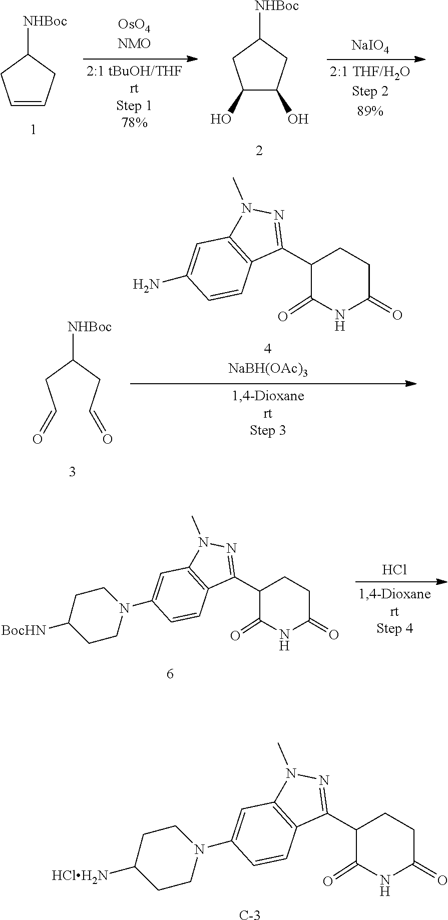

Step 1. Preparation of tert-Butyl N-[3-hydroxy-1-(2-hydroxyethyl)butyl]carbamate (2). Under nitrogen, a solution of 1 (3.0 g, 16.37 mmol, 1.0 eq.), t-BuOH (60.0 mL, 0.18 M) and THF (30.0 mL, 0.18 M) was stirred at room temperature for 5 minutes, then OsO₄ (1.04 mL, 4.0% w/w in H₂O, 0.16 mmol, 0.01 eq.) and NMO (2.30 g, 19.65 mml, 1.2 eq.) were successively added. The resulting mixture was stirred at room temperature for 4 h. Complete conversion of 1 was observed by TLC (heptanes/ethyl acetate 4:1, KMnO₄ stain). The mixture was concentrated to remove t-BuOH and THF, then the residue was dissolved in ethyl acetate (200 mL) and washed with 10% aq. Na₂SO₃ (2×25 mL), sat. aq. NaHCO₃ (25 mL) and sat. aq. NaCl (25 mL). The organic phase was dried over magnesium sulfate, filtered and concentrated to give a white solid. The residue was then purified by normal phase flash chromatography (80 g silica column, elution: 0 to 10% methanol/dichloromethane over 10 CV, product exited at 10% methanol). Fractions were combined and concentrated to give 2 (2.83 g, 78% yield) as a white solid.

¹H NMR (400 MHz, CDCl₃) δ ppm 1.44 (s, 9H), 1.57 (br. s, 1H), 1.70-1.82 (m, 2H), 2.12-2.33 (m, 3H), 2.79-3.05 (m, 1H), 3.79-3.92 (m, 0.5H), 4.00 (br. s, 1H), 4.23 (br. s, 1H), 4.38-4.60 (m, 0.5H), 5.03-5.25 (m, 0.5H).

Step 2. Preparation of tert-Butyl N-[3-oxo-1-(2-oxoethyl)propyl]carbamate (3). Under nitrogen, a solution of 2 (2.83 g, 13.0 mmol, 1.0 eq.), THF (40.0 mL, 0.21 M) and H₂O (20.0 mL, 0.21 M) was stirred at room temperature for 5 minutes, then NaIO₄ (3.34 g, 15.6 mmol, 1.2 eq.) was added. The resulting mixture was stirred at room temperature for 2 h. Complete conversion of 2 was observed by TLC (CH₂Cl₂/MeOH 95:5, KMnO₄ stain). The mixture was concentrated to remove THF. Ethyl acetate (200 mL) and brine (100 mL) were added, then phases were separated and the aqueous phase was extracted 3 times with ethyl acetate. The combined organic phases were washed with brine, dried over magnesium sulfate, filtered and concentrated to give a white semi-solid. The residue was dissolved in dichloromethane (125 mL) and dried for 16 h at room temperature in the presence of a large excess of magnesium sulfate. The solid was filtered, then the organic phase was concentrated and dried under high vacuum to give 3 (2.56 g, 89% yield) as an off-white semi-solid. The product was directly engaged in the next step without further characterization.

Step 3. Preparation of tert-Butyl N-[l-[3-(2,6-dioxo-3-piperidyl)-1-methyl-indazol-6-yl]-4-piperidyl]carbamate (5). To a solution of tert-butyl N-[3-oxo-1-(2-oxoethyl)propyl]carbamate 3 (500 mg, 2.32 mmol, 1.2 eq.) and 3-(6-amino-1-methyl-indazol-3-yl)piperidine-2,6-dione 4 (500 mg, 1.94 mmol, 1 eq.) in DCE (16 mL) was added sodium triacetoxyborohydride (492.4 mg, 2.32 mmol, 1.2 eq.) at rt. The reaction mixture was stirred at rt. After 16 h, LCMS showed 50% conversion. 0.5 eq. of sodium triacetoxyborohydride was additionally added, and stirring was resumed for 4 more hours. At this point, LCMS showed 80% conversion. The reaction mixture was partitioned between DCM and H₂O, then extracted two more times with DCM. Finally, the combined organic layers were dried over Na₂SO₄ and concentrated under reduced pressure. The residue was purified by reverse phase chromatography (150 g C18 RediSep Rf Gold column, liquid deposit (DMSO), elution: 5% MeCN/0.1% HCOOH over 5 CV, then 5 to 100% MeCN/0.1% HCOOH over 15 CV, then 100% MeCN/0.1% HCOOH over 3 CV). Pure fractions were evaporated to give 5 (330 mg, 38% yield) as a light orange solid.

LCMS method 2: 98.7% purity at 215 nm, [M+H]⁺=442.2.

¹H NMR (400 MHz, DMSO-d6) δ ppm 1.39 (s, 9H), 1.42-1.56 (m, 2H), 1.76-1.88 (m, 2H), 2.10-2.20 (m, 1H), 2.20-2.36 (m, 1H), 2.58-2.65 (m, 2H), 2.72-2.85 (m, 2H), 3.35-3.49 (m, 1H), 3.73 (d, J=12.2 Hz, 2H), 3.88 (s, 3H), 4.25 (dd, J=9.0, 5.1 Hz, 1H), 6.84 (s, 1H), 6.85-6.92 (m, 2H), 7.47 (d, J=9.0 Hz, 1H), 10.84 (s, 1H).

Step 4. Preparation of 3-[6-(4-Amino-1-piperidyl)-1-methyl-indazol-3-yl]piperidine-2,6-dione hydrochloride (C-3). To a solution of tert-butyl N-[1-[3-(2,6-dioxo-3-piperidyl)-1-methyl-indazol-6-yl]-4-piperidyl]carbamate 5 (330 mg, 0.75 mmol, 1 eq.) in DCM (5 mL) was added a solution of 4 M HCl in 1,4-dioxane (2.8 mL, 11.21 mmol, 15 eq.) at rt. After 16 h, LCMS showed complete conversion. Volatiles were removed under reduced pressure and excess HCl was co-evaporated four times with DCM, to give C-3 (320 mg, quantitative yield) as an off-white solid. The product was used without purification in the next step.

LCMS method 2: 98.9% purity at 215 nm, [M−HCl+H]⁺=342.2.

¹H NMR (400 MHz, DMSO-d6) δ ppm 1.84-2.01 (m, 2H), 2.06-2.21 (m, 3H), 2.28-2.40 (m, 1H), 2.56-2.74 (m, 2H), 3.07-3.28 (m, 2H), 3.28-3.40 (m, 1H), 3.83 (d, J=11.5 Hz, 2H), 3.94 (s, 3H), 4.32 (dd, J=9.7, 5.0 Hz, 1H), 7.21 (br s, 1H), 7.42 (br s, 1H), 7.66 (d, J=8.1 Hz, 1H), 8.32 (br s, 3H), 10.88 (s, 1H).

### Example I-5. Synthesis of Common Intermediate C-4

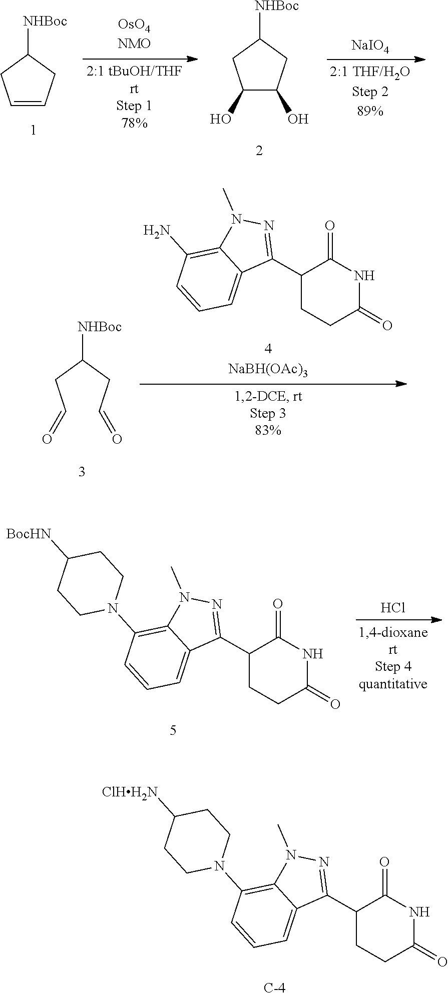

Step 1. Preparation of tert-Butyl N-[3-hydroxy-1-(2-hydroxyethyl)butyl]carbamate (2). Under nitrogen, a solution of 1 (3.0 g, 16.37 mmol, 1.0 eq.), t-BuOH (60.0 mL, 0.18 M) and THF (30.0 mL, 0.18 M) was stirred at room temperature for 5 minutes, then OsO₄ (1.04 mL, 4.0% w/w in H₂O, 0.16 mmol, 0.01 eq.) and NMO (2.30 g, 19.65 mml, 1.2 eq.) were successively added. The resulting mixture was stirred at room temperature for 4 h. Complete conversion of 1 was observed by TLC (heptanes/ethyl acetate 4:1, KMnO₄ stain). The mixture was concentrated to remove t-BuOH and THF, then the residue was dissolved in ethyl acetate (200 mL) and washed with 10% aq. Na₂SO₃ (2×25 mL), sat. aq. NaHCO₃ (25 mL) and sat. aq. NaCl (25 mL). The organic phase was dried over magnesium sulfate, filtered and concentrated to give a white solid. The residue was then purified by normal phase flash chromatography (80 g silica column, elution: 0 to 10% methanol/dichloromethane over 10 CV, product exited at 10% methanol). Fractions were combined and concentrated to give 2 (2.83 g, 78% yield) as a white solid.

¹H NMR (400 MHz, CDCl₃) δ ppm 1.44 (s, 9H), 1.57 (br. s, 1H), 1.70-1.82 (m, 2H), 2.12-2.33 (m, 3H), 2.79-3.05 (m, 1H), 3.79-3.92 (m, 0.5H), 4.00 (br. s, 1H), 4.23 (br. s, 1H), 4.38-4.60 (m, 0.5H), 5.03-5.25 (m, 0.5H).

Step 2. Preparation of tert-Butyl N-[3-oxo-1-(2-oxoethyl)propyl]carbamate (3). Under nitrogen, a solution of 2 (2.83 g, 13.0 mmol, 1.0 eq.), THF (40.0 mL, 0.21 M) and H₂O (20.0 mL, 0.21 M) was stirred at room temperature for 5 minutes, then NaIO₄ (3.34 g, 15.6 mmol, 1.2 eq.) was added. The resulting mixture was stirred at room temperature for 2 h. Complete conversion of 2 was observed by TLC (CH₂Cl₂/MeOH 95:5, KMnO₄ stain). The mixture was concentrated to remove THF. Ethyl acetate (200 mL) and brine (100 mL) were added, then phases were separated and the aqueous phase was extracted 3 times with ethyl acetate. The combined organic phases were washed with brine, dried over magnesium sulfate, filtered and concentrated to give a white semi-solid. The residue was dissolved in dichloromethane (125 mL) and dried for 16 h at room temperature in the presence of a large excess of magnesium sulfate. The solid was filtered, then the organic phase was concentrated and dried under high vacuum to give 3 (2.56 g, 89% yield) as an off-white semi-solid. The product was directly engaged in the next step without further characterization.

Step 3. Preparation of tert-Butyl N-[1-[3-(2,6-dioxo-3-piperidyl)-1-methyl-indazol-7-yl]-4-piperidyl]carbamate (5). Under nitrogen, a solution of 3 (400 mg, 1.86 mmol, 1.2 eq.), 4 (400 mg, 1.55 mmol, 1.0 eq.) and 1,2-DCE (15.5 mL, 0.1 M) was stirred at room temperature for 5 minutes, then NaBH(OAc)₃ (788 mg, 3.72 mmol, 2.4 eq.) was added and the mixture was stirred at room temperature for 16 h. Incomplete conversion (approximately 70%) was observed by LCMS (method 1), then a second portion of 3 (100 mg, 0.47 mmol, 0.3 eq.) and NaBH(OAc)₃ (788 mg, 3.72 mmol, 2.4 eq.) was added and stirring was resumed for 2.5 h. Complete conversion of 4 was observed by LCMS (method 1). The reaction was quenched by addition of sat. aq. NH₄Cl and was then extracted 3 times with dichloromethane. The combined organic phases were washed once with brine, dried over magnesium sulfate, filtered and concentrated to give a light-yellow oil. The residue was purified by reverse phase flash chromatography (50 g C18 RediSep Rf Gold column, liquid deposit (DMSO), elution: 5% MeCN/0.1% HCOOH over 4 CV, then 5% to 100% MeCN/0.1% HCOOH over 15 CV, product exited at 80% MeCN). Fractions were combined and concentrated to give 5 (572 mg, 83% yield) as an off-white solid.

LCMS method 1: retention time: 1.719 min, 99.9% purity at 215 nm, [M+H]⁺=442.2.

¹H NMR (400 MHz, CDCl₃) δ ppm 1.39-1.51 (m, 9H), 1.53-1.71 (m, 3H), 2.02 (s, 2H), 2.09-2.18 (m, 1H), 2.31-2.43 (m, 1H), 2.44-2.56 (m, 1H), 2.62-2.73 (m, 1H), 2.79-2.88 (m, 1H), 2.92-3.04 (m, 1H), 3.29 (br. d, J=10.8 Hz, 1H), 3.55-3.68 (m, 1H), 4.24-4.36 (m, 4H), 6.99-7.10 (m, 2H), 7.37 (d, J=7.8 Hz, 1H), 7.95 (s, 1H), 9.75 (s, 1H).

Step 4. Preparation of [1-[3-(2,6-Dioxo-3-piperidyl)-1-methyl-indazol-7-yl]-4-piperidyl]ammonium chloride (C-4). Under nitrogen, a solution of 5 (572 mg, 1.30 mmol, 1.0 eq.) and 4.0 M HCl in 1,4-dioxane (6.5 mL, 20 eq.) was stirred at room temperature for 2 h. Complete conversion of 5 was observed by LCMS (method 1). The mixture was concentrated under reduced pressure and co-evaporated 3 times with acetonitrile to give C-4 (525 mg, quantitative yield) as an off-white solid.

LCMS method 1: retention time: 1.149 min, 96.0% purity at 215 nm, [M+H]⁺=342.4.

¹H NMR (400 MHz, DMSO-d6) δ ppm 1.74-1.90 (m, 2H), 2.01-2.06 (m, 1H), 2.12-2.23 (m, 1H), 2.25-2.41 (m, 2H), 2.57-2.70 (m, 2H), 2.71-2.85 (m, 2H), 3.11-3.33 (m, 3H), 4.25 (s, 3H), 4.31-4.38 (m, 1H), 6.98-7.06 (m, 2H), 7.38-7.44 (m, 1H), 7.98-8.12 (m, 3H), 10.88 (s, 1H).

### Example I-6. Synthesis of Common Intermediate C-5

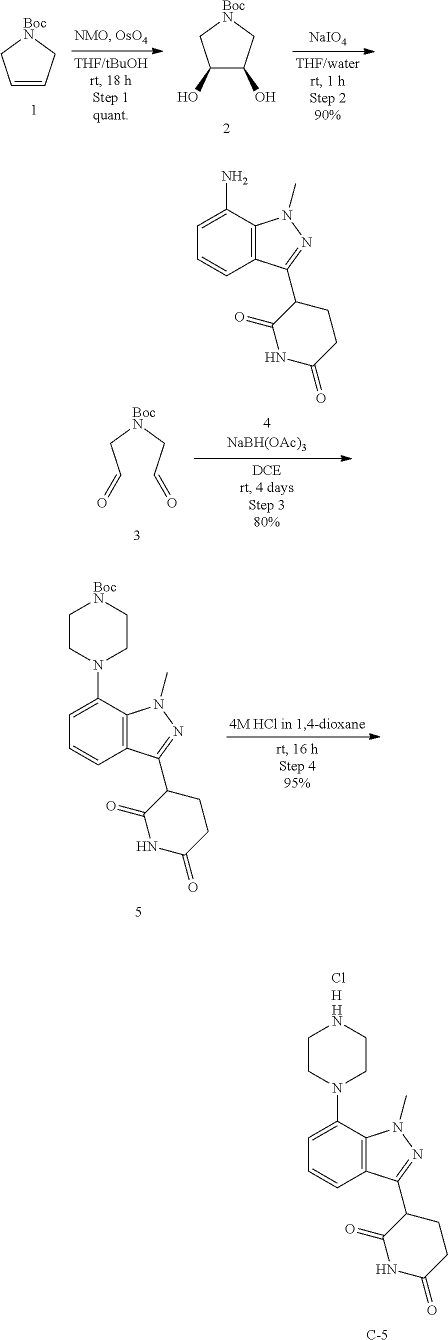

Step 1. Preparation of tert-Butyl (3R,4S)-3,4-dihydroxypyrrolidine-1-carboxylate (2). To a solution of tert-butyl 2,5-dihydropyrrole-1-carboxylate 1 (2.5 g, 14.77 mmol, 1 eq.) in tert-butanol (50 mL) and THF (25 mL) was added osmium tetroxide (0.94 mL, 0.15 mmol, 0.01 eq.) followed by NMO (2.08 g, 17.73 mmol, 1.2 eq.) at room temperature. The reaction was stirred at rt for 18 h then concentrated to dryness. The residue was dissolved in EtOAc (200 mL) and washed with 10% Na₂SO₃ (2×20 mL), sat. NaHCO₃ (20 mL) and brine (20 mL). The organic phase was dried over Na₂SO₄ and conc. in vacuo. The residue was then purified by normal phase flash chromatography (0 to 10% MeOH/CH₂Cl₂, 10 CV, 120 g silica) to give 2 (3.04 g, quant.) as a yellow oil.

¹H NMR (400 MHz, chloroform-d) δ ppm 1.46 (s, 9H), 2.76-2.83 (m, 1H), 2.87-2.95 (m, 1H), 3.28-3.40 (m, 2H), 3.52-3.64 (m, 2H), 4.20-4.28 (m, 2H).

Step 2. Preparation of tert-Butyl N,N-bis(2-oxoethyl)carbamate (3). To a solution of tert-butyl (3R,4S)-3,4-dihydroxypyrrolidine-1-carboxylate 2 (3.04 g, 14.94 mmol, 1 eq.) in THF (50 mL) and water (25 mL) was added sodium periodate (3.84 g, 17.93 mmol, 1.2 eq.). The reaction was stirred for 1 h at room temperature. TLC (5% MeOH/CH₂Cl₂, vanillin stain) showed complete conversion. THE was removed in vacuo and brine (50 mL) was added. The mixture was extracted with EtOAc (3×100 mL). The combined organics were dissolved over Na₂SO₄ and conc. in vacuo. The residue was dissolved in CH₂Cl₂ (150 mL) and stirred overnight over MgSO₄. The MgSO₄ was then filtered off and the CH₂Cl₂ conc. in vacuo to afford the desired product 3 (2.7 g, 90% yield) as a light-yellow oil.

¹H NMR (400 MHz, chloroform-d) δ ppm 1.46 (s, 9H), 3.97 (s, 2H), 4.18 (s, 2H), 9.65 (s, 1H), 9.67 (s, 1H).

Step 3. Preparation of tert-Butyl 4-[3-(2,6-dioxo-3-piperidyl)-1-methyl-indazol-7-yl]piperazine-1-carboxylate (5). Sodium triacetoxyborohydride (517.73 mg, 2.44 mmol, 2.4 eq.) was added to a solution of tert-butyl N,N-bis(2-oxoethyl)carbamate 3 (245.77 mg, 1.22 mmol, 1.2 eq.) and 3-(7-amino-1-methyl-indazol-3-yl)piperidine-2,6-dione 4 (262.89 mg, 1.02 mmol, 1 eq.) in DCE (10.2 mL), then the mixture was stirred for 24 h. LCMS showed 40% of 3. Another portion of sodium triacetoxyborohydride (517.73 mg, 2.44 mmol, 2.4 eq.) was added and the stirring was continued for 24 h, LCMS showed 46% of 3. After repeating the addition of sodium triacetoxyborohydride and stirring for 24 h two more times, LCMS showed 90% of 3. The reaction mixture was quenched with saturated NH₄Cl (aq) (20 mL) and extracted with CH₂Cl₂ (3×20 mL). The combined organic layers were dried over MgSO₄ and concentrated under reduced pressure. The crude was dissolved in the minimum amount of DMSO and purified by reverse phase flash chromatography (MeCN in 0.1% HCOOH(aq), 5% to 100%, 100 g C18 gold column, 20 CV, product with 65-70% MeCN). Evaporation of the fractions gave 5 (373 mg, 80% yield) as a white solid.

LCMS method 1: 89.0% purity at 215 nm [M+H]⁺=428.2

¹H NMR (400 MHz, DMSO-d6) δ ppm 1.43 (s, 9H), 2.11-2.22 (m, 1H), 2.26-2.40 (m, 1H), 2.55-2.79 (m, 4H), 3.00-3.23 (m, 4H), 3.88-4.07 (m, 2H), 4.25 (s, 3H), 4.34 (dd, J=9.7, 5.0 Hz, 1H), 7.00-7.07 (m, 2H), 7.41 (d, J=7.6 Hz, 1H), 10.88 (s, 1H).

Step 4. Preparation of 3-(1-methyl-7-piperazin-1-yl-indazol-3-yl)piperidine-2,6-dione (C-5). To tert-butyl 4-[3-(2,6-dioxo-3-piperidyl)-1-methyl-indazol-7-yl]piperazine-1-carboxylate 5 (373. mg, 0.87 mmol, 1 eq.) was added 4 M HCl in 1,4-dioxane (6.54 mL, 26.15 mmol, 30 eq.). The solution was stirred at room temperature for 16 h. Then solvent was removed under reduce pressure and water was added. The product was purified by reverse phase flash chromatography (MeCN in 0.1% HCOOH(aq), 5% to 40%, 100 g gold column C18, 20 CV) to give C-5 (270 mg, 95% yield) as a tan solid.

LCMS method 1: 99.9% purity at 215 nm [M+H]⁺=328.4.

¹H NMR (400 MHz, DMSO-d6) δ ppm 2.12-2.23 (m, 1H), 2.27-2.40 (m, 1H), 2.56-2.74 (m, 2H), 2.77-2.96 (m, 2H), 3.03-3.23 (m, 6H), 4.26 (s, 3H), 4.35 (dd, J=9.7, 5.0 Hz, 1H), 7.05 (d, J=4.4 Hz, 2H), 7.40-7.45 (m, 1H), 10.89 (s, 1H).

### Example I-7. Synthesis of Common Intermediate C-6

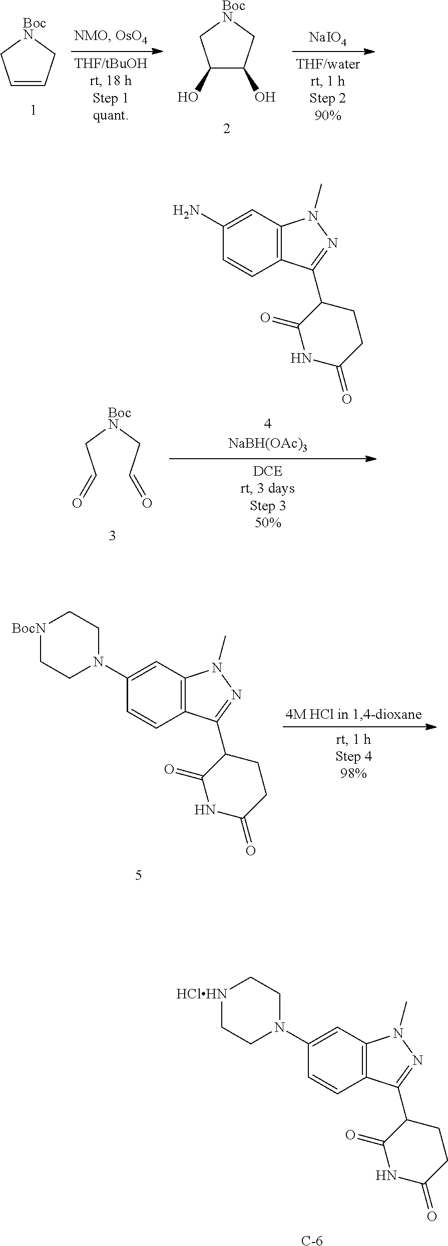

Step 1. Preparation of tert-Butyl (3R,4S)-3,4-dihydroxypyrrolidine-1-carboxylate (2). To a solution of tert-butyl 2,5-dihydropyrrole-1-carboxylate 1 (2.5 g, 14.77 mmol, 1 eq.) in tert-butanol (50 mL) and THF (25 mL) was added osmium tetroxide (0.94 mL, 0.15 mmol, 0.01 eq.) followed by NMO (2.08 g, 17.73 mmol, 1.2 eq.) at room temperature. The reaction was stirred at rt for 18 h then concentrated to dryness. The residue was dissolved in EtOAc (200 mL) and washed with 10% Na₂SO₃ (2×20 mL), sat. NaHCO₃ (20 mL) and brine (20 mL). The organic phase was dried over Na₂SO₄ and conc. in vacuo. The residue was then purified by normal phase flash chromatography (0 to 10% MeOH/CH₂Cl₂, 10 CV, 120 g silica) to give 2 (3.04 g, quant.) as a yellow oil.

¹H NMR (400 MHz, chloroform-d) δ ppm 1.46 (s, 9H), 2.76-2.83 (m, 1H), 2.87-2.95 (m, 1H), 3.28-3.40 (m, 2H), 3.52-3.64 (m, 2H), 4.20-4.28 (m, 2H).

Step 2. Preparation of tert-Butyl N,N-bis(2-oxoethyl)carbamate (3). To a solution of tert-butyl (3R,4S)-3,4-dihydroxypyrrolidine-1-carboxylate 2 (3.04 g, 14.94 mmol, 1 eq.) in THF (50 mL) and water (25 mL) was added sodium periodate (3.84 g, 17.93 mmol, 1.2 eq.). The reaction was stirred for 1 h at room temperature. TLC (5% MeOH/CH₂Cl₂, vanillin stain) showed complete conversion. THE was removed in vacuo and brine (50 mL) was added. The mixture was extracted with EtOAc (3×100 mL). The combined organics were dissolved over Na₂SO₄ and conc. in vacuo. The residue was dissolved in CH₂Cl₂ (150 mL) and stirred overnight over MgSO₄. The MgSO₄ was then filtered off and the CH₂Cl₂ conc. in vacuo to afford the desired product 3 (2.7 g, 90% yield) as a light-yellow oil.

¹H NMR (400 MHz, chloroform-d) δ ppm 1.46 (s, 9H), 3.97 (s, 2H), 4.18 (s, 2H), 9.65 (s, 1H), 9.67 (s, 1H).

Step 3. Preparation of tert-Butyl 4-[3-(2,6-dioxo-3-piperidyl)-1-methyl-indazol-6-yl]piperazine-1-carboxylate (5). Sodium triacetoxyborohydride (646.71 mg, 3.05 mmol, 2.4 eq.) was added to a solution of tert-butyl N,N-bis(2-oxoethyl)carbamate 3 (307.0 mg, 1.53 mmol, 1.2 eq.) and 3-(6-amino-1-methyl-indazol-3-yl)piperidine-2,6-dione 4 (328.38 mg, 1.27 mmol, 1 eq.) in DCE (12.7 mL), then the mixture was stirred at room temperature for 24 h. LCMS showed 26% of 5. Another portion of sodium triacetoxyborohydride (646.71 mg, 3.05 mmol, 2.4 eq.) was added to the mixture and the stirring was continued for 24 h, LCMS showed 60% of 5. Another portion of sodium triacetoxyborohydride (646.71 mg, 3.05 mmol, 2.4 eq.) was added to the mixture and the stirring was continued for 24 h, LCMS showed no change. The reaction mixture was quenched with saturated NH₄Cl (aq) (20 mL) and extracted with CH₂Cl₂ (3×20 mL). The combined organic layers were dried over MgSO₄ and concentrated under reduced pressure. The crude was dissolved in the minimum amount of DMSO and purified by reverse phase flash chromatography (MeCN in 0.1% HCOOH, 5% to 100%, 100 g gold column, 20 CV, product with 65-70% MeCN). Concentration of the fractions gave 5 (263 mg, 50% yield) as an off-white solid.

LCMS method 1: 99.9% purity at 215 nm [M+H]⁺=428.4.

¹H NMR (400 MHz, DMSO-d6) δ ppm 1.38-1.46 (m, 9H), 2.10-2.21 (m, 1H), 2.24-2.37 (m, 1H), 2.54-2.69 (m, 2H), 3.12-3.24 (m, 4H), 3.43-3.56 (m, 4H), 3.90 (s, 3H), 4.26 (dd, J=9.2, 5.0 Hz, 1H), 6.89 (s, 1H), 6.92 (br d, J=9.0 Hz, 1H), 7.52 (d, J=9.0 Hz, 1H), 10.85 (s, 1H).

Step 4. Preparation of 3-(1-methyl-6-piperazin-1-yl-indazol-3-yl)piperidine-2,6-dione hydrochloride (C-6). 4 M HCl in 1,4-dioxane (22.78 mL, 91.14 mmol, 150 eq.) was added to tert-butyl 4-[3-(2,6-dioxo-3-piperidyl)-1-methyl-indazol-6-yl]piperazine-1-carboxylate 5 (260.0 mg, 0.61 mmol, 1 eq.) at room temperature. Then, the mixture was sonicated for 30 minutes and then stirred for one hour at room temperature. Solvents were concentrated to dryness and the residue was purified by reversed-phase flash chromatography (MeCN in 0.02 M HCl(aq), 5% to 30%, 100 g gold column C18, 15 CV, product with 0-20% MeCN). The fractions containing the product were evaporated to dryness, then co-evaporated with water (3×10 mL) to completely remove any trace of HCl, and the residue was lyophilized to give C-6 (221 mg, 98%) as a light-yellow solid.

LCMS method 1: 99.9% purity at 215 nm [M−HCl+H]⁺=328.4.

¹H NMR (400 MHz, DMSO-d6) δ ppm 2.08-2.22 (m, 1H), 2.24-2.37 (m, 1H), 2.54-2.70 (m, 2H), 3.24 (br s, 4H), 3.45-3.50 (m, 4H), 3.92 (s, 3H), 4.28 (dd, J=9.4, 5.0 Hz, 1H), 6.93-6.99 (m, 2H), 7.56 (d, J=8.8 Hz, 1H), 9.37 (br s, 2H), 10.85 (s, 1H).

### Example I-8. Synthesis of Common Intermediate C-9

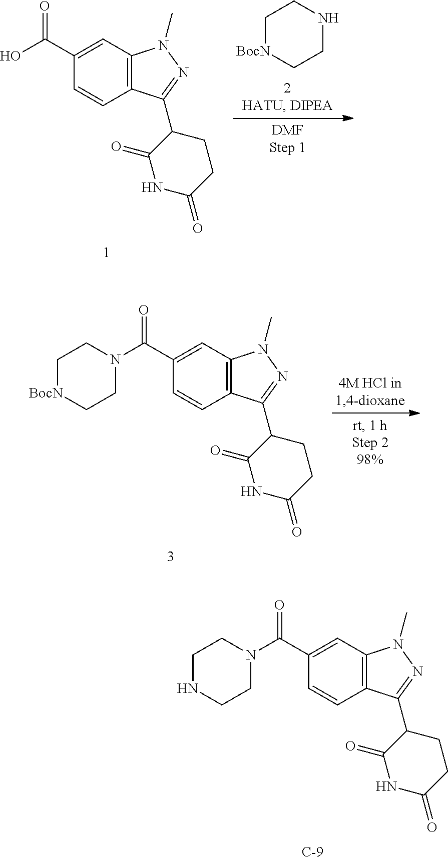

Step 1. Preparation of tert-butyl 4-(3-(2,6-dioxopiperidin-3-yl)-1-methyl-11H-indazole-6-carbonyl)piperazine-1-carboxylate (3). tert-butyl Piperazine-1-carboxylate 2 (67.43 mg, 0.3600 mmol) and 3-(2,6-dioxo-3-piperidyl)-1-methyl-indazole-6-carboxylic acid 1 (80. mg, 0.2800 mmol) were dissolved in DMF (1 mL) under nitrogen atmosphere, then DIPEA (485.09 μL, 2.78 mmol) and HATU (127.07 mg, 0.3300 mmol) were added in sequence and the reaction mixture was stirred overnight under nitrogen atmosphere. Upon observation of complete conversion by LCMS, the reaction mixture was injected directly into a column for purification by reversed-phase flash chromatography (MeCN in 0.1% HCOOH(aq), 5% to 50%, to afford product 3 (110 mg, 83% yield) as a white solid.

LCMS method 1: 96.3% purity at 215 nm, RT=1.55, [M+H]⁺=456.2, [M-t-Bu+H]⁺=400.2.

Step 2. Preparation of 3-(1-methyl-6-(piperazine-1-carbonyl)-1H-indazol-3-yl)piperidine-2,6-dione (C-9). HCl 4.0 M in dioxane (2.83 mL, 11.31 mmol) was added to tert-butyl 4-[3-(2,6-dioxo-3-piperidyl)-1-methyl-indazole-6-carbonyl]piperazine-1-carboxylate 3 (107. mg, 0.2300 mmol) and the mixture was sonicated for 30 minutes, then stirred for 1 hour. The volatiles were evaporated to dryness to yield product C-9 (90 mg, 99% yield) as a white solid.

LCMS method 1: 99.9% purity at 215 nm, RT=0.70, [M+H]⁺=356.2.

Synthesis of common intermediate C-10, C-11, C-12, C-13, C-18, and C-19

### Example I-9. Synthesis of Common Intermediate C-13

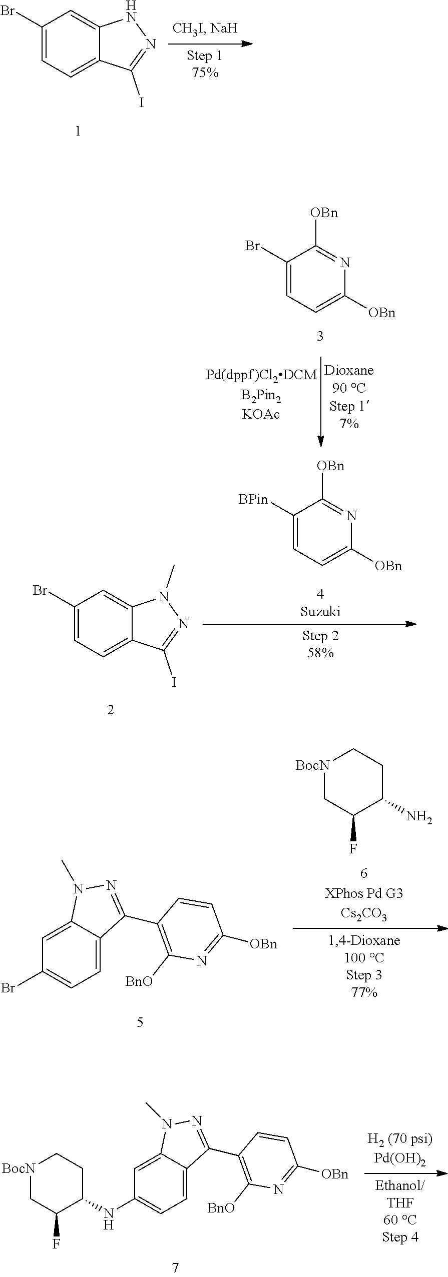

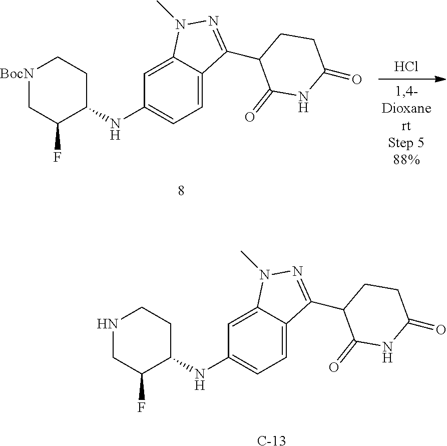

Step 1. Preparation of 6-bromo-3-iodo-1-methyl-indazole (2). To a flame-dried round bottom flask was added sodium hydride 60% w/w in mineral oil (127.45 mg, 3.19 mmol, 1.5 eq.) in DMF (15 mL, 0.14 M). Then, 6-bromo-3-iodo-1H-indazole 1 (700 mg, 2.12 mmol, 1 eq.) was added in one portion and the mixture was stirred for 30 minutes at room temperature. Iodomethane (264 μL, 4.25 mmol, 2 eq.) was then added to the reaction mixture which was stirred at room temperature under nitrogen atmosphere. LCMS after 20 h showed complete conversion into compound 2. Water (50 mL) was added to the reaction mixture and the aqueous phase was extracted with EtOAc (3×50 mL). The organic layers were combined, washed with water (2×50 mL), 1:1 water/brine (2×50 mL) and brine (50 mL), dried over MgSO₄ and evaporated to dryness. The residue was then purified by normal phase FC (80 g gold column, solid deposit on silica, 0 to 15% EtOAc/Heptane over 15 CV, product came out around 7% EtOAc). Fractions were combined and concentrated to give 3 (537 mg, 75% yield) as a white solid.

LCMS method 3: 99.9% purity at 215 nm, [M+H]⁺=336.9; 338.9.

¹H NMR (400 MHz, DMSO-d6) δ ppm 4.05 (s, 3H), 7.31-7.40 (m, 2H), 8.03-8.09 (m, 1H).

Step 1′. Preparation of 2,6-dibenzyloxy-3-(4,4,5,5-tetramethyl-1,3,2-dioxaborolan-2-yl)pyridine (4). To a sealed tube were added 2,6-dibenzyloxy-3-bromo-pyridine 3 (5.5 g, 14.86 mmol, 1 eq.), B2Pin2 (7.5 g, 29.53 mmol, 2 eq.) and KOAc (4.37 g, 44.57 mmol, 3 eq.) in 1,4-dioxane (90 mL, 0.17 M). The solution was bubbled with nitrogen for 15 minutes, then Pd(dppf)Cl₂ DCM (1.21 g, 1.49 mmol, 0.1 eq.) was added. Nitrogen was bubbled through the reaction mixture for 15 minutes under sonication. The tube was sealed and the reaction mixture was stirred at 90° C. After an overnight period, LCMS showed good conversion into compound 4. The reaction mixture was cooled to room temperature, and EtOAc and water were added. The aqueous phase was extracted 3× with EtOAc. The organic phases were combined, washed with brine, dried over Na₂SO₄, filtered and evaporated under reduce pressure. The crude material was then purified by normal phase flash chromatography (120 g silica column, solid deposit on silica, elution: 0 to 30% EtOAc/heptanes over 18 CV, product came around 15% EtOAc). Fractions were combined and concentrated to give 4 (452 mg, 7% yield) as a white solid.

LCMS method 4: 96.1% purity at 215 nm, [M+H]⁺=418.2.

¹H NMR (400 MHz, DMSO-d6) δ ppm 1.28 (s, 12H), 5.38 (d, J=5.9 Hz, 4H), 6.42 (d, J=7.8 Hz, 1H), 7.25-7.40 (m, 6H), 7.42 (d, J=7.6 Hz, 2H), 7.53 (d, J=7.1 Hz, 2H), 7.85 (d, J=8.1 Hz, 1H).

Step 2. Preparation of 6-bromo-3-(2,6-dibenzyloxy-3-pyridyl)-1-methyl-indazole (5). In a sealed tube were added 6-bromo-3-iodo-1-methyl-indazole 2 (0.88 g, 2.61 mmol, 1 eq.), 2,6-dibenzyloxy-3-(4,4,5,5-tetramethyl-1,3,2-dioxaborolan-2-yl)pyridine 4 (1.09 g, 2.61 mmol, 1 eq.), Na₂CO₃ (0.66 g, 6.26 mmol, 2.4 eq.) in 1,4-dioxane (7.8 mL, 0.25 M) and water (2.6 mL, 0.25 M) was added PdCl₂(dppf) CH₂Cl₂ (106 mg, 0.13 mmol, 0.05 eq.). The solution was degassed for 15 min with nitrogen, the tube was sealed, and the reaction mixture was stirred at 90° C. After an overnight period, LCMS showed complete conversion into compound 5. After cooling to room temperature, water was added to the reaction mixture. The reaction mixture was then extracted 3× with EtOAc, the organic phases were combined, washed with water, washed with brine, dried over Na₂SO₄, filtered and evaporated under reduce pressure. The crude material was then purified by normal phase flash chromatography (40 g silica column, solid deposit on silica, elution: 0 to 20% EtOAc/heptanes over 17 CV, product came around 17% EtOAc). Fractions were combined and concentrated to give 5 (810 mg, 58% yield) as a white solid.

LCMS method 3: 94.1% purity at 215 nm, [M+H]⁺=500.1.

¹H NMR (400 MHz, DMSO-d6) δ ppm 4.05 (s, 3H), 5.44 (d, J=8.1 Hz, 4H), 6.60 (d, J=8.1 Hz, 1H), 7.12 (dd, J=8.7, 1.6 Hz, 1H), 7.25-7.43 (m, 8H), 7.44-7.50 (m, 2H), 7.63 (d, J=8.8 Hz, 1H), 7.91 (d, J=8.1 Hz, 1H), 7.95-7.98 (m, 1H).

Step 3. Preparation of tert-butyl (3S,4S)-4-[[3-(2,6-dibenzyloxy-3-pyridyl)-1-methyl-indazol-6-yl]amino]-3-fluoro-piperidine-1-carboxylateindazole (7). In a sealed tube were added 6-bromo-3-(2,6-dibenzyloxy-3-pyridyl)-1-methyl-indazole 5 (300 mg, 0.60 mmol, 1 eq.), tert-butyl (3S,4S)-4-amino-3-fluoro-piperidine-1-carboxylate 6 (196.29 mg, 0.90 mmol, 1.5 eq.), XPhos Pd G3 (50.74 mg, 0.06 mmol, 0.1 eq.), and Cs₂CO₃ (390.68 mg, 1.2 mmol, 2 eq.) in 1,4-dioxane (3.0 mL, 0.2 M). The solution was degassed for 15 min with nitrogen, the tube was sealed and the reaction mixture was stirred at 100° C. After an overnight period, LCMS showed complete conversion into compound 7. After cooling to room temperature, the reaction mixture was filtered through a pad of celite, rinsed with DCM, the solution was evaporated and purified by normal phase flash chromatography (40 g silica column, solid deposit on silica, elution: 0 to 40% EtOAc/heptanes over 20 CV, product came around 20% EtOAc). Fractions were combined and concentrated to give 7 (294 mg, 77% yield) as a white solid.

LCMS method 3: 99.9% purity at 215 nm, product does not ionize.

¹H NMR (400 MHz, DMSO-d6) δ ppm 1.42 (s, 9H), 1.97-2.06 (m, 2H), 3.19-3.28 (m, 1H), 3.58-3.68 (m, 1H), 3.73-3.88 (m, 2H), 3.90 (s, 3H), 4.36-4.55 (m, 1H), 5.43 (d, J=11.7 Hz, 4H), 5.97 (d, J=8.3 Hz, 1H), 6.45-6.52 (m, 2H), 6.55 (d, J=8.1 Hz, 1H), 7.26-7.43 (m, 10H), 7.46 (d, J=7.2 Hz, 2H), 7.86 (d, J=8.1 Hz, 1H).

¹⁹F NMR (377 MHz, DMSO-d6) δ ppm −185.84-−185.48 (m, 1F).

Step 4. Preparation of tert-butyl (3S,4S)-4-[[3-(2,6-dibenzyloxy-3-pyridyl)-1-methyl-indazol-6-yl]amino]-3-fluoro-piperidine-1-carboxylateindazole (8). To a degassed solution of tert-butyl (3S,4S)-4-[[3-(2,6-dibenzyloxy-3-pyridyl)-1-methyl-indazol-6-yl]amino]-3-fluoro-piperidine-1-carboxylate 7 (294 mg, 0.46 mmol, 1 eq.) in THF (1.5 mL, 0.15 M) and Ethanol (1.5 mL, 0.15 M) was added Pd(OH)₂ (64.74 mg, 0.09 mmol, 0.2 eq.) in a reactor linked to a pressure valve. Purging cycles with N₂ and H₂ were performed. The mixture was stirred overnight at 60° C. under a pressure of 70 psi. The reaction mixture was filtered over a pad of celite, washed with EtOAc and EtOH, and concentrated under reduce pressure. The residue was directly loaded for reverse phase FC purification (50 g RediSep Rf Gold C18 column, liquid deposit (DMSO), 5% MeCN/0.1% HCOOH over 4 CV, then 5 to 95% MeCN/0.1% HCOOH over 20 CV, product came out at 65% MeCN). Pure fractions were combined and concentrated to give 8 (100 mg, 47% yield) as a blue solid.

LCMS method 3: 99.9% purity at 215 nm, [M+H]⁺=460.2.

¹H NMR (400 MHz, DMSO-d6) δ ppm 1.42 (s, 9H), 1.96-2.06 (m, 1H), 2.08-2.32 (m, 3H), 2.58-2.63 (m, 2H), 3.19-3.29 (m, 2H), 3.59-3.66 (m, 1H), 3.74-3.81 (m, 1H), 3.82 (s, 3H), 3.84-3.93 (m, 1H), 4.19 (dd, J=8.8, 4.9 Hz, 1H), 4.37-4.55 (m, 1H), 5.98 (d, J=8.3 Hz, 1H), 6.51 (s, 1H), 6.55-6.59 (m, 1H), 7.35 (d, J=8.6 Hz, 1H), 10.82 (s, 1H).

¹⁹F NMR (377 MHz, DMSO-d6) δ ppm −185.89-−185.48 (m, 1F).

Step 5. Preparation of 3-[1-methyl-6-[[(3S,4S)-3-fluoro-4-piperidyl]amino]indazol-3-yl]piperidine-2,6-dione;dihydrochloride (C-13). To tert-butyl (3S,4S)-4-[[3-(2,6-dioxo-3-piperidyl)-1-methyl-indazol-6-yl]amino]-3-fluoro-piperidine-1-carboxylate 8 (95 mg, 0.21 mmol, 1 eq.) was added 4 μM HCl in dioxane (0.4 mL, 1.65 mmol, 8.0 equiv.). The reaction mixture was stirred at rt for 2 h. Upon completion, the solvent was removed under reduced pressure, and the residue was co-evaporated with MeCN (2×). The residue was dried under high vacuum to give C-13 (114 mg, 88% yield) as a white solid as a dihydrochloride salt. The product was used in the next step without further purification.

LCMS method 3: 68.8% purity at 215 nm, [M+H]⁺=360.1.

The following compounds were synthesized via the same general routes with modifications to amine 6 in step 3 (Table 3).

TABLE 3

| | Amine | Structure |
|---|---|---|
| C-10 | 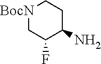 [SMILES: CC(C)(C)OC(=O)N1CC[C@@H](N)[C@H](F)C1] | 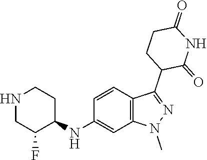 [SMILES: Cn1nc(C2CCC(=O)NC2=O)c2ccc(N[C@@H]3CCNC[C@H]3F)cc21] |
| C-11 | 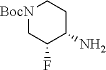 [SMILES: CC(C)(C)OC(=O)N1CC[C@H](N)[C@H](F)C1] | 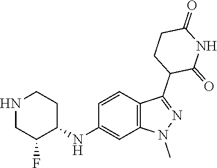 [SMILES: Cn1nc(C2CCC(=O)NC2=O)c2ccc(N[C@H]3CCNC[C@H]3F)cc21] |
| C-12 | 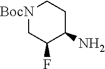 [SMILES: CC(C)(C)OC(=O)N1CC[C@@H](N)[C@@H](F)C1] | 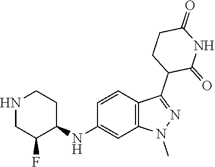 [SMILES: Cn1nc(C2CCC(=O)NC2=O)c2ccc(N[C@@H]3CCNC[C@@H]3F)cc21] |
| C-13 | 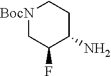 [SMILES: CC(C)(C)OC(=O)N1CC[C@H](N)[C@@H](F)C1] | 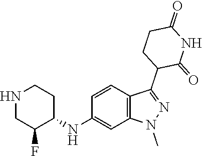 [SMILES: Cn1nc(C2CCC(=O)NC2=O)c2ccc(N[C@H]3CCNC[C@@H]3F)cc21] |
| C-18 | 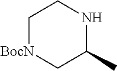 | 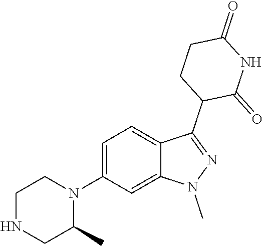 [SMILES: C[C@H]1CNCCN1c1ccc2c(C3CCC(=O)NC3=O)nn(C)c2c1] |
| C-19 | 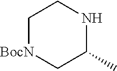 | 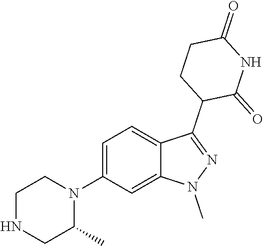 [SMILES: C[C@@H]1CNCCN1c1ccc2c(C3CCC(=O)NC3=O)nn(C)c2c1] |

### Example I-10. Synthesis of Common Intermediate C-14

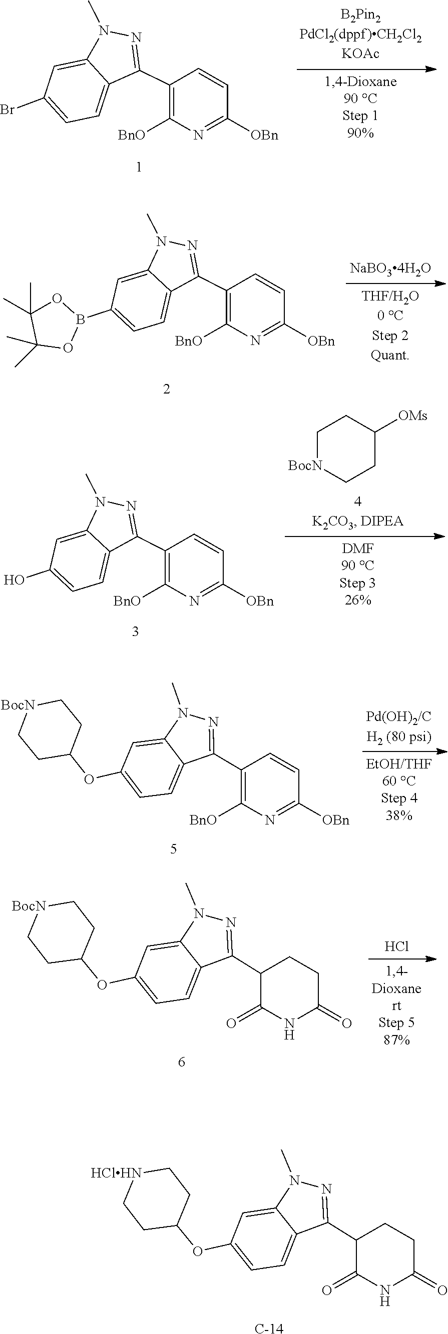

Step 1. 3-(2,6-dibenzyloxy-3-pyridyl)-1-methyl-6-(4,4,5,5-tetramethyl-1,3,2-dioxaborolan-2-yl)indazole (2). To a sealed tube were added 6-bromo-3-(2,6-dibenzyloxy-3-pyridyl)-1-methyl-indazole 1—intermediate 5 from synthesis of C-13—(300 mg, 0.58 mmol, 1 eq.), B2Pin2 (219.92 mg, 0.87 mmol, 1.5 eq.) and KOAc (283.31 mg, 2.89 mmol, 5 eq.) in 1,4-dioxane (1.44 mL, 0.4 M). The solution was bubbled with nitrogen for 15 minutes, then Pd(dppf)Cl₂ DCM (42.24 g, 0.06 mmol, 0.1 eq.) was added. Nitrogen was bubbled through the reaction mixture for 15 minutes under sonication. The tube was sealed and the reaction mixture was stirred at 80° C. After an overnight period, LCMS showed good conversion into compound 2. The reaction mixture was cooled to room temperature, filtered through a pad of celite, rinsed with methanol and evaporated. The crude material was then purified by normal phase flash chromatography (40 g silica column, solid deposit on silica, elution: 0 to 30% EtOAc/heptanes over 20 CV, product came around 10% EtOAc). Fractions were combined and concentrated to give 2 (297 mg, 90% yield) as a white solid.

LCMS method 3: 95.8% purity at 215 nm, product does not ionize.

¹H NMR (400 MHz, DMSO-d6) δ ppm 1.32 (s, 12H), 4.11 (s, 3H), 5.45 (d, J=10.5 Hz, 4H), 6.59 (d, J=8.3 Hz, 1H), 7.27-7.42 (m, 9H), 7.45-7.49 (m, 2H), 7.67 (dd, J=8.2, 0.9 Hz, 1H), 7.89-7.92 (m, 2H).

Step 2. Preparation of 3-(2,6-dibenzyloxy-3-pyridyl)-1-methyl-indazol-6-ol (3). In a round bottom flask was added 3-(2,6-dibenzyloxy-3-pyridyl)-1-methyl-6-(4,4,5,5-tetramethyl-1,3,2-dioxaborolan-2-yl)indazole 2 (297 mg, 0.51 mmol, 1 eq.) in THF (2.6 mL, 0.13 M) and water (1.3 mL, 0.13 M). To the solution was added sodium perborate tetrahydrate (117.19 mg, 0.76 mmol, 1.5 eq.) and the reaction mixture was stirred at r.t. After 1 h, LCMS showed complete conversion into compound 3. The reaction mixture was cooled to room temperature, and EtOAc and water were added. The aqueous phase was extracted 3× with EtOAc. The organic phases were combined, washed with water, brine, dried over Na₂SO₄, filtered and evaporated under reduce pressure to give 3 (270 mg, quantitative yield) as a tan solid.

LCMS method 3: 85.6% purity at 215 nm, [M+H]⁺=438.1.

¹H NMR (400 MHz, DMSO-d6) δ ppm 3.91 (s, 3H), 5.43 (d, J=9.0 Hz, 4H), 6.52-6.59 (m, 2H), 7.25-7.34 (m, 4H), 7.34-7.43 (m, 5H), 7.44-7.50 (m, 3H), 7.88 (d, J=8.1 Hz, 1H), 9.65 (s, 1H).

Step 3. Preparation of tert-butyl 4-[3-(2,6-dibenzyloxy-3-pyridyl)-1-methyl-indazol-6-yl]oxypiperidine-1-carboxylate (5). In a sealed tube were added 3-(2,6-dibenzyloxy-3-pyridyl)-1-methyl-indazol-6-ol 3 (270 mg, 0.62 mmol, 1 eq.) and tert-butyl 4-methylsulfonyloxypiperidine-1-carboxylate 4 (206.88 mg, 0.74 mmol, 1.2 eq.) in DMF (4 mL, 0.14 M) and toluene (0.4 mL, 0.14 M). To the reaction mixture was then added DIPEA (0.21 mL, 1.23 mmol, 2 eq.) and K₂CO₃ (178.85 mg, 1.3 mmol, 2.1 eq.). The tube was sealed and the reaction mixture was stirred at 95° C. After an overnight period, LCMS showed 56% conversion into compound 5. More tert-butyl 4-methylsulfonyloxypiperidine-1-carboxylate 4 (68.96 mg, 0.25 mmol, 0.4 eq.) was added, the tube sealed and the reaction stirred at 95° C. After another overnight period, LCMS showed major conversion. After cooling to room temperature, EtOAc and water was added to the reaction mixture, which was then extracted 3× with EtOAc. The organic phases were combined, washed with brine, dried over Na₂SO₄, filtered and evaporated under reduce pressure. The crude material was then purified by normal phase flash chromatography (40 g silica column, solid deposit on silica, elution: 0 to 40% EtOAc/heptanes over 15 CV, product came around 30% EtOAc). Fractions were combined and concentrated to give 5 (100 mg, 26% yield) as a yellow semi-solid.

LCMS method 3: 98.3% purity at 215 nm, product does not ionize.

¹H NMR (400 MHz, DMSO-d6) δ ppm 1.41 (s, 9H), 1.50-1.61 (m, 2H), 1.64-1.71 (m, 1H), 1.91-2.00 (m, 2H), 2.88-2.99 (m, 1H), 3.19-3.27 (m, 2H), 3.99 (s, 3H), 4.69 (d, J=4.2 Hz, 1H), 5.43 (d, J=9.0 Hz, 4H), 6.57 (d, J=8.1 Hz, 1H), 6.65 (dd, J=8.9, 2.1 Hz, 1H), 7.12 (d, J=2.0 Hz, 1H), 7.25-7.43 (m, 9H), 7.45-7.49 (m, 2H), 7.53 (d, J=8.8 Hz, 1H), 7.89 (d, J=8.1 Hz, 1H).

Step 4. Preparation of tert-butyl 4-[3-(2,6-dioxo-3-piperidyl)-1-methyl-indazol-6-yl]oxypiperidine-1-carboxylate (6). To a degassed solution of tert-butyl 4-[3-(2,6-dibenzyloxy-3-pyridyl)-1-methyl-indazol-6-yl]oxypiperidine-1-carboxylate 5 (100 mg, 0.16 mmol, 1 eq.) in THF (1 mL, 0.08 M) and ethanol (1 mL, 0.08 M) was added Pd(OH)₂ (22.62 mg, 0.03 mmol, 0.2 eq.) in a reactor linked to a pressure valve. Purging cycles with N₂ and H₂ were performed. The mixture was stirred overnight at 60° C. under a pressure of 70 psi. The reaction mixture was filtered over a pad of celite, washed with EtOAc and EtOH, and concentrated under reduce pressure. The residue was directly loaded for reverse phase FC purification (50 g C18 RediSep Rf Gold column, liquid deposit (DMSO), 5% MeCN/0.1% HCOOH over 4 CV, then 5 to 95% MeCN/0.1% HCOOH over 20 CV, product came out at 70% MeCN). Pure fractions were combined and concentrated to give 6 (30 mg, 38% yield) as a tan solid.

LCMS method 3: 98.3% purity at 215 nm, product does not ionize.

¹H NMR (400 MHz, DMSO-d6) δ ppm 1.41 (s, 9H), 1.51-1.61 (m, 2H), 1.92-2.00 (m, 2H), 2.12-2.21 (m, 1H), 2.27-2.37 (m, 1H), 2.59-2.70 (m, 2H), 3.18-3.27 (m, 2H), 3.64-3.71 (m, 2H), 3.92 (s, 3H), 4.29 (dd, J=9.7, 5.0 Hz, 1H), 4.65-4.73 (m, 1H), 6.76 (dd, J=9.3, 1.5 Hz, 1H), 7.13 (s, 1H), 7.57 (d, J=8.8 Hz, 1H), 10.86 (s, 1H).

Step 5. Preparation of 3-[1-methyl-6-(4-piperidyloxy)indazol-3-yl]piperidine-2,6-dione;hydrochloride (C-14). To tert-butyl 4-[3-(2,6-dioxo-3-piperidyl)-1-methyl-indazol-6-yl]oxypiperidine-1-carboxylate 6 (65 mg, 0.15 mmol, 1 eq.) was added 4 M HCl in dioxane (0.29 mL, 1.18 mmol, 8.0 eq.). The reaction mixture was stirred at rt for 20 h. Upon completion, the solvent was removed under reduced pressure, and the residue was co-evaporated with MeCN (2×). The residue was dried under high vacuum to give C-14 (53 mg, 87% yield) as a white solid as a dihydrochloride salt, which was used in the next step without further purification.

LCMS method 3: 71.0% purity at 215 nm, [M+H]*=343.1.

¹H NMR (400 MHz, DMSO-d6) δ ppm 1.82-1.90 (m, 2H), 2.09-2.22 (m, 4H), 2.25-2.38 (m, 1H), 2.59-2.65 (m, 1H), 3.04-3.19 (m, 4H), 3.93 (s, 3H), 4.30 (dd, J=9.4, 5.3 Hz, 1H), 4.73-4.80 (m, 1H), 6.79 (dd, J=8.7, 1.6 Hz, 1H), 7.15-7.19 (m, 1H), 7.59 (d, J=9.0 Hz, 1H), 8.77-8.87 (m, 2H), 10.87 (s, 1H).

### Example I-11. Synthesis of Common Intermediate C-15

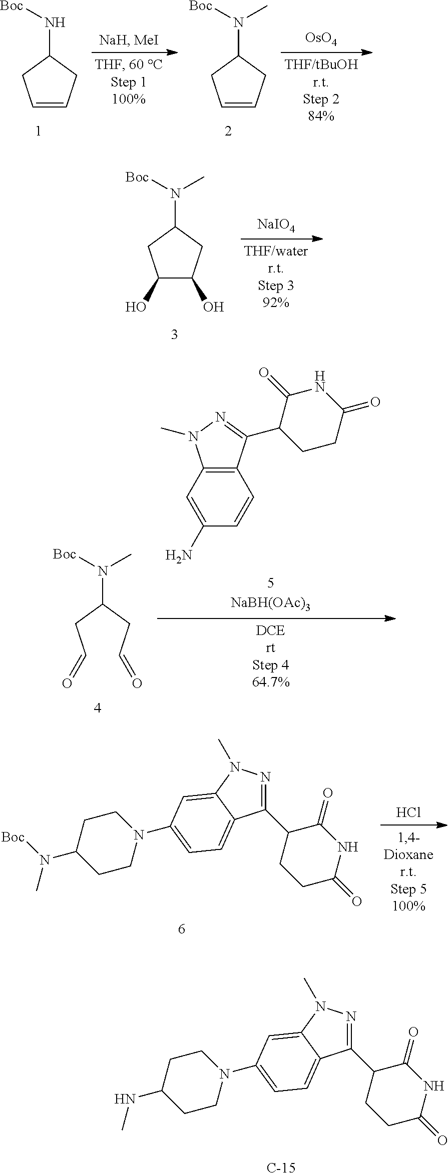

Step 1. Preparation of Tert-butyl N-cyclopent-3-en-1-yl-N-methyl-carbamate (2). Iodomethane (764.38 μL, 12.28 mmol, 5.0 eq.) was added to a suspension of tert-butyl N-cyclopent-3-en-1-ylcarbamate 1 (450. mg, 2.46 mmol, 1.0 eq.) and NaH (491.13 mg, 12.28 mmol, 5.0 eq.) in THF (16.3 mL, 0.15 M) at room temperature. The suspension was stirred 60° C. TLC (Hept·EtOAc 9:1) showed full conversion after overnight. The reaction was cooled down and was quenched by addition of water. Ethyl acetate was added and phases were separated. The aqueous phase was extracted 3 times with ethyl acetate, then the combined organic phases were washed once with water and once with brine, dried over magnesium sulfate and concentrated. The residue was then purified by normal phase flash chromatography (40 g silica column, elution: 0 to 10% EtOAc/heptanes over 15 CV, product exited at 7% EtOAc). Fractions were combined and concentrated to give tert-butyl N-cyclopent-3-en-1-yl-N-methyl-carbamate 2 (511 mg, 100% yield) as a colorless oil.

¹H NMR (400 MHz, chloroform-d) δ ppm 1.47 (s, 9H), 2.21-2.32 (m, 2H), 2.57-2.62 (m, 2H), 2.67 (s, 3H), 4.93 (br s, 1H), 5.67-5.72 (m, 2H).

Step 2. Preparation of Tert-butyl N-methyl-N-[rac-(3R,4S)-3,4-dihydroxycyclopentyl]carbamate (3). To a solution of tert-butyl N-cyclopent-3-en-1-yl-N-methyl-carbamate 2 (535. mg, 2.71 mmol, 1.0 eq.) in tert-butanol (9.0 mL) and THF (4.5 mL, 0.2 M) was added osmium tetroxide (0.17 mL, 0.03 mmol, 0.01 eq.) followed by NMO (381.26 mg, 3.25 mmol, 1.2 eq.). The reaction was stirred at rt and monitored by TLC. After 16 hours, TLC (10:90 MeOH/DCM, KMnO₄) showed complete conversion of 2 into two new spots. The mixture was concentrated to dryness. The residue was dissolved in EtOAc and washed with 10% Na₂SO₃ (2×), saturated NaHCO₃ and brine. The organic phase was dried over MgSO₄ and concentrated in vacuo. The residue was then purified by normal phase flash chromatography (40 g silica column, DCM injection, elution: 0 to 10% MeOH/DCM over 15 CV, product exited at 9% MeOH). Fractions selected by TLC in 10:90 MeOH/DCM, Vanillin stain. Collected fractions were concentrated to afford tert-butyl N-methyl-N-[rac-(3R,4S)-3,4-dihydroxycyclopentyl] carbamate 3 (523 mg, 83% yield) as a colorless oil.

¹H NMR (400 MHz, chloroform-d) δ ppm 1.47 (s, 9H), 1.83-1.95 (m, 2H), 1.95-2.06 (m, 2H), 2.21 (br d, J=2.7 Hz, 2H), 2.75 (s, 3H), 4.20-4.29 (m, 2H), 4.76 (dt, J=16.9, 8.4 Hz, 1H).

Step 3. Preparation of N-Methyl-N-[3-oxo-1-(2-oxoethyl)propyl]carbamate (4). To a solution of tert-butyl N-methyl-N-[rac-(3R,4S)-3,4-dihydroxycyclopentyl]carbamate 3 (523. mg, 2.26 mmol, 1.0 eq.) in THF (10 mL) and water (5 mL, 0.15 M) was added sodium periodate (0.58 g, 2.71 mmol, 1.2 eq.). The reaction was stirred at room temperature and monitored by TLC. After 2 hours, TLC (5% MeOH/DCM, vanillin stain) showed complete conversion. THE was removed in vacuo and NaCl solid/brine was added. The mixture was extracted with EtOAc (3×). The combined organics were dried over MgSO₄ and concentrated in vacuo to give tert-butyl N-methyl-N-[3-oxo-1-(2-oxoethyl)propyl]carbamate 4 (478 mg, 92% yield) as a white solid.

¹H NMR (400 MHz, chloroform-d) δ ppm 1.47 (s, 9H), 1.54-1.70 (m, 2H), 2.69-2.75 (m, 2H), 2.78 (s, 3H), 4.87-5.03 (m, 1H), 9.68-9.84 (m, 2H).

Step 4. Preparation of Tert-butyl N-[1-[3-(2,6-dioxo-3-piperidyl)-1-methyl-indazol-6-yl]-4-piperidyl]-N-methyl-carbamate (6). To a solution of 3-(6-amino-1-methyl-indazol-3-yl)piperidine-2,6-dione 4 (425.0 mg, 1.65 mmol, 1.0 eq.) and tert-butyl N-methyl-N-[3-oxo-1-(2-oxoethyl)propyl]carbamate 5 (452.7 mg, 1.97 mmol, 1.2 eq.) in DCE (8.5 mL, 0.2 M) was added sodium triacetoxyborohydride (837.0 mg, 3.95 mmol, 2.4 eq.). The reaction was stirred under N₂ atmosphere, at room temperature for 48 hours. The reaction was then quenched with saturated NH₄Cl and extracted with DCM (3×). The combined organics were dried over Na₂SO₄ and concentrated in vacuo. The residue was then purified by normal phase flash chromatography (40 g silica column, DCM injection, elution: 0 to 10% MeOH/DCM over 15 CV, product exited at 5% MeOH). Collected fractions were concentrated to afford tert-butyl N-[1-[3-(2,6-dioxo-3-piperidyl)-1-methyl-indazol-6-yl]-4-piperidyl]-N-methyl-carbamate 6 (600 mg, 64.7% yield) as a yellow solid.

LCMS method 3: retention time: 1.860 min, 95% purity at 254 nm, [M+H]⁺=456.3.

¹H NMR (400 MHz, chloroform-d) δ ppm 1.49 (s, 9H), 1.74-1.82 (m, 2H), 1.83-1.94 (m, 2H), 2.31-2.41 (m, 1H), 2.45-2.55 (m, 1H), 2.62-2.71 (m, 1H), 2.72-2.75 (m, 1H), 2.78 (s, 3H), 2.82-2.91 (m, 2H), 2.94-3.04 (m, 1H), 3.82 (br d, J=12.7 Hz, 2H), 3.95 (s, 3H), 4.25 (dd, J=7.0, 5.3 Hz, 1H), 6.64 (br s, 1H), 6.92 (br d, J=8.8 Hz, 1H), 7.51 (d, J=9.0 Hz, 1H), 7.97 (s, 1H).

Step 5. Preparation of 3-[1-Methyl-6-[4-(methylamino)-1-piperidyl]indazol-3-yl]piperidine-2,6-dione hydrochloride (C-15). HCl 4.0 M in dioxane (6.26 mL, 25.02 mmol, 20 eq.) was added to tert-butyl N-[1-[3-(2,6-dioxo-3-piperidyl)-1-methyl-indazol-6-yl]-4-piperidyl]-N-methyl-carbamate 6 (600. mg, 1.25 mmol, 1.0 eq.) and the mixture was sonicated for 1 minute, then stirred at room temperature for overnight. The solvent was evaporated to dryness and the residue was co-evaporated using MeCN (2×), which afforded 3-[1-methyl-6-[4-(methylamino)-1-piperidyl]indazol-3-yl]piperidine-2,6-dione;hydrochloride C-15 (595 mg, 100% yield) as a light brown solid.

LCMS method 3: retention time: 1.339 min, 96% purity at 215 nm, [M+H]⁺=356.3.

¹H NMR (400 MHz, DMSO-d6) δ ppm 1.79-1.95 (m, 2H), 2.12-2.24 (m, 3H), 2.28-2.40 (m, 2H), 2.56 (br t, J=5.4 Hz, 3H), 2.59-2.70 (m, 2H), 2.96-3.11 (m, 2H), 3.18-3.26 (m, 1H), 3.88 (br d, J=12.7 Hz, 1H), 3.93 (s, 3H), 4.30 (dd, J=9.5, 5.1 Hz, 1H), 7.04-7.31 (m, 2H), 7.54-7.69 (m, 1H), 9.12-9.30 (m, 2H), 10.87 (s, 1H).

### Example I-12. Synthesis of Common Intermediate C-16

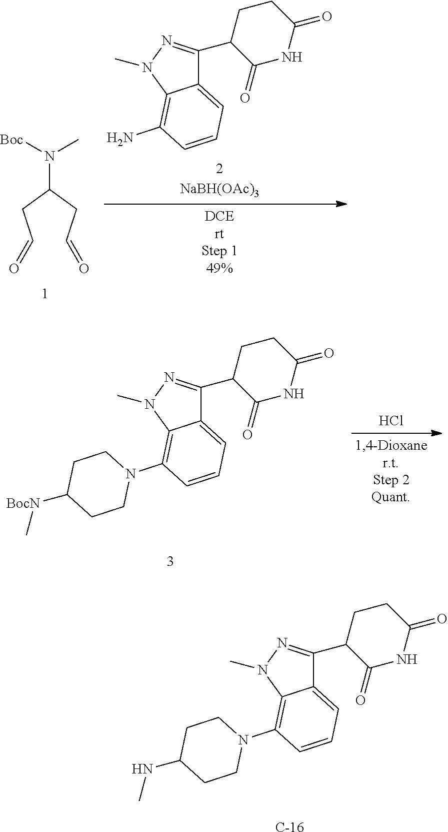

Step 1. Preparation of tert-butyl N-[1-[3-(2,6-dioxo-3-piperidyl)-1-methyl-indazol-6-yl]-4-piperidyl]-N-methyl-carbamate (3). To a solution of 3-(6-amino-1-methyl-indazol-3-yl)piperidine-2,6-dione 2 (161.47 mg, 0.63 mmol, 1.0 eq.) and tert-butyl N-methyl-N-[3-oxo-1-(2-oxoethyl)propyl]carbamate 1—intermediate 4 from synthesis of C-15 —(172 mg, 0.75 mmol, 1.2 eq.) in DCE (3.13 mL, 0.2 M) was added sodium triacetoxyborohydride (318.03 mg, 1.5 mmol, 2.4 eq.). The reaction was stirred under N₂ atmosphere at room temperature for 4 days. The reaction was then quenched with saturated NH₄Cl and extracted with DCM (3×). The combined organics were dried over Na₂SO₄ and concentrated in vacuo. The residue was then purified by normal phase flash chromatography (40 g silica column, DCM injection, elution: 0 to 10% MeOH/DCM over 20 CV, product came out at 5% MeOH). Collected fractions were concentrated to give 3 (148 mg, 49% yield) as a yellow solid.

LCMS method 3: 93.7% purity at 215 nm, [M+H]⁺=456.2.

¹H NMR (400 MHz, DMSO-d6) δ ppm 1.43 (br s, 9H), 1.62-1.75 (m, 2H), 1.90-2.08 (m, 2H), 2.12-2.22 (m, 1H), 2.23-2.38 (m, 2H), 2.60-2.72 (m, 2H), 2.77 (s, 3H), 2.78-2.83 (m, 1H), 3.24-3.30 (m, 2H), 3.38-3.56 (m, 1H), 4.26 (s, 3H), 4.31-4.37 (m, 1H), 6.97-7.09 (m, 2H), 7.39 (br d, J=7.3 Hz, 1H), 10.88 (s, 1H).

Step 2. Preparation of 3-[1-methyl-7-[4-(methylamino)-1-piperidyl]indazol-3-yl]piperidine-2,6-dione hydrochloride (C-16). To a solution of tert-butyl N-[1-[3-(2,6-dioxo-3-piperidyl)-1-methyl-indazol-7-yl]-4-piperidyl]-N-methyl-carbamate 3 (148 mg, 0.32 mmol, 1.0 eq.) in DCM (1.0 mL, 0.32 M) was added HCl 4.0 M in dioxane (2.44 mL, 9.75 mmol, 20 eq.) and the mixture was stirred at room temperature for overnight. After 90 minutes, LCMS showed complete conversion into compound 4. The solvent was evaporated to dryness and the residue was co-evaporated using MeCN (2×). The residue was dried under high vacuum to give C-16 (142 mg, quantitative yield) as a white solid as a hydrochloride salt.

LCMS method 3: 95.2% purity at 215 nm, [M−HCl+H]⁺=356.2.

¹H NMR (400 MHz, DMSO-d6) δ ppm 1.76-1.89 (m, 2H), 2.10-2.21 (m, 3H), 2.26-2.39 (m, 1H), 2.60 (br t, J=5.4 Hz, 3H), 2.62-2.68 (m, 2H), 2.68-2.82 (m, 2H), 3.08-3.22 (m, 1H), 3.25-3.35 (m, 2H), 4.25 (s, 3H), 4.34 (dd, J=9.7, 5.0 Hz, 1H), 6.99-7.07 (m, 2H), 7.40 (br d, J=1.7 Hz, 1H), 8.92 (br s, 2H), 10.89 (s, 1H).

### Example I-13. Synthesis of Common Intermediate C-17

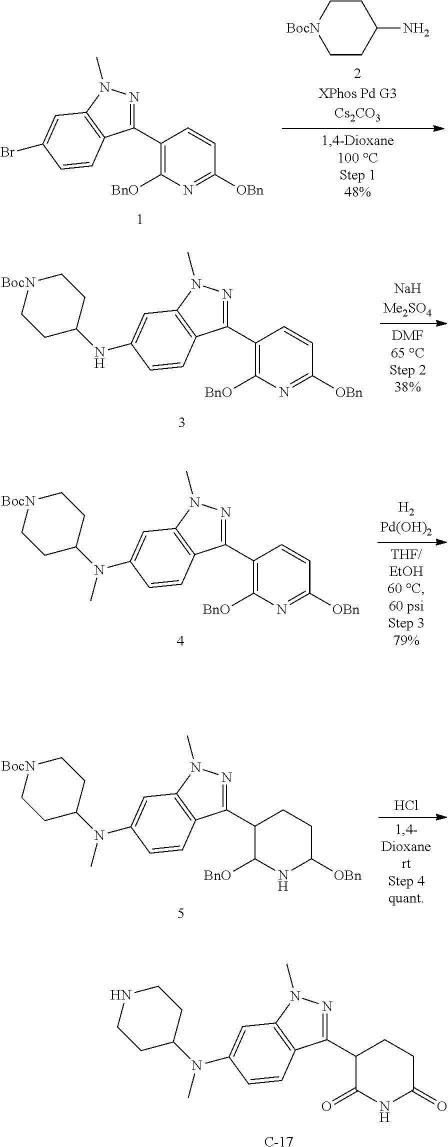

Step 1. Preparation of tert-butyl 4-[[3-(2,6-dibenzyloxy-3-pyridyl)-1-methyl-indazol-6-yl]amino]piperidine-1-carboxylate (3). A sealed tube was charged with 6-bromo-3-(2,6-dibenzyloxy-3-pyridyl)-1-methyl-indazole 1—intermediate 5 from synthesis of C-13 —(759 mg, 1.43 mmol), tert-butyl 4-aminopiperidine-1-carboxylate 2 (430 mg, 2.15 mmol), XPhos Pd G3 (121 mg, 0.14 mmol), and Cs₂CO₃ (931 mg, 2.87 mmol) in 1,4-dioxane (4.2 mL). The solution was degassed for 15 min with nitrogen, the tube was sealed and the reaction mixture was stirred at 100° C. After 18 h, reaction mixture was cooled to room temperature, filtered through a pad of celite, and filter cake was rinsed with DCM. The filtrate was concentrated to dryness under reduced pressure and the residue was purified by normal phase flash chromatography (EtOAc/heptanes) to afford tert-butyl 4-[[3-(2,6-dibenzyloxy-3-pyridyl)-1-methyl-indazol-6-yl]amino]piperidine-1-carboxylate 3 (471 mg, 48% yield) as a white solid.

LCMS method 1: 99.9% purity at 215 nm, product does not ionize.

¹H NMR (400 MHz, DMSO-d6) δ ppm 1.19-1.33 (m, 3H), 1.41 (s, 9H), 1.88-1.99 (m, 2H), 2.88-3.05 (m, 2H), 3.45-3.58 (m, 1H), 3.89 (s, 3H), 3.90-3.94 (m, 1H), 5.41 (s, 2H), 5.44 (s, 2H), 5.74-5.79 (m, 1H), 6.39-6.44 (m, 2H), 6.55 (d, J=8.1 Hz, 1H), 7.23-7.50 (m, 11H), 7.86 (d, J=8.1 Hz, 1H).

Step 2. Preparation of tert-butyl 4-[[3-(2,6-dibenzyloxy-3-pyridyl)-1-methyl-indazol-6-yl]-methyl-amino]piperidine-1-carboxylate (4). tert-butyl 4-[[3-(2,6-dibenzyloxy-3-pyridyl)-1-methyl-indazol-6-yl]amino]piperidine-1-carboxylate 3 (903 mg, 1.43 mmol) and 60% NaH (142 mg, 2.14 mmol) were dissolved in THF (5.7 mL) at 0° C. After 1 h, at 0° C. dimethyl sulfate (0.2 mL, 2.14 mmol) was added dropwise at 0° C. Then reaction mixture was heated at 70° C. After 2.5 h, volatiles were removed under reduced procedure. The residue was directly purified by reverse phase C18 column chromatography (MeCN/0.1% aqueous formic acid) to afford tert-butyl 4-[[3-(2,6-dibenzyloxy-3-pyridyl)-1-methyl-indazol-6-yl]-methyl-amino]piperidine-1-carboxylate 4 (345 mg, 38% yield) as a tan solid.

LCMS method 1: 99.9% purity at 215 nm, product does not ionize.

¹H NMR (400 MHz, CDCl₃) δ ppm 1.49 (s, 9H), 1.64-1.81 (m, 4H), 2.73-2.83 (m, 2H), 2.85 (s, 3H), 3.76-3.85 (m, 1H), 4.02 (s, 3H), 4.19-4.34 (m, 2H), 5.39 (s, 2H), 5.48 (s, 2H), 6.48 (d, J=1.7 Hz, 1H), 6.51 (d, J=8.1 Hz, 1H), 6.73 (dd, J=9.0, 2.0 Hz, 1H), 7.23-7.47 (m, 10H), 7.57 (d, J=9.0 Hz, 1H), 7.92 (d, J=8.1 Hz, 1H).

Step 3. Preparation of tert-butyl 4-[[3-(2,6-dioxo-3-piperidyl)-1-methyl-indazol-6-yl]-methyl-amino]piperidine-1-carboxylate (5). A solution of tert-butyl 4-[[3-(2,6-dibenzyloxy-3-pyridyl)-1-methyl-indazol-6-yl]-methyl-amino]piperidine-1-carboxylate 4 (507 mg, 0.80 mmol) in THF (2.7 mL) and ethanol (2.7 mL) was degassed for 15 minutes, then Pd(OH)₂ (89 mg, 0.13 mmol) was added and sparging was resumed for 5 minutes. The reaction mixture was heated overnight at 60° C. under a positive pressure of hydrogen (approx. 60 psi). After 18 h, reaction mixture was filtered over a pad of celite. The filter cake was washed with EtOAc and EtOH, and filtrate was concentrated under reduce pressure. The residue was directly purified by reverse phase C18 column chromatography (MeCN/0.1% aqueous formic acid) to afford tert-butyl 4-[[3-(2,6-dioxo-3-piperidyl)-1-methyl-indazol-6-yl]-methyl-amino]piperidine-1-carboxylate 5 (289 mg, 79% yield) as a light-pink solid.

LCMS method 1: 96.2% purity at 215 nm, [M+H]⁺=456.2.

¹H NMR (400 MHz, CDCl₃) δ ppm 1.49 (s, 9H), 1.65-1.82 (m, 4H), 2.29-2.41 (m, 1H), 2.50 (dtd, J=13.9, 7.1, 5.1 Hz, 1H), 2.61-2.72 (m, 1H), 2.73-2.81 (m, 2H), 2.85 (s, 3H), 2.99 (ddd, J=17.7, 8.8, 5.0 Hz, 1H), 3.74-3.87 (m, 1H), 3.93 (s, 3H), 4.18-4.35 (m, 3H), 6.45 (d, J=1.7 Hz, 1H), 6.85 (dd, J=9.3, 2.0 Hz, 1H), 7.50 (d, J=9.0 Hz, 1H), 7.89 (s, 1H).

Step 4. Preparation of 3-[1-methyl-6-[methyl(4-piperidyl)amino]indazol-3-yl]piperidine-2,6-dione (C-17). tert-Butyl 4-[[3-(2,6-dioxo-3-piperidyl)-1-methyl-indazol-6-yl]-methyl-amino]piperidine-1-carboxylate 5 (289.2 mg, 0.6300 mmol) was dissolved in 4 M HCl in dioxane (3.15 mL, 12.61 mmol). The reaction mixture was stirred at rt for 1 h. Upon completion, the volatiles were removed under reduced pressure, and the residue was co-evaporated with toluene (2×) and MTBE. The residue was dried under high vacuum to give 3-[1-methyl-6-[methyl(4-piperidyl)amino]indazol-3-yl]piperidine-2,6-dione dihydrochloride C-17 (282 mg, quant) as a white solid.

LCMS method 1: 98.0% purity at 215 nm, [M+H]⁺=356.2.

¹H NMR (400 MHz, DMSO-d6) δ ppm 1.80-1.92 (m, 1H), 1.93-2.06 (m, 2H), 2.11-2.21 (m, 1H), 2.26-2.39 (m, 1H), 2.55-2.71 (m, 2H), 2.83-3.06 (m, 4H), 3.29-3.40 (m, 2H), 3.44-3.52 (m, 1H), 3.62-3.78 (m, 2H), 3.93 (s, 3H), 4.00-4.15 (m, 1H), 4.26-4.39 (m, 1H), 6.96-7.24 (m, 1H), 7.49-7.76 (m, 1H), 8.62-8.83 (m, 1H), 8.94-9.20 (m, 1H), 10.87 (s, 1H).

### Example I-14. Synthesis of Common Intermediate C-20

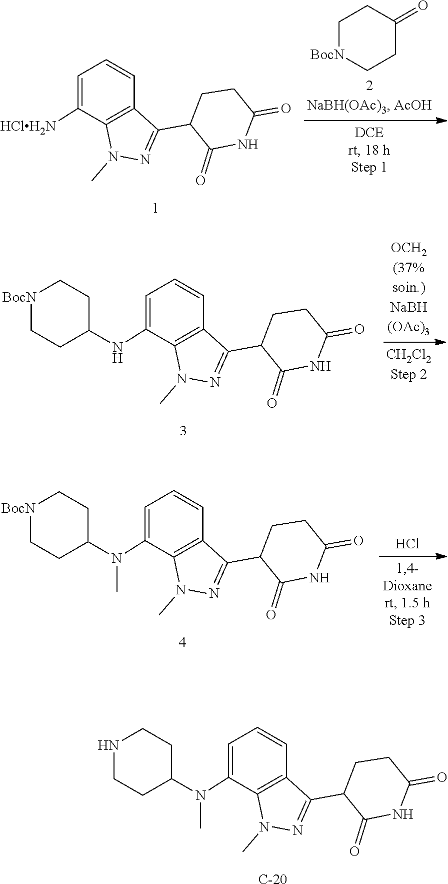

Step 1. Preparation of tert-butyl 4-[[3-(2,4-dioxohexahydropyrimidin-1-yl)-1-methyl-indazol-6-yl]amino]piperidine-1-carboxylate (3). Acetic acid (1.1 mL, 19.29 mmol, 20 eq.) was added to an ice-cooled mixture of 1-(6-amino-1-methyl-indazol-3-yl)hexahydropyrimidine-2,4-dione 1 (250 mg, 0.9600 mmol, 1.0 eq.), tert-butyl 4-oxopiperidine-1-carboxylate 2 (192 mg, 0.9600 mmol, 1.0 eq.) and NaBH(OAc)₃ (306 mg, 1.45 mmol, 1.5 eq.) in DCE (10 mL). The mixture was stirred at 0° C. for 20 minutes and at room temperature for 3 days. The phases were separated, then the aqueous phase was extracted with DCM (3×10 mL). The organic layers were combined, dried over MgSO₄, filtered and evaporated under reduced pressure. The crude was purified by reversed-phase flash chromatography (MeOH in 0.1% HCOOH(aq), 5%-60% over 20 CV). Evaporation of the fractions afforded 3 (105 mg, 0.225 mmol, quantitative yield) as a pink solid.

LCMS method 1: 100% purity at 215 nm, [M+H]⁺=443.2.

¹H NMR (400 MHz, DMSO-d6) δ ppm 1.19-1.35 (m, 2H), 1.42 (s, 9H), 1.91-2.00 (m, 2H), 2.72 (s, 2H), 2.86-3.07 (m, 2H), 3.48-3.61 (m, 1H), 3.82 (s, 3H), 3.84-3.93 (m, 4H), 5.81-5.90 (m, 1H), 6.42 (s, 1H), 6.47-6.57 (m, 1H), 7.28 (d, J=9.0 Hz, 1H), 10.42-10.53 (m, 1H).

Step 2. Preparation of tert-butyl 4-[[3-(2,4-dioxohexahydropyrimidin-1-yl)-1-methyl-indazol-6-yl]-methyl-amino]piperidine-1-carboxylate (4). tert-Butyl 4-[[3-(2,4-dioxohexahydropyrimidin-1-yl)-1-methyl-indazol-6-yl]amino]piperidine-1-carboxylate 3 (105 mg, 0.24 mmol, 1.0 eq.) was dissolved in DCM (5 mL) then an aqueous 37% formaldehyde solution (0.02 mL, 0.28 mmol, 1.2 eq.) was added followed by NaBH(OAc)₃ (75 mg, 0.36 mmol, 1.5 eq.) at room temperature. After 2 h, the LCMS showed complete conversion. Volatiles were removed and the residue was purified by reversed-phase flash chromatography (MeOH in 0.1% HCOOH(aq), 5%-60% over 20 CV). Evaporation of the fractions afforded 4 (118 mg, 0.2339 mmol, 90.5% yield) as a pink semi-solid.

LCMS method 1: 90.5% purity at 215 nm, [M+H]⁺=457.3.

¹H NMR (400 MHz, CDCl₃) δ ppm 1.41 (s, 9H), 1.55-1.72 (m, 4H), 1.94 (s, 1H), 2.77 (s, 3H), 2.87 (t, J=1.0 Hz, 2H), 3.69-3.81 (m, 1H), 3.84 (s, 3H), 3.93-4.02 (m, 2H), 4.11-4.26 (m, 2H), 5.34 (s, 2H), 6.33 (s, 1H), 6.76-6.82 (m, 1H), 7.40-7.47 (m, 1H).

Step 3. Preparation of [3-(2,4-dioxohexahydropyrimidin-1-yl)-1-methyl-1H-indazol-1-ium-6-yl]-methyl-piperidin-1-ium-4-yl-ammonium;trichloride (C-20). tert-Butyl 4-[[3-(2,4-dioxohexahydropyrimidin-1-yl)-1-methyl-indazol-6-yl]-methyl-amino]piperidine-1-carboxylate 4 (118 mg, 0.233 mmol, 1.0 eq.) was dissolved in 1,4-dioxane (2.5 mL), then 4.0 M HCl in 1,4-dioxane solution (1.16 mL, 4.65 mmol, 20 eq.) was added and the resulting solution was stirred at room temperature. After 4 h, LCMS showed full conversion. The solvent was evaporated in vacuo and chased twice with MeCN to afford C-20 (112 mg, 0.21 mmol, 90% yield) as a light-pink solid.

LCMS method 1: 87.1% purity at 215 nm, [M-3HCl+H]⁺=357.2.

¹H NMR (400 MHz, DMSO-d6) δ ppm 1.40 (s, 1H), 1.74-1.92 (m, 2H), 1.93-2.06 (m, 2H), 2.82-2.96 (m, 5H), 2.96-3.12 (m, 2H), 3.31-3.42 (m, 2H), 3.57 (s, 1H), 3.81-3.99 (m, 4H), 4.04-4.18 (m, 1H), 5.14 (s, 2H), 7.04 (br s, 1H), 7.55 (br s, 1H), 8.68-9.17 (m, 2H).

### Final Compound Synthesis

### Final Product General Method 1

### Example S1. Synthesis of P-4

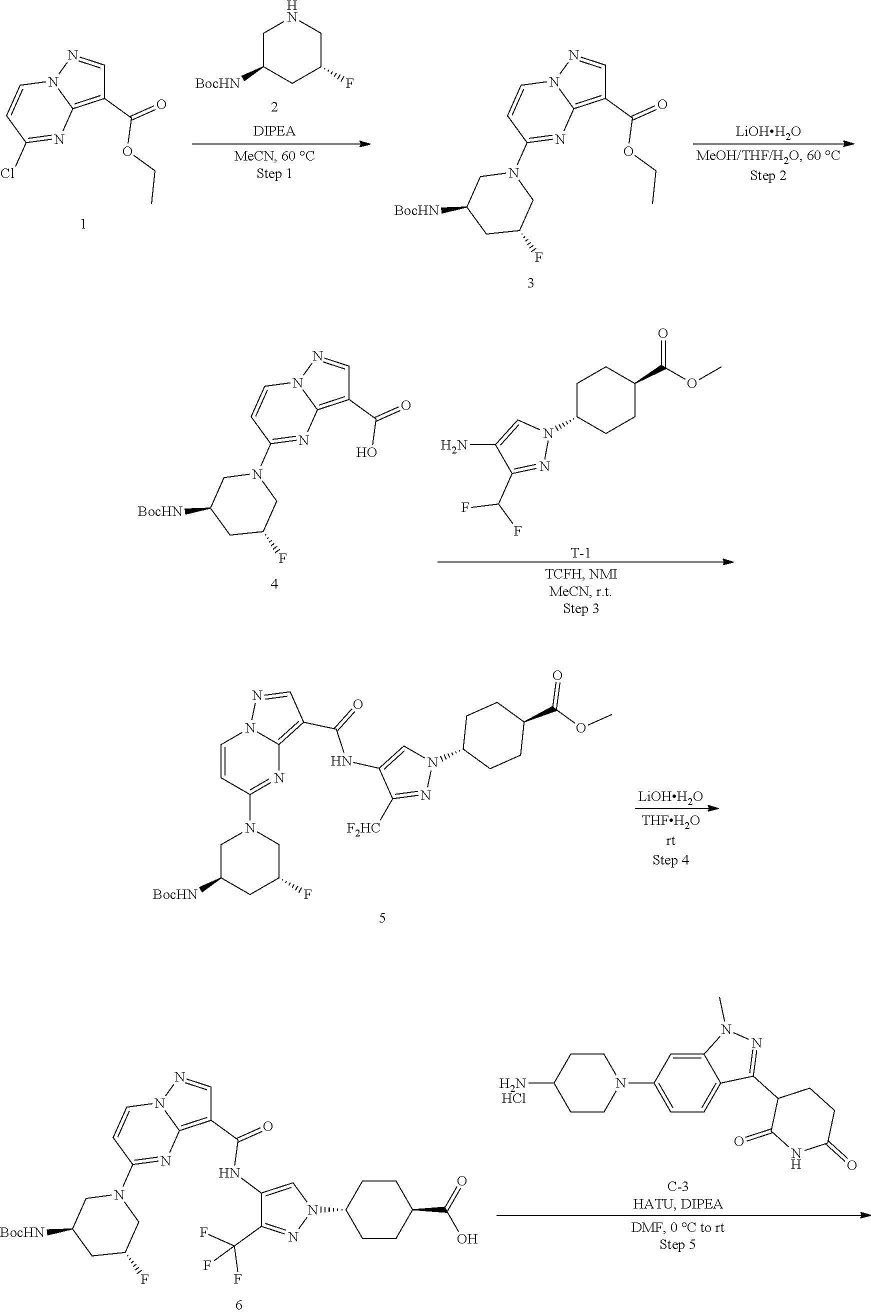

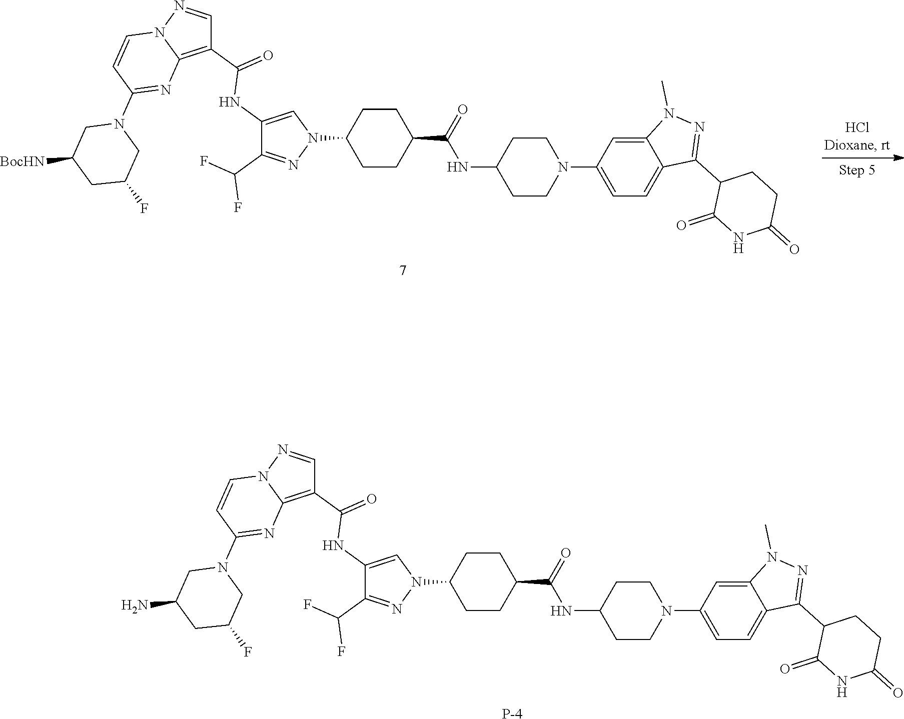

Step 1. Preparation of ethyl 5-[(3R,5R)-3-(tert-butoxycarbonylamino)-5-fluoro-1-piperidyl]pyrazolo[1,5-a]pyrimidine-3-carboxylate (3): To a solution of ethyl 5-chloropyrazolo[1,5-a]pyrimidine-3-carboxylate 1 (800 mg, 3.55 mmol, 1.0 eq.) in MeCN (17.7 mL) was added DIPEA (1.54 mL, 8.86 mmol, 2.5 eq.) and tert-butyl N-[(3R,5R)-5-fluoro-3-piperidyl]carbamate 2 (1.01 g, 4.61 mmol, 1.3 eq.). After stirring at 60° C. over the weekend, LCMS showed complete conversion into 3. The solvent was removed under reduced pressure and the residue was dried under high vacuum to give 3 (1.44 g, quantitative yield) as white solid. The crude product was used in the next step without further purification.

LCMS method 1: retention time: 1.656 min, 99.9% purity at 215 nm, [M+H]⁺=408.2.

¹H NMR (400 MHz, DMSO-d6) δ ppm 1.37-1.45 (m, 9H), 1.56-1.92 (m, 1H), 2.09-2.20 (m, 1H), 2.56-2.71 (m, 1H), 2.76-3.03 (m, 1H), 3.08-3.19 (m, 1H), 3.29 (s, 1H), 3.37 (br s, 1H), 3.56-3.73 (m, 2H), 4.11-4.25 (m, 2H), 4.41-4.73 (m, 1H), 4.99 (br s, 1 H), 5.11 (br s, 1H), 6.85 (br d, J=8.1 Hz, 1H), 7.10 (br d, J=7.8 Hz, 1H), 8.22 (s, 1H), 8.73 (d, J=7.8 Hz, 1H).

¹⁹F NMR (377 MHz, DMSO-d6) δ ppm −184.32 (s, 1F).

Step 2. Preparation of 5-[(3R,5R)-3-(tert-Butoxycarbonylamino)-5-fluoro-1-piperidyl]pyrazolo[1,5-a]pyrimidine-3-carboxylic acid (4). To a solution of ethyl 5-[(3R,5R)-3-(tert-butoxycarbonylamino)-5-fluoro-1-piperidyl]pyrazolo[1,5-a]pyrimidine-3-carboxylate 3 (1.44 mg, 3.53 mmol, 1.0 eq) in THF (5.89 mL) and methanol (5.89 mL) was added a solution of LiOH·H₂O (1.48 g, 35.34 mmol, 10.0 eq.) in water (5.89 mL). After stirring at 60° C. for 18 h, LCMS showed complete conversion into desired product 4. The reaction mixture was concentrated under vacuum to remove THF/MeOH and the crude mixture was diluted with water. Under vigorous agitation, the mixture was acidified with a 6N aqueous HCl solution until pH=3 (formation of a precipitate). The suspension was filtered on a Buchner funnel and the solid was rinsed with water. The solid was dried overnight in a stove under vacuum to give 4 (1.40 g, quantitative yield) as white solid. The crude product was used in the next without further purification.

LCMS method 1: retention time: 1.487 min, 99.9% purity at 215 nm, [M+H]⁺=380.1.

¹H NMR (400 MHz, DMSO-d6) δ ppm 1.41 (s, 9H), 1.66-1.95 (m, 1H), 2.05-2.26 (m, 1H), 2.92-3.11 (m, 1H), 3.28-3.30 (m, 1H), 3.34-3.49 (m, 1H), 3.59-3.74 (m, 1H), 4.52-4.75 (m, 1H), 4.99 (br s, 1H), 5.11 (br s, 1H), 6.82 (br d, J=7.6 Hz, 1H), 7.12 (br d, J=7.8 Hz, 1H), 8.19 (s, 1H), 8.73 (d, J=7.8 Hz, 1H).

¹⁹F NMR (377 MHz, DMSO-d6) δ ppm −184.32 (s, 1F).

Step 3. Preparation of 5-Methyl 4-[4-[[5-[(3R,5R)-3-(tert-butoxycarbonylamino)-5-fluoro-1-piperidyl]pyrazolo[1,5-a]pyrimidine-3-carbonyl]amino]-3-(difluoromethyl)pyrazol-1-yl]cyclohexanecarboxylate (5). To a solution of methyl 4-[4-amino-3-(difluoromethyl)pyrazol-1-yl]cyclohexanecarboxylate T-1 (239 mg, 0.870 mmol, 1.0 equiv.) and 5-[(3R,5R)-3-(tert-butoxycarbonylamino)-5-fluoro-1-piperidyl]pyrazolo[1,5-a]pyrimidine-3-carboxylic acid 4 (332 mg, 0.870 mmol, 1.0 equiv.) in MeCN (4.37 mL, 0.2 M) was added NMI (208 μL, 2.62 mmol, 3.0 equiv.), followed by TCFH (368 mg, 1.31 mmol, 1.5 equiv.). The resulting mixture was stirred at rt for 1 h. Nanopure water was added to the reaction mixture. The suspension was sonicated then filtered on a Buchner funnel. The solid was washed with nanopure water and dried under high vacuum to afford the pure 5 (466 mg, 84% yield) without further purification.

LCMS method 1: 99.90% purity at 215 nm, [M+H]⁺=635.2 m/z.

¹H NMR (400 MHz, DMSO-d6) δ ppm 1.34 (s, 9H), 1.40-1.44 (m, 1H), 1.48-1.59 (m, 2H), 1.76-1.94 (m, 3H), 1.98-2.17 (m, 5H), 2.36-2.45 (m, 1H), 2.98-3.12 (m, 1H), 3.37-3.52 (m, 1H), 3.62 (s, 3H), 3.64-3.71 (m, 1H), 4.20-4.29 (m, 1H), 4.58-4.80 (m, 1H), 4.94-5.12 (m, 1H), 6.85-6.95 (m, 1H), 7.02-7.23 (m, 2H), 8.23-8.39 (m, 2H), 8.81 (d, J=7.8 Hz, 1H), 9.31 (s, 1H).

¹⁹F NMR (377 MHz, DMSO-d6) δ ppm −184.13 (s, 1F), −111.44 (s, 2F).

Step 4. Preparation of 4-[3-(Difluoromethyl)-4-[[5-[(3R,5R)-3-(tert-butoxycarbonylamino)-5-fluoro-1-piperidyl]pyrazolo[1,5-a]pyrimidine-3-carbonyl]amino]pyrazol-1-yl]cyclohexanecarboxylic acid (6). To a solution of methyl 4-[3-(difluoromethyl)-4-[[5-[(3R,5R)-3-(tert-butoxycarbonylamino)-5-fluoro-1-piperidyl]pyrazolo[1,5-a]pyrimidine-3-carbonyl]amino]pyrazol-1-yl]cyclohexanecarboxylate 5 (466 mg, 0.730 mmol, 1.0 equiv.) in THF (2.45 mL, 0.15 M) was added a solution of LiOH H₂O (308 mg, 7.34 mmol, 10 equiv.) in water (2.45 mL, 0.15 M). The resulting mixture was stirred at rt overnight. The solvents were removed under reduced pressure and the residue was suspended in nanopure water and sonicated. Under vigorous agitation at 0° C., aqueous 6 M HCl was added until pH=3. The solid was filtered on a Buchner funnel, rinsing with nanopure water to afford pure 6 (362 mg, 79% yield).

LCMS method 1: 99.9% purity at 215 nm, [M+H]⁺=621.3 m/z.

¹H NMR (400 MHz, DMSO-d6) δ ppm 1.34 (s, 9H), 1.39-1.43 (m, 1H), 1.47-1.57 (m, 2H), 1.72-1.86 (m, 3H), 2.00-2.15 (m, 5H), 2.26-2.35 (m, 1H), 2.99-3.13 (m, 1H), 3.42-3.52 (m, 2H), 3.63-3.72 (m, 1H), 4.17-4.30 (m, 1H), 4.95-5.12 (m, 1H), 6.85-6.95 (m, 1H), 7.03-7.23 (m, 2H), 8.28 (s, 1H), 8.34 (s, 1H), 8.81 (d, J=8.1 Hz, 1H), 9.31 (s, 1H), 12.09-12.23 (m, 1H).

¹⁹F NMR (377 MHz, DMSO-d6) δ ppm −184.09 (s, 1F), −111.31-(s, 2F).

Step 5. Preparation of tert-butyl N-[(3R,5R)-1-[3-[[3-(difluoromethyl)-1-[4-[[1-[3-(2,6-dioxo-3-piperidyl)-1-methyl-indazol-6-yl]-4-piperidyl]carbamoyl]cyclohexyl]pyrazol-4-yl]carbamoyl]pyrazolo[1,5-a]pyrimidin-5-yl]-5-fluoro-3-piperidyl]carbamate (7). To a solution of 4-[3-(difluoromethyl)-4-[[5-[(3R,5R)-3-(tert-butoxycarbonylamino)-5-fluoro-1-piperidyl]pyrazolo[1,5-a]pyrimidine-3-carbonyl]amino]pyrazol-1-yl]cyclohexanecarboxylic acid 6 (90 mg, 0.15 mmol) and 3-[6-(4-amino-1-piperidyl)-1-methyl-indazol-3-yl]piperidine-2,6-dione hydrochloride C-3 (60.3 mg, 0.16 mmol) in DMF (1.2 mL) at 0° C. was added DIPEA (252 μL, 1.45 mmol). The mixture was stirred at 0° C. for 5 minutes, then HATU (55.1 mg, 0.15 mmol) was added. The resulting mixture was stirred at room temperature for 30 minutes. The mixture was directly loaded on a 30 g RediSep Rf Gold C18 chromatography column. Elution was done with MeCN/0.1% aqueous formic acid (5% for 5 CVs, then 5 to 35% over 15 CVs). The desired fractions were combined and concentrated to give 7 (119 mg, 82% yield) as a white solid.

LCMS method 1: 94.7% purity at 254 nm, [M+H]⁺=944.4, [M+2H]²⁺=473.0.

¹H NMR (400 MHz, DMSO-d6) δ ppm 1.34 (br s, 9H), 1.48-1.64 (m, 4H), 1.66-1.94 (m, 8H), 2.04-2.22 (m, 5H), 2.24-2.32 (m, 1H), 2.57-2.65 (m, 2H), 2.86 (br t, J=10.4 Hz, 2H), 2.96-3.15 (m, 1H), 3.37-3.53 (m, 2H), 3.60-3.70 (m, 1H), 3.75 (br d, J=11.7 Hz, 3H), 3.89 (s, 3H), 4.25 (br dd, J=9.2, 5.0 Hz, 2H), 4.93-5.14 (m, 1H), 6.81-7.24 (m, 5H), 7.49 (d, J=8.8 Hz, 1H), 7.78 (d, J=7.8 Hz, 1H), 8.28 (s, 1H), 8.35 (br s, 1H), 8.82 (d, J=8.1 Hz, 1H), 9.31 (br s, 1H), 10.84 (s, 1H).

Step 6. Preparation of 5-((3R,5R)-3-amino-5-fluoropiperidin-1-yl)-N-(3-(difluoromethyl)-1-((1r,4r)-4-((1-(3-(2,6-dioxopiperidin-3-yl)-1-methyl-1H-indazol-6-yl)piperidin-4-yl)carbamoyl)cyclohexyl)-1H-pyrazol-4-yl)pyrazolo[1,5-a]pyrimidine-3-carboxamide hydrochloride (P-4). In a round-bottom flask, tert-butyl N-[(3R,5R)-1-[3-[[3-(difluoromethyl)-1-[4-[[1-[3-(2,6-dioxo-3-piperidyl)-1-methyl-indazol-6-yl]-4-piperidyl]carbamoyl]cyclohexyl]pyrazol-4-yl]carbamoyl]pyrazolo[1,5-a]pyrimidin-5-yl]-5-fluoro-3-piperidyl]carbamate 7 (100 mg, 0.11 mmol) was solubilized in a 4 M HCl solution in dioxane (2 mL, 8.00 mmol). The solution was stirred at room temperature overnight. The solvent was evaporated under reduced pressure. The residue was dissolved in water, and the solution was loaded on a 30 g RediSep Rf Gold C18 chromatography column. Elution was done with MeCN/0.02 M aqueous HCl (5% for 3 CVs, then 5 to 20% over 20 CVs, then 20 to 30% over 15 CVs). Desired fractions were combined and concentrated. The residue was solubilized in water, frozen, and lyophilized to give P-4 (63.12 mg, 68% yield) as a light-yellow solid.

LCMS method 2: 98.4% purity at 215 nm, [M−HCl+H]⁺=844.5, [M−HCl+2H]²⁺=422.7.

¹H NMR (400 MHz, DMSO-d6) δ ppm 1.53-1.67 (m, 2H), 1.70-1.94 (m, 6H), 1.94-2.04 (m, 3H), 2.04-2.13 (m, 3H), 2.14-2.29 (m, 2H), 2.33-2.45 (m, 2H), 2.56-2.74 (m, 2H), 3.27-3.42 (m, 3H), 3.45-3.52 (m, 1H), 3.66-3.79 (m, 2H), 3.89-4.01 (m, 4H), 4.22-4.31 (m, 1H), 4.32-4.40 (m, 1H), 4.53-4.93 (m, 2H), 5.10 (d, J=46.5 Hz, 1H), 6.90 (d, J=8.1 Hz, 1H), 7.08 (t, J=54.5 Hz, 1H), 7.30-7.47 (m, 1H), 7.67-7.81 (m, 1H), 7.96-8.14 (m, 1H), 8.23-8.48 (m, 6H), 8.93 (d, J=7.8 Hz, 1H), 9.33 (s, 1H), 10.90 (s, 1H).

¹⁹F NMR (377 MHz, DMSO-d6) δ ppm −184.59 (s, 1F), −111.26 (s, 2F).

### Example S2. Synthesis of P-9

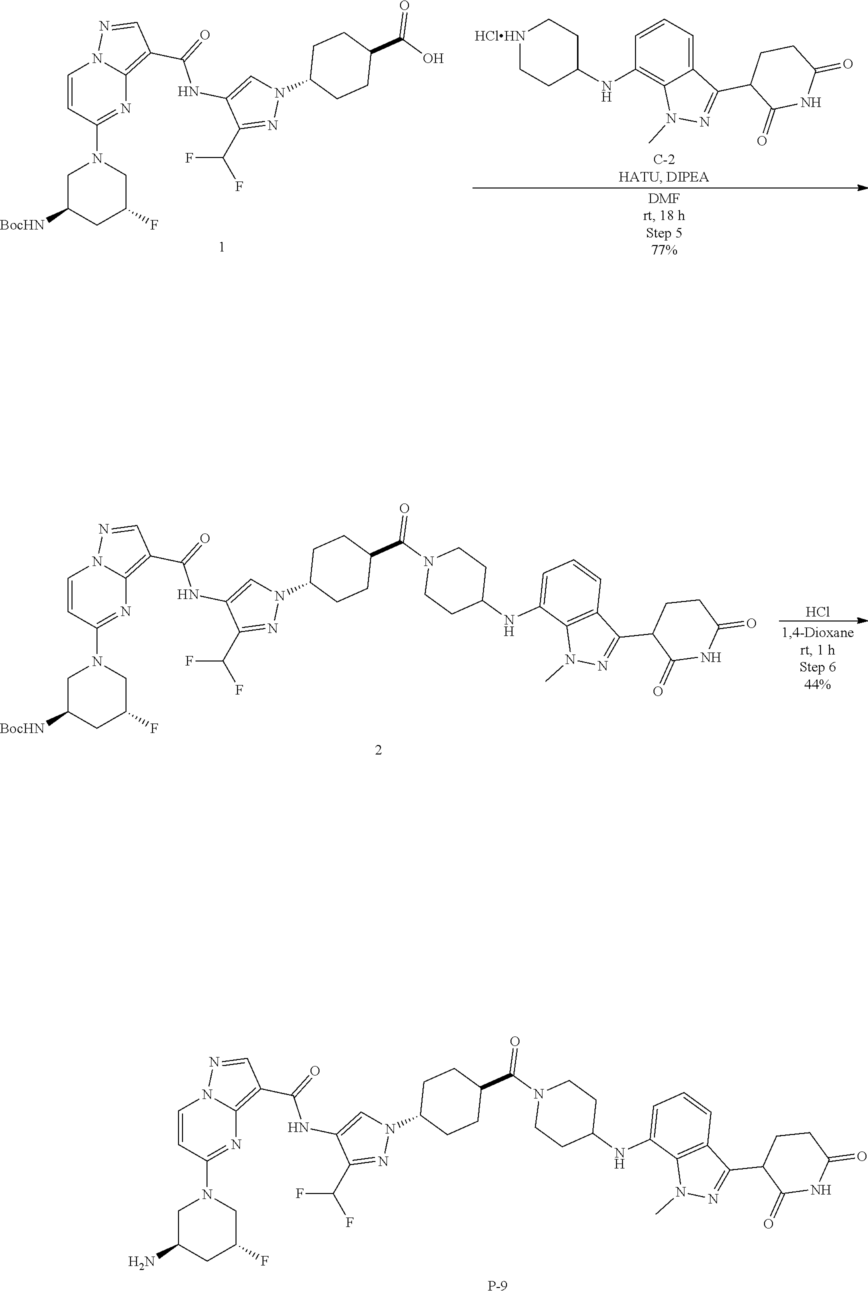

Step 5. Preparation of tert-butyl N-[(3R,5R)-1-[3-[[3-(difluoromethyl)-1-[4-[4-[[3-(2,6-dioxo-3-piperidyl)-1-methyl-indazol-7-yl]amino]piperidine-1-carbonyl]cyclohexyl]pyrazol-4-yl]carbamoyl]pyrazolo[1,5-a]pyrimidin-5-yl]-5-fluoro-3-piperidyl]carbamate (2). 4-[3-(difluoromethyl)-4-[[5-[(3R,5R)-3-(tert-butoxycarbonylamino)-5-fluoro-1-piperidyl]pyrazolo[1,5-a]pyrimidine-3-carbonyl]amino]pyrazol-1-yl]cyclohexanecarboxylic acid 1 (70.0 mg, 0.113 mmol, 1.0 equiv) and 3-[1-methyl-7-(4-piperidylamino)indazol-3-yl]piperidine-2,6-dione hydrochloride C-2 (55.4 mg, 0.146 mmol, 1.3 equiv) were dissolved in DMF (0.4 mL), then DIPEA (196 μL, 1.13 mmol, 10.0 equiv) and HATU (51.4 mg, 0.135 mmol, 1.3 equiv) were added in sequence. The reaction mixture was stirred overnight at room temperature. The reaction mixture was injected directly in a column for reversed-phase flash chromatography purification (MeCN in 0.1% HCOOH(aq), 5% (3 CV)→70%, 50 g RediSep Rf Gold® C18, 20 CV, λ=214-254 nm, product with 60-65% MeCN). Evaporation of the fractions yielded 2 (82 mg, 0.087 mmol, 77% yield) as a white solid.

LCMS method 1: 99.9% purity at 215 nm, [M+H]⁺=945.4, [M+2H]²⁺=473.0.

¹H NMR (400 MHz, DMSO-d6) δ ppm 1.34 (br s, 9H), 1.39-1.47 (m, 1H), 1.48-1.68 (m, 3H), 1.82 (br d, J=15.2 Hz, 3H), 1.86-1.98 (m, 3H), 1.99-2.07 (m, 4H), 2.14 (br dd, J=13.3, 5.4 Hz, 2H), 2.23-2.36 (m, 1H), 2.56-2.71 (m, 2H), 2.78 (br t, J=11.6 Hz, 1H), 2.85-2.96 (m, 1H), 2.99-3.13 (m, 1H), 3.41-3.54 (m, 2H), 3.55-3.76 (m, 2H), 3.99 (br d, J=12.5 Hz, 1H), 4.24 (s, 4H), 4.26-4.35 (m, 2H), 4.50-4.92 (m, 1H), 4.94-5.17 (m, 2H), 6.61 (d, J=7.3 Hz, 1H), 6.84-6.95 (m, 2H), 6.96-7.27 (m, 3H), 8.29 (s, 1H), 8.35 (br s, 1H), 8.81 (br d, J=7.8 Hz, 1H), 9.32 (br s, 1H), 10.85 (s, 1H).

¹⁹F NMR (377 MHz, DMSO-d6) δ ppm −185.51-−183.43 (m, 1F), −112.85-−109.73 (m, 2F).

Step 6. Preparation of 5-((3R,5R)-3-amino-5-fluoropiperidin-1-yl)-N-(3-(difluoromethyl)-1-((1r,4r)-4-(4-((3-(2,6-dioxopiperidin-3-yl)-1-methyl-1H-indazol-7-yl)amino)piperidine-1-carbonyl)cyclohexyl)-1H-pyrazol-4-yl)pyrazolo[1,5-a]pyrimidine-3-carboxamide hydrochloride (P-9). HCl 4.0 M in 1,4-dioxane (3.17 mL, 12.7 mmol, 150 equiv) was added to tert-butyl N-[(3R,5R)-1-[3-[[3-(difluoromethyl)-1-[4-[4-[[3-(2,6-dioxo-3-piperidyl)-1-methyl-indazol-7-yl]amino]piperidine-1-carbonyl]cyclohexyl]pyrazol-4-yl]carbamoyl]pyrazolo[1,5-a]pyrimidin-5-yl]-5-fluoro-3-piperidyl]carbamate 2 (80.0 mg, 0.085 mmol, 1.0 equiv) and the mixture was sonicated for 30 minutes, then stirred for 1 hour. The solvent was evaporated to dryness and the residue was purified by reversed-phase flash chromatography (MeCN in 0.02 M HCl(aq), 5% (3 CV)→50%, 50 g RediSep Rf Gold® C18, 15 CV, λ=214-254 nm, product with 33-39% MeCN) to yield the impure product (50 mg). The pure product was obtained after prep HPLC purification (MeCN in HCl(aq)): the fractions containing the product were evaporated to dryness, then co-evaporated with water (3×10 mL) to completely remove any trace of residual HCl, and the residue was freeze-dried overnight to afford P-9 (31.48 mg, 0.0371 mmol, 43.842% yield) as a white solid.

LCMS method 2: 99.5% purity at 215 nm, [M+H]⁺=844.5, [M+2H]²⁺=422.7.

¹H NMR (400 MHz, DMSO-d6) δ ppm 1.34-1.70 (m, 5H), 1.72-2.10 (m, 10H), 2.10-2.21 (m, 1H), 2.22-2.35 (m, 1H), 2.35-2.45 (m, 1H), 2.53-2.71 (m, 2H), 2.78 (t, J=11.4 Hz, 1H), 2.90 (t, J=11.3 Hz, 1H), 3.20-3.35 (m, 2H), 3.35-3.55 (m, 2H), 3.55-3.71 (m, 1H), 3.94-4.05 (m, 1H), 4.25-4.34 (m, 5H), 4.61 (br s, 1H), 4.83 (br s, 1H), 5.10 (d, J=45.0 Hz, 1H), 6.65 (d, J=6.7 Hz, 1H), 6.83-6.96 (m, 2H), 6.97-7.32 (m, 2H), 8.29 (br s, 3H), 8.33 (s, 1H), 8.39 (s, 1H), 8.93 (d, J=7.8 Hz, 1H), 9.34 (s, 1H), 10.86 (s, 1H).

¹⁹F NMR (377 MHz, DMSO-d6) δ ppm −184.68 (s, 1F), −111.29 (s, 2F).

The following compounds were synthesized via the same general routes with modifications to CBM (C—X) in step 5 (Table 4).

TABLE 4

| Compound | | | |
|---|---|---|---|
| No. | CBM | Structure | Characterization Data |
| P-1 | C-1 | 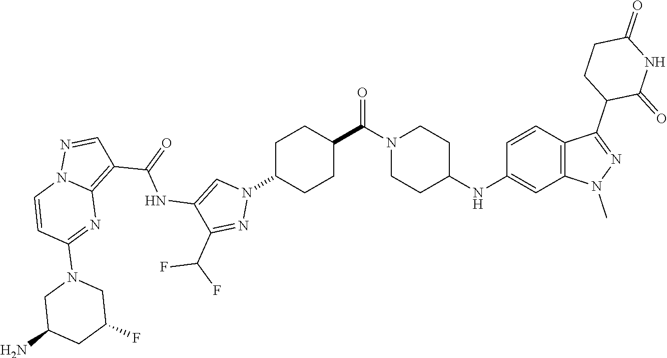 | 63% yield as a white solid. LCMS method 2: retention time: 2.169 min, 99% purity at 215 nm, [M − HCl + H]⁺ = 844.4; [M − HCl + 2H]²⁺ = 422.8. ¹H NMR (400 MHz, DMSO-d6 ) δ ppm 1.23-1.41 (m, 2 H), 1.52- 1.67 (m, 2 H), 1.78-2.09 (m, 9 H), 2.11-2.19 (m, 1 H), 2.22- 2.44 (m, 3 H), 2.58-2.68 (m, 2 H), 2.74-2.87 (m, 2 H), 3.20-3.33 (m, 2 H), 3.36-3.52 (m, 3 H), 3.62-3.68 (m, 1 H), 3.97-4.05 (m, 2 H), 4.17-4.37 (m, 4 H), 4.50-4.70 (m, 1 H), 4.77-4.95 (m, 1 H), 5.10 (d, J = 45.0 Hz, 1 H), 6.49-6.69 (m, 2 H), 6.88 (d, J = 8.1 Hz, 1 H), 7.13 (t, J = 54.0 Hz, 1 H), 7.35-7.43 (m, 1 H), 8.16-8.27 (m, 3 H), 8.33 (s, 1 H), 8.39 (s, 1 H), 8.93 (d, J = 8.1 Hz, 1 H), 9.34 (s, 1 H), 10.83 (s, 1 H). - Three protons partially under the peak of water in the ¹H NMR ¹⁹F NMR (377 MHz, DMSO-d6 ) δ ppm −185.20 (s, 1 F), −111.24 (s, 2 F). |
| P-4 | C-3 | 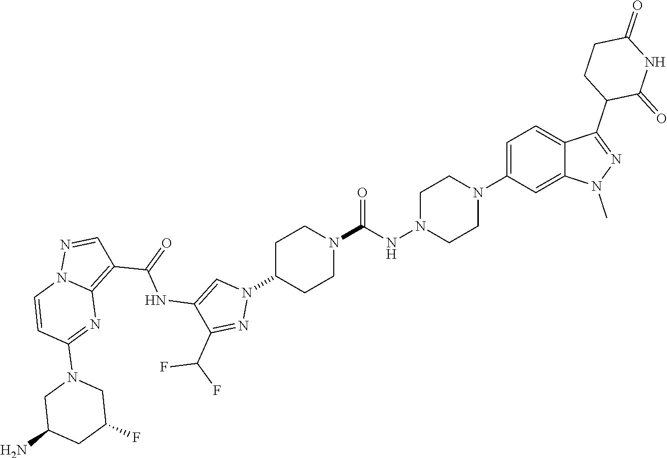 | 70% yield as a light yellow solid. LCMS method 2: retention time: 2.093 min, 98% purity at 215 nm, [M − HCl + H]⁺ = 844.5, [M − HCl + 2H]²⁺ = 422.7. ¹H NMR (400 MHz, DMSO-d₆) δ ppm 1.53-1.67 (m, 2 H), 1.70- 1.94 (m, 6 H), 1.94-2.04 (m, 3 H), 2.04-2.13 (m, 3 H), 2.14-2.29 (m, 2 H), 2.33-2.45 (m, 2 H), 2.56-2.74 (m, 2 H), 3.27-3.42 (m, 3 H), 3.45-3.52 (m, 1 H), 3.66-3.79 (m, 2 H), 3.89-4.01 (m, 4 H), 4.22-4.31 (m, 1 H), 4.32-4.40 (m, 1 H), 4.53-4.93 (m, 2 H), 5.10 (d, J = 46.5 Hz, 1 H), 6.90 (d, J = 8.1 Hz, 1 H), 7.08 (t, J = 54.5 Hz, 1 H), 7.30-7.47 (m, 1 H), 7.67-7.81 (m, 1 H), 7.96-8.14 (m, 1 H), 8.23-8.48 (m, 6 H), 8.93 (d, J = 7.8 Hz, 1 H), 9.33 (s, 1 H), 10.90 (s, 1 H) ¹⁹F NMR (377 MHz, DMSO-d6) δ ppm −184.59 (s, 1 F), −111.26 (s, 2 F). |
| P-6 | C-4 | 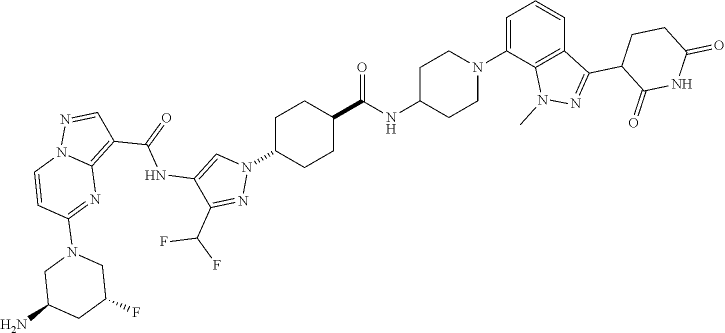 | 80% yield as a off-white solid. LCMS method 2: retention time: 2.336 min, 99% purity at 215 nm, [M + 2H]²⁺ = 422.8, [M + H]⁺ = 844.4. ¹H NMR (400 MHz, DMSO-d₆) δ ppm 1.54-1.82 (m, 6 H), 1.83- 2.03 (m, 5 H), 2.03-2.13 (m, 2 H), 2.13-2.26 (m, 2 H), 2.28-2.45 (m, 2 H), 2.56-2.68 (m, 2 H), 2.68-2.87 (m, 2 H), 3.14-3.27 (m, 2 H), 3.28-3.53 (m, 3 H), 3.61-3.80 (m, 1 H), 4.20-4.31 (m, 4 H), 4.34 (dd, J = 9.0, 5.1 Hz, 1 H), 4.50-4.73 (m, 1 H), 4.74-4.97 (m, 1 H), 5.03-5.19 (m, 1 H), 6.91 (d, J = 7.6 Hz, 1 H), 6.97-7.31 (m, 3 H), 7.38 (d, J = 7.1 Hz, 1 H), 7.86 (d, J = 6.4 Hz, 1 H), 8.33 (s, 1 H), 8.34-8.48 (m, 4 H), 8.93 (d, J = 7.8 Hz, 1 H), 9.33 (s, 1 H), 10.88 (s, 1 H). ¹⁹F NMR (377 MHz, DMSO-d6) δ ppm −184.60 (s, 1 F), −111.27 (s, 2 F). |
| P-9 | C-2 | 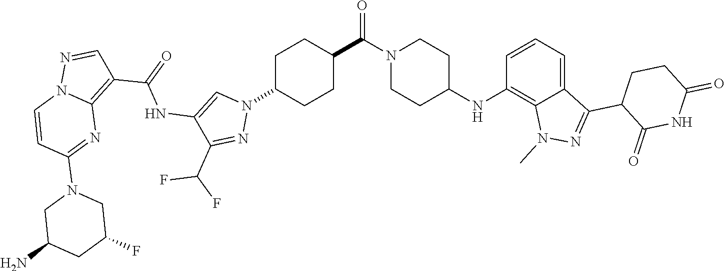 | 44% yield as a white solid. LCMS method 2: retention time: 2.295 min, 99% purity at 215 nm, [M + H]⁺ = 844.5, [M + 2H]²⁺ = 422.7. ¹H NMR (400 MH_{Z}, DMSO-d6) δ ppm 1.34-1.70 (m, 5 H), 1.72- 2.10 (m, 10 H), 2.10-2.21 (m, 1 H), 2.22-2.35 (m, 1 H), 2.35- 2.45 (m, 1 H), 2.53-2.71 (m, 2 H), 2.78 (t, J = 11.4 Hz, 1 H), 2.90 (t, J = 11.3 Hz, 1 H), 3.20-3.35 (m, 2 H), 3.35-3.55 (m, 2 H), 3.55- 3.71 (m, 1 H), 3.94-4.05 (m, 1 H), 4.25-4.34 (m, 5 H), 4.61 (br s, 1 H), 4.83 (br s, 1 H), 5.10 (d, J = 45.0 Hz, 1 H), 6.65 (d, J = 6.7 Hz, 1 H), 6.83-6.96 (m, 2 H), 6.97-7.32 (m, 2 H), 8.29 (br s, 3 H), 8.33 (s, 1 H), 8.39 (s, 1 H), 8.93 (d, J = 7.8 Hz, 1 H), 9.34 (s, 1 H), 10.86 (s, 1 H). ¹⁹F NMR (377 MHz, DMSO-d6) δ ppm −184.68 (s, 1 F), −111.29 (s, 2 F). |

### Final Product General Method 2

### Example S3. Synthesis of P-2

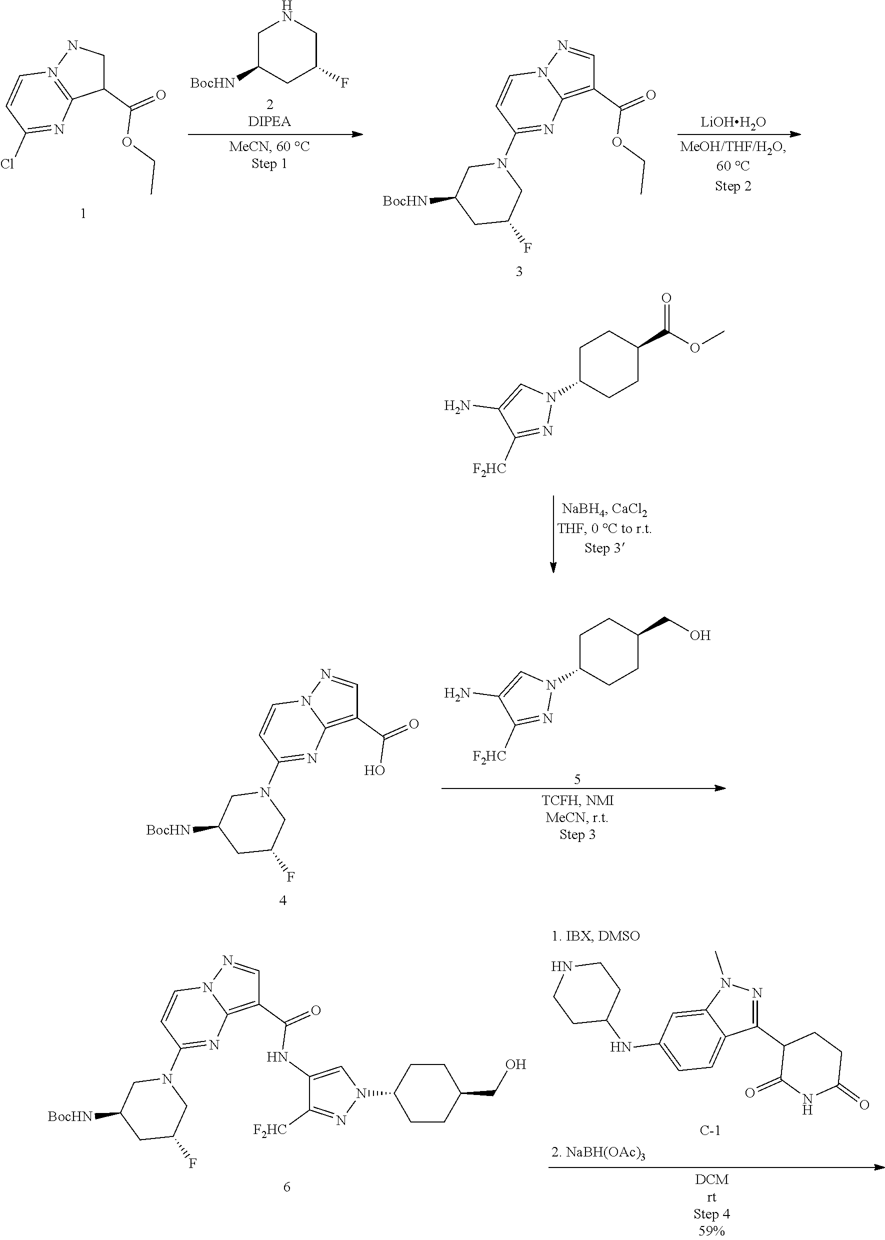

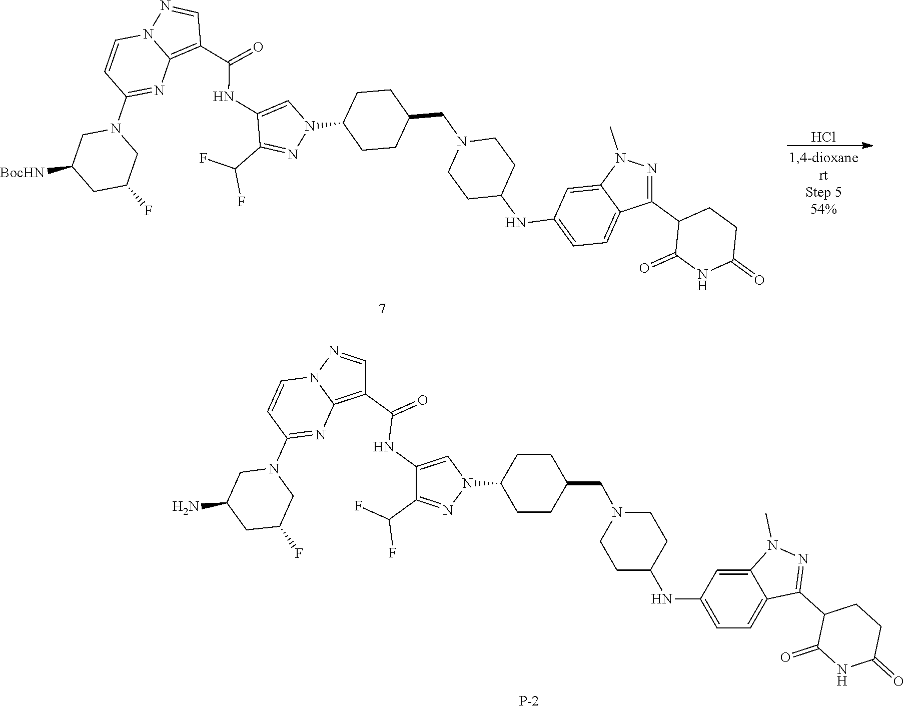

Step 1. Preparation of ethyl 5-[(3R,5R)-3-(tert-butoxycarbonylamino)-5-fluoro-1-piperidyl]pyrazolo[1,5-a]pyrimidine-3-carboxylate (3). To a solution of ethyl 5-chloropyrazolo[1,5-a]pyrimidine-3-carboxylate 1 (800 mg, 3.55 mmol, 1.0 eq.) in MeCN (17.7 mL) was added DIPEA (1.54 mL, 8.86 mmol, 2.5 eq.) and tert-butyl N-[(3R,5R)-5-fluoro-3-piperidyl]carbamate 2 (1.01 g, 4.61 mmol, 1.3 eq.). After stirring at 60° C. over the weekend, LCMS showed complete conversion into 3. The solvent was removed under reduced pressure and the residue was dried under high vacuum to give 3 (1.44 g, quantitative yield) as white solid. The crude product was used in the next step without further purification.

LCMS method 1: retention time: 1.656 min, 99.900 purity at 215 nm, [M+H]=408.2.

¹H NMR (400 MHz, DMSO-d6) δ ppm 1.37-1.45 (m, 9H), 1.56-1.92 (m, 1H), 2.09-2.20 (m, 1H), 2.56-2.71 (m, 1H), 2.76-3.03 (m, 1H), 3.08-3.19 (m, 1H), 3.29 (s, 1H), 3.37 (br s, 1H), 3.56-3.73 (m, 2H), 4.11-4.25 (m, 2H), 4.41-4.73 (m, 1H), 4.99 (br s, 1H), 5.11 (br s, 1H), 6.85 (br d, J=8.1 Hz, 1H), 7.10 (br d, J=7.8 Hz, 1H), 8.22 (s, 1H), 8.73 (d, J=7.8 Hz, 1H).

¹⁹F NMR (377 MHz, DMSO-d6) δ ppm −184.32 (s, 1F).

Step 2. Preparation of 5-[(3R,5R)-3-(tert-Butoxycarbonylamino)-5-fluoro-1-piperidyl]pyrazolo[1,5-a]pyrimidine-3-carboxylic acid (4). To a solution of ethyl 5-[(3R,5R)-3-(tert-butoxycarbonylamino)-5-fluoro-1-piperidyl]pyrazolo[1,5-a]pyrimidine-3-carboxylate 3 (1.44 mg, 3.53 mmol, 1.0 eq) in THF (5.89 mL) and methanol (5.89 mL) was added a solution of LiOH·H₂O (1.48 g, 35.34 mmol, 10.0 eq.) in water (5.89 mL). After stirring at 60° C. for 18 h, LCMS showed complete conversion into desired product 4. The reaction mixture was concentrated under vacuum to remove THF/MeOH and the crude mixture was diluted with water. Under vigorous agitation, the mixture was acidified with a 6N aqueous HCl solution until pH=3 (formation of a precipitate). The suspension was filtered on a Buchner funnel and the solid was rinsed with water. The solid was dried overnight in a stove under vacuum to give 4 (1.40 g, quantitative yield) as white solid. The crude product was used in the next without further purification.

LCMS method 1: retention time: 1.487 min, 99.9% purity at 215 nm, [M+H]⁺=380.1.

¹H NMR (400 MHz, DMSO-d6) δ ppm 1.41 (s, 9H), 1.66-1.95 (m, 1H), 2.05-2.26 (m, 1H), 2.92-3.11 (m, 1H), 3.28-3.30 (m, 1H), 3.34-3.49 (m, 1H), 3.59-3.74 (m, 1H), 4.52-4.75 (m, 1H), 4.99 (br s, 1H), 5.11 (br s, 1H), 6.82 (br d, J=7.6 Hz, 1H), 7.12 (br d, J=7.8 Hz, 1H), 8.19 (s, 1H), 8.73 (d, J=7.8 Hz, 1H).

¹⁹F NMR (377 MHz, DMSO-d6) δ ppm −184.32 (s, 1F).

Step 3′. Preparation of [4-[4-Amino-3-(difluoromethyl)pyrazol-1-yl]cyclohexyl]methanol (5). To a solution of methyl 4-[4-amino-3-(difluoromethyl)pyrazol-1-yl]cyclohexanecarboxylate T-1 (1.23 g, 4.5 mmol, 1.0 eq.) in THF (15.0 mL, 0.1 M) and ethanol (30.0 mL) at 0° C. was added CaCl₂) (1.0 g, 9.0 mmol, 2.0 eq,), followed by NaBH₄ (0.68 g, 18.0 mmol, 4.0 eq.). The resulting mixture was stirred overnight, allowing it to warm up to room temperature. After overnight water was added and the reaction mixture was stirred at room temperature for 1 hour. The aqueous phase was extracted with EtOAc (3×). The organics were washed with brine, dried over Na₂SO₄ and concentrated to dryness, which afforded [4-[4-amino-3-(difluoromethyl)pyrazol-1-yl]cyclohexyl]methanol 5 (1.1 g, 99% yield) as an orange oil.

LCMS method 1: retention time: 0.989 min, 99.0% purity at 215 nm, [M+H]⁺=246.2.

¹H NMR (400 MHz, DMSO-d6) δ ppm 1.04 (qd, J=12.9, 3.2 Hz, 2H), 1.32-1.43 (m, 1H), 1.62 (qd, J=12.6, 3.3 Hz, 2H), 1.82 (br d, J=11.5 Hz, 2H), 1.93-1.98 (m, 2H), 3.24 (t, J=5.7 Hz, 2H), 3.94 (tt, J=11.9, 3.8 Hz, 1H), 4.00-4.06 (m, 2H), 4.44 (t, J=5.3 Hz, 1H), 6.72-7.02 (m, 1H), 7.14 (s, 1H).

¹⁹F NMR (377 MHz, DMSO-d6) δ ppm −111.21 (s, 2F).

Step 3. Preparation of tert-Butyl N-[(3r,5r)-1-[3-[[3-(difluoromethyl)-1-[4-(hydroxymethyl)cyclohexyl]pyrazol-4-yl]carbamoyl]pyrazolo[1,5-a]pyrimidin-5-yl]-5-fluoro-3-piperidyl]carbamate (6). To a solution of [4-[4-amino-3-(difluoromethyl)pyrazol-1-yl]cyclohexyl]methanol 5 (511.8 mg, 1.88 mmol, 1.5 eq.), 5-[(3r,5r)-3-(tert-butoxycarbonylamino)-5-fluoro-1-piperidyl]pyrazolo[1,5-a]pyrimidine-3-carboxylic acid 4 (475.0 mg, 1.25 mmol, 1.0 eq.) and NMI (362.72 μL, 4.58 mmol, 3.6 eq.) in MeCN (12.5 mL, 0.1 M) was added TCFH (440.43 mg, 1.57 mmol, 1.2 eq.). The resulting mixture was stirred at room temperature overnight. Water was added and the reaction mixture was stirred at room temperature for 1 hour. The solid was filtered on a Buchner funnel and rinsed with a water/MeCN mixture. Purification by reverse phase chromatography (50 g C18 RediSep Rf Gold column, liquid deposit (DMSO), elution: 5% MeOH/0.1% HCOOH over 4 CV, then 5% to 100% MeOH/0.1% HCOOH over 15 CV). The pure fractions were combined, concentrated under reduced pressure to afford tert-butyl N-[(3r,5r)-1-[3-[[3-(difluoromethyl)-1-[4-(hydroxymethyl)cyclohexyl]pyrazol-4-yl]carbamoyl]pyrazolo[1,5-a]pyrimidin-5-yl]-5-fluoro-3-piperidyl]carbamate 6 (421 mg, 55% yield) as a tan solid.

LCMS method 1: retention time: 1.629 min, 99.9% purity at 215 nm, [M+H]⁺=607.2.

¹H NMR (400 MHz, DMSO-d6) δ ppm 1.03-1.15 (m, 2H), 1.34 (br s, 9H), 1.64-1.80 (m, 3H), 1.82-1.93 (m, 3H), 1.99-2.07 (m, 2H), 2.08-2.18 (m, 1H), 2.95-3.14 (m, 1H), 3.26 (t, J=5.7 Hz, 2H), 3.37-3.52 (m, 1H), 3.62-3.71 (m, 1H), 4.12-4.21 (m, 1H), 4.47 (t, J=5.3 Hz, 1H), 4.69 (br s, 1H), 4.95-5.12 (m, 1H), 6.86-6.94 (m, 1H), 7.01-7.22 (m, 2H), 8.28 (s, 1H), 8.34 (br s, 1H), 8.82 (d, J=7.8 Hz, 1H), 9.31 (br s, 1H).

¹⁹F NMR (377 MHz, DMSO-d6) δ ppm −186.02-−179.79 (m, 1F), −113.10-−108.69 (m, 2F).

Step 4. Preparation of tert-butyl N-[(3R,5R)-1-[3-[[3-(difluoromethyl)-1-[4-[[4-[[3-(2,6-dioxo-3-piperidyl)-1-methyl-indazol-6-yl]amino]-1-piperidyl]methyl]cyclohexyl]pyrazol-4-yl]carbamoyl]pyrazolo[1,5-a]pyrimidin-5-yl]-5-fluoro-3-piperidyl]carbamateformic acid salt (7). To a solution of tert-butyl N-[(3R,5R)-1-[3-[[3-(difluoromethyl)-1-[4-(hydroxymethyl)cyclohexyl]pyrazol-4-yl]carbamoyl]pyrazolo[1,5-a]pyrimidin-5-yl]-5-fluoro-3-piperidyl]carbamate 6 (328.7 mg, 0.54 mmol, 1.0 eq.) in dry DMSO (2.7 mL, 0.1 M) was added IBX (182.07 mg, 0.65 mmol, 1.2 eq.). The resulting mixture was stirred at room temperature overnight. After an overnight stirring, LCMS showed complete conversion into the aldehyde. To the reaction mixture was added 3-[1-methyl-6-(4-piperidylamino)indazol-3-yl]piperidine-2,6-dione C-1 (344.51 mg, 1.63 mmol, 1.2 eq.), DCM (2.71 mL, 0.1 M) and DIPEA (0.94 mL, 5.42 mmol, 10.0 eq.). The mixture was stirred at room temperature for 10 minutes, then sodium triacetoxyborohydride (344.51 mg, 1.63 mmol, 3 eq.) was added. After 1 h, LCMS showed complete conversion into compound 3. DCM was evaporated under reduced pressure and the crude mixture was purified by reverse phase FC purification (50 g C18 RediSep Rf Gold column, liquid deposit (DMSO), 5% MeCN/0.1% HCOOH over 4 CV, then 5 to 70% MeCN/0.1% HCOOH over 20 CV, product came out at 50% MeCN). Pure fractions were combined and concentrated to give 7 (313 mg, 59% yield) as a tan solid as a formic acid salt. The desired product contained IBX residue.

LCMS method 3: 95.5% purity at 215 nm, [M−HCOOH+H]⁺=930.4.

¹H NMR (400 MHz, DMSO-d6) δ ppm 1.00-1.09 (m, 2H), 1.34 (br s, 9H), 1.40-1.51 (m, 3H), 1.62 (br d, J=6.1 Hz, 1H), 1.70-1.83 (m, 3H), 1.85-1.95 (m, 3H), 1.95-2.01 (m, 2H), 2.01-2.07 (m, 2H), 2.15 (br t, J=5.1 Hz, 3H), 2.18-2.35 (m, 4H), 2.60 (br t, J=6.6 Hz, 2H), 2.83-2.92 (m, 2H), 3.29-3.40 (m, 2H), 3.61-3.73 (m, 1H), 3.81 (s, 3H), 4.11-4.24 (m, 3H), 4.95-5.13 (m, 1H), 5.67-5.84 (m, 1H), 6.38 (s, 1H), 6.52 (br d, J=8.8 Hz, 1H), 6.84-6.95 (m, 1H), 6.99-7.22 (m, 2H), 7.32 (d, J=8.8 Hz, 1H), 8.16 (s, 1H), 8.29 (s, 1H), 8.31-8.36 (m, 1H), 8.82 (br d, J=8.1 Hz, 1H), 9.31 (br s, 1H), 10.82 (s, 1H).

Step 5. Preparation of N-[3-(difluoromethyl)-1-[4-[[4-[[3-(2,6-dioxo-3-piperidyl)-1-methyl-indazol-6-yl]amino]-1-piperidyl]methyl]cyclohexyl]pyrazol-4-yl]-5-[(3R,5R)-3-amino-5-fluoro-1-piperidyl]pyrazolo[1,5-a]pyrimidine-3-carboxamide (P-2). To a round bottom flask was added tert-butyl N-[(3R,5R)-1-[3-[[3-(difluoromethyl)-1-[4-[[4-[[3-(2,6-dioxo-3-piperidyl)-1-methyl-indazol-6-yl]amino]-1-piperidyl]methyl]cyclohexyl]pyrazol-4-yl]carbamoyl]pyrazolo[1,5-a]pyrimidin-5-yl]-5-fluoro-3-piperidyl]carbamate 7 (308.35 mg, 0.33 mmol, 1 eq.) and 4 M HCl in dioxane (10.28 mL, 41.11 mmol, 124 eq.). The reaction was stirred at r.t. LCMS after 18 h showed complete conversion into P-2. The reaction mixture was concentrated in vacuo and the residue was purified by reverse-phase FC (50 g C18 RediSep Rf Gold column, liquid deposit (H₂O), elution: 5% MeCN/0.02 M HCl over 4 CV, then 5% to 30% MeCN/0.02 M HCl over 20 CV, product exited at 25% MeCN). Fractions were combined and concentrated, affording P-2 (149.83 mg, 54% yield) as a tan solid as a hydrochloride salt.

LCMS method 4: 99.9% purity at 215 nm, [M−HCl+H]⁺=830.3; [M−HCl+2H]²⁺=415.7.

¹H NMR (400 MHz, DMSO-d6) δ ppm 1.14-1.30 (m, 2H), 1.76-2.46 (m, 15H), 2.58-2.65 (m, 2H), 2.93-3.17 (m, 4H), 3.26-3.51 (m, 4H), 3.54-3.65 (m, 2H), 4.18-4.30 (m, 2H), 4.55-4.72 (m, 1H), 4.75-4.96 (m, 1H), 5.02-5.19 (m, 1H), 6.53-6.69 (m, 2H), 6.91 (br d, J=8.1 Hz, 1H), 7.14 (t, J=54.0 Hz, 1H), 7.41 (br d, J=8.8 Hz, 1H), 8.31-8.34 (m, 1H), 8.40 (s, 4H), 8.93 (d, J=7.8 Hz, 1H), 9.34 (s, 1H), 9.93 (br s, 1H), 10.84 (s, 1H).

¹⁹F NMR (377 MHz, DMSO-d6) δ ppm −184.68 (s, 1F), −111.54-−110.76 (m, 2F).

### Example S4. Synthesis of P-5

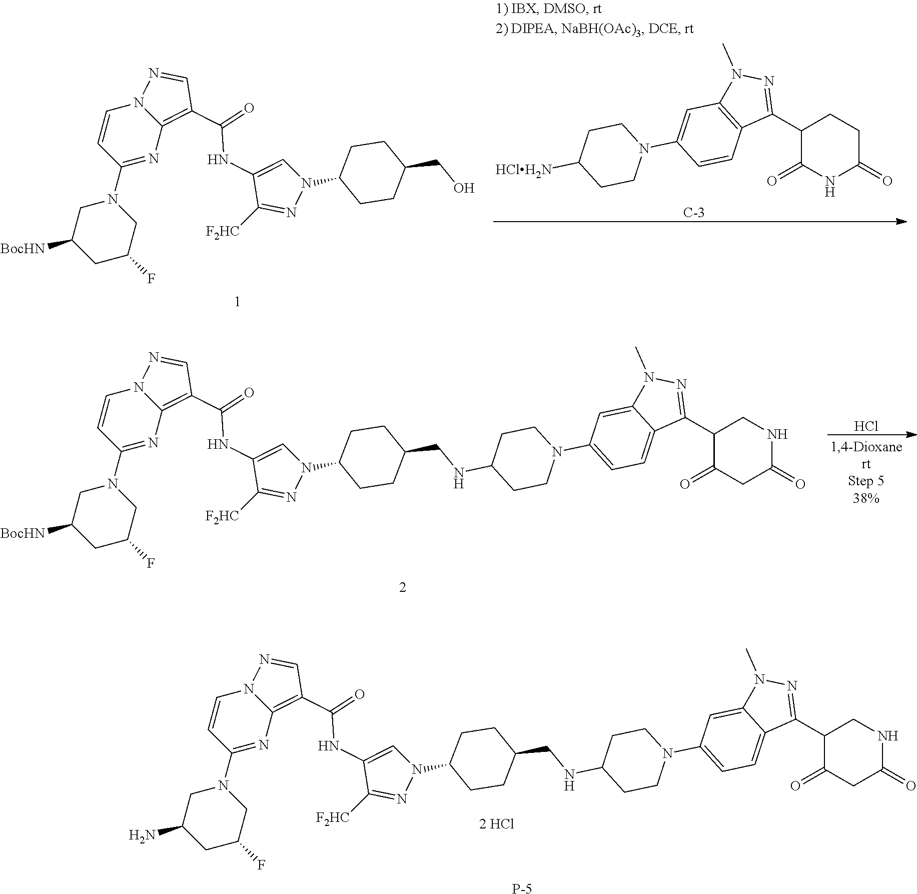

Step 4. Preparation of tert-Butyl N-[(3R,5R)-1-[3-[[3-(difluoromethyl)-1-[4-[[[1-[3-(2,6-dioxo-3-piperidyl)-1-methyl-indazol-6-yl]-4-piperidyl]amino]methyl]cyclohexyl]pyrazol-4-yl]carbamoyl]pyrazolo[1,5-a]pyrimidin-5-yl]-5-fluoro-3-piperidyl]carbamate (2). To a solution of tert-butyl N-[(3R,5R)-1-[3-[[3-(difluoromethyl)-1-[4-(2-hydroxyethyl)cyclohexyl]pyrazol-4-yl]carbamoyl]pyrazolo[1,5-a]pyrimidin-5-yl]-5-fluoro-3-piperidyl]carbamate 1—intermediate 6 from synthesis of P-2 —(58.6 mg, 0.097 mmol, 1 eq.) in dry DMSO (1.0 mL) was added IBX (32.6 mg, 0.116 mmol, 1.2 eq.). The resulting mixture was stirred at rt. After 16 h, LCMS showed total conversion toward the aldehyde. The aldehyde in DMSO was then added to a solution of 3-[6-(4-amino-1-piperidyl)-1-methyl-indazol-3-yl]piperidine-2,6-dione hydrochloride C-3 (44.2 mg, 0.107 mmol, 1.1 eq.) and DIPEA (168 μL, 0.969 mmol, 10 eq.) in DCE (1 mL). The mixture was stirred at rt for 10 minutes and NaBH(OAc)₃ (26.7 mg, 0.126 mmol, 1.3 eq.) was added. The resulting mixture was then stirred at room temperature. After 1 h, LCMS showed total completion. DCE was removed under reduced pressure and the residue was purified by reverse phase flash chromatography (50 g C18 RediSep Rf Gold column, liquid deposit (DMSO), elution: 5% MeCN/0.1% HCOOH over 3 CV, then 5 to 35% MeCN/0.1% HCOOH over 15 CV, then 35 to 100% MeCN/0.1% HCOOH over 1 CV, then 100% MeCN/0.1% HCOOH over 3 CV (desired product was released around 30% MeCN). Fractions were combined and concentrated to give 2 (48.4 mg, 54% yield) as a white solid. The product was used without purification in the next step.

LCMS method 1: 99.9% purity at 215 nm, [M+2H]²⁺=466.

Step 5. Preparation of 5-((3R,5R)-3-Amino-5-fluoropiperidin-1-yl)-N-(3-(difluoromethyl)-1-((1r,4r)-4-(((1-(3-(2,6-dioxopiperidin-3-yl)-1-methyl-1H-indazol-6-yl)piperidin-4-yl)amino)methyl)cyclohexyl)-1H-pyrazol-4-yl)pyrazolo[1,5-a]pyrimidine-3-carboxamide dihydrochloride (P-5). tert-Butyl N-[(3R,5R)-1-[3-[[3-(difluoromethyl)-1-[4-[[[[1-[3-(2,6-dioxo-3-piperidyl)-1-methyl-indazol-6-yl]-4-piperidyl]amino]methyl]cyclohexyl]pyrazol-4-yl]carbamoyl]pyrazolo[1,5-a]pyrimidin-5-yl]-5-fluoro-3-piperidyl]carbamate 2 (48.4 mg, 0.052 mmol, 1 eq.) was solubilized in a 4 M HCl solution in 1,4-dioxane (1.95 mL, 7.80 mmol, 150 eq.). The mixture was stirred at rt. After 2 h, LCMS showed total completion. Volatiles were evaporated under vacuum. The residue was purified by reverse phase flash chromatography (30 g C18 RediSep Rf Gold column, liquid deposit (Water), elution: 5% MeCN/0.02 M HCl over 5 CV, then 5 to 20% MeCN/0.02 M HCl over 15 CV, then 20% MeCN/0.02 M HCl over 5 CV, then 20 to 100% MeCN/0.02 M HCl over 2 CV then 100% MeCN/0.02 M HCl over 3 CV). Fractions were combined and concentrated to give P-5 (16.61 mg, 38% yield) as a white solid.

LCMS method 2: 99.6% purity at 215 nm, [M-2HCl+2H]²⁺=415.7, [M-2HCl+H]⁺=830.4.

¹H NMR (400 MHz, DMSO-d6) δ ppm 1.12-1.32 (m, 2H), 1.58-1.86 (m, 5H), 1.88-2.02 (m, 2H), 2.04-2.23 (m, 5H), 2.26-2.36 (m, 2H), 2.58-2.68 (m, 3H), 2.77-2.97 (m, 3H), 3.22-3.53 (m, 5H), 3.86-3.92 (m, 3H), 3.92-4.03 (m, 2H), 4.16-4.36 (m, 2H), 4.47-4.93 (m, 2H), 5.10 (d, J=46.2 Hz, 1H), 6.78-6.99 (m, 3H), 6.99-7.31 (m, 1H), 7.48-7.57 (m, 1H), 8.06-8.29 (m, 3H), 8.33 (s, 1H), 8.42 (s, 1H), 8.47-8.69 (m, 2H), 8.91-9.00 (m, 1H), 9.33 (s, 1H), 10.85 (s, 1H).

¹⁹F NMR (377 MHz, DMSO-d6) δ ppm −184.50 (s, 1F), −111.28 (s, 2F).

### Example S5. Synthesis of P-18

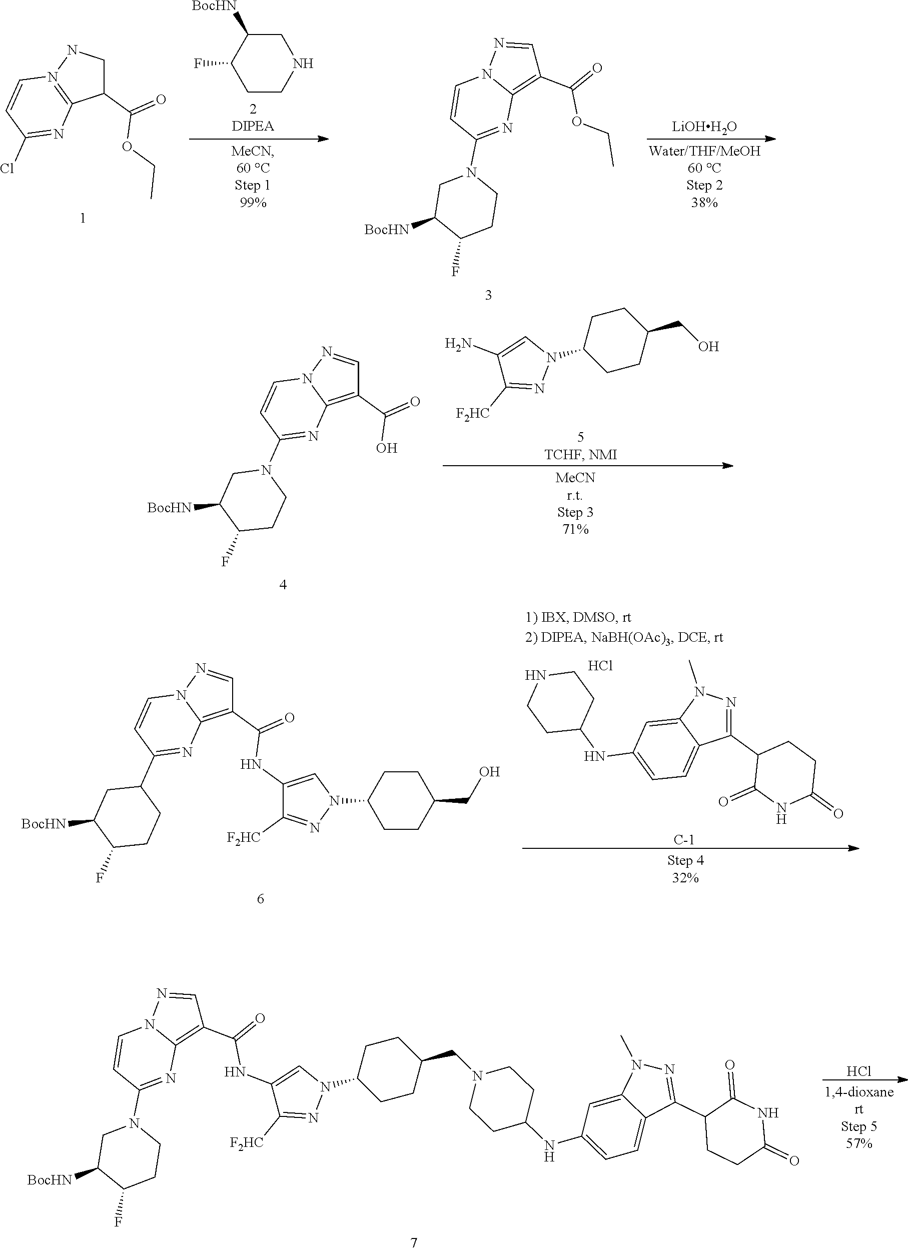

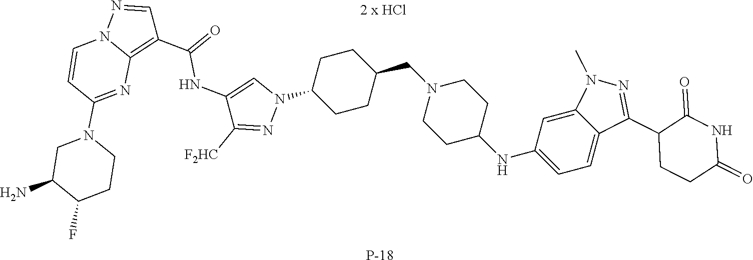

Step 1. Preparation of Ethyl 5-[(3S,4S)-3-(tert-butoxycarbonylamino)-4-fluoro-1-piperidyl]pyrazolo[1,5-a]pyrimidine-3-carboxylate (3). To a solution of ethyl 5-chloropyrazolo[1,5-a]pyrimidine-3-carboxylate 1 (94 mg, 0.42 mmol, 1 eq.) in MeCN (2.0 mL) was added DIPEA (181.36 μL, 1.04 mmol, 3 eq.), followed by tert-butyl N-[(3S,4S)-4-fluoro-3-piperidyl]carbamate 2 (100 mg, 0.46 mmol, 1.1 eq.). The resulting mixture was then stirred at 90° C. After 16 h, LCMS showed total completion. MeCN was evaporated under vacuum, to give 3 (169.7 mg, quantitative yield) as a brown semi-solid, which was directly used in the next step.

LCMS method 1: 99.900 purity at 215 nm, [M+H]⁺=408.2.

Step 2. Preparation of 5-[(3S,4S)-3-(tert-Butoxycarbonylamino)-4-fluoro-1-piperidyl]pyrazolo[1,5-a]pyrimidine-3-carboxylic acid (4). To a solution of ethyl 5-[(3S,4S)-3-(tert-butoxycarbonylamino)-4-fluoro-1-piperidyl]pyrazolo[1,5-a]pyrimidine-3-carboxylate 3 (169.7 mg, 0.42 mmol, 1 eq.) in THF (694.2 μL) and methanol (694.2 μL) was added a solution of LiOH·H₂O (174.93 mg, 4.17 mmol, 10 eq.) in water (694.2 mL). The resulting mixture was stirred at 60° C. After 2 h, LCMS showed total completion. Volatiles were evaporated under vacuum, and the mixture was diluted in water. Then under vigorous stirring, the solution was acidified to pH 3 with a 6 N HCl aqueous solution. The white precipitate was isolated via filtration, and the solid was washed three times with cold water. The solid was then solubilized in THF and co-evaporated three times with THF to give 4 (16.61 mg, 38% yield) as an off-white solid.

LCMS method 1: 99.9% purity at 215 nm, [M-tBu+H]⁺=324.2.

¹H NMR (400 MHz, DMSO-d6) δ ppm 1.40 (s, 9H), 1.59-1.77 (m, 2H), 3.12-3.21 (m, 1H), 3.51-3.64 (m, 2H), 4.17-4.39 (m, 2H), 4.55-4.74 (m, 2H), 6.80-6.92 (m, 1H), 7.19-7.33 (m, 1H), 8.73 (d, J=7.8 Hz, 1H), 11.44-11.96 (m, 1H).

Step 3. Preparation of tert-butyl ((3S,4S)-1-(3-((3-(difluoromethyl)-1-((1r,4S)-4-(hydroxymethyl)cyclohexyl)-1H-pyrazol-4-yl)carbamoyl)pyrazolo[1,5-a]pyrimidin-5-yl)-4-fluoropiperidin-3-yl)carbamate (6). To a solution of [4-[4-amino-3-(difluoromethyl)pyrazol-1-yl]cyclohexyl]methanol 5—intermediate 6 from synthesis of P-2 —(110 mg, 0.40 mmol, 1.5 eq.), 5-[(3S,4S)-3-(tert-butoxycarbonylamino)-4-fluoro-1-piperidyl]pyrazolo[1,5-a]pyrimidine-3-carboxylic acid 4 (102.09 mg, 0.27 mmol, 1 eq.) and NMI (78 μL, 0.98 mmol, 3.65 eq.) in MeCN (4.03 mL) was added TCFH (94.66 mg, 0.34 mmol, 1.25 eq.). The resulting mixture was stirred at room temperature. After 16 h, LCMS showed completion. Volatiles were evaporated under vacuum. The resultant residue was purified by reverse phase flash chromatography (50 g C18 RediSep Rf Gold column, liquid deposit (DMSO), elution: 5% MeCN/0.1% HCOOH over 3 CV, then 5 to 100% MeCN/0.1% HCOOH over 15 CV, then 100% MeCN/0.1% HCOOH over 3 CV, desired product was released around 75% MeCN). Fractions were combined and concentrated to give 6 (116.2 mg, 71% yield) as an orange solid.

LCMS method 1: 99.9% purity at 215 nm, [M+H]⁺=607.3.

¹H NMR (400 MHz, DMSO-d6) δ ppm 1.02-1.09 (m, 2H), 1.21-1.28 (m, 2H), 1.35 (s, 9H), 1.66-1.78 (m, 3H), 1.81-1.92 (m, 2H), 2.00-2.10 (m, 2H), 2.13-2.25 (m, 1H), 3.07 (s, 1H), 3.24-3.28 (m, 2H), 3.42-3.52 (m, 1H), 3.53-3.68 (m, 1H), 4.11-4.22 (m, 2H), 4.42-4.51 (m, 1H), 4.58-4.78 (m, 1H), 6.85-6.92 (m, 1H), 6.93-7.21 (m, 1H), 7.22-7.30 (m, 1H), 8.28 (s, 1H), 8.35 (s, 1H), 8.81 (d, J=7.8 Hz, 1H), 9.33 (br s, 1H).

¹⁹F NMR (377 MHz, DMSO-d6) δ ppm −183.02-−179.35 (m, 1F), −112.72-−109.70 (m, 2F).

Step 4. Preparation of tert-Butyl N-[(3S,4S)-1-[3-[[3-(difluoromethyl)-1-[4-[[4-[[3-(2,6-dioxo-3-piperidyl)-1-methyl-indazol-6-yl]amino]-1-piperidyl]methyl]cyclohexyl]pyrazol-4-yl]carbamoyl]pyrazolo[1,5-a]pyrimidin-5-yl]-4-fluoro-3-piperidyl]carbamate (7). To a solution of tert-butyl ((3S,4S)-1-(3-((3-(difluoromethyl)-1-((1r,4S)-4-(hydroxymethyl)cyclohexyl)-1H-pyrazol-4-yl)carbamoyl)pyrazolo[1,5-a]pyrimidin-5-yl)-4-fluoropiperidin-3-yl)carbamate 6 (75 mg, 0.12 mmol, 1 eq.) in dry DMSO (1.0 mL) was added IBX (41.5 mg, 0.15 mmol, 1.2 eq.). The resulting mixture was stirred at rt. After 16 h, LCMS showed total conversion toward the aldehyde. The aldehyde solution was then added to a solution of 3-[1-methyl-6-(4-piperidylamino)indazol-3-yl]piperidine-2,6-dione hydrochloride C-1 (64.97 mg, 0.14 mmol, 1.1 eq.) and DIPEA (215 μL, 1.24 mmol, 10 eq.) in DCE (1.0 mL). The mixture was stirred at rt for 10 minutes and NaBH(OAc)₃ (34.05 mg, 0.16 mmol, 1.3 eq.) was added. The resulting mixture was then stirred at room temperature. After 1 h, LCMS showed total completion. DCE was removed under reduced pressure and the residue was purified by reverse phase flash chromatography (50 g C18 RediSep Rf Gold column, liquid deposit (DMSO), elution: 5% MeCN/0.1% HCOOH over 3 CV, then 5 to 35% MeCN/0.1% HCOOH over 15 CV, then 35 to 100% MeCN/0.1% HCOOH over 1 CV, then 100% MeCN/0.1% HCOOH over 3 CV (desired product was released around 30% MeCN). Fractions were combined and concentrated to give 7 (37 mg, 32% yield) as a light yellow solid. The product was used without purification in the next step.

LCMS method 1: [M+2H]²⁺=465.8.

Step 5. N-[3-(Difluoromethyl)-1-[4-[[4-[[3-(2,6-dioxo-3-piperidyl)-1-methyl-indazol-6-yl]amino]-1-piperidyl]methyl]cyclohexyl]pyrazol-4-yl]-5-[(3S,4S)-3-amino-4-fluoro-1-piperidyl]pyrazolo[1,5-a]pyrimidine-3-carboxamide (P-18). tert-Butyl N-[(3S,4S)-1-[3-[[3-(difluoromethyl)-1-[4-[[4-[[3-(2,6-dioxo-3-piperidyl)-1-methyl-indazol-6-yl]amino]-1-piperidyl]methyl]cyclohexyl]pyrazol-4-yl]carbamoyl]pyrazolo[1,5-a]pyrimidin-5-yl]-4-fluoro-3-piperidyl]carbamate 7 (35 mg, 0.038 mmol, 1 eq.) was solubilized in a 4 M HCl solution in 1,4-dioxane (1.41 mL, 5.64 mmol, 150 eq.). The mixture was stirred at rt. After 2 h, LCMS showed total completion. Volatiles were evaporated under vacuum. The residue was purified by reverse phase flash chromatography (30 g C18 RediSep Rf Gold column, liquid deposit (water), elution: 5% MeCN/0.02 M HCl over 5 CV, then 5 to 20% MeCN/0.02 M HCl over 15 CV, then 20% MeCN/0.02 M HCl over 5 CV, then 20 to 100% MeCN/0.02 M HCl over 2 CV then 100% MeCN/0.02 M HCl over 3 CV). Fractions were combined and concentrated to give P-18 (18.22 mg, 57% yield) as a tan solid as a bis HCl salt.

LCMS method 2: 97.7% purity at 215 nm, [M-2HCl+2H]²⁺=415.8; [M-2HCl+H]⁺=830.5.

¹H NMR (400 MHz, DMSO-d6) δ ppm 1.16-1.32 (m, 2H), 1.57-2.04 (m, 8H), 2.04-2.12 (m, 2H), 2.13-2.30 (m, 4H), 2.59-2.65 (m, 2H), 2.96-3.16 (m, 3H), 3.19-3.30 (m, 1H), 3.31-3.52 (m, 4H), 3.53-3.67 (m, 3H), 3.84 (s, 3H), 4.17-4.31 (m, 3H), 4.41-4.65 (m, 2H), 4.84-5.09 (m, 1H), 6.33-6.73 (m, 2H), 6.87 (d, J=7.8 Hz, 1H), 6.98-7.30 (m, 1H), 7.32-7.42 (m, 1H), 8.33 (s, 1H), 8.40 (s, 1H), 8.44-8.67 (m, 3H), 8.94 (d, J=8.1 Hz, 1H), 9.35 (s, 1H), 9.41-9.59 (m, 1H), 10.83 (s, 1H).

¹⁹F NMR (377 MHz, DMSO-d6) δ ppm −181.60 (s, 1F), −111.38 (s, 2F).

### Example S6. Synthesis of P-27

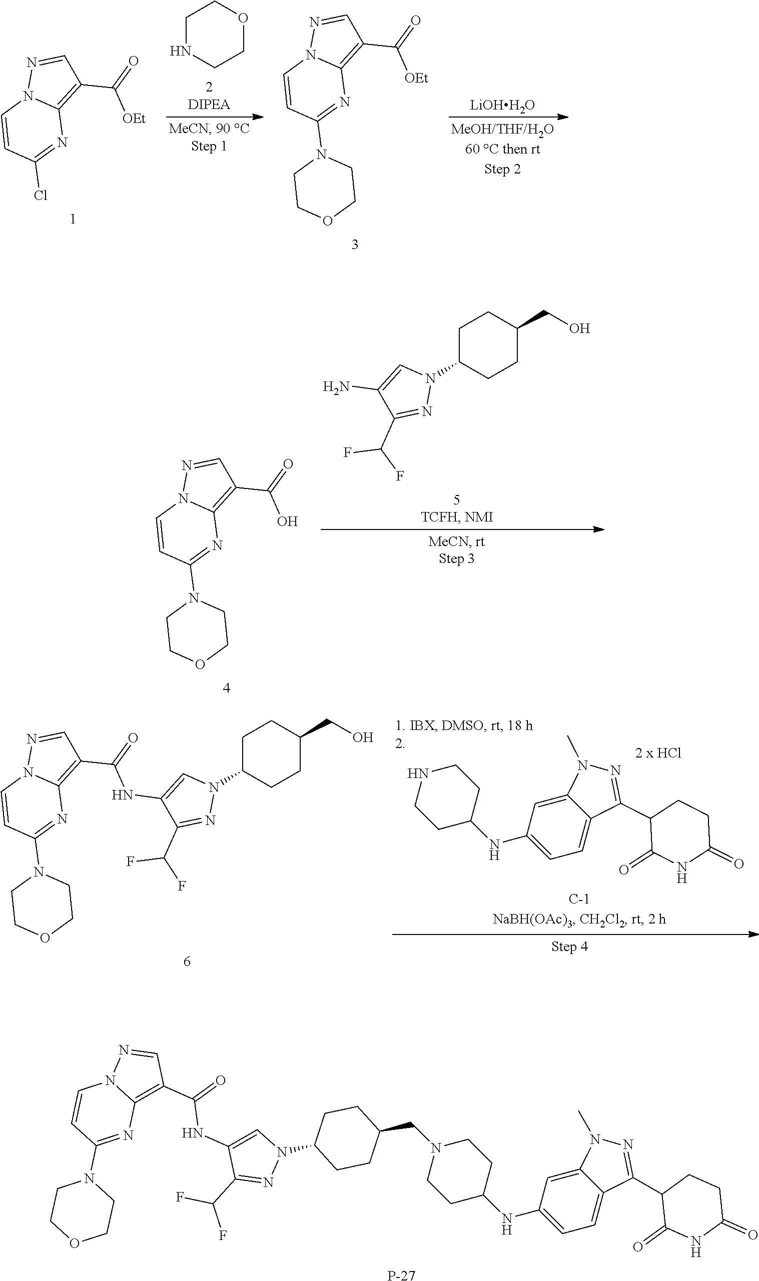

Step 1. Preparation of Ethyl 5-morpholinopyrazolo[1,5-a]pyrimidine-3-carboxylate (3). To a sealed tube are introduced ethyl 5-chloropyrazolo[1,5-a]pyrimidine-3-carboxylate 1 (1.5 g, 6.65 mmol, 1 eq.), MeCN (33.24 mL, 0.2 M) and DIPEA (2.89 mL, 16.62 mmol, 2.5 eq.), followed by morpholine 2 (639.65 μL, 7.31 mmol, 1.1 eq.). The tube was sealed and the mixture was stirred at 90° C. for 16 h. The solvents are evaporated under reduced pressure and dried under high vacuum to give ethyl 5-morpholinopyrazolo[1,5-a]pyrimidine-3-carboxylate 3 (2.96 g, 99% yield) as an orange solid.

LCMS method 1: retention time: 1.432 min, 99% purity at 215 nm, [M+H]⁺ 277.2.

¹H NMR (400 MHz, chloroform-d) δ ppm 1.39 (t, J=7.1 Hz, 3H), 3.76-3.86 (m, 8H), 4.35 (q, J=7.1 Hz, 2H), 6.41 (d, J=8.1 Hz, 1H), 8.29-8.34 (m, 2H).

Step 2. Preparation of 5-Morpholinopyrazolo[1,5-a]pyrimidine-3-carboxylic acid (4). To a solution of ethyl 5-morpholinopyrazolo[1,5-a]pyrimidine-3-carboxylate 3 (1.84 g, 6.66 mmol, 1.0 eq.) in THF (17.8 mL, 0.1 M) and methanol (17.8 mL), after 5 minutes of stirring was added a solution of LiOH H₂O (2.8 g, 66.6 mmol, 10 eq.) in water (17.8 mL). The mixture was stirred at 60° C. for 3 h, then the oil bath was removed and the mixture was allowed to stir over weekend (72 h). The reaction mixture was concentrated under reduced pressure, then the crude mixture was diluted with a small quantity of water and acidified to pH 3 with 6 N HCl. The resulting precipitate was filtered and rinsed with water. The solid was dried under high vacuum to give 5-morpholinopyrazolo[1,5-a]pyrimidine-3-carboxylic acid 4 (1.65 g, 99% yield) as a tan solid.

LCMS method 1: retention time: 1.192 min, 99% purity at 215 nm, [M+H]=249.2.

¹H NMR (400 MHz, DMSO-d6) δ ppm 3.72 (br d, J=5.9 Hz, 8H), 6.84 (d, J=8.1 Hz, 1H), 8.19 (s, 1H), 8.74 (d, J=7.8 Hz, 1H), 11.73 (s, 1H).

Step 3. Preparation of N-[3-(Difluoromethyl)-1-[4-(hydroxymethyl)cyclohexyl]pyrazol-4-yl]-5-morpholino-pyrazolo[1,5-a]pyrimidine-3-carboxamide (6). To a solution of [4-[4-amino-3-(difluoromethyl)pyrazol-1-yl]cyclohexyl]methanol 5—intermediate 6 from synthesis of P-2 —(403.12 mg, 1.64 mmol, 1.2 eq.), 5-morpholinopyrazolo[1,5-a]pyrimidine-3-carboxylic acid 4 (340.0 mg, 1.37 mmol, 1.0 eq.) and NMI (0.43 mL, 5.48 mmol, 4.0 eq.) in MeCN (9 mL, 0.15 M) was added TCFH (480.37 mg, 1.71 mmol, 1.25 eq.). The resulting mixture was stirred at room temperature for 1 h. After 1 h, volatiles were evaporated and the resulting mixture was purification by reverse phase chromatography (50 g C18 RediSep Rf Gold column, liquid deposit (DMSO), elution: 5% MeOH/0.1% HCOOH over 4 CV, then 5% to 50% MeOH/0.1% HCOOH over 15 CV, product exited at 50% MeOH). The pure fractions were combined, concentrated under reduced pressure to afford N-[3-(difluoromethyl)-1-[4-(hydroxymethyl)cyclohexyl]pyrazol-4-yl]-5-morpholino-pyrazolo[1,5-a]pyrimidine-3-carboxamide 6 (338 mg, 52% yield) as a tan solid.

LCMS method 3: retention time: 1.707 min, 99.9% purity at 215 nm, [M+H]⁺=476.2.

¹H NMR (400 MHz, DMSO-d6) δ ppm 1.02-1.15 (m, 2H), 1.38-1.48 (m, 1H), 1.66-1.79 (m, 2H), 1.80-1.90 (m, 2H), 1.99-2.08 (m, 2H), 3.26 (t, J=5.7 Hz, 2H), 3.68-3.74 (m, 4H), 3.76-3.83 (m, 4H), 4.12-4.23 (m, 1H), 4.47 (t, J=5.4 Hz, 1H), 6.90 (d, J=7.8 Hz, 1H), 6.95-7.25 (m, 1H), 8.28 (s, 1H), 8.38 (s, 1H), 8.82 (d, J=8.1 Hz, 1H), 9.39 (s, 1H).

¹⁹F NMR (377 MHz, DMSO-d6) δ ppm −111.19 (s, 2F).

Step 4. Preparation of N-[3-(Difluoromethyl)-1-[4-[[4-[[3-(2,6-dioxo-3-piperidyl)-1-methyl-indazol-6-yl]amino]-1-piperidyl]methyl]cyclohexyl]pyrazol-4-yl]-5-morpholino-pyrazolo[1,5-a]pyrimidine-3-carboxamide formic acid salt (P-27). To a solution of N-[3-(difluoromethyl)-1-[4-(hydroxymethyl)cyclohexyl]pyrazol-4-yl]-5-morpholino-pyrazolo[1,5-a]pyrimidine-3-carboxamide 6 (120.0 mg, 0.25 mmol, 1 eq.) in DMSO (1.5 mL) was added IBX (84.0 mg, 0.3 mmol, 1.2 eq.) at room temperature. Then the mixture was stirred at room temperature. After 18 h, LCMS showed full conversion to the aldehyde. Then the solution of aldehyde in DMSO was slowly added to a solution of 3-[1-methyl-6-(4-piperidylamino)indazol-3-yl]piperidine-2,6-dione dihydrochloride C-1 (115.1 mg, 0.28 mmol, 1.1 eq.), DIPEA (0.44 mL, 2.5 mmol, 10 eq.) and NaBH(OAc)₃ (158.95 mg, 0.75 mmol, 3 eq.) in CH₂Cl₂ (1.5 mL). The resulting mixture was stirred at room temperature. After 2 h, LCMS showed full conversion of the aldehyde. Solvents were removed under reduced pressure, then it was purified by reverse phase FC (50 g C18 RediSep Rf Gold column, liquid deposit (DMSO), elution: 5% MeCN/0.1% HCOOH over 5 CV, then 5 to 100% MeCN/0.1% HCOOH over 20 CV, then 100% MeCN/0.1% HCOOH over 5 CV, product came out around 35% MeCN). Fractions were combined, concentrated and lyophilised to give crude P-27 (67 mg, 82% purity at 215 nm), which was then purified by preparative LCMS (acidic conditions using formic acid as the modifier) to give P-27 (50 mg, 24% yield) as a pink solid as a formic acid salt.

LCMS method 2: 97.4% purity at 215 nm, [M−HCOOH+H]⁺=799.4.

¹H NMR (400 MHz, DMSO-d6) δ ppm 0.99-1.12 (m, 2H), 1.34-1.48 (m, 2H), 1.53-1.67 (m, 1H), 1.68-1.82 (m, 2H), 1.87-2.00 (m, 4H), 2.01-2.31 (m, 9H), 2.56-2.63 (m, 2H), 2.79-2.87 (m, 2H), 3.69-3.75 (m, 4H), 3.75-3.83 (m, 7H), 4.14-4.24 (m, 2H), 5.74 (br d, J=7.8 Hz, 1H), 6.38 (s, 1H), 6.52 (dd, J=8.8, 1.7 Hz, 1H), 6.91 (d, J=8.1 Hz, 1H), 7.09 (t, J=54.0 Hz, 1H), 7.32 (d, J=8.8 Hz, 1H), 8.16 (s, 1H), 8.29 (s, 1H), 8.38 (s, 1H), 8.83 (d, J=8.1 Hz, 1H), 9.40 (s, 1H), 10.81 (s, 1H).

### Example S7. Synthesis of P-30

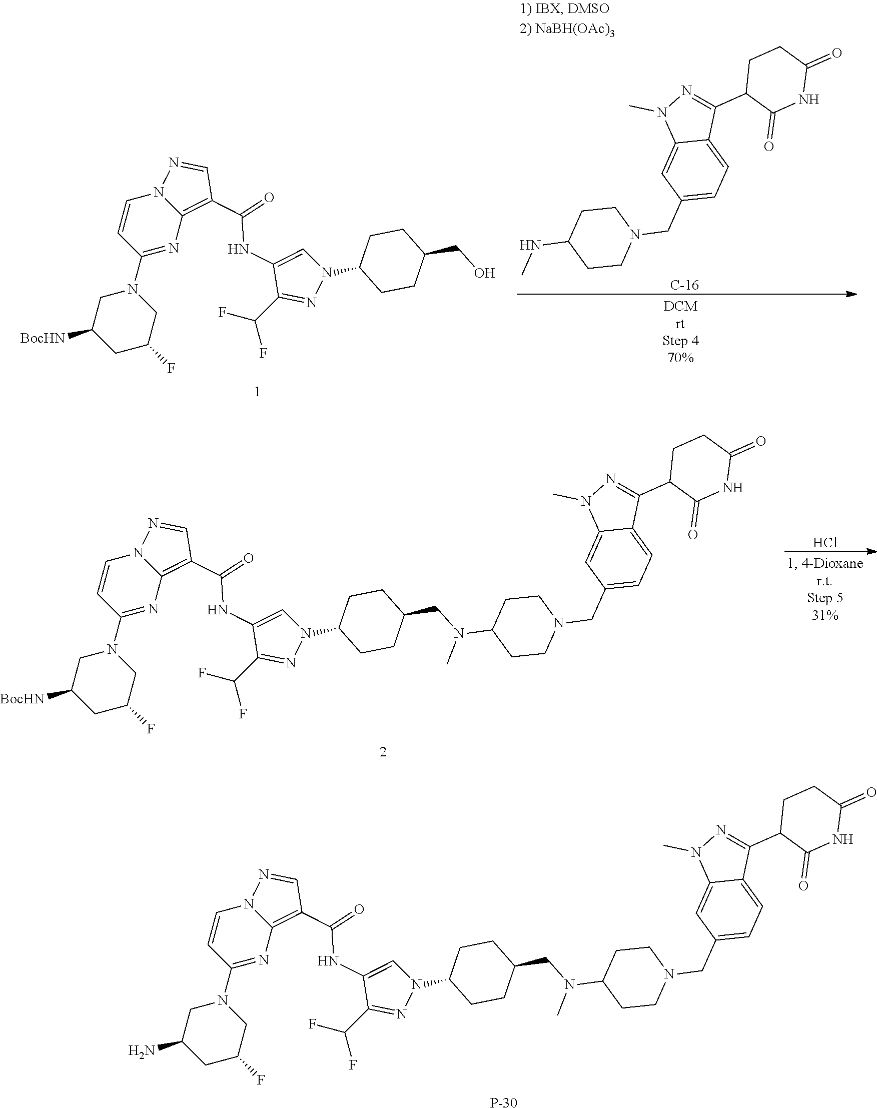

Step 4. Preparation of tert-butyl N-[rac-(3R,5R)-1-[3-[[3-(difluoromethyl)-1-[4-[[[1-[3-(2,6-dioxo-3-piperidyl)-1-methyl-indazol-7-yl]-4-piperidyl]-methyl-amino]methyl]cyclohexyl]pyrazol-4-yl]carbamoyl]pyrazolo[1,5-a]pyrimidin-5-yl]-5-fluoro-3-piperidyl]carbamate formic acid salt (2). To a solution of tert-butyl N-[(3R,5R)-1-[3-[[3-(difluoromethyl)-1-[4-(hydroxymethyl)cyclohexyl]pyrazol-4-yl]carbamoyl]pyrazolo[1,5-a]pyrimidin-5-yl]-5-fluoro-3-piperidyl]carbamate 1—intermediate 6 from synthesis of P-2 —(125 mg, 0.21 mmol, 1.0 eq.) in dry DMSO (1 mL, 0.10 M) was added IBX (69.24 mg, 0.25 mmol, 1.2 eq.). The resulting mixture was stirred at room temperature overnight. After an overnight stirring, LCMS showed complete conversion into the aldehyde. To the reaction mixture was added 3-[1-methyl-7-[4-(methylamino)-1-piperidyl]indazol-3-yl]piperidine-2,6-dione hydrochloride C-16 (97.09 mg, 0.23 mmol, 1.2 eq.), DCM (1 mL, 0.1 M) and DIPEA (0.36 mL, 2.06 mmol, 10.0 eq.). The mixture was stirred at room temperature for 10 minutes and then sodium triacetoxyborohydride (131.01 mg, 0.62 mmol, 3 eq.) was added. After 1 h, LCMS showed complete conversion into compound 6. DCM was evaporated under reduced pressure and the crude mixture was purified by reverse phase FC purification (50 g C18 RediSep Rf Gold column, liquid deposit (DMSO), 5% MeCN/0.1% HCOOH over 4 CV, then 5 to 60% MeCN/0.1% HCOOH over 20 CV, product came out at 50% MeCN). Pure fractions were combined and concentrated to give 2 (152 mg, 70% yield) as a tan solid as a formic acid salt. The desired product contained IBX residue.

LCMS method 3: 90.2% purity at 254 nm, [M−HCOOH+H]⁺=944.4.

¹H NMR (400 MHz, DMSO-d6) δ ppm 0.99-1.12 (m, 2H), 1.34 (s, 9H), 1.48-1.59 (m, 1H), 1.64-1.98 (m, 10H), 2.00-2.07 (m, 2H), 2.11-2.21 (m, 2H), 2.28 (s, 3H), 2.30-2.35 (m, 3H), 2.57-2.66 (m, 2H), 2.68-2.77 (m, 2H), 3.18-3.25 (m, 3H), 3.39-3.52 (m, 5H), 3.63-3.72 (m, 1H), 4.14-4.23 (m, 1H), 4.26 (s, 3H), 4.33 (dd, J=9.7, 5.0 Hz, 1H), 4.94-5.12 (m, 1H), 6.85-6.94 (m, 1H), 6.95-7.19 (m, 4H), 7.32-7.41 (m, 1H), 8.15 (s, 1H), 8.29 (s, 1H), 8.34 (br s, 1H), 8.82 (br d, J=8.1 Hz, 1H), 9.31 (br s, 1H), 10.88 (s, 1H).

Step 5. Preparation of N-[3-(difluoromethyl)-1-[4-[[[1-[3-(2,6-dioxo-3-piperidyl)-1-methyl-indazol-7-yl]-4-piperidyl]-methyl-amino]methyl]cyclohexyl]pyrazol-4-yl]-5-[(3R,5R)-3-amino-5-fluoro-1-piperidyl]pyrazolo[1,5-a]pyrimidine-3-carboxamide (P-30). To a round bottom flask was added tert-butyl N-[(3R,5R)-1-[3-[[3-(difluoromethyl)-1-[4-[[[1-[3-(2,6-dioxo-3-piperidyl)-1-methyl-indazol-7-yl]-4-piperidyl]-methyl-amino]methyl]cyclohexyl]pyrazol-4-yl]carbamoyl]pyrazolo[1,5-a]pyrimidin-5-yl]-5-fluoro-3-piperidyl]carbamate 2 (152 mg, 0.16 mmol, 1 eq.) and 4 M HCl in dioxane (4.99 mL, 19.97 mmol, 124 eq.). The reaction was stirred at r.t. LCMS after 1 h showed complete conversion into compound P-30. The reaction mixture was concentrated in vacuo and the residue was purified by reverse-phase FC (50 g C18 RediSep Rf Gold column, liquid deposit (H₂O), elution: 5% MeCN/0.02 M HCl over 4 CV, then 5% to 30% MeCN/0.02 M HCl over 20 CV, product exited at 25% MeCN). Fractions were combined and concentrated, affording P-30 (42.38 mg, 31% yield) as a white solid as a dihydrochloride salt.

LCMS method 2: 99.9% purity at 215 nm, [M-2HCl+H]⁺=844.4; [M-2HCl+2H]²⁺=422.8.

¹H NMR (400 MHz, DMSO-d6) δ ppm 1.15-1.35 (m, 2H), 1.77-2.03 (m, 7H), 2.03-2.25 (m, 7H), 2.34-2.44 (m, 1H), 2.57-2.66 (m, 2H), 2.79-2.88 (m, 5H), 2.92-3.01 (m, 1H), 3.16-3.25 (m, 1H), 3.51 (br s, 6H), 4.22-4.29 (m, 4H), 4.35 (dd, J=9.5, 5.1 Hz, 1H), 4.51-4.67 (m, 1H), 4.76-4.95 (m, 1H), 5.10 (br d, J=48.0 Hz, 1H), 6.89 (br d, J=7.6 Hz, 1H), 6.99-7.28 (m, 3H), 7.43 (dd, J=6.6, 2.4 Hz, 1H), 8.26 (br s, 3H), 8.33 (s, 1H), 8.41 (s, 1H), 8.94 (d, J=8.1 Hz, 1H), 9.34 (s, 1H), 9.56 (br s, 1H), 10.89 (s, 1H).

¹⁹F NMR (377 MHz, DMSO-d6) δ ppm −184.64 (br s, 1F), −111.82-−110.85 (m, 2 F).

The following compounds were synthesized via the same general routes with modifications to amine 2 in step 1 and CBM (C—X) in step 4 (Table 5).

TABLE 5

| Com- | | | | |
|---|---|---|---|---|
| pound | | | | |
| No. | Amine | CBM | Structure | Characterization Data |
| P-2 | 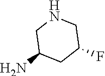 | C-1 | 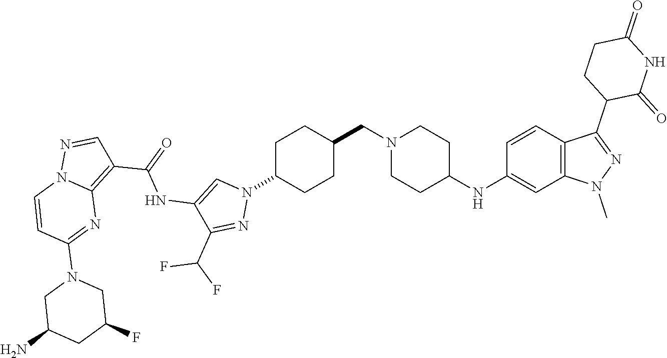 | 54% yield as a tan solid. LCMS method 4: retention time: 1.924 min, 99% purity at 215 nm, [M − HCl + H]⁺ = 830.3; [M − HCl + 2H]²⁺ = 415.7. ¹H NMR (400 MHz, DMSO-d6) δ ppm 1.14-1.30 (m, 2 H), 1.76-2.46 (m, 15 H), 2.58-2.65 (m, 2 H), 2.93-3.17 (m, 4 H), 3.26-3.51 (m, 4 H), 3.54-3.65 (m, 2 H), 4.18- 4.30 (m, 2 H), 4.55-4.72 (m, 1 H), 4.75-4.96 (m, 1 H), 5.02-5.19 (m, 1 H), 6.53-6.69 (m, 2 H), 6.91 (br d, J = 8.1 Hz, 1 H), 7.14 (t, J = 54.0 Hz, 1 H), 7.41 (br d, J = 8.8 Hz, 1 H), 8.31-8.34 (m, 1 H), 8.40 (s, 4 H), 8.93 (d, J = 7.8 Hz, 1 H), 9.34 (s, 1 H), 9.93 (br s, 1 H), 10.84 (s, 1 H). - Three protons are not observed in ¹H NMR. ¹⁹F NMR (377 MHz, DMSO-d6) δ ppm −184.68 (s, 1 F), −111.54-110.76 (m, 2 F). |
| P-3 | 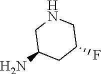 | C-2 | 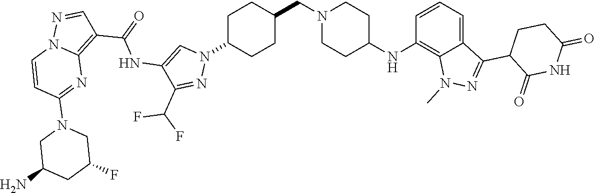 | 70% yield as a light-grey solid. LCMS method 2: retention time: 1.886 min, 97% purity at 215 nm, [M − 2HCl + H]⁺ = 830.5, [M − 2HCl +2 H]²⁺ = 415.8. ¹H NMR (400 MHz, DMSO-d6) δ ppm 1.16-1.29 (m, 2 H), 1.76-2.10 (m, 10 H), 2.12-2.24 (m, 3 H), 2.24-2.35 (m, 1 H), 2.35-2.46 (m, 1 H), 2.53-2.72 (m, 2 H), 2.94- 3.00 (m, 1 H), 3.02-3.14 (m, 2 H), 3.17-3.27 (m, 1 H), 3.28-3.42 (m, 3 H), 3.46-3.62 (m, 2 H), 3.76-3.83 (m, 1 H), 4.22-4.34 (m, 5 H), 4.54-4.93 (m, 2 H), 5.01-5.31 (m, 2 H), 6.65 (dd, J = 11.0, 7.6 Hz, 1 H), 6.87-6.98 (m, 2 H), 6.98-7.32 (m, 2 H), 8.26-8.44 (m, 5 H), 8.93 (d, J = 8.1 Hz, 1 H), 9.34 (s, 1 H), 9.59-9.80 (m, 1 H), 10.87 (s, 1 |
| | | | | H). |
| | | | | ¹⁹F NMR (377 MHz, DMSO-d6) δ ppm −184.60 (s, 1 F), |
| | | | | −111.30 (d, J = 15.0 Hz, 2 F). |
| P-5 | 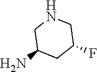 | C-3 | 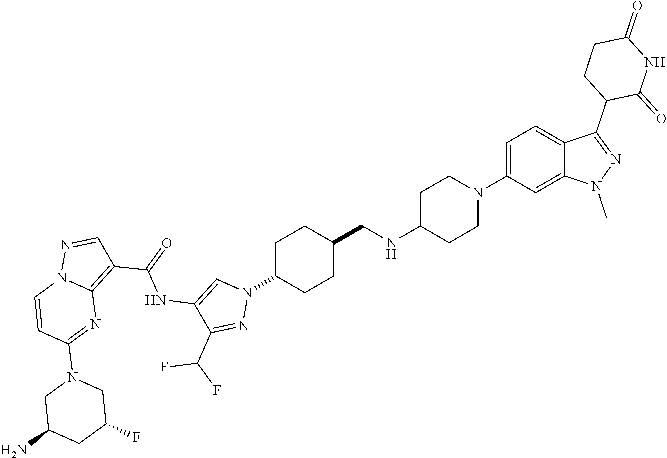 | 38% yield as a white solid. LCMS method 2: retention time: 1.876 min, 99% purity at 215 nm, [M − 2HCl + 2H]²⁺ = 415.7, [M − 2HCl + H]⁺ = 830.4. ¹H NMR (400 MHz, DMSO-d6) δ ppm 1.12-1.32 (m, 2 H), 1.58-1.86 (m, 5 H), 1.88-2.02 (m, 2 H), 2.04-2.23 (m, 5 H), 2.26-2.36 (m, 2 H), 2.58-2.68 (m, 3 H), 2.77- 2.97 (m, 3 H), 3.22-3.53 (m, 5 H), 3.86-3.92 (m, 3 H), 3.92-4.03 (m, 2 H), 4.16-4.36 (m, 2 H), 4.47-4.93 (m, 2 H), 5.10 (d, J = 46.2 Hz, 1 H), 6.78-6.99 (m, 3 H), 6.99- 7.31 (m, 1 H), 7.48-7.57 (m, 1 H), 8.06-8.29 (m, 3 H), 8.33 (s, 1 H), 8.42 (s, 1 H), 8.47-8.69 (m, 2 H), 8.91-9.00 (m, 1 H), 9.33 (s, 1 H), 10.85 (s, 1 H). ¹⁹F NMR (377 MHz, DMSO-d6) δ ppm −184.50 (s, 1 F), −111.28 (s, 2 F). |
| P-8 | 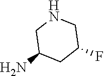 | C-5 | 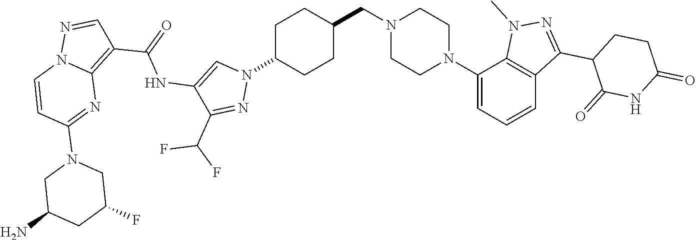 | 56% yield as a white solid. LCMS method 2: retention time: 1.850 min, 99% purity at 215 nm, [M − 2HCl + H]⁺ = 816.4; [M − 2HCl + 2H]²⁺ = 408.8. ¹H NMR (400 MHz, DMSO-d6) δ ppm 1.20-1.33 (m, 2 H), 1.75-2.25 (m, 9 H), 2.27-2.45 (m, 2 H), 2.57-2.75 (m, 3 H), 3.07-3.22 (m, 2 H), 3.46-3.54 (m, 1 H), 3.58- 3.75 (m, 2 H), 4.22-4.32 (m, 4 H), 4.36 (dd, J = 9.0, 5.5 Hz, 1 H), 4.50-4.72 (m, 1 H), 4.76-4.96 (m, 1 H), 5.10 (d, J = 48.0 Hz, 1 H), 6.89 (d, J = 8.0 Hz, 1 H), 6.98-7.31 (m, 3 H), 7.49 (d, J = 8.3 Hz, 1 H), 8.21-8.36 (m, 4 H), 8.41 (s, 1 H), 8.94 (d, J = 7.8 Hz, 1 H), 9.34 (s, 1 H), 9.89- 10.05 (m, 1 H), 10.90 (s, 1 H). - Seven protons obscured by the water peak in ¹H NMR. ¹⁹F NMR (377 MHz, DMSO-d6) δ ppm −184.32 (s, 1 F), −111.35 (s, 2 F). |
| P-12 | 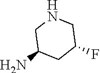 | C-7 | 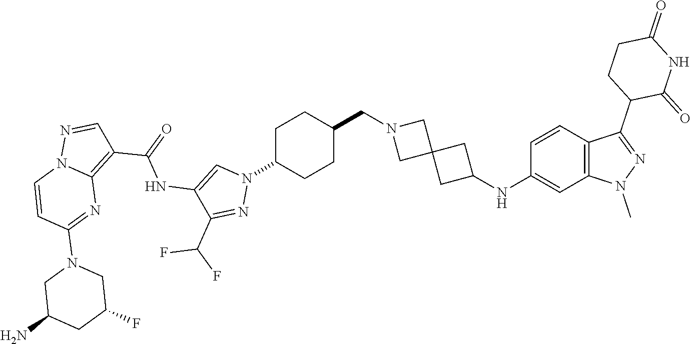 | 50% yield as a light pink solid. LCMS method 2: retention time: 1.852 min, 98% purity at 215 nm, [M − HCOOH + H]⁺ = 842.4 m/z, [M − HCOOH + 2H]²⁺ = 421.8 m/z, [M − HCOOH + 3H]3+ = 281.5 m/z. ¹H NMR (400 MHz, DMSO-d6) δ ppm 0.96-1.19 (m, 2 H), 1.27-1.47 (m, 1 H), 1.51-1.90 (m, 6 H), 1.91-2.08 (m, 4 H), 2.08-2.38 (m, 5 H), 2.56-2.69 (m, 4 H), 2.88- 3.01 (m, 1 H), 3.02-3.17 (m, 1 H), 3.17-3.49 (m, 6 H), 3.72-3.87 (m, 4 H), 4.12-4.22 (m, 2 H), 4.23-4.57 (m, 1 H), 4.65-4.88 (m, 1 H), 5.03 (d, J = 47.2 Hz, 1 H), 6.15 (d, J = 6.1 Hz, 1 H), 6.22 (d, J = 1.0 Hz, 1 H), 6.46 (dd, J = 8.8, 1.5 Hz, 1 H), 6.93 (d, J = 7.8 Hz, 1 H), 6.99-7.29 (m, 1 H), 7.32 (d, J = 8.8 Hz, 1 H), 8.14-8.32 (m, 2 H), 8.36-8.42 (m, 1 H), 8.82 (d, J = 7.8 Hz, 1 H), 9.33 (s, 1 H), 10.82 (s, 1 H). - One proton is not apparent in the ¹H NMR spectra. ¹⁹F NMR (377 MHz, DMSO-d6) δ ppm −183.10 (s, 1 F), −111.05 (s, 2 F). |
| P-13 | 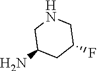 | C-8 | 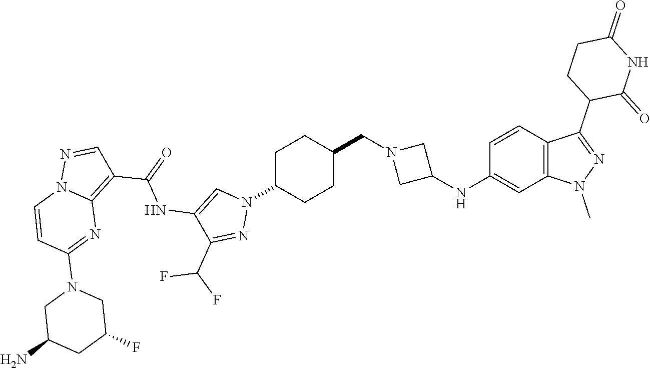 | 76% yield as a white solid. LCMS method 2: retention time: 1.805 min, 99% purity at 215 nm, [M − HCOOH + 2H]²⁺ = 401.8 ; [M − HCOOH + H]⁺ = 802.3. ¹H NMR (400 MHz, DMSO-d6 ) δ ppm 1.02-1.18 (m, 2 H), 1.34-1.48 (m, 1 H), 1.56-1.99 (m, 6 H), 2.00-2.07 (m, 2 H), 2.09-2.31 (m, 3 H), 2.34-2.40 (m, 2 H), 2.57- 2.64 (m, 2 H), 2.84-2.93 (m, 2 H), 2.96-3.08 (m, 1 H), 3.12-3.21 (m, 1 H), 3.29-3.46 (m, 2 H), 3.77-3.84 (m, 5 H), 4.00-4.11 (m, 1 H), 4.15-4.30 (m, 2 H), 4.34-4.60 (m, 1 H), 4.69-4.89 (m, 1 H), 5.04 (d, J = 46.2 Hz, 1 H), 6.28 (d, J = 1.2 Hz, 1 H), 6.37 (d, J = 6.6 Hz, 1 H), 6.51 (dd, J = 8.8, 1.7 Hz, 1 H), 6.91 (d, J = 8.1 Hz, 1 H), 6.98-7.30 (m, 1 H), 7.35 (d, J = 8.8 Hz, 1 H), 8.18 (s, 1 H), 8.29 (s, 1 H), 8.35-8.43 (m, 1 H), 8.84 (d, J = 8.1 Hz, 1 H), 9.33 (s, 1 H), 10.82 (s, 1 H). - One proton is not apparent in the ¹H NMR spectra. ¹⁹F NMR (377 MHz, DMSO-d6 ) δ ppm −183.42 (s, 1 F), −111.07 (s, 2 F). |
| P-14 | 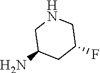 | C-6 | 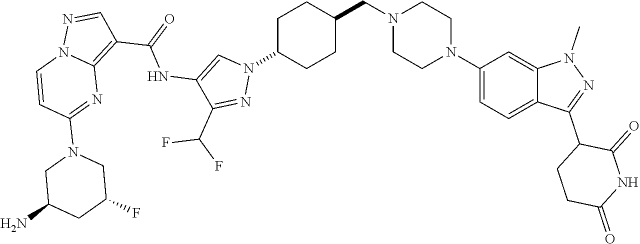 [SMILES: Cn1nc(C2CCC(=O)NC2=O)c2ccc(N3CCN(C[C@H]4CC[C@H](n5cc(NC(=O)c6cnn7ccc(N8C[C@H](N)C[C@@H](F)C8)nc67)c(C(F)F)n5)CC4)CC3)cc21] | 60% yield as a white solid. LCMS method 2: retention time: 1.833 min, 99% purity at 215 nm, [M − 2HCl + H]⁺ = 816.4, [M − 2HCl + 2H]²⁺ = 408.7. ¹H NMR (400 MHz, DMSO-d6) δ ppm 1.09-1.35 (m, 3 H), 1.76-1.91 (m, 2 H), 1.91-2.13 (m, 6 H), 2.13-2.25 (m, 1 H), 2.25-2.36 (m, 1 H), 2.36-2.46 (m, 1 H), 2.56- 2.75 (m, 2 H), 3.01-3.13 (m, 2 H), 3.13-3.25 (m, 2 H), 3.27-3.32 (m, 2 H), 3.40-3.55 (m, 2 H), 3.55-3.77 (m, 2 H), 3.78-4.09 (m, 5 H), 4.17-4.40 (m, 2 H), 4.47-4.73 (m, 1 H), 4.75-5.01 (m, 1 H), 5.10 (d, J = 45.7 Hz, 1 H), 6.90 (d, J = 8.1 Hz, 1 H), 6.94-7.00 (m, 2 H), 7.15 (t, J = 54.2 Hz, 1 H), 7.57 (d, J = 9.7 Hz, 1 H), 8.24-8.39 (m, 4 H), 8.41 (s, 1 H), 8.94 (d, J = 7.9 Hz, 1 H), 9.34 (s, 1 H), 10.10 (br s, 1 H), 10.86 (s, 1 H). - Four protons partially obscured by the water peak in ¹H NMR spectrum. |
| | | | | ¹⁹F NMR (377 MHz, DMSO-d6) δ ppm −184.57 (s, 1 F), |
| | | | | −111.28 (s, 2 F). |
| P-15 | 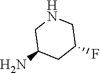 | C-4 | 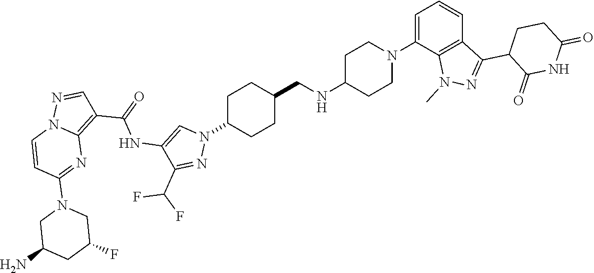 | 78% yield as a white solid. LCMS method 2: retention time: 1.942 min, 99% purity at 215 nm, [M − 2HCl + H]⁺ = 830.4, [M − 2HCl + 2H]²⁺ = 415.8. ¹H NMR (400 MHz, DMSO-d6) δ ppm 1.14-1.40 (m, 2 H), 1.66-1.89 (m, 3 H), 1.92-2.06 (m, 4 H), 2.06-2.15 (m, 3 H), 2.15-2.26 (m, 3 H), 2.27-2.46 (m, 3 H), 2.56- 2.69 (m, 2 H), 2.69-2.85 (m, 2 H), 2.86-3.01 (m, 2 H), 3.12-3.38 (m, 5 H), 4.10-4.31 (m, 4 H), 4.35 (dd, J = 9.6, 5.1 Hz, 1 H), 4.50-4.74 (m, 1 H), 4.75-4.99 (m, 1 H), 5.10 (d, J = 46.2 Hz, 1 H), 6.91 (d, J = 7.9 Hz, 1 H), 6.98- 7.32 (m, 3 H), 7.42 (dd, J = 6.8, 2.0 Hz, 1 H), 8.33 (s, 1 H), 8.35-8.57 (m, 4 H), 8.93 (d, J = 7.8 Hz, 3 H), 9.34 (s, 1 H), 10.89 (s, 1 H). ¹⁹F NMR (377 MHz, DMSO-d6) δ ppm −184.61 (s, 1 F), −111.29 (s, 2 F). |
| P-17 | 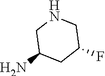 | C-9 | 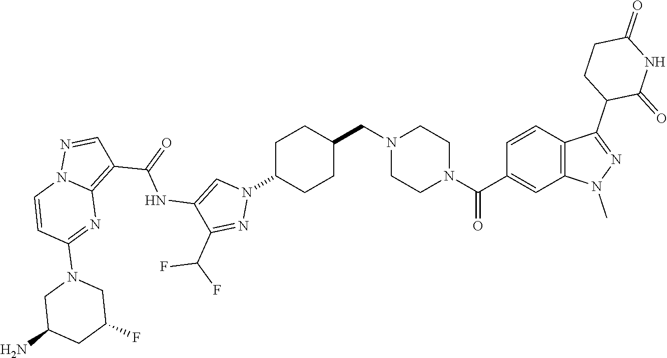 | 73% yield as a off-white solid. LCMS method 5: retention time: 2.205 min, 96% purity at 215 nm, [M − 2HCl + H]⁺ = 844.3, [M − 2HCl + 2H]²⁺ = 422.8. ¹H NMR (400 MHz, DMSO-d6) δ ppm 1.12-1.34 (m, 2 H), 1.72-1.86 (m, 2 H), 1.87-1.99 (m, 2 H), 1.99-2.14 (m, 5 H), 2.14-2.27 (m, 1 H), 2.34-2.47 (m, 2 H), 2.57- 2.79 (m, 3 H), 2.94-3.05 (m, 2 H), 3.05-3.23 (m, 3 H), 3.26-3.45 (m, 4 H), 3.45-3.53 (m, 2 H), 4.05 (s, 3 H), 4.18-4.31 (m, 1 H), 4.43 (dd, J = 10.1, 5.0 Hz, 1 H), 4.56- 4.74 (m, 1 H), 4.75-5.00 (m, 1 H), 5.10 (d, J = 46.0 Hz, 1 H), 6.91 (d, J = 8.1 Hz, 1 H), 6.98-7.31 (m, 2 H), 7.76 (s, 1 H), 7.82 (d, J = 8.3 Hz, 1 H), 8.32 (s, 1 H), 8.39 (s, 1 H), 8.42 (br s, 3 H), 8.93 (d, J = 7.9 Hz, 1 H), 9.33 (s, 1 H), 10.80 (br s, 1 H), 10.92 (s, 1 H). ¹⁹F NMR (377 MHz, DMSO-d6) δ ppm −184.62 (s, 1 F), −111.32 (s, 2 F) |
| P-18 | 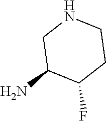 | C-1 | 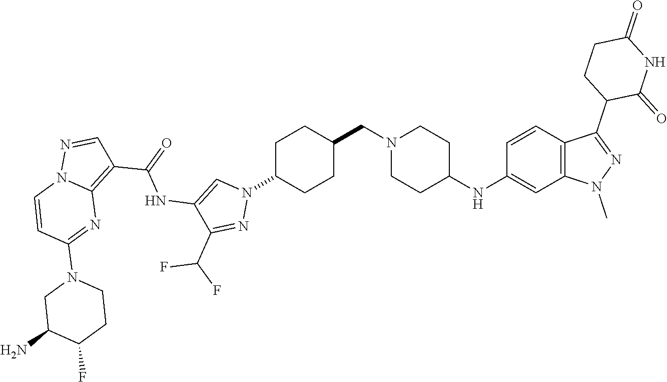 | 57% yield as a tan solid. LCMS method 2: retention time: 1.840 min, 98% purity at 215 nm, [M − 2HCl + 2H]²⁺ = 415.8; [M − 2HCl + H]⁺ = 830.5. ¹H NMR (400 MHz, DMSO-d6) δ ppm 1.16-1.32 (m, 2 H), 1.57-2.04 (m, 8 H), 2.04-2.12 (m, 2 H), 2.13-2.30 (m, 4 H), 2.59-2.65 (m, 2 H), 2.96-3.16 (m, 3 H), 3.19- 3.30 (m, 1 H), 3.31-3.52 (m, 4 H), 3.53-3.67 (m, 3 H), 3.84 (s, 3 H), 4.17-4.31 (m, 3 H), 4.41-4.65 (m, 2 H), 4.84-5.09 (m, 1 H), 6.33-6.73 (m, 2 H), 6.87 (d, J = 7.8 Hz, 1 H), 6.98-7.30 (m, 1 H), 7.32-7.42 (m, 1 H), 8.33 (s, 1 H), 8.40 (s, 1 H), 8.44-8.67 (m, 3 H), 8.94 (d, J = 8.1 Hz, 1 H), 9.35 (s, 1 H), 9.41-9.59 (m, 1 H), 10.83 (s, 1 H). ¹⁹F NMR (377 MHz, DMSO-d6) δ ppm −181.60 (s, 1 F), −111.38 (s, 2 F). |
| P-19 | 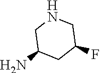 | C-1 | 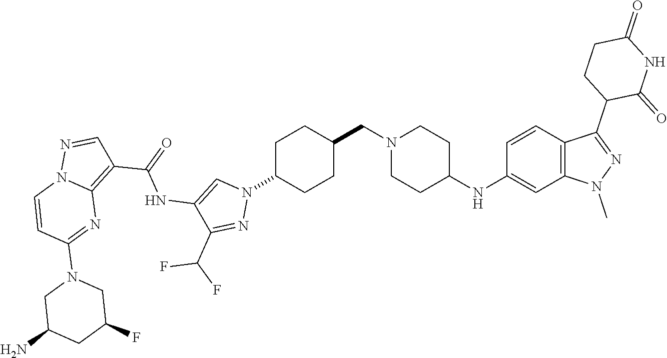 | 50% yield as a pink solid. LCMS method 2: retention time: 1.819 min, 99% purity at 215 nm, [M − 2HCl + H]⁺ = 830.4 m/z, [M − 2HCl + 2H]²⁺ = 415.8 m/z. ¹H NMR (400 MHz, DMSO-d6) δ ppm 1.14-1.29 (m, 2 H), 1.76-1.89 (m, 2 H), 1.90-2.10 (m, 8 H), 2.11-2.22 (m, 3 H), 2.24-2.35 (m, 1 H), 2.36-2.47 (m, 1 H), 2.55- 2.67 (m, 2 H), 2.89-3.12 (m, 4 H), 3.32-3.41 (m, 3 H), 3.43-3.52 (m, 1 H), 3.57-3.67 (m, 2 H), 3.79-3.94 (m, 4 H), 4.20-4.30 (m, 2 H), 4.57-4.72 (m, 1 H), 4.79-4.96 (m, 1 H), 5.10 (d, J = 46.7 Hz, 1 H), 6.69-6.81 (m, 2 H), 6.92 (d, J = 8.1 Hz, 1 H), 7.14 (t, J = 54.3 Hz, 1 H), 7.44- 7.53 (m, 1 H), 8.32 (s, 1 H), 8.39 (s, 1 H), 8.44 - 8.56 (m, 3 H), 8.93 (d, J = 7.8 Hz, 1 H), 9.34 (s, 1 H), 10.10-10.26 (m, 1 H), 10.83-10.87 (m, 1 H). - Conformers confirmed by high temperature (80 °C) ¹H NMR. ¹⁹F NMR (377 MHz, DMSO-d6) δ ppm −184.61 (s, 1 F), −111.32 (s, 2 F). |
| P-20 | 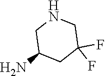 | C-1 | 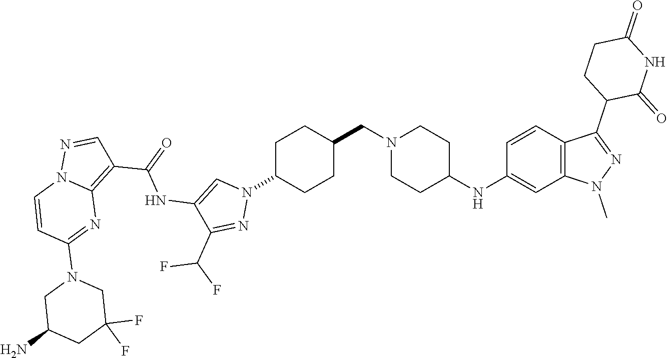 | 63% yield as a white solid. LCMS method 2: retention time: 1.840 min, 99% purity at 215 nm, [M + H]⁺ = 848.3, [M + 2H]²⁺ = 424.7. ¹H NMR (400 MHz, DMSO-d6) δ ppm 1.13-1.31 (m, 2 H), 1.72-1.87 (m, 2 H), 1.88-1.98 (m, 3 H), 1.98-2.11 (m, 4 H), 2.12-2.23 (m, 3 H), 2.24-2.44 (m, 2 H), 2.56- 2.75 (m, 3 H), 2.90-3.13 (m, 3 H), 3.22-3.42 (m, 1 H), 3.47-3.64 (m, 3 H), 3.68-3.79 (m, 2 H), 3.85 (s, 3 H), 3.95-4.02 (m, 2 H), 4.17-4.33 (m, 2 H), 4.34-4.51 (m, 1 H), 4.53-4.81 (m, 1 H), 6.43-6.81 (m, 2 H), 6.86-7.33 (m, 2 H), 7.41 (d, J = 8.3 Hz, 1 H), 8.37 (s, 1 H), 8.41 (s, 1 H), 8.46-8.82 (m, 3 H), 9.00 (d, J = 7.7 Hz, 1 H), 9.27 (s, 1 H), 10.06 (br s, 1 H), 10.84 (s, 1 H). ¹⁹F NMR (377 MHz, DMSO-d6) δ ppm −112.84-−108.91 (m, 2 F), −99.85 (d, J = 246.6 Hz, 1 F), −97.51 (d, J = 246.6 Hz, 1 F). |
| P-21 | 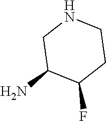 | C-1 | 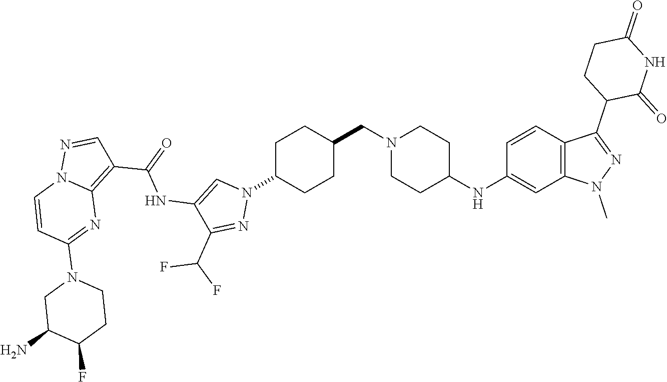 | 76% yield as a light pink solid. LCMS method 2: retention time: 1.841 min, 99% purity at 215 nm, [M − 2HCl + H]⁺ = 830.4, [M − 2HCl + 2H]²⁺ = 415.8. ¹H NMR (400 MHz, DMSO-d6) δ ppm 1.15-1.30 (m, 2 H), 1.75-1.88 (m, 2 H), 1.88-2.32 (m, 13 H), 2.56-2.66 (m, 2 H), 2.92-2.98 (m, 1 H), 3.00-3.12 (m, 2 H), 3.25- 3.42 (m, 1 H), 3.47-3.75 (m, 6 H), 3.77-3.93 (m, 4 H), 5.19 (d, J = 47.9 Hz, 1 H), 6.51-6.77 (m, 2 H), 6.94 (d, J = 8.1 Hz, 1 H), 7.14 (t, J = 54.0 Hz, 1 H), 7.43 (d, J = 8.8 Hz, 1 H), 8.32 (s, 1 H), 8.40 (s, 1 H), 8.65 (br s, 3 H), 8.92 (d, J = 8.1 Hz, 1 H), 9.35 (s, 1 H), 10.06 (br s, 1 H), 10.84 (s, 1 H). - Four protons are obscured by the water peak in the ¹H NMR spectrum. ¹⁹F NMR (377 MHz, DMSO-d6) δ ppm −198.83 (s, 1 F), −111.33 (s, 2 F). |
| P-22 | 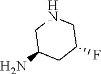 | C-10 | 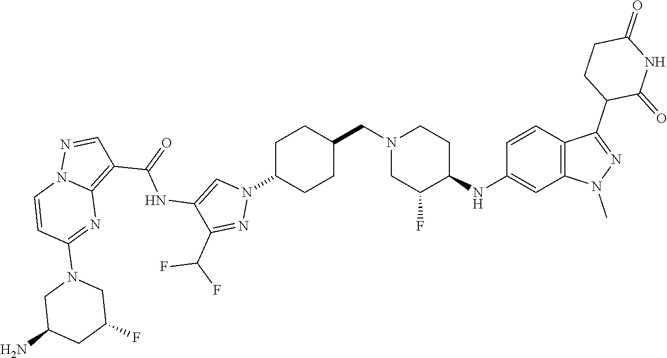 | 17% yield as a tan solid. LCMS method 2: retention time: 1.891 min, 98% purity at 215 nm, [M + H]⁺ = 848.5; [M + 2H]²⁺ = 424.8. ¹H NMR (400 MHz, DMSO-d6) δ ppm 1.18-1.30 (m, 2 H), 1.77-2.02 (m, 6 H), 2.02-2.20 (m, 5 H), 2.22-2.31 (m, 1 H), 2.34-2.44 (m, 1 H), 2.55-2.65 (m, 3 H), 2.98- 3.35 (m, 5 H), 3.35-3.44 (m, 3 H), 3.81-3.94 (m, 5 H), 4.17-4.30 (m, 2 H), 4.59 (s, 1 H), 4.87 (s, 1 H), 4.96-5.19 (m, 2 H), 6.49-6.71 (m, 2 H), 6.89 (br d, J = 7.8 Hz, 1 H), 7.00-7.29 (m, 1 H), 7.35-7.45 (m, 1 H), 8.25 (br s, 2 H), 8.32-8.34 (m, 1 H), 8.40 (s, 1 H), 8.94 (d, J = 7.8 Hz, 1 H), 9.34 (s, 1 H), 9.57 (br s, 1 H), 10.47 (br s, 1 H), 10.80-10.87 (m, 1 H). - Conformers confirmed by high temperature (80 °C) ¹H NMR. ¹⁹F NMR (377 MHz, DMSO-d6) δ ppm −187.77-−186.39 (m, 1 F), −185.18-−183.39 (m, 1 F), −111.54-−110.93 (m, 2 F). |
| P-23 | 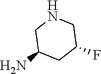 | C-11 | 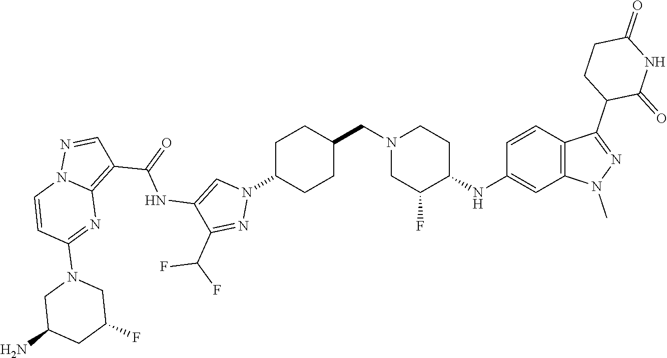 | 66% yield as a off-white solid. LCMS method 2: retention time: 1.800 min, 98% purity at 215 nm, [M + H]⁺ = 848.4 m/z, [M + H]²⁺ = 424.7 m/z. ¹H NMR (400 MHz, DMSO-d6) δ ppm 1.01-1.11 (m, 2 H), 1.56-1.68 (m, 2 H), 1.71-1.81 (m, 5 H), 1.88-1.95 (m, 2 H), 2.02-2.08 (m, 2 H), 2.11- 2.37 (m, 9 H), 2.57- 2.63 (m, 2 H), 2.84-2.90 (m, 1 H), 2.96-3.03 (m, 1 H), 3.08-3.17 (m, 2 H), 3.30-3.44 (m, 2 H), 3.54-3.65 (m, 1 H), 3.82 (s, 3 H), 4.16-4.23 (m, 2 H), 4.76-5.11 (m, 2 H), 5.79 (d, J = 8.3 Hz, 1 H), 6.52 (s, 1 H), 6.62-6.66 (m, 1 H), 6.92 (d, J = 8.1 Hz, 1 H), 7.00-7.29 (m, 1 H), 7.34 (d, J = 8.8 Hz, 1 H), 8.29 (s, 1 H), 8.38 (s, 1 H), 8.83 (d, J = 8.1 Hz, 1 H), 9.34 (s, 1 H), 10.82 (s, 1 H). ¹⁹F NMR (377 MHz, DMSO-d6) δ ppm −198.58 (s, 1 F), −183.30 (s, 1 F), −111.12 (s, 1 F), −110.99 (s, 1 F). 31% yield as a tan solid. |
| P-24 | 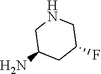 | C-12 | 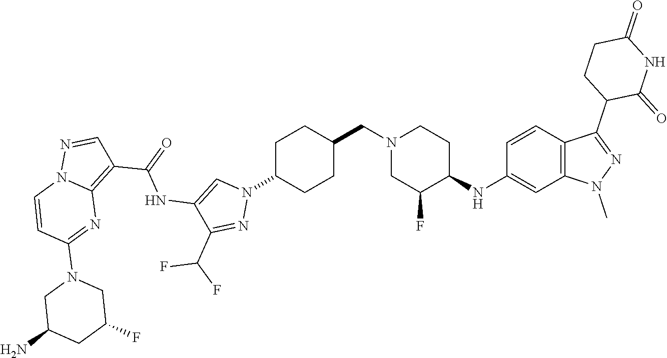 | 31% yield as a tan solid. LCMS method 2: retention time: 2.070 min, 95% purity at 215 nm, [M + H]⁺ = 848.3; [M + 2H]²⁺ = 424.7. ¹H NMR (400 MHz, DMSO-d6) δ ppm 1.19-1.34 (m, 2 H), 1.75-1.88 (m, 2 H), 1.88-2.02 (m, 4 H), 2.02-2.20 (m, 6 H), 2.21-2.31 (m, 1 H), 2.35-2.46 (m, 1 H), 2.56- 2.65 (m, 2 H), 2.97-3.13 (m, 2 H), 3.15-3.28 (m, 1 H), 3.31-3.57 (m, 5 H), 3.85 (s, 3 H), 3.90-3.98 (m, 1 H), 4.18-4.23 (m, 1 H), 4.23-4.30 (m, 1 H), 4.61 (br s, 1 H), 4.87 (br s, 1 H), 5.02-5.28 (m, 2 H), 6.55 (s, 1 H), 6.62- 6.69 (m, 1 H), 6.89 (br d, J = 7.8 Hz, 1 H), 6.99-7.29 (m, 1 H), 7.36-7.43 (m, 1 H), 8.21-8.37 (m, 4 H), 8.40 (s, 1 H), 8.94 (d, J = 8.1 Hz, 1 H), 9.34 (s, 1 H), 9.55 (br s, 1 H), 10.83 (s, 1 H). - Conformers confirmed by high temperature (80 °C) ¹H NMR. ¹⁹F NMR (377 MHz, DMSO-d6) δ ppm −200.76-−200.27 (m, 1 F), −184.28-−184.03 (m, 1 F), −111.35 (br d, J = 8.2 Hz, 2 F). |
| P-25 | 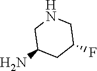 | C-13 | 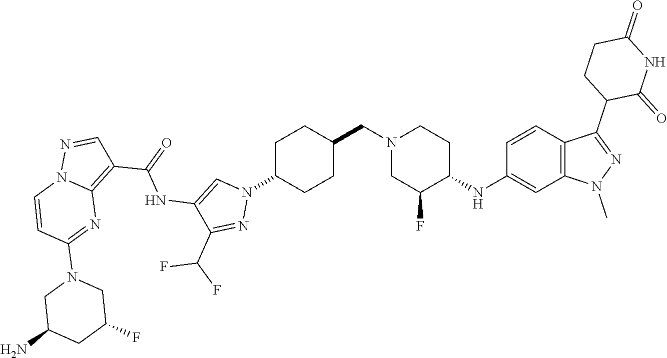 | 16% yield as a white solid. 215 nm, [M + H]⁺ = 848.3; [M + 2H]²⁺ = 424.8. ¹H NMR (400 MHz, DMSO-d6) δ ppm 1.05-1.18 (m, 2 H), 1.39-1.54 (m, 1 H), 1.55-1.68 (m, 2 H), 1.68-1.86 (m, 3 H), 1.94 (br d, J = 12.5 Hz, 2 H), 2.02-2.30 (m, 11 H), 2.56-2.65 (m, 2 H), 2.74-2.80 (m, 2 H), 3.82 (s, 3 H), 4.13-4.23 (m, 2 H), 4.33-4.39 (m, 1 H), 4.40-4.58 (m, 1 H), 4.58-4.69 (m, 1 H), 5.01 (br d, J = 46.0 Hz, 1 H), 5.68 (br d, J = 8.3 Hz, 1 H), 6.46-6.50 (m, 1 H), 6.59 (dd, J = 8.8, 1.7 Hz, 1 H), 6.88 (d, J = 8.1 Hz, 1 H), 6.93-7.22 (m, 1 H), 7.35 (d, J = 8.8 Hz, 1 H), 8.15 (s, 2 H), 8.25 (s, 1 H), 8.34 (s, 1 H), 8.70 (d, J = 7.8 Hz, 1 H), 9.28 (s, 1 H), 10.50 (br s, 1 H). - Three protons are not obsereved by ¹H NMR. ¹⁹F NMR (377 MHz, DMSO-d6) δ ppm −184.10 (br d, J = 49.0 Hz, 1 F), −182.76-−182.33 (m, 1 F), −111.08 (br d, J = 54.5 Hz, 2 F). |
| P-26 | 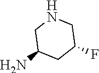 | C-14 | 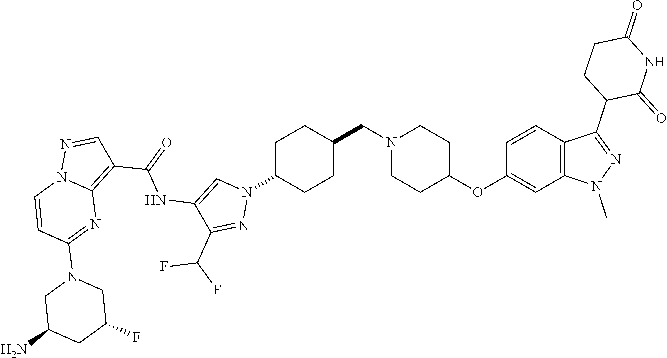 | 38% yield as a white solid. LCMS method 2: retention time: 2.128 min, 98% purity at 215 nm, [M − 2HCl + 2H]²⁺ = 416.2. ¹H NMR (400 MHz, DMSO-d6) δ ppm 1.14-1.30 (m, 3 H), 1.76-1.88 (m, 2 H), 2.31 (br s, 12 H), 2.36-2.42 (m, 1 H), 2.58-2.65 (m, 1 H), 3.00-3.12 (m, 3 H), 3.13-3.28 (m, 3 H), 3.41-3.53 (m, 3 H), 3.58-3.69 (m, 1 H), 3.94 (d, J = 4.2 Hz, 3 H), 4.20-4.28 (m, 1 H), 4.31 (dd, J = 9.7, 5.0 Hz, 1 H), 4.48-4.72 (m, 1 H), 4.81-4.92 (m, 1 H), 5.10 (d, J = 46.0 Hz, 1 H), 6.76-6.92 (m, 2 H), 6.99-7.32 (m, 2 H), 7.61 (t, J = 8.8 Hz, 1 H), 8.17-8.28 (m, 3 H), 8.33 (s, 1 H), 8.40 (s, 1 H), 8.94 (d, J = 7.8 Hz, 1 H), 9.34 (s, 1 H), 9.52-9.72 (m, 1 H), 10.87 (s, 1 H). - Conformers confirmed by high temperature (80 °C) ¹H NMR. ¹⁹F NMR (377 MHz, DMSO-d6) δ ppm −184.65 (br s, 1 F), −111.29 (br dd, J = 51.8, 19.1 Hz, 2 F). |
| P-27 | 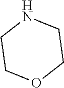 | C-1 | 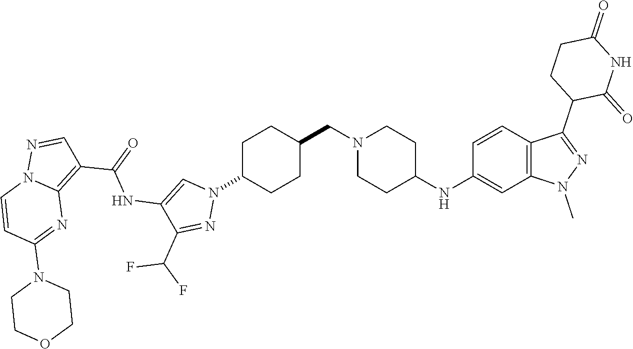 | 49% yield as a pink solid. LCMS method 2: retention time: 2.280 min, 97% purity at 215 nm, [M − HCOOH + H]⁺ = 799.4. ¹H NMR (400 MHz, DMSO-d6) δ ppm 0.99-1.12 (m, 2 H), 1.34-1.48 (m, 2 H), 1.53-1.67 (m, 1 H), 1.68-1.82 (m, 2 H), 1.87-2.00 (m, 4 H), 2.01-2.31 (m, 9 H), 2.56- 2.63 (m, 2 H), 2.79-2.87 (m, 2 H), 3.69-3.75 (m, 4 H), 3.75-3.83 (m, 7 H), 4.14-4.24 (m, 2 H), 5.74 (br d, J = 7.8 Hz, 1 H), 6.38 (s, 1 H), 6.52 (dd, J = 8.8, 1.7 Hz, 1 H), 6.91 (d, J = 8.1 Hz, 1 H), 7.09 (t, J = 54.0 Hz, 1 H), 7.32 (d, J = 8.8 Hz, 1 H), 8.16 (s, 1 H), 8.29 (s, 1 H), 8.38 (s, 1 H), 8.83 (d, J = 8.1 Hz, 1 H), 9.40 (s, 1 H), 10.81 (s, 1 H). - One proton no observed by ¹H NMR. ¹⁹F NMR (377 MHz, DMSO-d6) δ ppm −111.20 (d, J = 53.1 Hz, 2 F). |
| P-30 | 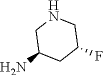 | C-16 | 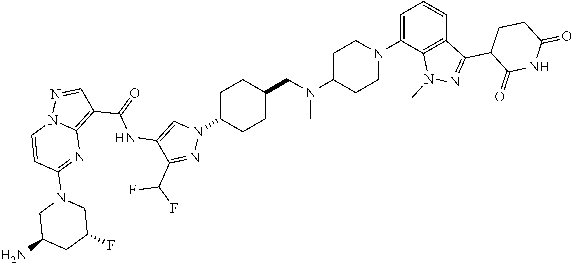 | 31% yield as a white solid. LCMS method 2: retention time: 2.010 min, 99% purity at 215 nm, [M − 2HCl + H]⁺ = 844.4; [M − 2HCl+ 2H]²⁺ = 422.8. ¹H NMR (400 MHz, DMSO-d6) δ ppm 1.15-1.35 (m, 2 H), 1.77-2.03 (m, 7 H), 2.03-2.25 (m, 7 H), 2.34-2.44 (m, 1 H), 2.57-2.66 (m, 2 H), 2.79-2.88 (m, 5 H), 2.92- 3.01 (m, 1 H), 3.16-3.25 (m, 1 H), 3.51 (br s, 6 H), 4.22- 4.29 (m, 4 H), 4.35 (dd, J = 9.5, 5.1 Hz, 1 H), 4.51-4.67 (m, 1 H), 4.76-4.95 (m, 1 H), 5.10 (br d, J = 48.0 Hz, 1 H), 6.89 (br d, J = 7.6 Hz, 1 H), 6.99-7.28 (m, 3 H), 7.43 (dd, J = 6.6, 2.4 Hz, 1 H), 8.26 (br s, 3 H), 8.33 (s, 1 H), 8.41 (s, 1 H), 8.94 (d, J = 8.1 Hz, 1 H), 9.34 (s, 1 H), 9.56 (br s, 1 H), 10.89 (s, 1 H). ¹⁹F NMR (377 MHz, DMSO-d6) δ ppm −184.64 (br s, 1 F), −111.82-−110.85 (m, 2 F). |
| P-31 | 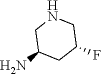 | C-17 | 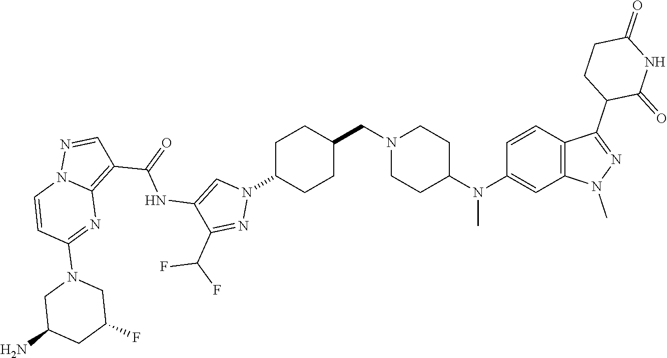 | 60% yield as a tan solid. LCMS method 2: retention time: 1.840 min, 99% purity at 215 nm, [M − 2HCl + H]⁺ = 844.5; [M − 2HCl + 2H]²⁺ = 422.8; [M − 2HCl + 3H]3+ = 282.3. ¹H NMR (400 MHz, DMSO-d6) δ ppm 1.16-1.33 (m, 2 H), 1.77-1.92 (m, 5 H), 1.93-2.04 (m, 1 H), 2.05-2.18 (m, 6 H), 2.18-2.26 (m, 1 H), 2.27-2.36 (m, 1 H), 2.36- 2.46 (m, 3 H), 2.61-2.69 (m, 2 H), 2.88 (s, 3 H), 2.92- 2.99 (m, 2 H), 3.08-3.20 (m, 2 H), 3.43-3.48 (m, 3 H), 3.87-3.93 (m, 3 H), 4.01-4.18 (m, 1 H), 4.18-4.30 (m, 2 H), 4.57-4.66 (m, 1 H), 4.71-4.82 (m, 1 H), 5.03-5.20 (m, 1 H), 6.80 (s, 1 H), 6.86-6.94 (m, 2 H), 6.94-7.23 (t, J = 54,29 Hz, 1 H), 7.53 (d, J = 8.8 Hz, 1 H), 8.30 (s, 1 H), 8.34-8.38 (m, 1 H), 8.41 (br s, 3 H), 8.83 (d, J = 7.8 Hz, 1 H), 9.27 (s, 1 H), 10.04-10.20 (m, 1 H), 10.53 (s, 1 H). - ¹H NMR was recorded at 80 °C. ¹⁹F NMR (377 MHz, DMSO-d6) δ ppm −184.62 (br s, 1 F), −111.32 (br s, 2 F). |
| P-32 | 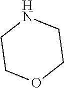 | C-17 | 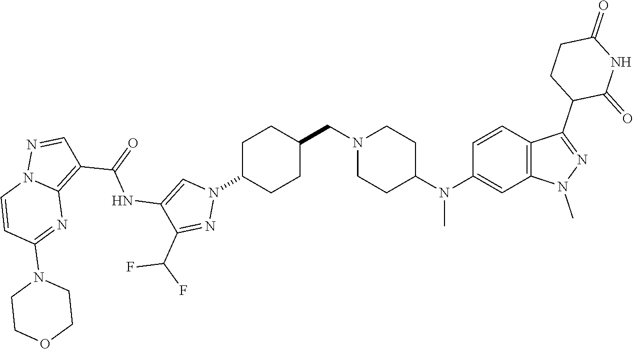 | 35% yield as a light pink solid. LCMS method 2: retention time: 2.320 min, 99% purity at 215 nm, [M+ 2H]²⁺ = 407.2; [M + H]⁺ = 813.4. ¹H NMR (400 MHz, DMSO-d6) δ ppm 0.99-1.15 (m, 2 H), 1.53-1.67 (m, 3 H), 1.69-1.83 (m, 4 H), 1.86-2.01 (m, 2 H), 2.01-2.11 (m, 4 H), 2.11-2.21 (m, 3 H), 2.22- 2.34 (m, 1 H), 2.56-2.68 (m, 2 H), 2.79 (s, 3 H), 2.89- 3.02 (m, 2 H), 3.68-3.76 (m, 5 H), 3.76-3.82 (m, 4 H), 3.87 (s, 3 H), 4.15-4.25 (m, 32 H), 6.63 (d, J = 1.5 Hz, 1 H), 6.84 (dd, J = 9.2, 1.8 Hz, 1H), 6.91 (d, J = 8.1 Hz, 1 H), 7.10 (t, J = 53.8 Hz, 1 H), 7.46 (d, J = 9.0 Hz, 1 H), 8.29 (s, 1 H), 8.38 (s, 1 H), 8.83 (d, J = 7.8 Hz, 1 H), 9.40 (s, 1 H), 10.84 (s, 1 H). 19F NMR (377 MHz, DMSO-d6) δ ppm −11.19 (s, 2 F). |

### Final Product General Method 3

### Example S8. Synthesis of P-7

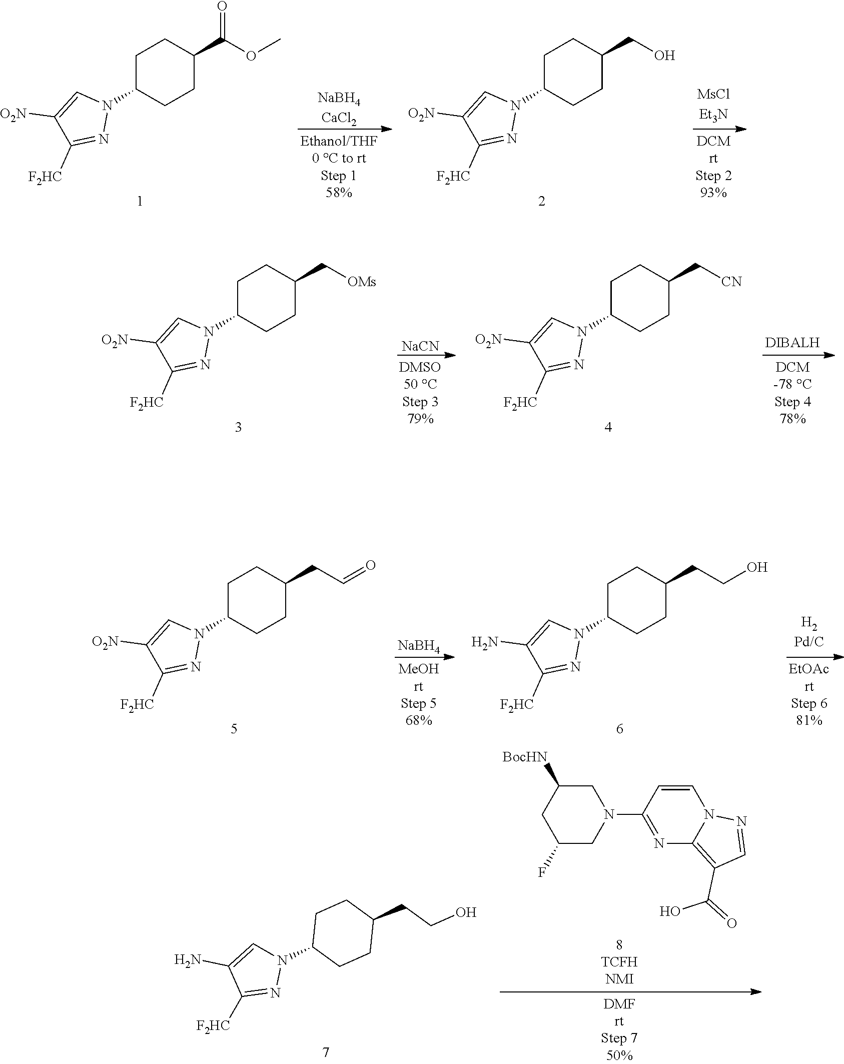

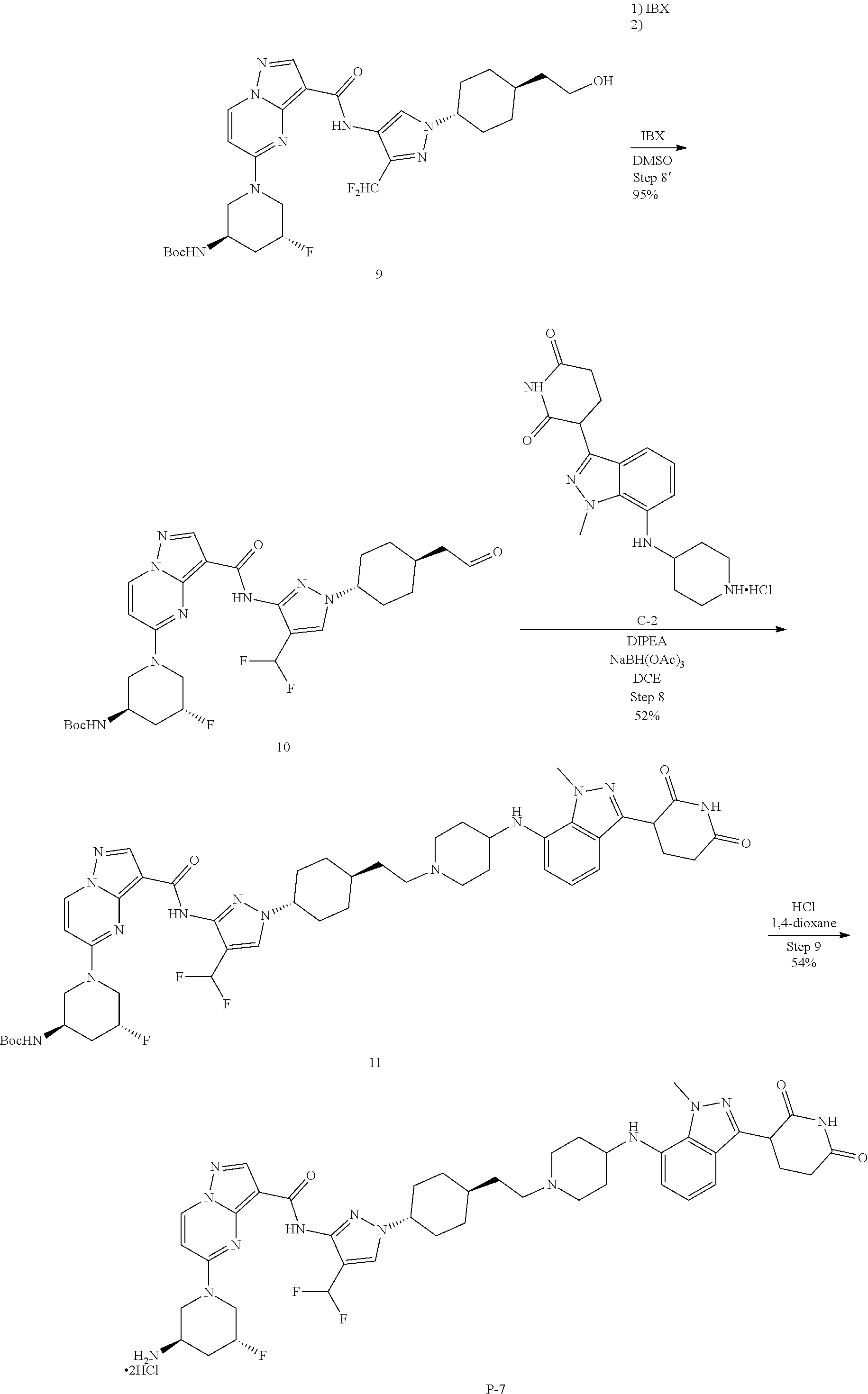

Step 1. Preparation of [4-[3-(Difluoromethyl)-4-nitro-pyrazol-1-yl]cyclohexyl]methanol (2). To a solution of methyl 4-[3-(difluoromethyl)-4-nitro-pyrazol-1-yl]cyclohexanecarboxylate 1—intermediate 4 from synthesis of T-1 —(730.0 mg, 2.26 mmol, 1 eq.) in 1:2 THF (3.8 mL)/ethanol (7.5 mL) at 0° C. was added CaCl₂) (501.2 mg, 4.52 mmol, 2 eq.) followed by the addition of NaBH₄ (341.6 mg, 9.03 mmol, 4 eq.). The resulting mixture was stirred at rt. After 16 h, at that temperature, LCMS showed total conversion. Reaction was quenched dropwise with water, and this was stirred at rt for 1 h. Then, product was extracted three times with EtOAc, and the organics were combined and washed once with water and once with brine, and finally dried over MgSO₄. EtOAc was removed under reduced pressure and the residue was purified by normal phase flash chromatography (80 g silica column, liquid deposit (DCM), elution: 0% EtOAc/Heptane over 3 CV, then 0 to 100% EtOAc/Heptane over 10 CV, then 100% EtOAc/Heptane over 3 CV (desired product was released around 50% EtOAc). Fractions were combined and concentrated to give 2 (371.9 mg, 58% yield) as a colorless oil.

LCMS method 1: 97.5% purity at 215 nm, [M+H]⁺=276.2.

¹H NMR (400 MHz, CDCl₃-d) δ ppm 1.14-1.30 (m, 3H), 1.70-1.87 (m, 2H), 1.97-2.09 (m, 2H), 2.25-2.34 (m, 2H), 3.54 (d, J=6.1 Hz, 2H), 4.18 (tt, J=12.1, 3.9 Hz, 1H), 7.10 (t, J=53.3 Hz, 1H), 8.22 (s, 1H).

¹⁹F NMR (377 MHz, CDCl₃-d) δ ppm −117.64 (d, J=53.13, 2F).

Step 2. Preparation of [4-[3-(Difluoromethyl)-4-nitro-pyrazol-1-yl]cyclohexyl]methyl methanesulfonate (3). To a solution of [4-[3-(difluoromethyl)-4-nitro-pyrazol-1-yl]cyclohexyl]methanol 2 (428.5 mg, 1.52 mmol) and Et₃N (0.28 mL, 1.98 mmol) in DCM (7.62 mL) at 0° C. was added dropwise MsCl (0.13 mL, 1.68 mmol). Then, the reaction slowly warm up to rt and stirred at this temperature. After 3 h, TLC (3:7 Heptane/EtOAc) showed completion of the reaction. Then, the reaction was partitioned between water and EtOAc. The phases were separated and the organic phase were washed three times with water, once with 1 N HCl and once with brine, before being dried over MgSO₄. EtOAc was removed under reduced pressure to give 3 (529 mg, 93% yield) as a light-yellow oil. The product was used without further purification in the next step.

LCMS method 1: 94.8% purity at 215 nm, [M+H]⁺=354.0.

¹H NMR (400 MHz, CDCl₃-d) δ ppm 1.22-1.37 (m, 3H), 1.80-1.90 (m, 2H), 2.05-2.13 (m, 2H), 2.27-2.36 (m, 2H), 3.04 (s, 3H), 4.08-4.14 (m, 2H), 4.15-4.24 (m, 1H), 7.11 (t, J=53.4 Hz, 1H), 8.22 (s, 1H).

¹⁹F NMR (377 MHz, CDCl₃-d) δ ppm −117.64 (d, J=53.13, 2F).

Step 3. Preparation of 2-[4-[3-(Difluoromethyl)-4-nitro-pyrazol-1-yl]cyclohexyl]acetaldehyde (4). A solution of [4-[3-(difluoromethyl)-4-nitro-pyrazol-1-yl]cyclohexyl]methyl methanesulfonate 3 (529.0 mg, 1.5 mmol, 1 eq.) and NaCN (183.4 mg, 3.74 mmol, 2.5 eq.) in DMSO (7.49 mL) was heated to 50° C. and stirred at this temperature. After 16 h at 50° C., LCMS showed total completion. Reaction was cooled down to rt, and reaction was quenched with water under vigorous agitation. Then, EtOAc was added, and the product was extracted two other times with EtOAc. The combined organic layers were then washed once with concentrated NaHCO₃ aqueous solution and once with brine. Finally, the latter was dried over MgSO₄ and EtOAc was removed under reduced pressure to give 4 (342 mg, 79% yield) as a yellow oil.

LCMS method 1: 98.8% purity at 215 nm, [M+H]⁺=285.2.

¹H NMR (400 MHz, CDCl₃-d) δ ppm 1.30-1.46 (m, 2H), 1.76-1.96 (m, 3H), 2.06-2.16 (m, 2H), 2.29-2.37 (m, 2H), 2.37-2.40 (m, 2H), 4.20 (tt, J=12.0, 3.9 Hz, 1H), 7.12 (t, J=54.2 Hz, 1H), 8.22 (s, 1H).

¹⁹F NMR (377 MHz, CDCl₃-d) δ ppm −117.68 (s, 2F).

Step 4. Preparation of 2-[4-[3-(Difluoromethyl)-4-nitro-pyrazol-1-yl]cyclohexyl]acetaldehyde (5). To a solution of 2-[4-[3-(difluoromethyl)-4-nitro-pyrazol-1-yl]cyclohexyl]acetonitrile 4 (342 mg, 1.18 mmol, 1 eq.) in DCM (5.88 mL) was added a 1 M solution of DIBAL-H in DCM (3.53 mL, 3.53 mmol, 3 eq.) at −78° C. The reaction was then stirred at that temperature. After 2 h, LCMS showed total completion. Reaction was slowly quenched with a solution of Rochelle salt at −78° C., and this solution was stirred for 1 h at rt. Then, the product was extracted three times with EtOAc, and the combined organic layers were washed two times with 1 M HCl and once with brine. The organic layer was then dried over MgSO₄ and EtOAc was removed under reduced pressure to give 5 (264 mg, 78% yield) as a yellow oil.

LCMS method 1: 99.9% purity at 215 nm, [M+H]⁺=288.2.

¹H NMR (400 MHz, DMSO-d6) δ ppm 1.36 (q, J=6.5 Hz, 2H), 1.75 (qd, J=12.5, 3.2 Hz, 2H), 1.85 (d, J=12.5 Hz, 2H), 2.02-2.15 (m, 2H), 3.40-3.50 (m, 2H), 4.19-4.32 (m, 1H), 4.32-4.40 (m, 1H), 7.30 (t, J=52.3 Hz, 1H), 9.06 (s, 1H).

¹⁹F NMR (377 MHz, DMSO-d6) δ ppm −117.36 (s, 2F).

Step 5. Preparation of 2-[4-[3-(Difluoromethyl)-4-nitro-pyrazol-1-yl]cyclohexyl]ethanol (6). To a solution of 2-[4-[3-(difluoromethyl)-4-nitro-pyrazol-1-yl]cyclohexyl]acetaldehyde 5 (264 mg, 0.92 mmol, 1 eq.) in methanol (4.59 mL) at 0° C. was added NaBH₄ (69.52 mg, 1.84 mmol). The resulting mixture was stirred at rt. After 16 h, LCMS showed total conversion. Water was added and this solution was stirred at rt for 1 h. Product was extracted three times with EtOAc and the combined organics were washed twice with brine. Finally, the organics was dried over MgSO₄ and EtOAc was removed under reduced pressure to give 6 (216 mg, 68% yield) as a yellow oil.

LCMS method 1: 83.3% purity at 215 nm, [M+H]⁺=290.2

¹H NMR (400 MHz, DMSO-d6) δ ppm 1.03-1.15 (m, 2H), 1.36 (q, J=6.5 Hz, 2H), 1.41-1.52 (m, 1H), 1.69-1.81 (m, 2H), 1.85 (br d, J=12.5 Hz, 2H), 2.02-2.12 (m, 2H), 3.37-3.52 (m, 2H), 4.22-4.32 (m, 1H), 4.33-4.39 (m, 1H), 7.30 (t, J=53.2 Hz, 1H), 9.06 (s, 1H).

¹⁹F NMR (377 MHz, DMSO-d6) δ ppm −117.36 (s, 2F).

Step 6. Preparation of 2-[4-[4-Amino-3-(difluoromethyl)pyrazol-1-yl]cyclohexyl]ethanol (7). N₂ was bubbled through a solution of 2-[4-[3-(difluoromethyl)-4-nitro-pyrazol-1-yl]cyclohexyl]ethanol 6 (216.1 mg, 0.75 mmol, 1 eq.) in EtOAc (3.74 mL) for 5 min. Then 10% Pd/C (238.5 mg, 0.22 mmol, 0.3 eq.) was added and N₂ was bubbled through the solution for another 5 min. Then H₂ was bubbled through the solution for 5 min and the resulting mixture was stirred at rt under 1 atm H₂. After 3 h, LCMS showed total conversion. The solution was filtered through a celite pad, and washed thoroughly with EtOAc. Finally, the filtrate was concentrated under reduced pressure to give 7 (169 mg, 81% yield) as a yellow oil. The product was used without purification in the next step.

LCMS method 1: 92.9% purity at 215 nm, [M+H]⁺=260.2

Step 7. Preparation of tert-Butyl N-[(3R,5R)-1-[3-[[3-(difluoromethyl)-1-[4-(2-hydroxyethyl)cyclohexyl]pyrazol-4-yl]carbamoyl]pyrazolo[1,5-a]pyrimidin-5-yl]-5-fluoro-3-piperidyl]carbamate (9). To a solution of 2-[4-[4-Amino-3-(difluoromethyl)pyrazol-1-yl]cyclohexyl]ethanol 7 (168.8 mg, 0.65 mmol, 1.3 eq.), 5-[(3R,5R)-3-(tert-butoxycarbonylamino)-5-fluoro-1-piperidyl]pyrazolo[1,5-a]pyrimidine-3-carboxylic acid 8—intermediate 4 from synthesis of P-4 —(190 mg, 0.50 mmol, 1 eq.) and NMI (0.14 mL, 1.75 mmol, 3.5 eq.) in MeCN (2.50 mL) was added TCFH (168.6 mg, 0.60 mmol, 1.2 eq.) at 0° C. The reaction was then stirred at rt. After 16 h, LCMS showed total completion. MeCN was removed under reduced pressure and the residue was purified by reverse phase flash chromatography (30 g C18 column, liquid deposit (DMSO), elution: 5% MeOH/0.1% HCOOH over 3 CV, then 5 to 100% MeOH/0.1% HCOOH over 15 CV, then 100% MeOH/0.1% HCOOH over 3 CV (desired product was released around 80% MeCN). Fractions were combined and concentrated to give 9 (155.6 mg, 50% yield) as an off-white solid.

LCMS method 1: 99.9% purity at 215 nm, [M+H]⁺=621.2

¹H NMR (400 MHz, DMSO-d6) δ ppm 1.04-1.17 (m, 3H), 1.29-1.44 (m, 12H), 1.65-1.77 (m, 3H), 1.80-1.93 (m, 3H), 1.97-2.07 (m, 2H), 2.10-2.18 (m, 1H), 2.94-3.14 (m, 1H), 3.36-3.53 (m, 3H), 3.61-3.72 (m, 1H), 4.11-4.23 (m, 1H), 4.31-4.40 (m, 1H), 4.94-5.13 (m, 1H), 6.83-6.90 (m, 1H), 6.91-7.23 (m, 2H), 8.28 (s, 1H), 8.33 (br s, 1H), 8.82 (d, J=8.1 Hz, 1H), 9.31 (br s, 1H).

¹⁹F NMR (377 MHz, DMSO-d6) δ ppm −184.27-−183.87 (m, 1F), −112.01-−110.10 (m, 2F).

Step 8′. Preparation of tert-butyl N-[(3R,5R)-1-[3-[[3-(difluoromethyl)-1-[4-(2-oxoethyl)cyclohexyl]pyrazol-4-yl]carbamoyl]pyrazolo[1,5-a]pyrimidin-5-yl]-5-fluoro-3-piperidyl]carbamate (10). To a solution of tert-butyl N-[(3R,5R)-1-[3-[[3-(difluoromethyl)-1-[4-(2-hydroxyethyl)cyclohexyl]pyrazol-4-yl]carbamoyl]pyrazolo[1,5-a]pyrimidin-5-yl]-5-fluoro-3-piperidyl]carbamate 9 (140 mg, 0.23 mmol, 1.0 eq.) in dry DMSO (2.2 mL) was added IBX (76 mg, 0.27 mmol, 1.2 eq.). The resulting mixture was stirred at room temperature overnight. LCMS analysis showed complete conversion. The reaction mixture was used without purification in the next step. Since the product 10 was not isolated, the reaction yield was estimated to be 95%.

LCMS method 1: 99.9% purity at 254 nm, [M+H]⁺=619.2; IBX residue visible at 215 nm.

Step 8. Preparation of tert-butyl N-[(3R,5R)-1-[3-[[3-(difluoromethyl)-1-[4-[2-[4-[[3-(2,6-dioxo-3-piperidyl)-1-methyl-indazol-7-yl]amino]-1-piperidyl]ethyl]cyclohexyl]pyrazol-4-yl]carbamoyl]pyrazolo[1,5-a]pyrimidin-5-yl]-5-fluoro-3-piperidyl]carbamate (11). To a suspension of 3-[1-methyl-7-(4-piperidylamino)indazol-3-yl]piperidine-2,6-dione hydrochloride C-2 (50 mg, 0.13 mmol, 1.1 eq.) in DCE (1.1 mL) was added DIPEA (210 μL, 1.21 mmol, 10.0 eq.), and the mixture was stirred at room temperature for 10 min, then tert-butyl N-[(3R,5R)-1-[3-[[3-(difluoromethyl)-1-[4-(2-oxoethyl)cyclohexyl]pyrazol-4-yl]carbamoyl]pyrazolo[1,5-a]pyrimidin-5-yl]-5-fluoro-3-piperidyl]carbamate 10 (75 mg, 0.12 mmol, 1.0 eq.) in 1.1 mL of DMSO was added. The mixture was stirred again for 10 min and then NaBH(OAc)₃ (33 mg, 0.16 mmol, 1.3 eq.) and the reaction was stirred at room temperature for 1 h. LCMS showed complete conversion. DCE was evaporated under reduce pressure and the resulting solution was directly loaded on a C18 RediSep Rf Gold column and purified by reverse phase flash chromatography (5% MeCN in 0.1% formic acid FA for 5 CVs then 5 to 45% MeCN in 0.1% formic acid for 20 CVs). The fractions containing the product were evaporated to give tert-butyl N-[(3R,5R)-1-[3-[[3-(difluoromethyl)-1-[4-[2-[4-[[3-(2,6-dioxo-3-piperidyl)-1-methyl-indazol-7-yl]amino]-1-piperidyl]ethyl]cyclohexyl]pyrazol-4-yl]carbamoyl]pyrazolo[1,5-a]pyrimidin-5-yl]-5-fluoro-3-piperidyl]carbamate 11 as 71.1 mg (52% yield) as a tan solid.

LCMS method 1: 84.3% purity at 215 nm, and 97.4% at 254 nm; [M+H]⁺=947.3; [M+2H]²⁺=472.8.

¹H NMR (400 MHz, DMSO-d6) δ ppm 0.83-0.92 (m, 4H), 1.11 (s, 9H), 1.19 (s, 2H), 1.22-1.45 (m, 12H), 1.62 (br s, 2H), 1.73 (br d, J=13.2 Hz, 2H), 1.82-1.93 (m, 2H), 1.96-2.08 (m, 2H), 2.09-2.31 (m, 3H), 2.40-2.46 (m, 1H), 2.56-2.69 (m, 2H), 2.85-2.96 (m, 1H), 3.08 (s, 3H), 4.14 (dd, J=5.7, 3.3 Hz, 1H), 4.24 (s, 3H), 4.96-5.01 (m, 1H), 6.54 (d, J=7.3 Hz, 1H), 6.89 (t, J=7.7 Hz, 1H), 6.99 (d, J=8.1 Hz, 1H), 7.65-7.74 (m, 2H), 8.28 (s, 1H), 8.33 (s, 1H), 8.82 (d, J=7.8 Hz, 1H), 9.31 (s, 1H), 10.85 (s, 1H).

¹⁹F NMR (377 MHz, DMSO-d6): the spectrum does not show any signal strong enough to be clearly assigned.

Step 9. Preparation of N-[3-(difluoromethyl)-1-[4-[2-[4-[[3-(2,6-dioxo-3-piperidyl)-1-methyl-indazol-7-yl]amino]-1-piperidyl]ethyl]cyclohexyl]pyrazol-4-yl]-5-[(3R,5R)-3-amino-5-fluoro-1-piperidyl]pyrazolo[1,5-a]pyrimidine-3-carboxamide bis-HCl salt (P-7). To tert-butyl N-[(3R,5R)-1-[3-[[3-(difluoromethyl)-1-[4-[2-[4-[[3-(2,6-dioxo-3-piperidyl)-1-methyl-indazol-7-yl]amino]-1-piperidyl]ethyl]cyclohexyl]pyrazol-4-yl]carbamoyl]pyrazolo[1,5-a]pyrimidin-5-yl]-5-fluoro-3-piperidyl]carbamate 11 (65 mg, 0.07 mmol, 1.0 eq.) was added a 4.0 M solution of HCl (2.6 mL, 10.3 mmol, 150 eq.) in 1,4-dioxane. The mixture was stirred at room temperature for 1 h. HPLC indicated complete conversion. The solvent was removed under reduced pressure and water and roughly 10 drops of DMSO were added. The solution was then loaded on a C18 RediSep Rf Gold column and purified by reverse phase flash chromatography (5% MeCN in HCl 0.02M for 5 CVs then 5 to 20% MeCN in 0.02 M HCl for 20 CVs). Product eluted out at 22% MeCN. Pure fractions were combined, evaporated under reduce pressure and freeze-dried to give 31.77 mg (54% yield) of N-[3-(difluoromethyl)-1-[4-[2-[4-[[3-(2,6-dioxo-3-piperidyl)-1-methyl-indazol-7-yl]amino]-1-piperidyl]ethyl]cyclohexyl]pyrazol-4-yl]-5-[(3R,5R)-3-amino-5-fluoro-1-piperidyl]pyrazolo[1,5-a]pyrimidine-3-carboxamide (P-7) as a bis-HCl salt.

LCMS method 2: 99.5% purity at 215 nm; [M+H]⁺=844.5; [M+2H]²⁺=422.8.

¹H NMR (400 MHz, DMSO-d6) δ ppm 1.10-1.25 (m, 2H), 1.35-1.48 (m, 1H), 1.62-1.92 (m, 7H), 1.93-2.25 (m, 7H), 2.25-2.35 (m, 1H), 2.36-2.46 (m, 1H), 2.53-2.72 (m, 3H), 2.99-3.23 (m, 4H), 3.28-3.59 (m, 6H), 4.18-4.35 (m, 5H), 4.54-4.70 (m, 1H), 4.80-4.95 (m, 1H), 5.10 (d, J=46.0 Hz, 1H), 6.61-6.70 (m, 1H), 6.87-6.96 (m, 2H), 6.99-7.29 (m, 2H), 8.28-8.45 (m, 5H), 8.93 (d, J=8.1 Hz, 1H), 9.33 (s, 1H), 10.18-10.35 (m, 1H), 10.86 (s, 1H).

¹⁹F NMR (377 MHz, DMSO-d6) δ ppm −184.63 (s, 1F), −111.26 (s, 2F).

### Example S9. Synthesis of P-34

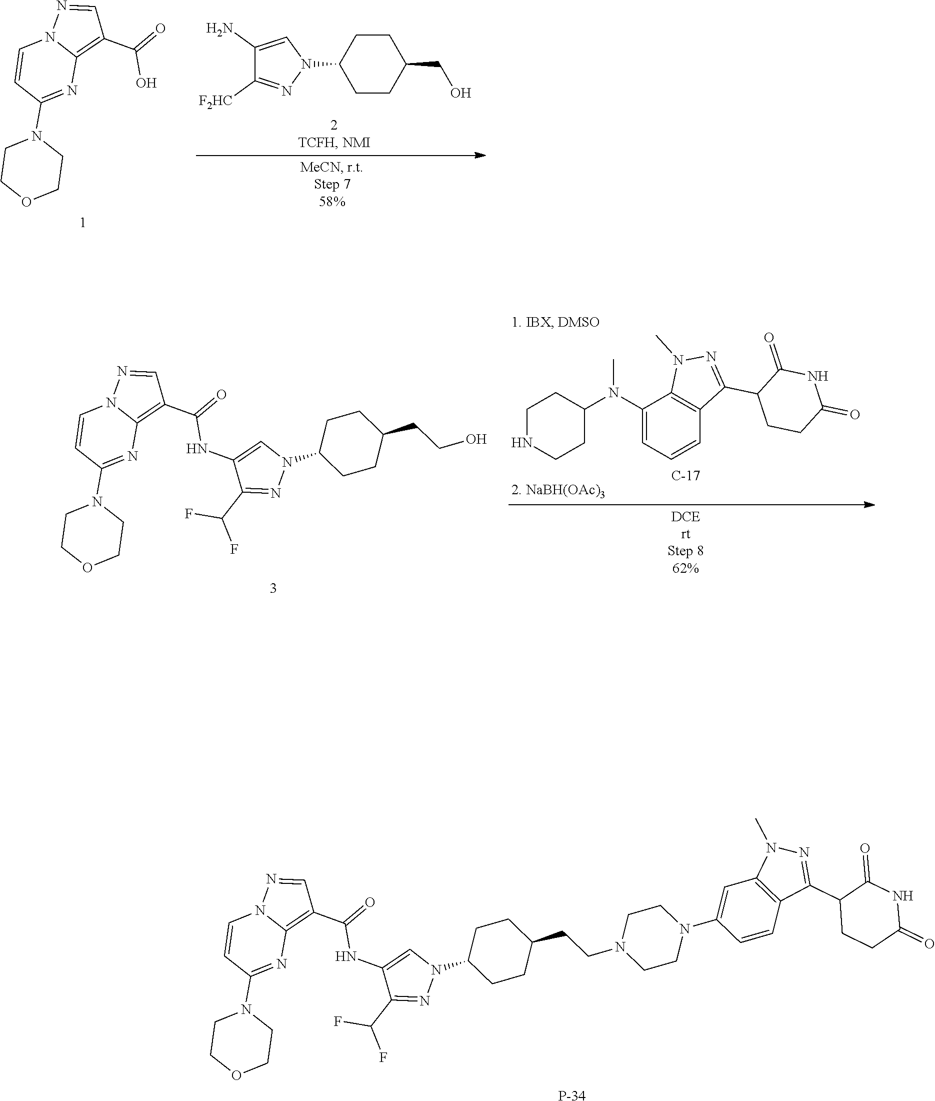

Step 7. Preparation of N-[3-(difluoromethyl)-1-[4-(2-hydroxyethyl)cyclohexyl]pyrazol-4-yl]-5-morpholino-pyrazolo[1,5-a]pyrimidine-3-carboxamide (3). To a round-bottom flask were introduced 2-[4-[4-amino-3-(difluoromethyl)pyrazol-1-yl]cyclohexyl]ethanol 2—intermediate 7 from synthesis of P-7 —(60 mg, 0.17 mmol, 1.1 eq.), 5-morpholinopyrazolo[1,5-a]pyrimidine-3-carboxylic acid 1—intermediate 4 from synthesis of P-27 —(37.7 mg, 0.15 mmol, 1 eq.), MeCN (1.2 mL) and NMI (50 μL, 0.63 mmol, 3.7 eq.), and the mixture was stirred 0° C. After 5 min, TCFH (57 mg, 0.20 mmol, 1.2 eq.) was added and the reaction was stirred at 0° C. After 5 min, the reaction was allowed to stir at room temperature. After 3 h, LCMS showed complete conversion. Water was added to the mixture and MeCN was removed under reduced pressure until a persistent precipitate appeared. The suspension was then extracted with EtOAc (2×). The combined organic layers were washed with brine (2×), dried over Na₂SO₄, filtered and concentrated to dryness. The residue was purified by normal phase flash chromatography (24 g silica column, preabsorbed, elution: 0 to 5% DCM/MeOH over 15 CV). Fractions were combined and concentrated to give 3 (60 mg, 58% yield) as a white solid.

LCMS method 3: 80.5% purity at 215 nm, [M+H]⁺=490.2.

Step 8. Preparation of N-[3-(difluoromethyl)-1-[4-[2-[4-[[3-(2,6-dioxo-3-piperidyl)-1-methyl-indazol-7-yl]-methyl-amino]-1-piperidyl]ethyl]cyclohexyl]pyrazol-4-yl]-5-morpholino-pyrazolo[1,5-a]pyrimidine-3-carboxamide (P-34). To a solution of N-[3-(difluoromethyl)-1-[4-(2-hydroxyethyl)cyclohexyl]pyrazol-4-yl]-5-morpholino-pyrazolo[1,5-a]pyrimidine-3-carboxamide 3 (28 mg, 0.05 mmol, 1 eq.) in anhydrous DMSO (1 mL) was added IBX (15 mg, 0.06 mmol, 1.2 eq.). The resulting mixture was stirred at room temperature. After 3 h, LCMS showed complete conversion toward the corresponding aldehyde. To the reaction mixture was added 3-[1-methyl-7-[methyl(4-piperidyl)amino]indazol-3-yl]piperidine-2,6-dione hydrochloride C-17 (39 mg, 0.09 mmol, 2 eq.), DCE (1 mL) and DIPEA (50 μL, 0.29 mmol, 6.2 eq.). The mixture was stirred at room temperature and after 10 min, NaBH(OAc)₃ (15 mg, 0.07 mmol, 1.5 eq.) was added. After 2 h, LCMS showed complete conversion. DCM was evaporated under reduced pressure and the crude mixture was purified by reverse phase FC (30 g C18 RediSep Rf Gold column, liquid deposit (DMSO), 5% MeOH/0.1% HCOOH over 4 CV, then 5 to 80% MeOH/0.1% HCOOH over 15 CV). Fractions were combined and concentrated to afford an impure material, which was then co-evaporated with MeOH and water (1×), then with DCM containing a small amount of triethylamine to afford the corresponding free base. The resulting material was then dissolved in a minimum amount of DCM and injected for purification by normal phase flash chromatography (24 g silica column, elution: 0 to 10% DCM/MeOH over 20 CV). Pure fractions were combined and concentrated to give P-34 (23 mg, 61% yield) as a white solid.

LCMS method 2: retention time: 2.391 min, 99% purity at 215 nm, [M+2H]²⁺=414.2; [M+H]⁺=827.5.

¹H NMR (400 MHz, DMSO-d6) δ ppm 1.03-1.17 (m, 2H), 1.28-1.40 (m, 3H), 1.51-1.64 (m, 2H), 1.65-1.78 (m, 4H), 1.79-1.94 (m, 4H), 1.98-2.06 (m, 2H), 2.14-2.22 (m, 1H), 2.24-2.32 (m, 2H), 2.35-2.41 (m, 1H), 2.52-2.53 (m, 1H), 2.58-2.66 (m, 1H), 2.70 (s, 3H), 2.80-2.89 (m, 2H), 2.93-3.02 (m, 1H), 3.70-3.76 (m, 4H), 3.76-3.84 (m, 4H), 4.11-4.22 (m, 1H), 4.24 (s, 3H), 4.35 (dd, J=9.5, 5.1 Hz, 1H), 6.91 (d, J=8.1 Hz, 1H), 6.95-7.24 (m, 3H), 7.43 (dd, J=8.1, 0.5 Hz, 1H), 8.29 (s, 1H), 8.37 (s, 1H), 8.83 (d, J=8.1 Hz, 1H), 9.40 (s, 1H), 10.89 (s, 1H).

¹⁹F NMR (377 MHz, DMSO-d6) δ ppm −111.20 (s, 2F).

The following compounds were synthesized via the same general routes with modifications to acid (8) in step 7 and CBM (C—X) in step 8 (Table 6).

TABLE 6

| Com- | | | | |
|---|---|---|---|---|
| pound | | | | |
| No. | Amine | CBM | Structure | Characterization Data |
| P-7 | 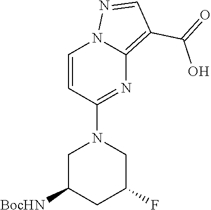 [SMILES: CC(C)(C)OC(=O)N[C@@H]1C[C@@H](F)CN(c2ccn3ncc(C(=O)O)c3n2)C1] | C-2 | 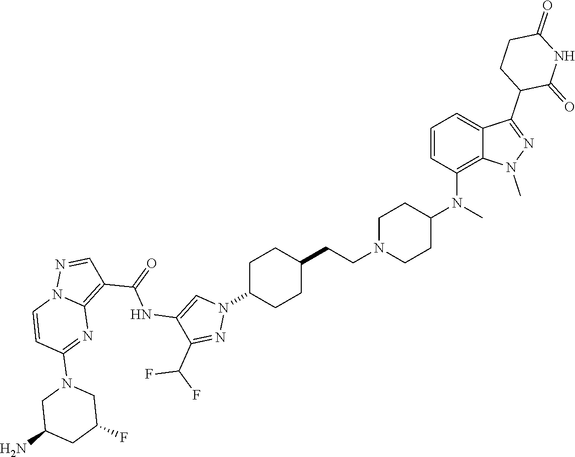 | 54% yield as a off-white solid. LCMS method 2: retention time: 1.941 min, 99% purity at 215 nm, [M + H]⁺ = 844.5; [M + 2H]²⁺ = 422.8. ¹H NMR (400 MHz, DMSO-d6) δ ppm 1.10- 1.25 (m, 2 H), 1.35-1.48 (m, 1 H), 1.62-1.92 (m, 7 H), 1.93-2.25 (m, 7 H), 2.25-2.35 (m, 1 H), 2.36-2.46 (m, 1 H), 2.53-2.72 (m, 3 H), 2.99-3.23 (m, 4 H), 3.28-3.59 (m, 6 H), 4.18- 4.35 (m, 5 H), 4.54-4.70 (m, 1 H), 4.80-4.95 (m, 1 H), 5.10 (d, J = 46.0 Hz, 1 H), 6.61-6.70 (m, 1 H), 6.87-6.96 (m, 2 H), 6.99-7.29 (m, 2 H), 8.28-8.45 (m, 5 H), 8.93 (d, J = 8.1 Hz, 1 H), 9.33 (s, 1 H), 10.18-10.35 (m, 1 H), 10.86 (s, 1 H). ¹⁹F NMR (377 MHz, DMSO-d6) δ ppm −184.63 (s, 1 F), −111.26 (s, 2 F). |
| P-10 | 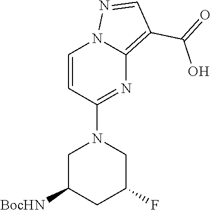 [SMILES: CC(C)(C)OC(=O)N[C@@H]1C[C@@H](F)CN(c2ccn3ncc(C(=O)O)c3n2)C1] | C-6 | 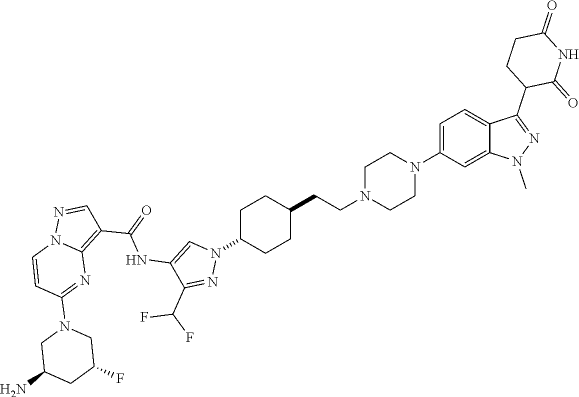 | 49% yield as a white solid. LCMS method 2: retention time: 1.882 min, 99% purity at 215 nm, [M − 2HCl + 2H]²⁺ = 415.8; [M − 2HCl + H]⁺ = 830.4. ¹H NMR (400 MHz, DMSO-d6) δ ppm 1.13- 1.25 (m, 2 H), 1.37-1.50 (m, 1 H), 1.65-1.74 (m, 2 H), 1.74-1.83 (m, 2 H), 1.84-1.98 (m, 3 H), 2.03-2.10 (m, 2 H), 2.12-2.22 (m, 1 H), 2.27-2.44 (m, 2 H), 2.56-2.72 (m, 2 H), 3.11- 3.28 (m, 7 H), 3.28-3.34 (m, 1 H), 3.36-3.41 (m, 1 H), 3.59-3.64 (m, 2 H), 3.91-3.97 (m, 5 H), 4.19-4.32 (m, 2 H), 4.49-4.69 (m, 1 H), 4.77-4.97 (m, 1 H), 5.10 (d, J = 46.2 Hz, 1 H), 6.88 (d, J = 7.8 Hz, 1 H), 6.94-7.31 (m, 3 H), 7.57 (d, J = 8.8 Hz, 1 H), 8.16-8.30 (m, 3 H), 8.33 (s, 1 H), 8.40 (s, 1 H), 8.94 (d, J = 7.8 Hz, 1 H), 9.33 (s, 1 H), 10.18-10.32 (m, 1 H), 10.86 (s, 1 H). ¹⁹F NMR (377 MHz, DMSO-d6) δ ppm −184.60 (s, 1 F), −111.25 (s, 2 F). |
| P-11 | 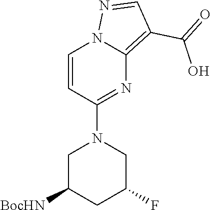 [SMILES: CC(C)(C)OC(=O)N[C@@H]1C[C@@H](F)CN(c2ccn3ncc(C(=O)O)c3n2)C1] | C-5 | 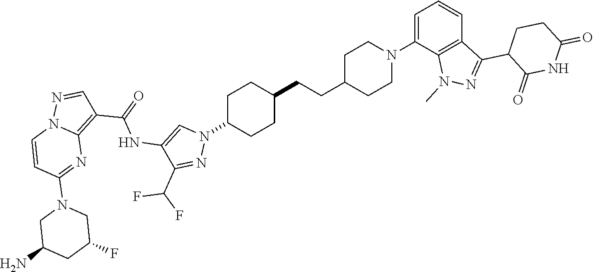 | 46% yield as a off-white solid. LCMS method 2: retention time: 1.920 min, 99% purity at 215 nm, [M − 2HCl + H]⁺ = 830.5. ¹H NMR (400 MHz, DMSO-d6) δ ppm 1.12- 1.26 (m, 2 H), 1.40-1.51 (m, 1 H), 1.68-1.84 (m, 4 H), 1.85-1.95 (m, 2 H), 1.95-2.13 (m, 3 H), 2.14-2.22 (m, 1 H), 2.27-2.47 (m, 2 H), 2.57-2.73 (m, 2 H), 3.21-3.52 (m, 11 H), 3.55- 3.66 (m, 2 H), 4.19-4.30 (m, 4 H), 4.36 (dd, J = 9.8, 5.1 Hz, 1 H), 4.56-4.72 (m, 1 H), 4.78- 4.96 (m, 1 H), 5.10 (d, J = 46.0 Hz, 1 H), 6.92 (d, J = 8.1 Hz, 1 H), 7.00-7.30 (m, 3 H), 7.45-7.50 (m, 1 H), 8.32 (s, 1 H), 8.40 (s, 1 H), 8.43-8.54 (m, 3 H), 8.93 (d, J = 8.1 Hz, 1 H), 9.33 (s, 1 H), 10.89 (s, 1 H), 10.97 (br s, 1 H). ¹⁹F NMR (377 MHz, DMSO-d6) δ ppm −184.59 (s, 1 F), −111.26 (s, 2 F). |
| P-16 | 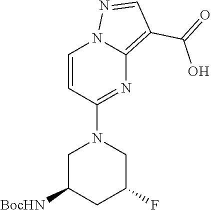 [SMILES: CC(C)(C)OC(=O)N[C@@H]1C[C@@H](F)CN(c2ccn3ncc(C(=O)O)c3n2)C1] | C-1 | 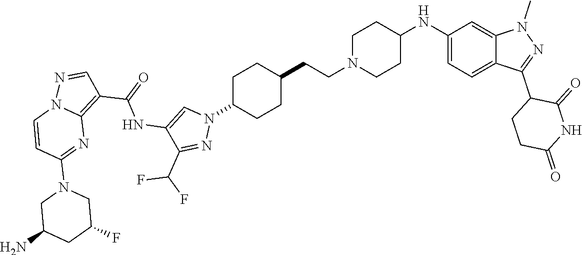 [SMILES: Cn1nc(C2CCC(=O)NC2=O)c2ccc(NC3CCN(CC[C@H]4CC[C@H](n5cc(NC(=O)c6cnn7ccc(N8C[C@H](N)C[C@@H](F)C8)nc67)c(C(F)F)n5)CC4)CC3)cc21] | 75% yield as a purple solid. LCMS method 2: retention time: 1.888 min, 97% purity at 215 nm, [M − 2HCl + H]⁺ = 844.5; [M − 2HCl + 2H]²⁺ = 422.8. ¹H NMR (400 MHz, DMSO-d6) δ ppm 1.11- 1.27 (m, 2 H), 1.35-1.49 (m, 1 H), 1.61-1.99 (m, 8 H), 2.00-2.30 (m, 6 H), 2.34-2.45 (m, 1 H), 2.56-2.65 (m, 2 H), 2.99-3.16 (m, 3 H), 3.19-3.62 (m, 8 H), 3.83 (s, 3 H), 4.14-4.31 (m, 2 H), 4.50-4.70 (m, 1 H), 4.76-4.98 (m, 1 H), 5.10 (d, J = 45.0 Hz, 1 H), 6.05 (br s, 1 H), 6.44-6.64 (m, 2 H), 6.89 (br d, J = 7.8 Hz, 1 H), 7.14 (t, J = 55.0 Hz, 1 H), 7.34-7.41 (m, 1 H), 8.20-8.30 (m, 3 H), 8.32 (s, 1 H), 8.40 (s, 1 H), 8.94 (d, J = 8.1 Hz, 1 H), 9.33 (s, 1 H), 9.92 (br s, 1 H), 10.83 (s, 1 H). ¹⁹F NMR (377 MHz, DMSO-d6) δ ppm −184.43 (s, 1 F), −111.27 (s, 2 F). |
| P-33 | 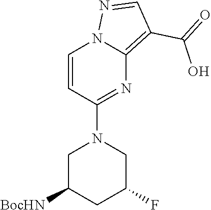 [SMILES: CC(C)(C)OC(=O)N[C@@H]1C[C@@H](F)CN(c2ccn3ncc(C(=O)O)c3n2)C1] | C-17 | 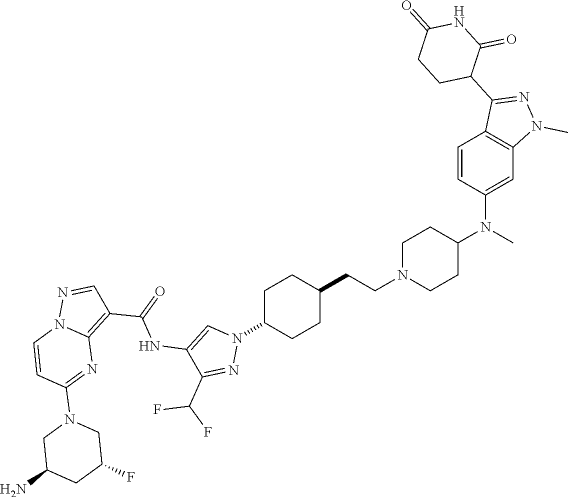 | 35% yield as a yellow solid. LCMS method 2: retention time: 1.909 min, 99% purity at 215 nm, [M − 2HCl + H]⁺ = 858.5; [M − 2HCl + 2H]²⁺ = 429.7; [M − 2HCl + 3H]3+ = 286.9. ¹H NMR (400 MHz, DMSO-d6) δ ppm 1.15- 1.28 (m, 2 H), 1.41-1.54 (m, 1 H), 1.67-1.76 (m, 2 H), 1.76-1.85 (m, 2 H), 1.87-1.94 (m, 3 H), 1.96-2.07 (m, 1 H), 2.07-2.17 (m, 3 H), 2.17-2.35 (m, 4 H), 2.40-2.46 (m, 1 H), 2.61- 2.69 (m, 2 H), 2.83-2.92 (m, 3 H), 3.04-3.18 (m, 4 H), 3.37-3.48 (m, 2 H), 3.52-3.60 (m, 2 H), 3.87-3.94 (m, 3 H), 4.03-4.14 (m, 1 H), 4.16-4.29 (m, 2 H), 4.58-4.59 (m, 1 H), 4.61- 4.63 (m, 1 H), 4.73-4.81 (m, 1 H), 5.03-5.20 (m, 1 H), 6.78 (s, 1 H), 6.86-6.94 (m, 2 H), 7.08 (t, J = 54.3 Hz, 1 H), 7.53 (d, J = 9.0 Hz, 1 H), 8.28-8.33 (m, 1 H), 8.35 (s, 4 H), 8.84 (d, J = 7.8 Hz, 1 H), 9.26 (s, 1 H), 10.40 (s, 1 H), 10.53 (br s, 1 H), -¹H NMR was acquired at 80 °C. ¹⁹F NMR (377 MHz, DMSO-d6) δ ppm −184.68 (br s, 1 F), −111.24 (br s, 2 F). |
| P-34 | 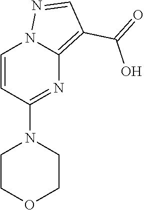 [SMILES: O=C(O)c1cnn2ccc(N3CCOCC3)nc12] | C-17 | 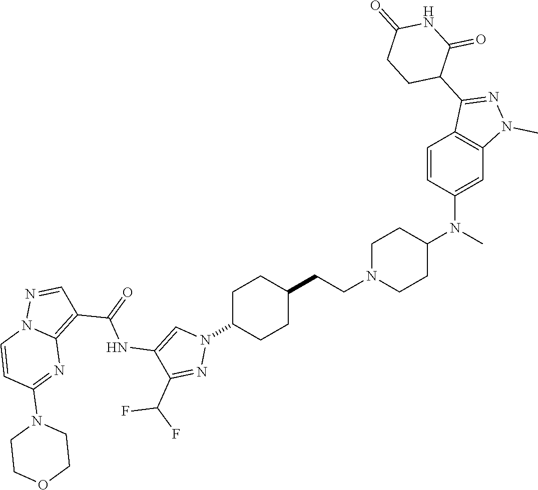 | 23% yield as a white solid. LCMS method 2: retention time: 2.391 min, 99% purity at 215 nm, [M + 2H]²⁺ = 414.2; [M + H]⁺ = 827.5. ¹H NMR (400 MHz, DMSO-d6) δ 1.09- 1.18 (m, 2 H), 1.33-1.43 (m, 3 H), 1.57-1.65 (m, 2 H), 1.67-1.81 (m, 4 H), 1.82-1.91 (m, 2 H), 1.99-2.07 (m, 4 H), 2.10-2.21 (m, 1 H), 2.24-2.38 (m, 3 H), 2.59-2.63 (m, 2 H), 2.79 (s, 3 H), 2.91-3.00 (m, 2 H), 3.68-3.76 (m, 5 H), 3.76-3.82 (m, 4 H), 3.86 (s, 3 H), 4.12-4.20 (m, 1 H), 4.23 (dd, J = 9.0, 5.1 Hz, 1 H), 6.62 (d, J = 2.0 Hz, 1 H), 6.83 (dd, J = 9.2, 2.1 Hz, 1 H), 6.91 (d, J = 8.1 Hz, 1 H), 7.10 (t, J = 53.1 Hz, 1 H), 7.45 (d, J = 9.0 Hz, 1 H), 8.29 (s, 1 H), 8.38 (s, 1 H), 8.83 (d, J = 7.8 Hz, 1 H), 9.40 (s, 1 H), 10.84 (s, 1 H). ¹⁹F NMR (377 MHz, DMSO-d6) δ ppm −111.19 (s, 2 F). |
| P-35 | 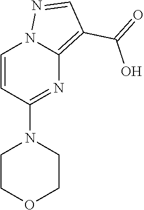 [SMILES: O=C(O)c1cnn2ccc(N3CCOCC3)nc12] | C-16 | 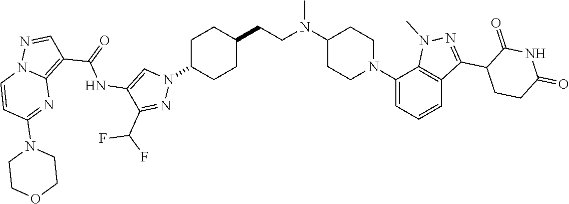 | 22% yield as a white solid. LCMS method 4: retention time: 2.523 min, 99% purity at 215 nm, [M + H]⁺ = 827.4, [M + 2H]²⁺ = 414.2. ¹H NMR (400 MHz, DMSO-d6) δ ppm 1.08- 1.25 (m, 3 H), 1.33-1.43 (m, 3 H), 1.67-1.80 (m, 4 H), 1.81-1.92 (m, 4 H), 2.00-2.08 (m, 2 H), 2.13-2.21 (m, 1 H), 2.24 (s, 3 H), 2.28-2.32 (m, 1 H), 2.34-2.38 (m, 1 H), 2.55-2.65 (m, 5 H), 2.69-2.77 (m, 2 H), 3.23-3.27 (m, 1 H), 3.69-3.75 (m, 4 H), 3.76-3.84 (m, 3 H), 4.14- 4.22 (m, 1 H), 4.25 (s, 3 H), 4.33 (dd, J = 9.5, 5.1 Hz, 1 H), 6.91 (d, J = 7.8 Hz, 1 H), 6.95-7.24 |
| | | | | (m, 3 H), 7.34-7.40 (m, 1 H), 8.29 (s, 1 H), 8.38 |
| | | | | (s, 1 H), 8.83 (d, J = 8.1 Hz, 1 H), 9.39 (s, 1 H), |
| | | | | 10.88 (s, 1 H). |
| | | | | ¹⁹F NMR (377 MHz, DMSO-d6) δ ppm −111.19 |
| | | | | (s, 2 F). |
| P-38 | 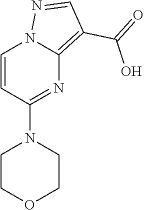 [SMILES: O=C(O)c1cnn2ccc(N3CCOCC3)nc12] | C-15 | 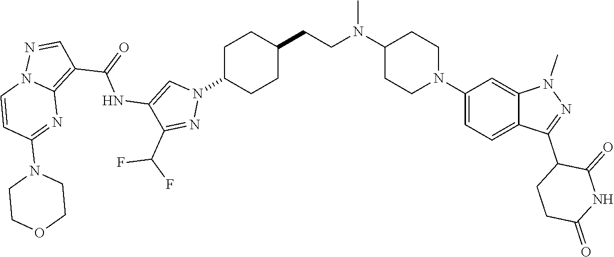 [SMILES: CN(CC[C@H]1CC[C@H](n2cc(NC(=O)c3cnn4ccc(N5CCOCC5)nc34)c(C(F)F)n2)CC1)C1CCN(c2ccc3c(C4CCC(=O)NC4=O)nn(C)c3c2)CC1] | 23% yield as a white solid. LCMS method 4: retention time: 2.480 min, 99% purity at 215 nm, [M + H]⁺ = 827.4, [M + 2H]²⁺ = 414.3. ¹H NMR (400 MHz, DMSO-d6) δ ppm 0.97 (t, J = 7.2 Hz, 1 H), 1.05-1.19 (m, 2 H), 1.32-1.42 (m, 3 H), 1.51-1.65 (m, 2 H), 1.67-1.82 (m, 4 H), 1.83-1.90 (m, 2 H), 1.99-2.07 (m, 2 H), 2.11-2.18 (m, 1 H), 2.20 (s, 3 H), 2.24-2.32 (m, 1 H), 2.52-2.58 (m, 2 H), 2.58-2.65 (m, 2 H), 2.69-2.78 (m, 2 H), 3.70-3.75 (m, 4 H), 3.76-3.86 (m, 6 H), 3.89 (s, 3 H), 4.13-4.22 (m, 1 H), 4.25 (dd, J = 9.0, 5.1 Hz, 1 H), 6.84 (s, 1 H), 6.91 (d, J = 7.8 Hz, 2 H), 7.09 (t, J = 53.6 Hz, 1 H), 7.48 (d, J = 8.8 Hz, 1 H), 8.29 (s, 1 H), 8.38 (s, 1 H), 8.83 (d, J = 8.1 Hz, 1 H), 9.39 (s, 1 H), 10.84 (s, 1 H). |
| | | | | ¹⁹F NMR (377 MHz, DMSO-d6) δ ppm −111.20 |
| | | | | (d, J = 53.1 Hz, 2 F). |
| P-39 | 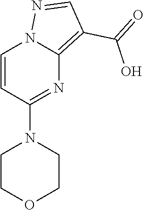 [SMILES: O=C(O)c1cnn2ccc(N3CCOCC3)nc12] | C-20 | 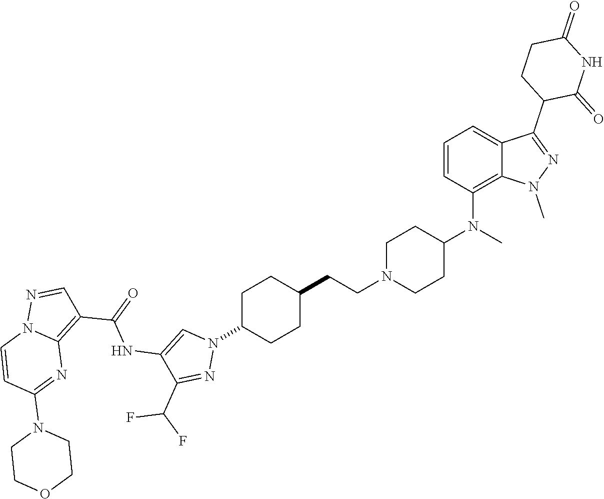 | 61% yield as a white solid. LCMS method 4: retention time: 2.581 min, 99% purity at 215 nm, [M + H]⁺ = 827.4, [M + 2H]²⁺ = 414.2. ¹H NMR (400 MHz, DMSO-d6) δ ppm 1.03- 1.17 (m, 2 H), 1.28-1.40 (m, 3 H), 1.51-1.64 (m, 2 H), 1.65-1.78 (m, 4 H), 1.79-1.94 (m, 4 H), 1.98-2.06 (m, 2 H), 2.14-2.22 (m, 1 H), 2.24-2.32 (m, 2 H), 2.35-2.41 (m, 1 H), 2.52- 2.53 (m, 1 H), 2.58-2.66 (m, 1 H), 2.70 (s, 3 H), 2.80-2.89 (m, 2 H), 2.93-3.02 (m, 1 H), 3.70- 3.76 (m, 4 H), 3.76-3.84 (m, 4 H), 4.11-4.22 (m, 1 H), 4.24 (s, 3 H), 4.35 (dd, J = 9.5, 5.1 Hz, 1 H), 6.91 (d, J = 8.1 Hz, 1 H), 6.95-7.24 (m, 3 H), 7.43 (dd, J = 8.1, 0.5 Hz, 1 H), 8.29 (s, 1 H), 8.37 (s, 1 H), 8.83 (d, J = 8.1 Hz, 1 H), 9.40 (s, 1 H), 10.89 (s, 1 H). 19 F NMR (377 MHz, DMSO-d6) δ ppm −111.20 (s, 2 F). |

### Final Product General Method 4

### Example S10. Synthesis of P-28

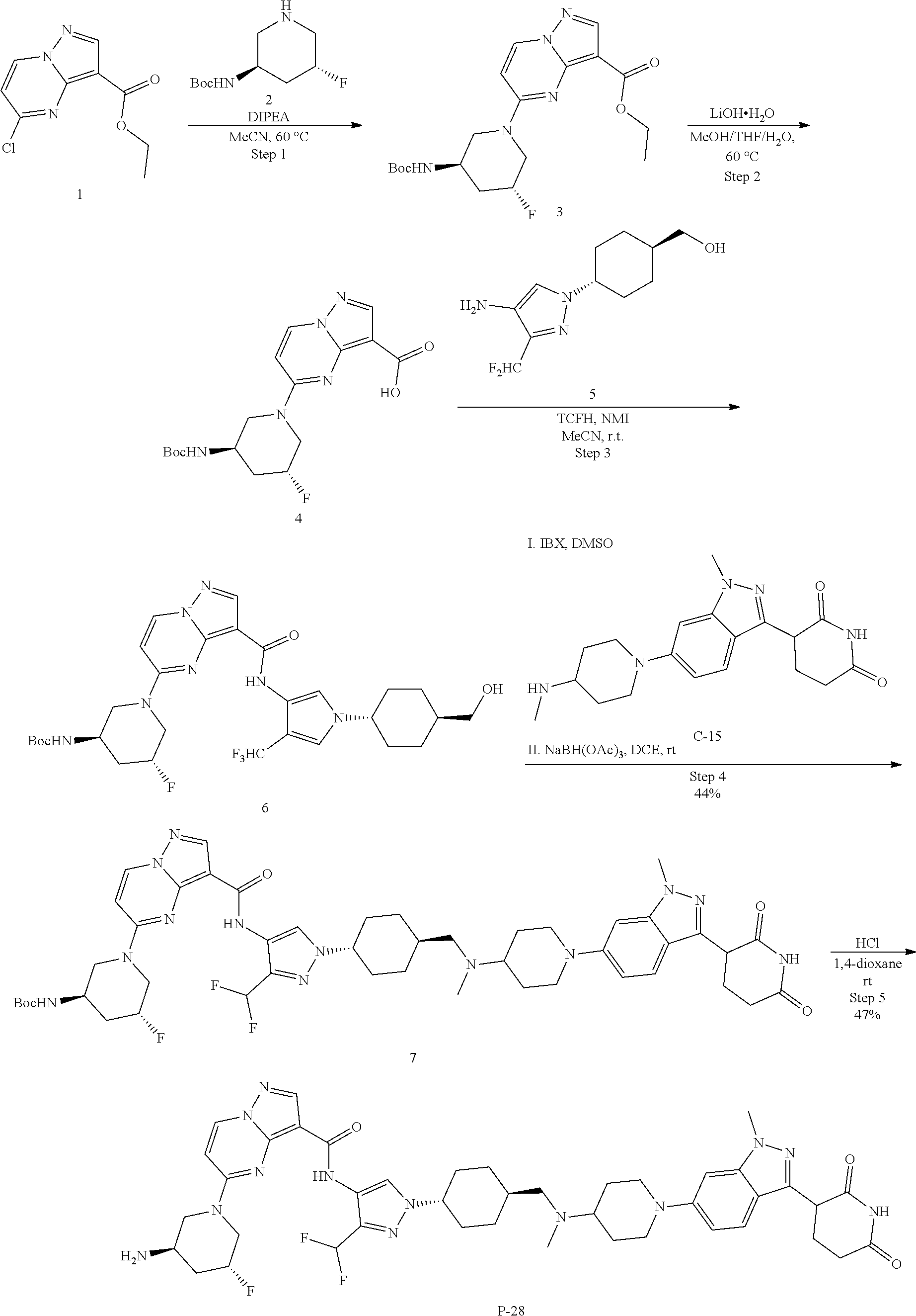

Step 1. Preparation of ethyl 5-[(3R,5R)-3-(tert-butoxycarbonylamino)-5-fluoro-1-piperidyl]pyrazolo[1,5-a]pyrimidine-3-carboxylate (3). To a solution of ethyl 5-chloropyrazolo[1,5-a]pyrimidine-3-carboxylate 1 (800 mg, 3.55 mmol, 1.0 eq.) in MeCN (17.7 mL) was added DIPEA (1.54 mL, 8.86 mmol, 2.5 eq.) and tert-butyl N-[(3R,5R)-5-fluoro-3-piperidyl]carbamate 2 (1.01 g, 4.61 mmol, 1.3 eq.). After stirring at 60° C. over the weekend, LCMS showed complete conversion into 3. The solvent was removed under reduced pressure and the residue was dried under high vacuum to give 3 (1.44 g, quantitative yield) as white solid. The crude product was used in the next step without further purification.

LCMS method 1: retention time: 1.656 min, 99.9% purity at 215 nm, [M+H]⁺=408.2.

¹H NMR (400 MHz, DMSO-d6) δ ppm 1.37-1.45 (m, 9H), 1.56-1.92 (m, 1H), 2.09-2.20 (m, 1H), 2.56-2.71 (m, 1H), 2.76-3.03 (m, 1H), 3.08-3.19 (m, 1H), 3.29 (s, 1H), 3.37 (br s, 1H), 3.56-3.73 (m, 2H), 4.11-4.25 (m, 2H), 4.41-4.73 (m, 1H), 4.99 (br s, 1H), 5.11 (br s, 1H), 6.85 (br d, J=8.1 Hz, 1H), 7.10 (br d, J=7.8 Hz, 1H), 8.22 (s, 1H), 8.73 (d, J=7.8 Hz, 1H).

¹⁹F NMR (377 MHz, DMSO-d6) δ ppm −184.32 (s, 1F).

Step 2. Preparation of 5-[(3R,5R)-3-(tert-Butoxycarbonylamino)-5-fluoro-1-piperidyl]pyrazolo[1,5-a]pyrimidine-3-carboxylic acid (4). To a solution of ethyl 5-[(3R,5R)-3-(tert-butoxycarbonylamino)-5-fluoro-1-piperidyl]pyrazolo[1,5-a]pyrimidine-3-carboxylate 3 (1.44 mg, 3.53 mmol, 1.0 eq) in THF (5.89 mL) and methanol (5.89 mL) was added a solution of LiOH·H₂O (1.48 g, 35.34 mmol, 10.0 eq.) in water (5.89 mL). After stirring at 60° C. for 18 h, LCMS showed complete conversion into desired product 4. The reaction mixture was concentrated under vacuum to remove THF/MeOH and the crude mixture was diluted with water. Under vigorous agitation, the mixture was acidified with a 6N aqueous HCl solution until pH=3 (formation of a precipitate). The suspension was filtered on a Buchner funnel and the solid was rinsed with water. The solid was dried overnight in a stove under vacuum to give 4 (1.40 g, quantitative yield) as white solid. The crude product was used in the next without further purification.

LCMS method 1: retention time: 1.487 min, 99.9% purity at 215 nm, [M+H]⁺=380.1.

¹H NMR (400 MHz, DMSO-d6) δ ppm 1.41 (s, 9H), 1.66-1.95 (m, 1H), 2.05-2.26 (m, 1H), 2.92-3.11 (m, 1H), 3.28-3.30 (m, 1H), 3.34-3.49 (m, 1H), 3.59-3.74 (m, 1H), 4.52-4.75 (m, 1H), 4.99 (br s, 1H), 5.11 (br s, 1H), 6.82 (br d, J=7.6 Hz, 1H), 7.12 (br d, J=7.8 Hz, 1H), 8.19 (s, 1H), 8.73 (d, J=7.8 Hz, 1H).

¹⁹F NMR (377 MHz, DMSO-d6) δ ppm −184.32 (s, 1F).

Step 3. Preparation of tert-Butyl N-[(3r,5r)-1-[3-[[3-(difluoromethyl)-1-[4-(hydroxymethyl)cyclohexyl]pyrazol-4-yl]carbamoyl]pyrazolo[1,5-a]pyrimidin-5-yl]-5-fluoro-3-piperidyl]carbamate (6). To a solution of [4-[4-amino-3-(difluoromethyl)pyrazol-1-yl]cyclohexyl]methanol 5—intermediate 6 from synthesis of P-2 —(511.8 mg, 1.88 mmol, 1.5 eq.), 5-[(3r,5r)-3-(tert-butoxycarbonylamino)-5-fluoro-1-piperidyl]pyrazolo[1,5-a]pyrimidine-3-carboxylic acid 4 (475.0 mg, 1.25 mmol, 1.0 eq.) and NMI (362.72 μL, 4.58 mmol, 3.6 eq.) in MeCN (12.5 mL, 0.1 M) was added TCFH (440.43 mg, 1.57 mmol, 1.2 eq.). The resulting mixture was stirred at room temperature overnight. Water was added and the reaction mixture was stirred at room temperature for 1 hour. The solid was filtered on a Buchner funnel and rinsed with a water/MeCN mixture. Purification by reverse phase chromatography (50 g C18 RediSep Rf Gold column, liquid deposit (DMSO), elution: 5% MeOH/0.1% HCOOH over 4 CV, then 5% to 100% MeOH/0.1% HCOOH over 15 CV). The pure fractions were combined, concentrated under reduced pressure to afford tert-butyl N-[(3r,5r)-1-[3-[[3-(difluoromethyl)-1-[4-(hydroxymethyl)cyclohexyl]pyrazol-4-yl]carbamoyl]pyrazolo[1,5-a]pyrimidin-5-yl]-5-fluoro-3-piperidyl]carbamate 6 (421 mg, 55% yield) as a tan solid.

LCMS method 1: retention time: 1.629 min, 99.9% purity at 215 nm, [M+H]⁺=607.2.

¹H NMR (400 MHz, DMSO-d6) δ ppm 1.03-1.15 (m, 2H), 1.34 (br s, 9H), 1.64-1.80 (m, 3H), 1.82-1.93 (m, 3H), 1.99-2.07 (m, 2H), 2.08-2.18 (m, 1H), 2.95-3.14 (m, 1 H), 3.26 (t, J=5.7 Hz, 2H), 3.37-3.52 (m, 1H), 3.62-3.71 (m, 1H), 4.12-4.21 (m, 1H), 4.47 (t, J=5.3 Hz, 1H), 4.69 (br s, 1H), 4.95-5.12 (m, 1H), 6.86-6.94 (m, 1H), 7.01-7.22 (m, 2H), 8.28 (s, 1H), 8.34 (br s, 1H), 8.82 (d, J=7.8 Hz, 1H), 9.31 (br s, 1H).

¹⁹F NMR (377 MHz, DMSO-d6) δ ppm −186.02-−179.79 (m, 1F), −113.10-−108.69 (m, 2F).

Step 4. Preparation of Tert-butyl N-[(3r,5r)-1-[3-[[3-(difluoromethyl)-1-[4-[[[1-[3-(2,6-dioxo-3-piperidyl)-1-methyl-indazol-6-yl]-4-piperidyl]-methyl-amino]methyl]cyclohexyl]pyrazol-4-yl]carbamoyl]pyrazolo[1,5-a]pyrimidin-5-yl]-5-fluoro-3-piperidyl]carbamate (7). To a solution of tert-butyl tert-butyl N-[rac-(3R,5R)-1-[3-[[3-(difluoromethyl)-1-[4-(hydroxymethyl)cyclohexyl]pyrazol-4-yl]carbamoyl]pyrazolo[1,5-a]pyrimidin-5-yl]-5-fluoro-3-piperidyl]carbamate 6 (86.0 mg, 0.14 mmol, 1.0 eq.) in dry DMSO (1 mL, 0.14 M) was added IBX (47.6 mg, 0.17 mmol, 1.2 eq.). The resulting mixture was stirred at room temperature overnight. LCMS (method 3) after an overnight stirring showed complete conversion to the desired product tert-butyl N-[rac-(3R,5R)-1-[3-[[3-(difluoromethyl)-1-(4-formylcyclohexyl)pyrazol-4-yl]carbamoyl]pyrazolo[1,5-a]pyrimidin-5-yl]-5-fluoro-3-piperidyl]carbamate. To the reaction mixture was added 3-[1-methyl-6-[4-(methylamino)-1-piperidyl]indazol-3-yl]piperidine-2,6-dione hydrochloride C-15 (66.11 mg, 0.17 mmol, 1.2 eq.), DCE (1 mL) and DIPEA (0.24 mL, 1.41 mmol, 10.0 eq.). The mixture was stirred at room temperature for 10 minutes, sodium triacetoxyborohydride (38.74 mg, 0.18 mmol, 1.3 eq.) was added, and the mixture was stirred at r.t. for overnight. The reaction mixture was concentrated in vacuo. Purification was performed by reverse phase chromatography (C18 RediSep Rf Gold 50 g, 5 to 40% MeCN/0.1% formic acid in water, 20 CV). Pure fractions were combined and concentrated to give tert-butyl N-[rac-(3R,5R)-1-[3-[[3-(difluoromethyl)-1-[4-[[[1-[3-(2,6-dioxo-3-piperidyl)-1-methyl-indazol-6-yl]-4-piperidyl]-methyl-amino]methyl]cyclohexyl]pyrazol-4-yl]carbamoyl]pyrazolo[1,5-a]pyrimidin-5-yl]-5-fluoro-3-piperidyl]carbamate 7 (66 mg, 44% yield) as a tan solid. The desired product contained IBX residue.

LCMS method 3: retention time: 1.736 min, 89.3% purity at 215 nm, [M+2H]²⁺=472.8

¹H NMR (400 MHz, DMSO-d6) δ ppm 1.00-1.17 (m, 3H), 1.27-1.41 (m, 8H), 1.48-1.64 (m, 3H), 1.67-1.83 (m, 4H), 1.86-1.97 (m, 2H), 1.99-2.07 (m, 2H), 2.12-2.18 (m, 1H), 2.23-2.34 (m, 5H), 2.59-2.67 (m, 2H), 2.70-2.78 (m, 4H), 2.89 (s, 3H), 3.63-3.72 (m, 1H), 3.82-3.91 (m, 5H), 4.15-4.28 (m, 2H), 4.94-5.01 (m, 1H), 5.09 (br s, 1H), 6.78-6.96 (m, 3H), 7.03-7.20 (m, 2H), 7.48 (d, J=9.0 Hz, 1H), 7.96 (s, 1H), 8.29 (s, 1H), 8.34 (br s, 1H), 8.82 (d, J=7.8 Hz, 1H), 9.31 (br s, 1H), 10.85 (s, 1H).

Step 7. Preparation of N-[3-(Difluoromethyl)-1-[4-[[[1-[3-(2,6-dioxo-3-piperidyl)-1-methyl-indazol-6-yl]-4-piperidyl]-methyl-amino]methyl]cyclohexyl]pyrazol-4-yl]-5-[(3r,5r)-3-amino-5-fluoro-1-piperidyl]pyrazolo[1,5-a]pyrimidine-3-carboxamide (P-28). HCl 4.0 M in dioxane (2.62 mL, 10.49 mmol, 150.0 eq.) was added to tert-butyl N-[rac-(3R,5R)-1-[3-[[3-(difluoromethyl)-1-[4-[[[1-[3-(2,6-dioxo-3-piperidyl)-1-methyl-indazol-6-yl]-4-piperidyl]-methyl-amino]methyl]cyclohexyl]pyrazol-4-yl]carbamoyl]pyrazolo[1,5-a]pyrimidin-5-yl]-5-fluoro-3-piperidyl]carbamate 7 (66. mg, 0.07 mmol, 1.0 eq.). The reaction mixture was stirred for 2 h at room temperature. The reaction mixture was concentrated in vacuo. Purification was performed by reverse phase chromatography (50 g C18 RediSep Rf Gold column, liquid deposit (H₂O), elution: 5% MeOH/0.02 M HCl over 4 CV, then 5% to 50% MeCN/0.02 M HCl over 15 CV). The pure fractions were combined, concentrated under reduced pressure and lyophilised overnight to afford N-[3-(difluoromethyl)-1-[4-[[[1-[3-(2,6-dioxo-3-piperidyl)-1-methyl-indazol-6-yl]-4-piperidyl]-methyl-amino]methyl]cyclohexyl]pyrazol-4-yl]-5-[rac-(3R,5R)-3-amino-5-fluoro-1-piperidyl]pyrazolo[1,5-a]pyrimidine-3-carboxamide P-28 (28 mg, 47% yield).

LCMS method 2: retention time: 1.911 min, 99.6% purity at 215 nm, [M−HCl+H]⁺=844; [M-2HCl+2H]²⁺=422.6.

¹H NMR (400 MHz, DMSO-d6) δ ppm 1.13-1.31 (m, 2H), 1.75-1.96 (m, 6H), 2.03-2.20 (m, 6H), 2.26-2.34 (m, 1H), 2.37-2.44 (m, 1H), 2.56-2.69 (m, 2H), 2.75-2.96 (m, 6H), 3.10-3.18 (m, 1H), 3.28-3.51 (m, 5H), 3.88-3.93 (m, 3H), 3.98 (br d, J=12.2 Hz, 2H), 4.19-4.30 (m, 2H), 4.59 (br s, 1H), 4.78-4.95 (m, 1H), 5.01-5.18 (m, 1H), 6.87-6.98 (m, 3H), 7.00-7.28 (m, 1H), 7.53 (d, J=8.8 Hz, 1H), 8.27 (br d, J=4.2 Hz, 2H), 8.33 (s, 1H), 8.41 (s, 1H), 8.94 (d, J=7.8 Hz, 1H), 9.34 (s, 1H), 9.52 (br s, 1H), 10.86 (s, 1H).

¹⁹F NMR (377 MHz, DMSO-d6) δ ppm −183.67 (s, 2F), −111.29 (br s, 1F).

### Example S11. Synthesis of P-29

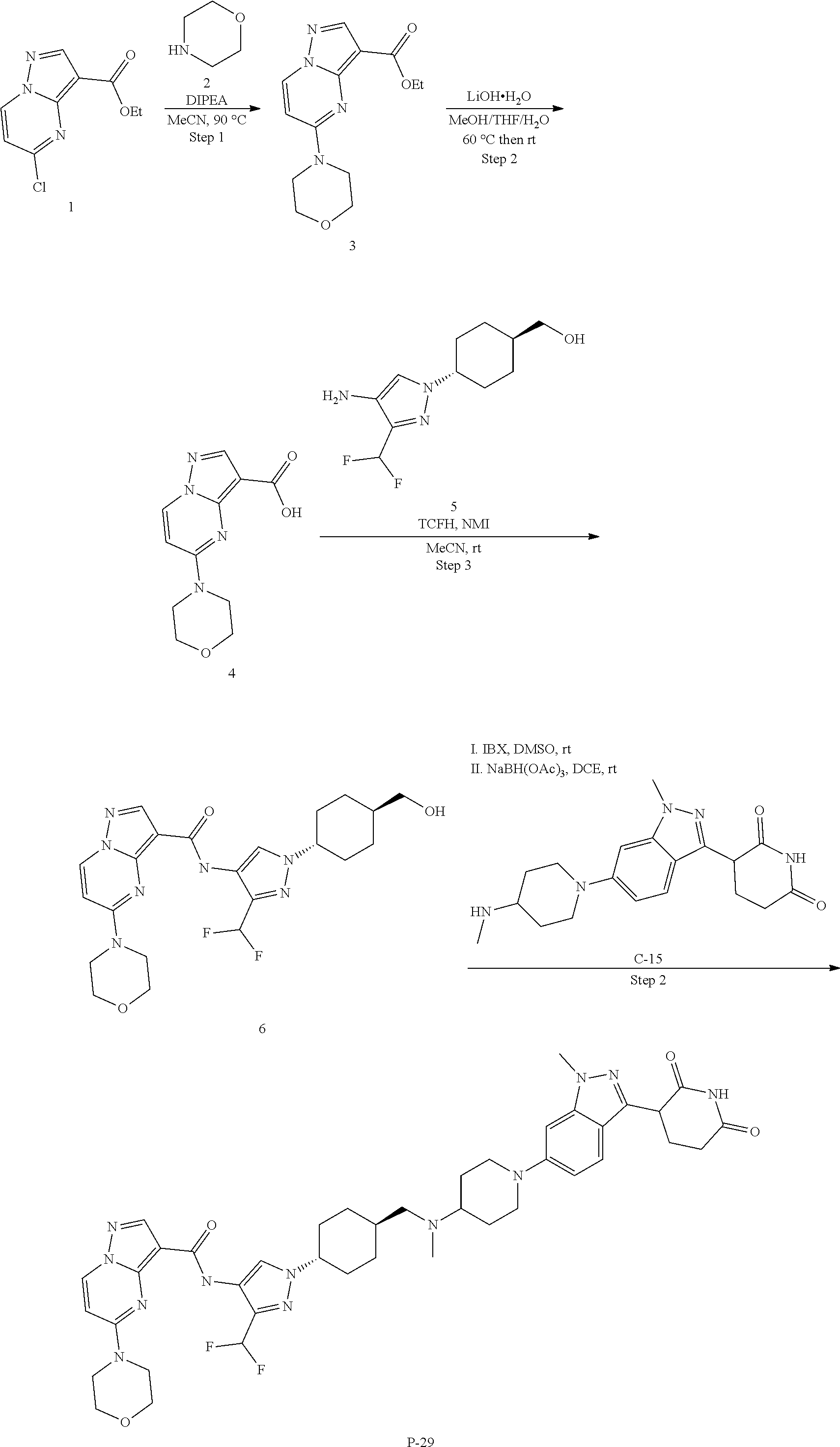

Step 1. Preparation of Ethyl 5-morpholinopyrazolo[1,5-a]pyrimidine-3-carboxylate (3). To a sealed tube were introduced ethyl 5-chloropyrazolo[1,5-a]pyrimidine-3-carboxylate 1 (1.5 g, 6.65 mmol, 1 eq.), MeCN (33.24 mL, 0.2 M) and DIPEA (2.89 mL, 16.62 mmol, 2.5 eq.), followed by morpholine 2 (639.65 μL, 7.31 mmol, 1.1 eq.). The tube was sealed and the mixture was stirred at 90° C. for 16 h. The solvents were evaporated under reduced pressure and dried under high vacuum to give ethyl 5-morpholinopyrazolo[1,5-a]pyrimidine-3-carboxylate 3 (2.96 g, 99% yield) as an orange solid.

LCMS method 1: retention time: 1.432 min, 99% purity at 215 nm, [M+H]⁺=277.2.

¹H NMR (400 MHz, chloroform-d) δ ppm 1.39 (t, J=7.1 Hz, 3H), 3.76-3.86 (m, 8H), 4.35 (q, J=7.1 Hz, 2H), 6.41 (d, J=8.1 Hz, 1H), 8.29-8.34 (m, 2H).

Step 2. Preparation of 5-Morpholinopyrazolo[1,5-a]pyrimidine-3-carboxylic acid (4). To a solution of ethyl 5-morpholinopyrazolo[1,5-a]pyrimidine-3-carboxylate 3 (1.84 g, 6.66 mmol, 1.0 eq.) in THF (17.8 mL, 0.1 M) and methanol (17.8 mL), after 5 minutes of stirring, was added a solution of LiOH H₂O (2.8 g, 66.6 mmol, 10 eq.) in water (17.8 mL). The mixture was stirred at 60° C. for 3 h, then the oil bath was removed and the mixture was allowed to stir over the weekend (72 h). The reaction mixture was concentrated under reduced pressure, then the crude mixture was diluted with a small quantity of water and acidified to pH 3 with 6 N HCl. The resulting precipitate was filtered and rinsed with water. The solid was dried under high vacuum to give 5-morpholinopyrazolo[1,5-a]pyrimidine-3-carboxylic acid 4 (1.65 g, 99% yield) as a tan solid.

LCMS method 1: retention time: 1.192 min, 99% purity at 215 nm, [M+H]⁺ 249.2.

¹H NMR (400 MHz, DMSO-d6) δ ppm 3.72 (br d, J=5.9 Hz, 8H), 6.84 (d, J=8.1 Hz, 1H), 8.19 (s, 1H), 8.74 (d, J=7.8 Hz, 1H), 11.73 (s, 1H).

Step 3. Preparation of N-[3-(Difluoromethyl)-1-[4-(hydroxymethyl)cyclohexyl]pyrazol-4-yl]-5-morpholino-pyrazolo[1,5-a]pyrimidine-3-carboxamide (6). To a solution of [4-[4-amino-3-(difluoromethyl)pyrazol-1-yl]cyclohexyl]methanol 5—intermediate 6 from synthesis of P-2 —(403.12 mg, 1.64 mmol, 1.2 eq.), 5-morpholinopyrazolo[1,5-a]pyrimidine-3-carboxylic acid 4 (340.0 mg, 1.37 mmol, 1.0 eq.) and NMI (0.43 mL, 5.48 mmol, 4.0 eq.) in MeCN (9 mL, 0.15 M) was added TCFH (480.37 mg, 1.71 mmol, 1.25 eq.). The resulting mixture was stirred at room temperature for 1 h. After 1 h, the volatiles were evaporated and the resulting mixture was purification by reverse phase chromatography (50 g C18 RediSep Rf Gold column, liquid deposit (DMSO), elution: 5% MeOH/0.1% HCOOH over 4 CV, then 5% to 50% MeOH/0.1% HCOOH over 15 CV, product exited at 50% MeOH). The pure fractions were combined, concentrated under reduced pressure to afford N-[3-(Difluoromethyl)-1-[4-(hydroxymethyl)cyclohexyl]pyrazol-4-yl]-5-morpholino-pyrazolo[1,5-a]pyrimidine-3-carboxamide 6 (338 mg, 52% yield) as a tan solid.

LCMS method 3: retention time: 1.707 min, 99.9% purity at 215 nm, [M+H]⁺=476.2.

¹H NMR (400 MHz, DMSO-d6) δ ppm 1.02-1.15 (m, 2H), 1.38-1.48 (m, 1H), 1.66-1.79 (m, 2H), 1.80-1.90 (m, 2H), 1.99-2.08 (m, 2H), 3.26 (t, J=5.7 Hz, 2H), 3.68-3.74 (m, 4H), 3.76-3.83 (m, 4H), 4.12-4.23 (m, 1H), 4.47 (t, J=5.4 Hz, 1H), 6.90 (d, J=7.8 Hz, 1H), 6.95-7.25 (m, 1H), 8.28 (s, 1H), 8.38 (s, 1H), 8.82 (d, J=8.1 Hz, 1H), 9.39 (s, 1H).

¹⁹F NMR (377 MHz, DMSO-d6) δ ppm −111.19 (s, 2F).

Step 4. Preparation of N-[3-(Difluoromethyl)-1-[4-[[[1-[3-(2,6-dioxo-3-piperidyl)-1-methyl-indazol-6-yl]-4-piperidyl]-methyl-amino]methyl]cyclohexyl]pyrazol-4-yl]-5-morpholino-pyrazolo[1,5-a]pyrimidine-3-carboxamide (P-29). To a solution of N-[3-(difluoromethyl)-1-[4-(hydroxymethyl)cyclohexyl]pyrazol-4-yl]-5-morpholino-pyrazolo[1,5-a]pyrimidine-3-carboxamide 6 (100.0 mg, 0.21 mmol, 1.0 eq.) in dry DMSO (2 mL, 0.1 M) was added IBX (70.0 mg, 0.25 mmol, 1.2 eq.). The resulting mixture was stirred at room temperature overnight. LCMS (method 3) after an overnight stirring showed complete conversion to the desired product N-[3-(difluoromethyl)-1-(4-formylcyclohexyl)pyrazol-4-yl]-5-morpholino-pyrazolo[1,5-a]pyrimidine-3-carboxamide. To the reaction mixture was added [1-[3-(2,6-dioxo-3-piperidyl)-1-methyl-indazol-6-yl]-4-piperidyl]-methyl-ammonium chloride C-15 (98.91 mg, 0.25 mmol, 1.2 eq.), DCE (2.1 mL, 0.05 M) and DIPEA (0.37 mL, 2.10 mmol, 10 eq.). The mixture was stirred at rt for 10 minutes. Sodium triacetoxyborohydride (133.72 mg, 0.63 mmol, 3.0 eq.) was added and the mixture was stirred at room temperature for 3 h. The reaction mixture was concentrated in vacuo. Purification was performed by reverse phase chromatography (50 g C18 RediSep Rf Gold column, liquid deposit (DMSO), elution: 5% MeCN/0.1% HCOOH over 4 CV, then 5% to 50% MeCN/0.1% HCOOH over 15 CV). The pure fractions were combined, concentrated under reduced pressure and lyophilised overnight to afford N-[3-(difluoromethyl)-1-[4-[[[1-[3-(2,6-dioxo-3-piperidyl)-1-methyl-indazol-6-yl]-4-piperidyl]-methyl-amino]methyl]cyclohexyl]pyrazol-4-yl]-5-morpholino-pyrazolo[1,5-a]pyrimidine-3-carboxamide P-29 (25.4 mg, 14% yield) as a yellow solid.

LCMS method 4: retention time: 2.399 min, 96.0% purity at 215 nm, [M+H]⁺=813.4; [M+2H]²⁺=407.3.

¹H NMR (400 MHz, DMSO-d6) δ ppm 0.94-1.16 (m, 3H), 1.49-1.64 (m, 3H), 1.69-1.81 (m, 4H), 1.86-1.96 (m, 2H), 2.01-2.09 (m, 2H), 2.12-2.20 (m, 1H), 2.22 (s, 3H), 2.25 (br d, J=6.8 Hz, 2H), 2.55-2.65 (m, 3H), 2.70-2.78 (m, 2H), 3.70-3.75 (m, 4H), 3.78-3.86 (m, 6H), 3.89 (s, 3H), 4.13-4.21 (m, 1H), 4.25 (dd, J=8.9, 5.0 Hz, 1H), 6.84 (d, J=1.5 Hz, 1H), 6.91 (d, J=8.1 Hz, 2H), 7.10 (s, 1H), 7.48 (d, J=9.0 Hz, 1H), 8.29 (s, 1H), 8.38 (s, 1H), 8.83 (d, J=7.8 Hz, 1H), 9.40 (s, 1H), 10.85 (s, 1H).

¹⁹F NMR (377 MHz, DMSO-d6) δ ppm −111.19 (s, 2F).

The following compounds were synthesized via the same general routes with modifications to amine 2 in step 1 (Table 7).

TABLE 7

| Com- | | | |
|---|---|---|---|
| pound | | | |
| No. | Amine | Structure | Characterization Data |
| P-28 | 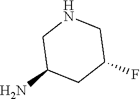 | 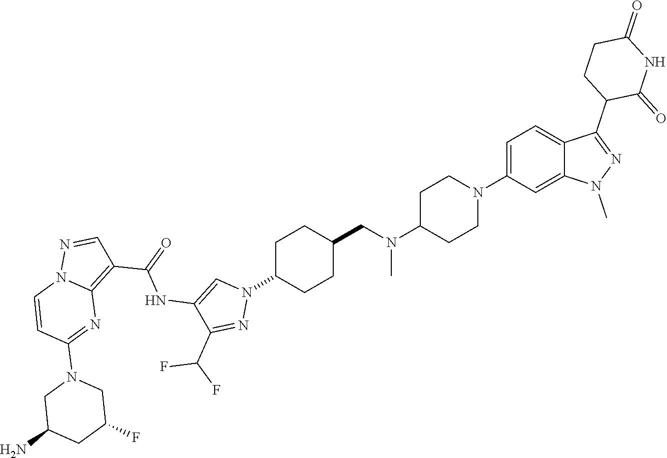 | 47% yield as a tan solid. LCMS method 2: retention time: 1.911 min, 99% purity at 215 nm, [M − HCl + H]⁺ = 844; [M − 2HCl + 2H]²⁺ = 422.6. ¹H NMR (400 MHz, DMSO-d6) δ ppm 1.13-1.31 (m, 2 H), 1.75-1.96 (m, 6 H), 2.03-2.20 (m, 6 H), 2.26-2.34 (m, 1 H), 2.37-2.44 (m, 1 H), 2.56-2.69 (m, 2 H), 2.75- 2.96 (m, 6 H), 3.10-3.18 (m, 1 H), 3.28-3.51 (m, 5 H), 3.88-3.93 (m, 3 H), 3.98 (br d, J = 12.2 Hz, 2 H), 4.19- 4.30 (m, 2 H), 4.59 (br s, 1 H), 4.78-4.95 (m, 1 H), 5.01- 5.18 (m, 1 H), 6.87-6.98 (m, 3 H), 7.00-7.28 (m, 1 H), 7.53 (d, J = 8.8 Hz, 1 H), 8.27 (br d, J = 4.2 Hz, 2 H), 8.33 (s, 1 H), 8.41 (s, 1 H), 8.94 (d, J = 7.8 Hz, 1 H), 9.34 (s, 1 H), 9.52 (br s, 1 H), 10.86 (s, 1 H). ¹⁹F NMR (377 MHz, DMSO-d6) δ ppm −183.67 (s, 2 F), −111.29 (br s, 1 F). |
| P-29 | 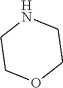 | 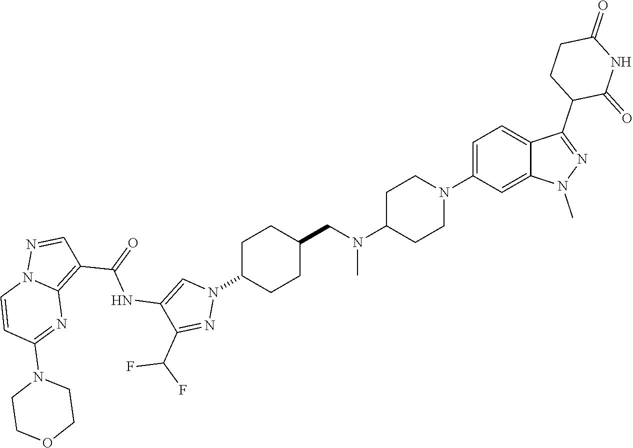 | 14% yield as a yellow solid. LCMS method 4: retention time: 2.399 min, 96% purity at 215 nm, [M + H]⁺ = 813.4; [M + 2H]²⁺ = 407.3. ¹H NMR (400 MHz, DMSO-d6) δ ppm 0.94-1.16 (m, 3 H), 1.49-1.64 (m, 3 H), 1.69-1.81 (m, 4 H), 1.86-1.96 (m, 2 H), 2.01-2.09 (m, 2 H), 2.12-2.20 (m, 1 H), 2.22 (s, 3 H), 2.25 (br d, J = 6.8 Hz, 2 H), 2.55-2.65 (m, 3 H), 2.70-2.78 (m, 2 H), 3.70-3.75 (m, 4 H), 3.78-3.86 (m, 6 H), 3.89 (s, 3 H), 4.13-4.21 (m, 1 H), 4.25 (dd, J = 8.9, 5.0 Hz, 1 H), 6.84 (d, J = 1.5 Hz, 1 H), 6.91 (d, J = 8.1 Hz, 2 H), 7.10 (s, 1 H), 7.48 (d, J = 9.0 Hz, 1 H), 8.29 (s, 1 H), 8.38 (s, 1 H), 8.83 (d, J = 7.8 Hz, 1 H), 9.40 (s, 1 H), 10.85 (s, 1 H). ¹⁹F NMR (377 MHz, DMSO-d6) δ ppm −111.19 (s, 2 F). |
| P-41 | 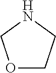 | 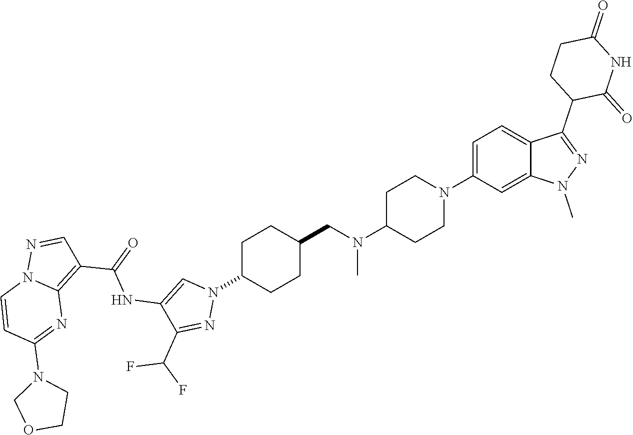 | 30% yield as an off-white solid. LCMS method 6: retention time: 1.543 min, 91% purity at 215 nm, [M + H]⁺ = 799.0. ¹H NMR (400 MHz, DMSO-d6): δ ppm 1.00-1.08 (m, 2H), 1.52-1.62 (m, 3H), 1.71-1.79 (m, 4H), 1.90-1.93 (m, 2H), 2.03-2.06 (m, 2H), 2.15-2.29 (m, 6H), 2.60-2.63 (m, 2H), 2.67-2.71 (m, 2H), 3.70 (t, J = 5.2 Hz, 2H), 3.83-3.86 (m, 2H), 3.89 (s, 3H), 4.18-4.27 (m, 4H), 5.12 (s, 2H), 6.60 (brs, 1H), 6.84 (s, 1H), 6.91 (dd, J = 1.6 Hz and 9.2 Hz, 1H), 7.15 (t, J = 53.2 Hz, 1H), 7.48 (d, J = 8.80 Hz, 1H), 8.26 (s, 1H), 8.30 (s, 1H), 8.40 (s, 1H), 8.87 (d, J = 7.60 Hz, 1H), 10.87 (s, 1H), -Two protons are missing by ¹H NMR. |
| P-42 | 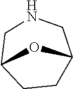 | 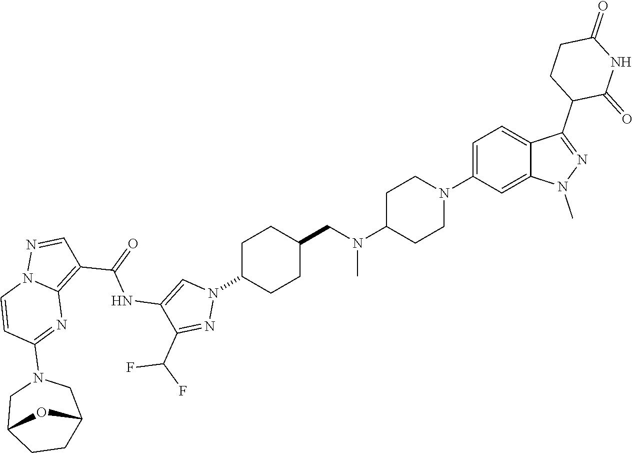 | 48% yield as an off-white solid. LCMS method 6: retention time: 1.360 min, 97% purity at 215 nm, [M + H]⁺ = 839.3. ¹H NMR (400 MHz, DMSO-d6): δ ppm 1.02-1.11 (m, 2H), 1.54-1.61 (m, 3H), 1.72-1.79 (m, 6H), 1.86-1.94 (m, 4H), 2.03-2.06 (m, 2H), 2.15-2.29 (m, 6H), 2.61-2.77 (m, 4H), 3.83-3.86 (m, 2H), 3.89 (s, 3H), 4.14-4.27 (m, 2H), 4.45-4.47 (m, 2H), 6.82-6.84 (m, 2H), 6.91 (d, J = 9.20 Hz, 1H), 7.11 (t, J = 53.6 Hz, 1H), 7.49 (d, J = 8.80 Hz, 1H), 8.29 (d, J = 4.80 Hz, 1H), 8.39 (s, 1H), 8.83 (d, J = 8.00 Hz, 1H), 9.42 (s, 1H), 10.87 (s, 1H). -Six protons are missing by ¹H NMR. |
| P-43 | 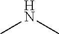 | 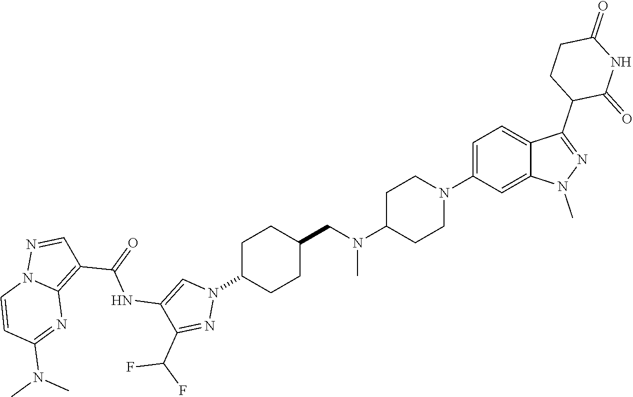 | 32% yield as an off-white solid. LCMS method 7: retention time: 1.719 min, 93% purity at 215 nm, (M + H)⁺ = 771.4. ¹H NMR (400 MHz, DMSO-d6): δ 1.15-1.19 (m, 2H), 1.68-1.80 (m, 6H), 1.92-1.99 (m, 4H), 2.06-2.09 (m, 2H), 2.17-2.29 (m, 2H), 2.61-2.64 (m, 2H), 2.76-2.81 (m, 2H), 3.28 (s, 6H), 3.90 (s, 3H), 3.88-3.94 (m, 2H), 4.19-4.28 (m, 2H), 6.76 (d, J = 8.0 Hz, 1H), 6.88 (s, 1H), 6.93 (d, J = 8.8 Hz, 1H), 7.10 (t, J = 53.6 Hz, 1H), 7.51 (d, J = 8.8 Hz, 1H), 8.25 (s, 1H), 8.40 (s, 1H), 8.76 (d, J = 8.0 Hz, 1H), 9.49 (s, 1H), 10.85 (s, 1H). -Five protons are missing by ¹H NMR. |
| P-44 | 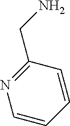 | 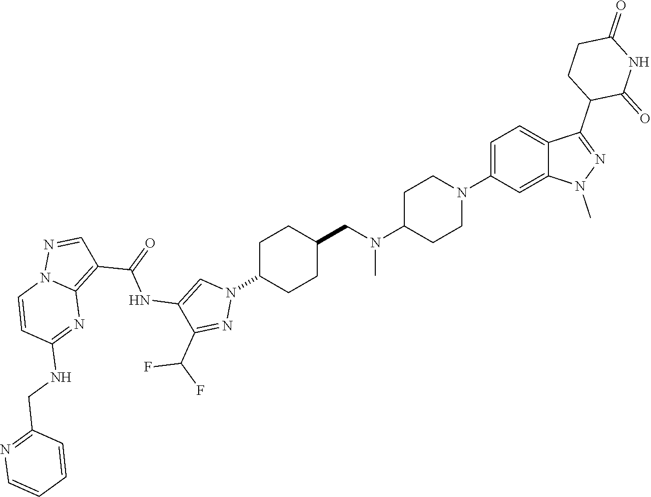 | 38% yield as an off-white solid. LCMS method 6: retention time: 1.095 min, 96% purity at 215 nm, (M + H)+ = 834.3. ¹H NMR (400 MHz, DMSO-d6): : δ 0.98-1.12 (m, 2H), 1.51-1.62 (m, 3H), 1.73-1.79 (m, 4H), 1.91-1.94 (m, 2H), 2.04-2.06 (m, 2H), 2.13-2.37 (m, 7H), 2.60-2.62 (m, 2H), 2.71-2.74 (m, 2H), 3.83-3.89 (m, 3H), 3.89 (s, 3H), 4.18- 4.27 (m, 2H), 4.80 (d, J = 5.2 Hz, 2H), 6.64 (d, J = 7.6 Hz, 1H), 6.84 (s, 1H), 6.91 (d, J = 8.8 Hz, 1H), 7.08 (t, J = 52.0 Hz, 1H), 7.32 (dd, J = 5.2 Hz and 6.8 Hz, 1H), 7.40 (d, J = 8.0 Hz, 1H), 7.48 (d, J = 9.2 Hz, 1H), 7.77-7.81 (m, 1H), 8.23 (s, 1H), 8.34 (s, 1H), 8.55 (d, J = 4.0 Hz, 1H), 8.64-8.66 (m, 2H), 9.56 (s, 1H), 10.9 (s, 1H). |
| P-45 | 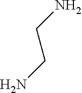 | 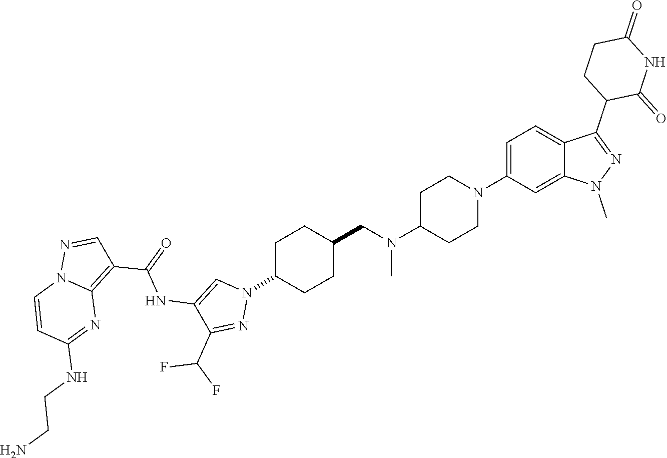 | 43% yield as an off-white solid. LCMS method 6: retention time: 2.372 min, 93% purity at 215 nm, [M + H]⁺ = 786.2. ¹H NMR (400 MHz, DMSO-d6): δ ppm 1.02-1.09 (m, 2H), 1.52-1.61 (m, 3H), 1.71-1.80 (m, 4H), 1.91-1.94 (m, 2H), 2.04-2.07 (m, 2H), 2.15-2.34 (m, 7H), 2.60-2.63 (m, 1H), 2.71-2.77 (m, 2H), 2.97 (m, 2H), 3.64 (m, 2H), 3.83- 3.86 (m, 2H), 3.90 (s, 3H), 4.16-4.27 (m, 2H), 6.48 (d, J = 7.60 Hz, 1H), 6.84 (d, J = 1.2 Hz, 1H), 6.91 (dd, J = 1.60 Hz and 8.8 Hz, 1H), 7.12 (t, J = 54 Hz, 1H), 7.49 (d, J = 9.20 Hz, 1H), 8.22 (s, 1H), 8.35 (brs, 2H), 8.37 (s, 1H), 8.61 (d, J = 7.60 Hz, 1H), 9.06 (s, 1H), 9.52 (s, 1H). -Three protons are missing by ¹H NMR. |
| P-46 | 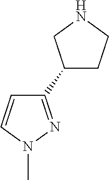 | 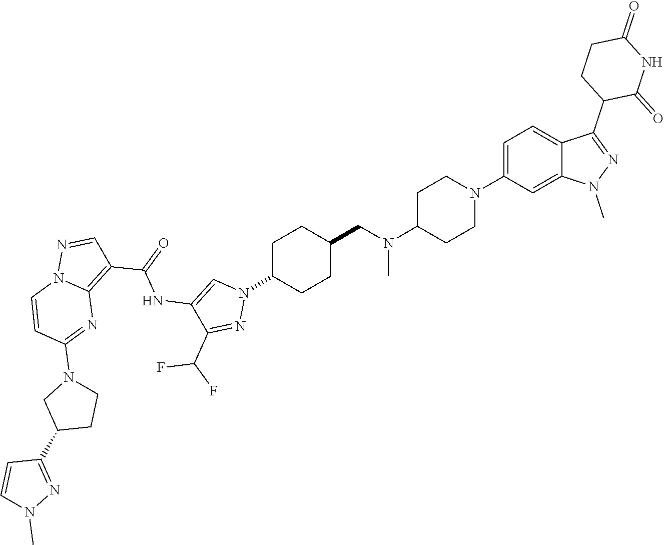 [SMILES: CN(C[C@H]1CC[C@H](n2cc(NC(=O)c3cnn4ccc(N5CC[C@H](c6ccn(C)n6)C5)nc34)c(C(F)F)n2)CC1)C1CCN(c2ccc3c(C4CCC(=O)NC4=O)nn(C)c3c2)CC1] | 8% yield as an off-white solid. LCMS method 6: retention time: 1.410 min, 90% purity at 215 nm, (M + H)+ = 877.4. ¹H NMR (400 MHz, DMSO-d6): δ 0.98-1.19 (m, 2H), 1.53-1.59 (m, 3H), 1.73-1.79 (m, 4H), 1.91-1.93 (m, 2H), 2.03-2.06 (m, 2H), 2.11-2.29 (m, 6H), 2.55-2.61 (m, 2H), 2.69-2.74 (m, 2H), 3.63-3.70 (m, 3H), 3.79 (s, 3H), 3.83-3.86 (m, 2H), 3.89 (s, 3H), 3.91-4.05 (m, 1H), 4.15- 4.26 (m, 2H), 6.13 & 6.19 (s, 1H), 6.60 (dd, J = 8.0 Hz and 15.2 Hz, 1H), 6.84 (s, 1H), 6.91 (d, J = 8.8 Hz, 1H), 6.96- 7.21 (m, 1H), 7.48 (d, J = 8.8 Hz, 1H), 7.60 (d, J = 6.4 Hz, 1H), 8.25 (s, 1H), 8.38 (d, J = 8.8 Hz, 1H), 8.74-8.78 (m, 1H), 9.55 (d, J = 8.0 Hz, 1H), 10.84 (s, 1H). -Five protons are missing by ¹H NMR. |
| P-47 | 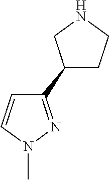 | 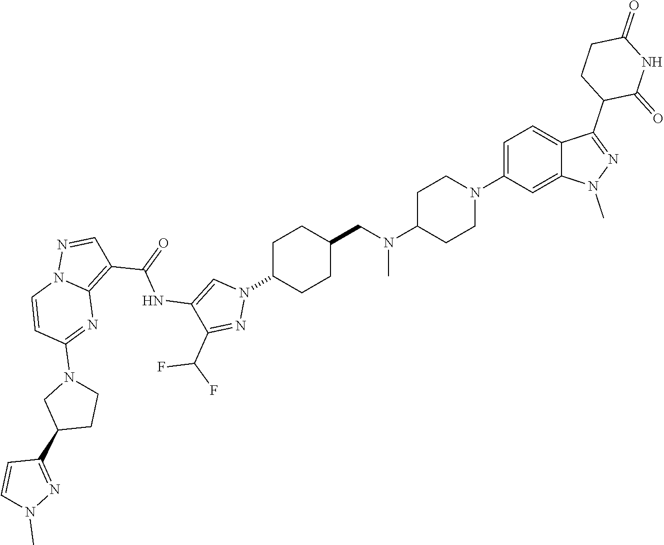 [SMILES: CN(C[C@H]1CC[C@H](n2cc(NC(=O)c3cnn4ccc(N5CC[C@@H](c6ccn(C)n6)C5)nc34)c(C(F)F)n2)CC1)C1CCN(c2ccc3c(C4CCC(=O)NC4=O)nn(C)c3c2)CC1] | 69% yield as a pink solid. LCMS method 6: retention time: 1.374 min, 93% purity at 215 nm, (M + H)+ = 877.2. ¹H NMR (400 MHz, DMSO-d6): δ 0.98-1.09 (m, 2H), 1.51-1.58 (m, 3H), 1.71-1.79 (m, 4H), 1.90-1.93 (m, 2H), 2.03-2.06 (m, 2H), 2.14-2.29 (m, 8H), 2.59-2.61 (m, 2H), 2.62-2.74 (m, 2H), 3.51-3.62 (m, 2H), 3.64-3.78 (m, 2H), 3.79 (s, 3H), 3.83-3.88 (m, 3H), 3.89 (s, 3H), 3.95- 4.06 (m, 1H), 4.15-4.27 (m, 2H), 6.14 & 6.19 (s, 1H), 6.57-6.63 (m, 1H), 6.85 (s, 1H), 6.90-6.92 (m, 1H), 6.97- 7.23 (m, 1H), 7.49 (d, J = 8.0 Hz, 1H), 7.60-7.62 (m, 1H), 8.25 (s, 1H), 8.38 (d, J = 8.4 Hz, 1H), 8.74-8.78 (m, 1H), 9.55 (d, J = 7.2 Hz, 1H), 10.86 (s, 1H). |
| P-48 | 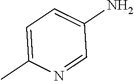 | 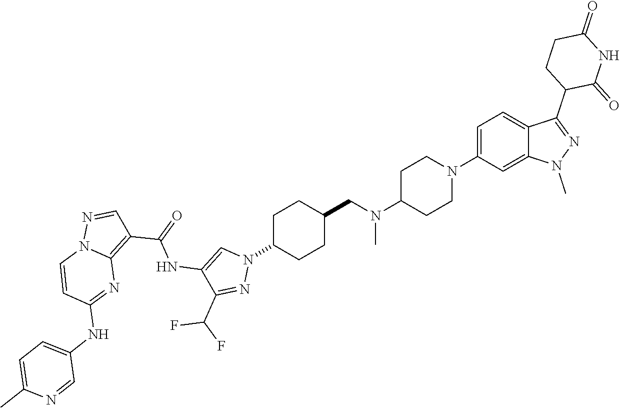 | 1.6% yield as an off-white solid. LCMS method 6: retention time: 3.160 min, 91% purity at 215 nm, [M + H]⁺ = 834.2. ¹H NMR (400 MHz, DMSO-d6): δ ppm 1.09-1.18 (m, 2H), 1.64-1.96 (m, 7H), 2.08-2.18 (m, 4H), 2.25-2.33 (m, 1H), 2.45 (s, 3H), 2.58-2.63 (m, 2H), 2.72-2.82 (m, 2H), 3.40-3.50 (m, 4H), 3.90 (m, 5H), 4.21-4.28 (m, 2H), 4.58 (t, J = 5.2 Hz, 1H), 6.66 (d, J = 7.6 Hz, 1H), 6.87-6.94 (m, 2H), 6.91 (t, J = 54.8 Hz, 1H), 7.23 (d, J = 8.0 Hz, 1H), 7.50 (d, J = 8.8 Hz, 1H), 7.95 (dd, J = 2.8 Hz and 8.4 Hz, 1H), 8.15 (d, J = 4.0 Hz, 1H), 8.33 (s, 1H), 8.75 (d, J = 2.4 Hz, 1H), 8.84 (d, J = 7.6 Hz, 1H), 9.22 (s, 1H), 10.19 (s, 1H), 10.89 (s, 1H). -Two protons are missing by ¹H NMR. |
| P-49 | 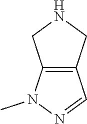 | 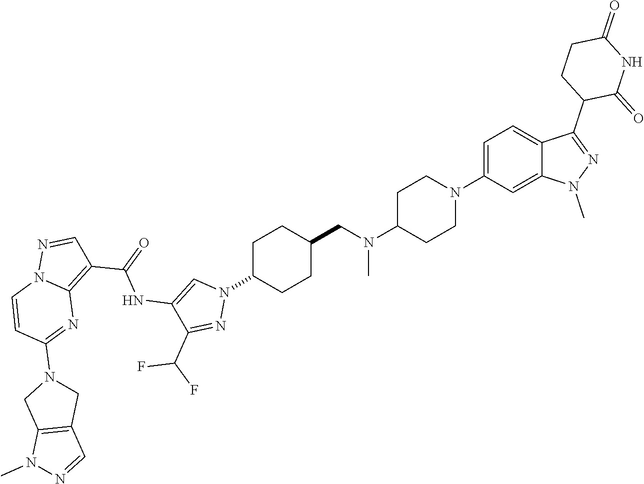 | 18% yield as an off-white solid. LCMS method 6: retention time: 1.244 min, 91% purity at 215 nm, (M + H)+ = 849.2. ¹H NMR (400 MHz, DMSO-d6, 80 °C): δ 1.02-1.15 (m, 2H), 1.59-1.63 (m, 3H), 1.76-1.84 (m, 4H), 1.95-1.98 (m, 2H), 2.10-2.13 (m, 2H), 2.22-2.39 (m, 8H), 2.64-2.68 (m, 2H), 2.77-2.83 (m, 2H), 3.84-3.89 (m, 8H), 4.17-4.27 (m, 2H), 4.73 (s, 2H), 4.87 (s, 2H), 6.63 (d, J = 6.8 Hz, 1H), 6.82 (s, 1H), 6.89-6.92 (m, 1H), 7.15 (t, J = 53.6 Hz, 1H), 7.32 (s, 1H), 7.49 (d, J = 8.8 Hz, 1H), 8.19 (s, 1H), 8.38 (s, 1H), 8.80 (d, J = 7.6 Hz, 1H), 9.51 (s, 1H), 10.54 (s, 1H). |
| P-50 | 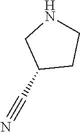 | 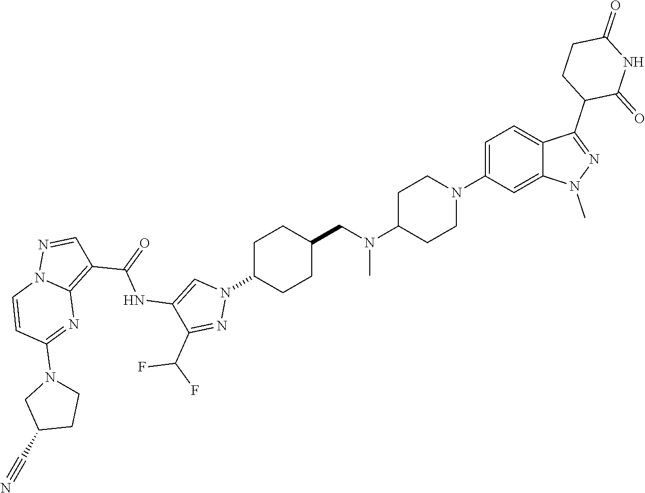 | 26% yield as an off-white solid. LCMS method 6: retention time: 1.360 min, 93% purity at 215 nm, (M + H)+ = 822.4. ¹H NMR (400 MHz, DMSO-d6): δ 1.04-1.16 (m, 2H), 1.56-1.61 (m, 3H), 1.71-1.83 (m, 4H), 1.91-1.94 (m, 2H), 2.04-2.08 (m, 2H), 2.15-2.19 (m, 1H), 2.30-2.34 (m, 8H), 2.60-2.66 (m, 2H), 2.72-2.78 (m, 2H), 3.68-3.78 (m, 4H), 3.83-3.89 (m, 6H), 3.91-4.01 (m, 1H), 4.16-4.27 (m, 2H), 6.63 (d, J = 8.0 Hz, 1H), 6.85 (s, 1H), 6.90-6.93 (m, 1H), 7.13 (t, J = 54.0 Hz, 1H), 7.49 (d, J = 8.8 Hz 1H), 8.29 (s, 1H), 8.39 (s, 1H), 8.83 (d, J = 8.0 Hz, 1H), 9.51 (s, 1H), 10.86 (s, 1H). |

### Final Product General Method 5

### Example S12. Synthesis of P-37

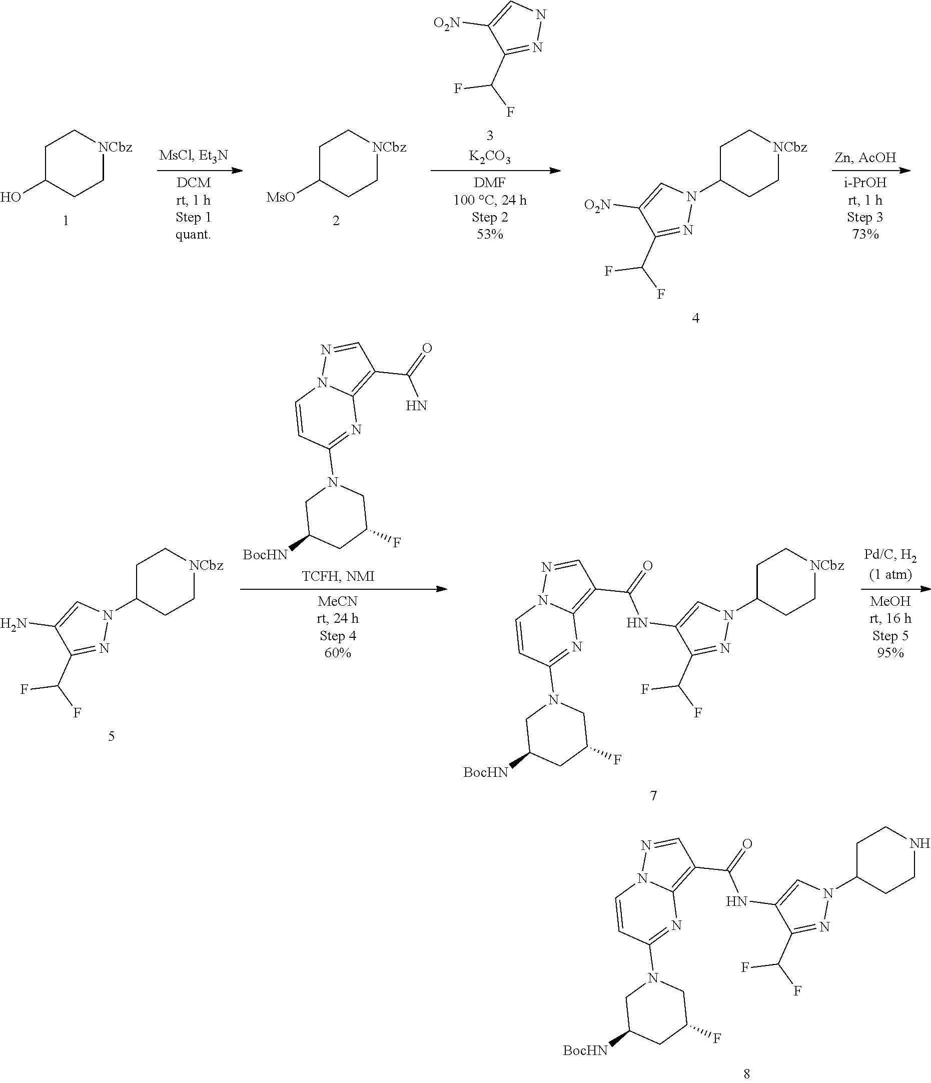

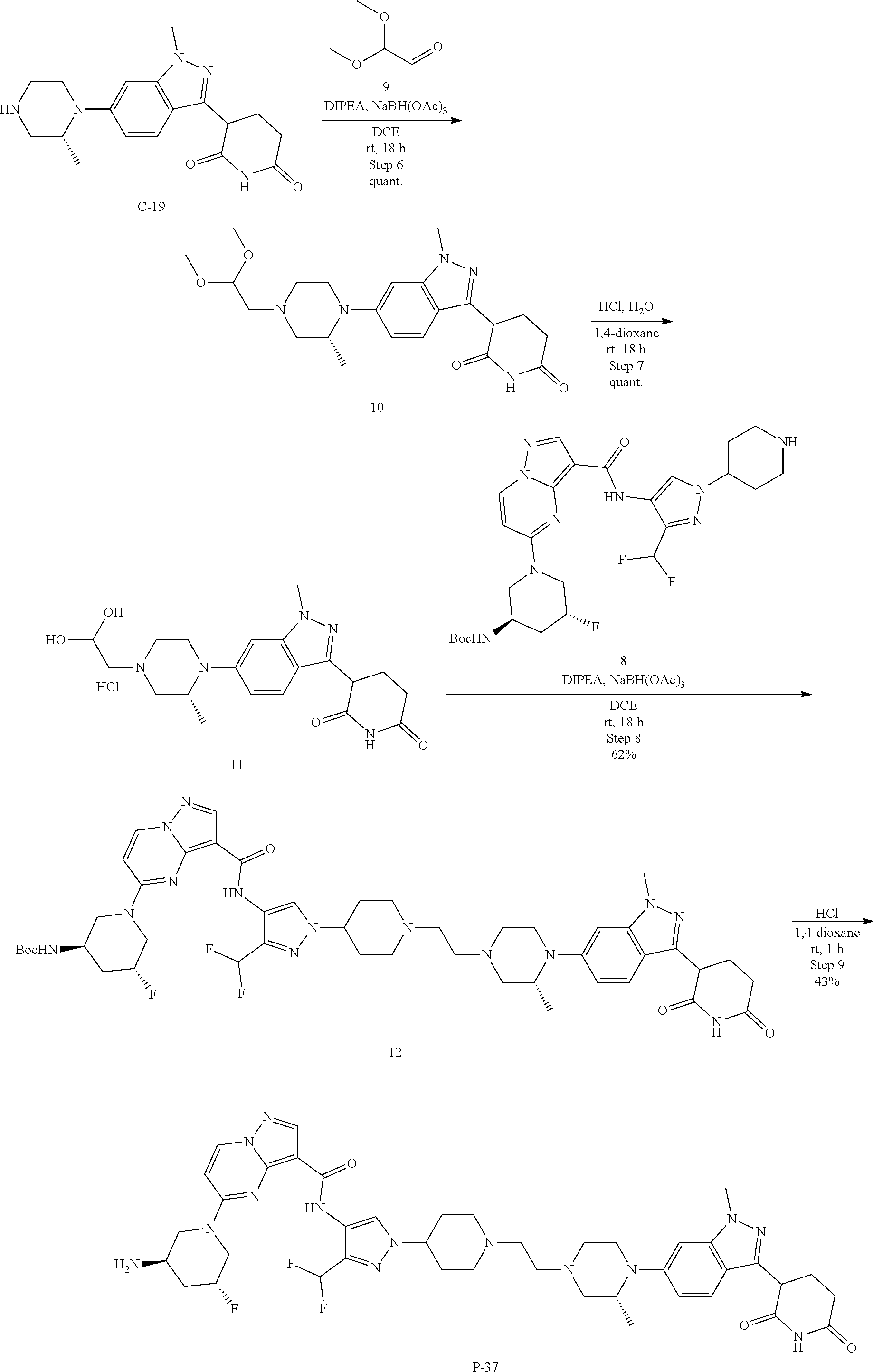

Step 1. Preparation of benzyl 4-methylsulfonyloxypiperidine-1-carboxylate (2). Methanesulfonyl chloride (0.821 mL, 10.6 mmol, 1.25 eq.) was added dropwise to an ice-cooled solution of 4-hydroxypiperidine-1-carboxylate 1 (2.00 g, 8.50 mmol, 1.0 eq.) and triethylamine (1.78 mL, 12.8 mmol, 1.5 eq.) in DCM (100 mL). The reaction mixture was stirred at room temperature. After 1 h, LCMS showed full conversion. Water (50 mL) and saturated NaHCO₃(aq) (50 mL) were added to the reaction mixture, the phases were separated, then the aqueous layer was extracted with DCM (3×50 mL). The combined organics were washed with brine (50 mL), then dried over MgSO₄, filtered and evaporated under reduced pressure to afford 2 (2.80 g, 8.50 mmol, quantitative yield) as a light-orange oil.

LCMS method 1: 95.8% purity at 215 nm, [M+H]⁺=314.1.

¹H NMR (400 MHz, CDCl₃) δ ppm 1.77-1.92 (m, 2H), 1.93-2.08 (m, 2H), 3.05 (s, 3H), 3.43 (ddd, J=13.7, 7.9, 3.9 Hz, 2H), 3.70-3.85 (m, 2H), 4.91 (tt, J=7.5, 3.8 Hz, 1H), 5.14 (s, 2H), 7.27-7.43 (m, 5H).

Step 2. Preparation of benzyl 4-[3-(difluoromethyl)-4-nitro-pyrazol-1-yl]piperidine-1-carboxylate (4). A mixture of benzyl 4-methylsulfonyloxypiperidine-1-carboxylate 2 (1.00 g, 3.19 mmol, 1.2 eq.), 3-(difluoromethyl)-4-nitro-1H-pyrazole 3 (450 mg, 2.76 mmol, 1.0 eq.), and potassium carbonate (800 mg, 5.80 mmol, 2.1 eq.) in DMF (22 mL) was stirred at 100° C. under nitrogen atmosphere. After 24 h, LCMS showed full conversion. Water (100 mL) was added to the reaction mixture and the aqueous phase was extracted with EtOAc (3×50 mL). The combined organics were washed with 1:1 water/brine (3×50 mL) and brine (50 mL), dried over MgSO₄ and evaporated to dryness. The crude was purified by normal phase flash chromatography (80 g silica column, preabsorbed, elution: Heptanes/EtOAc, 95:5 to 70:30, 12 CV) to afford 4 (644 mg, 1.46 mmol, 53% yield) as a light-yellow oil.

LCMS method 1: 86.00% purity at 215 nm, [M+H]⁺=381.1.

¹H NMR (400 MHz, CDCl₃) δ ppm 1.90-2.01 (m, 2H), 2.22 (br d, J=12.1 Hz, 2H), 2.88-3.04 (m, 2H), 4.37 (tt, J=11.7, 4.0 Hz, 3H), 5.16 (s, 2H), 7.12 (t, J=53.3 Hz, 1H), 7.32-7.43 (m, 5H), 8.21 (s, 1H).

¹⁹F NMR (377 MHz, CDCl₃) δ ppm −117.79 (s, 2F).

Step 3. Preparation of benzyl 4-[4-amino-3-(difluoromethyl)pyrazol-1-yl]piperidine-1-carboxylate (5). Zinc (2.21 g, 33.9 mmol, 20.0 eq.; activated with 1.0 M HCl(aq)) was added to a solution of benzyl 4-[3-(difluoromethyl)-4-nitro-pyrazol-1-yl]piperidine-1-carboxylate 4 (644 mg, 1.46 mmol, 1.0 eq.) in i-PrOH (17 mL). Acetic acid (0.970 mL, 16.9 mmol, 10.0 eq.) was then added to the solution and the reaction mixture was stirred at room temperature. After 1 h, LCMS showed full conversion. The reaction mixture was filtered through a celite pad and evaporated to dryness. The residue was taken up in EtOAc (50 mL), washed with saturated NaHCO₃(aq) (10 mL) and brine (10 mL), dried over MgSO₄ and evaporated to dryness to afford 5 (500 mg, 1.24 mmol, 73% yield) as a brown oil.

LCMS method 1: 86.90% purity at 215 nm, [M+H]⁺=351.1.

¹H NMR (400 MHz, DMSO-d6) δ ppm 1.73 (qd, J=12.2, 4.5 Hz, 2H), 1.91-2.01 (m, 2H), 2.86-3.10 (m, 2H), 4.08 (br d, J=12.8 Hz, 4H), 4.17-4.30 (m, 1H), 5.09 (s, 2H), 6.88 (t, J=54.2 Hz, 1H), 7.18 (s, 1H), 7.31-7.39 (m, 5H).

¹⁹F NMR (377 MHz, DMSO-d6) δ ppm −111.51 (s, 2F).

Step 4. Preparation of benzyl 4-[3-(difluoromethyl)-4-[[5-[(3R,5R)-3-(tert-butoxycarbonylamino)-5-fluoro-1-piperidyl]pyrazolo[1,5-a]pyrimidine-3-carbonyl]amino]pyrazol-1-yl]piperidine-1-carboxylate (7). TCFH (614 mg, 2.19 mmol, 1.2 eq.) was added in one portion to a solution of 5-[(3R,5R)-3-(tert-butoxycarbonylamino)-5-fluoro-1-piperidyl]pyrazolo[1,5-a]pyrimidine-3-carboxylic acid 6—intermediate 4 from synthesis of P-2 —(692 mg, 1.82 mmol, 1.0 eq.), benzyl 4-[4-amino-3-(difluoromethyl)pyrazol-1-yl]piperidine-1-carboxylate 5 (708 mg, 1.92 mmol, 1.05 eq.) and NMI (506 μL, 6.38 mmol, 3.5 eq.) in MeCN (12.1 mL), then the reaction mixture was stirred at room temperature. After 24 h, LCMS showed full conversion. The volatiles were evaporated under reduced pressure. The residue was purified by reversed phase flash chromatography purification (150 g C18 RediSep Rf Gold column, liquid deposit (DMSO), MeOH in 0.1% HCOOH(aq), 40%→90%, 15 CV). Fractions were combined and concentrated to afford 7 (823 mg, 1.10 mmol, 60% yield) as an off-white solid.

LCMS method 1: 95.0% purity at 215 nm, [M+H]⁺=712.3.

¹H NMR (400 MHz, DMSO-d6) δ ppm 1.33 (br s, 9H), 1.68-1.97 (m, 4H), 2.03 (br d, J=10.6 Hz, 2H), 2.09-2.22 (m, 1H), 2.88-3.18 (m, 3H), 3.36-3.56 (m, 1H), 3.67 (br d, J=7.0 Hz, 1H), 4.12 (br d, J=13.2 Hz, 2H), 4.42-4.56 (m, 1H), 4.57-4.92 (m, 1H), 4.97 (d, J=45.4 Hz, 1H), 5.10 (s, 2H), 6.89 (br d, J=8.1 Hz, 1H), 6.92-7.26 (m, 2H), 7.29-7.36 (m, 1H), 7.38 (s, 2H), 7.39 (s, 2H), 8.29 (s, 1H), 8.38 (br s, 1H), 8.82 (d, J=7.8 Hz, 1H), 9.31 (br s, 1H).

¹⁹F NMR (377 MHz, DMSO-d6) δ ppm −186.27-−182.64 (m, 1F), −114.40-−109.21 (m, 2F).

Step 5. Preparation of tert-butyl N-[(3R,5R)-1-[3-[[3-(difluoromethyl)-1-(4-piperidyl)pyrazol-4-yl]carbamoyl]pyrazolo[1,5-a]pyrimidin-5-yl]-5-fluoro-3-piperidyl]carbamate (8). Benzyl 4-[3-(difluoromethyl)-4-[[5-[(3R,5R)-3-(tert-butoxycarbonylamino)-5-fluoro-1-piperidyl]pyrazolo[1,5-a]pyrimidine-3-carbonyl]amino]pyrazol-1-yl]piperidine-1-carboxylate 7 (739 mg, 1.04 mmol, 1.0 eq.) was dissolved in methanol (4.0 mL), then the solution was sparged with nitrogen under sonication. After 20 min, Pd/C 10% w/w (200 mg, 0.181 mmol, 0.18 eq.) was added, then the mixture was further sparged with nitrogen under sonication. After 20 min, the nitrogen balloon was switched with one filled with hydrogen and the reaction was sparged. After 10 min, the reaction mixture was stirred under hydrogen atmosphere. After 18 h, LCMS showed full conversion. The reaction mixture was filtered through celite, then the celite was washed thoroughly with MeOH. The filtrate was evaporated under reduced pressure and the residue was taken up in EtOAc. Heptanes were added until the formation of a precipitate. The suspension was evaporated to dryness to give 8 (620 mg, 0.987 mmol, 95% yield) as a pink solid.

LCMS method 1: 92.0% purity at 215 nm, [M+H]⁺=578.3.

¹H NMR (400 MHz, DMSO-d6) δ ppm 0.81-0.87 (m, 1H), 1.20-1.27 (m, 1H), 1.34 (br s, 9H), 1.69-1.82 (m, 3H), 1.88-1.95 (m, 2H), 2.07-2.23 (m, 1H), 2.53-2.61 (m, 2H), 3.03 (br d, J=12.8 Hz, 3H), 3.37-3.51 (m, 1H), 3.67 (br d, J=2.1 Hz, 1H), 4.19-4.33 (m, 1H), 4.45-4.87 (m, 1H), 5.04 (d, J=46.3 Hz, 1H), 6.89 (br d, J=8.1 Hz, 1H), 6.92-7.33 (m, 2H), 8.29 (s, 1H), 8.33 (br s, 1H), 8.82 (d, J=7.8 Hz, 1H), 9.32 (br s, 1H).

¹⁹F NMR (377 MHz, DMSO-d6) δ ppm −187.05-−180.57 (m, 1F), −113.62-−108.43 (m, 2F).

Step 6. Preparation of 3-[1-methyl-6-[(2R)-4-(2,2-dimethoxyethyl)-2-methyl-piperazin-1-yl]indazol-3-yl]piperidine-2,6-dione (10). DIPEA (397 μL, 2.28 mmol, 10.0 eq.) was added to a suspension of 3-[1-methyl-6-[(2R)-2-methylpiperazin-4-ium-1-yl]indazol-3-yl]piperidine-2,6-dione chloride C-19 (99 mg, 0.228 mmol, 1.0 eq.) in DCE (4.5 mL), then 2,2-dimethoxyacetaldehyde 9 (60% w/w solution in H₂O, 52 μL, 0.342 mmol, 1.5 eq.) was added and the mixture was stirred at room temperature. After 10 min, sodium triacetoxyborohydride (96.6 mg, 0.456 mmol, 2.0 eq.) was added in one portion, then the reaction mixture was stirred at room temperature. After 18 h, LCMS showed full conversion. Water (10 mL) was added to the reaction mixture, the phases were separated, then the aqueous phase was extracted with DCM (3×15 mL). The combined organics were washed with brine (10 mL), dried over MgSO₄, filtered and evaporated under reduced pressure to afford 10 (98 mg, 0.228 mmol, quantitative yield) as a tan oil, which was used without purification in the next step.

LCMS method 2: 99.9% purity at 215 nm, [M+H]⁺=430.4.

¹H NMR (400 MHz, DMSO-d6) δ ppm 1.01 (d, J=6.5 Hz, 3H), 2.09-2.21 (m, 1H), 2.24-2.36 (m, 2H), 2.45-2.48 (m, 2H), 2.56-2.66 (m, 2H), 2.76 (br d, J=11.1 Hz, 1H), 2.93 (br d, J=10.0 Hz, 1H), 2.98-3.08 (m, 1H), 3.09-3.17 (m, 2H), 3.29 (d, J=3.9 Hz, 6H), 3.43-3.53 (m, 1H), 3.64-3.74 (m, 1H), 3.88 (s, 3H), 4.03-4.15 (m, 1H), 4.25 (dd, J=8.9, 5.0 Hz, 1H), 4.53 (t, J=5.2 Hz, 1H), 6.78 (s, 1H), 6.89 (dd, J=9.1, 1.4 Hz, 1H), 7.49 (d, J=9.0 Hz, 1H), 8.65 (br s, 1H), 10.84 (s, 1H).

Step 7. Preparation of 3-[1-methyl-6-[(2R)-4-(2,2-dihydroxyethyl)-2-methyl-piperazin-4-ium-1-yl]indazol-3-yl]piperidine-2,6-dione chloride (11). HCl 4.0 M in 1,4-dioxane (2.84 mL, 11.4 mmol, 50.0 eq.) and water (82 μL, 4.56 mmol, 20.0 eq.) were added to a solution of 3-[1-methyl-6-[(2R)-4-(2,2-dimethoxyethyl)-2-methyl-piperazin-1-yl]indazol-3-yl]piperidine-2,6-dione 10 (98 mg, 0.228 mmol, 1.0 eq.) in 1,4-dioxane (2.2 mL), then the reaction mixture was stirred at room temperature. After 18 h, LCMS showed full conversion. The volatiles were evaporated under reduced pressure to yield 11 (99 mg, 0.228 mmol, quantitative yield) as a yellow solid, which was used without purification in the next step.

LCMS method 2: 99.9% purity at 215 nm, [M−HCl+H]⁺=402.2.

Step 8. Preparation of tert-butyl N-[(3R,5R)-1-[3-[[3-(difluoromethyl)-1-[1-[2-[(3R)-4-[3-(2,6-dioxo-3-piperidyl)-1-methyl-indazol-6-yl]-3-methyl-piperazin-1-yl]ethyl]-4-piperidyl]pyrazol-4-yl]carbamoyl]pyrazolo[1,5-a]pyrimidin-5-yl]-5-fluoro-3-piperidyl]carbamate (12). DIPEA (556 μL, 3.19 mmol, 20.0 eq.) was added to a suspension of tert-butyl N-[(3R,5R)-1-[3-[[3-(difluoromethyl)-1-(4-piperidyl)pyrazol-4-yl]carbamoyl]pyrazolo[1,5-a]pyrimidin-5-yl]-5-fluoro-3-piperidyl]carbamate 8 (92.2 mg, 0.160 mmol, 1.0 eq.) and 3-[1-methyl-6-[(2R)-4-(2,2-dihydroxyethyl)-2-methyl-piperazin-4-ium-1-yl]indazol-3-yl]piperidine-2,6-dione chloride 11 (83.9 mg, 0.191 mmol, 1.2 eq.) in DCE (3.2 mL), then the mixture was stirred at room temperature. After 10 min, sodium triacetoxyborohydride (101.4 mg, 0.479 mmol, 3.0 eq.) was added in one portion, then the reaction mixture was stirred at room temperature. After 18 h, LCMS showed full conversion. The volatiles were evaporated under reduced pressure and the residue was purified by reverse phase flash chromatography (100 g C18 RediSep Rf Gold column, liquid deposid (DMSO), MeCN in 0.1% HCOOH(aq), 5% (3 CV)→50%, 20 CV). Fractions were combined and concentrated to give 12 (102 mg, 0.0994 mmol, 62% yield) as a pink solid.

LCMS method 2: 92.1% purity at 215 nm, [M+2H]²⁺=473.4.

¹H NMR (400 MHz, DMSO-d6) δ ppm 1.02 (br d, J=6.4 Hz, 3H), 1.34 (br s, 9H), 1.86-2.03 (m, 4H), 2.11-2.23 (m, 4H), 2.26-2.34 (m, 1H), 2.35-2.43 (m, 1H), 2.61-2.65 (m, 1H), 2.78 (br d, J=10.4 Hz, 1H), 2.96 (br d, J=10.4 Hz, 2H), 3.06 (br d, J=10.3 Hz, 3H), 3.32 (br d, J=12.0 Hz, 3H), 3.37-3.52 (m, 4H), 3.63-3.75 (m, 4H), 3.88 (s, 3H), 4.06-4.16 (m, 1H), 4.25 (br dd, J=9.3, 5.1 Hz, 2H), 4.47-4.92 (m, 2H), 4.95-5.16 (m, 1H), 6.78 (s, 1H), 6.90 (br d, J=7.6 Hz, 2H), 6.93-7.29 (m, 2H), 7.49 (d, J=8.9 Hz, 1H), 8.29 (s, 1H), 8.36 (br s, 1H), 8.82 (br d, J=7.9 Hz, 1H), 9.32 (br s, 1H), 10.84 (s, 1H).

¹⁹F NMR (377 MHz, DMSO-d6) δ ppm −186.79-−182.12 (m, 1F), −113.88-−107.91 (m, 2F).

Step 9. Preparation of 5-((3R,5R)-3-amino-5-fluoropiperidin-1-yl)-N-(3-(difluoromethyl)-1-(1-(2-((3R)-4-(3-(2,6-dioxopiperidin-3-yl)-1-methyl-1H-indazol-6-yl)-3-methylpiperazin-1-yl)ethyl)piperidin-4-yl)-1H-pyrazol-4-yl)pyrazolo[1,5-a]pyrimidine-3-carboxamide (P-37). HCl 4.0 M in 1,4-dioxane (3.65 mL, 14.6 mmol, 150 eq.) was added to tert-butyl N-[(3R,5R)-1-[3-[[3-(difluoromethyl)-1-[1-[2-[(3R)-4-[3-(2,6-dioxo-3-piperidyl)-1-methyl-indazol-6-yl]-3-methyl-piperazin-1-yl]ethyl]-4-piperidyl]pyrazol-4-yl]carbamoyl]pyrazolo[1,5-a]pyrimidin-5-yl]-5-fluoro-3-piperidyl]carbamate 12 (92.2 mg, 0.0976 mmol, 1.0 eq.). The mixture was sonicated for 30 minutes and then stirred at room temperature. After 1 h, LCMS showed full conversion. The solvent was evaporated to dryness and the residue was purified by reverse phase flash chromatography (100 g C18 RediSep Rf Gold column, liquid deposid (DMSO), MeCN in 0.02 M HCl(aq), 5% (3 CV)→40%, 20 CV). Fractions were combined, concentrated and lyophilised to give P-37 (33.27 mg, 0.0387 mmol, 40% yield) as a white solid.

LCMS method 3: 98.3% purity at 215 nm, [M+H]⁺=845.5, [M+2H]²⁺=423.2, [M+3H]³⁺=282.6.

¹H NMR (400 MHz, DMSO-d6) δ ppm 0.97-1.30 (m, 4H), 1.98 (t, J=12.8 Hz, 1H), 2.08 (t, J=11.9 Hz, 1H), 2.12-2.23 (m, 1H), 2.31-2.43 (m, 5H), 2.57-2.72 (m, 2H), 3.12-3.28 (m, 3H), 3.32-3.45 (m, 5H), 3.65-3.77 (m, 4H), 3.78-3.88 (m, 3H), 3.89-4.04 (m, 4H), 4.23-4.42 (m, 1H), 4.42-4.57 (m, 1H), 4.58-4.74 (m, 2H), 4.75-5.01 (m, 1H), 5.10 (d, J=46.6 Hz, 1H), 6.85-7.06 (m, 3H), 7.21 (t, J=53.7 Hz, 1H), 7.51-7.77 (m, 1H), 8.33 (s, 1H), 8.42 (br s, 3H), 8.47 (br s, 1H), 8.94 (d, J=7.8 Hz, 1H), 9.37 (s, 1H), 10.55-10.80 (m, 1H), 10.87 (br s, 1H), 10.97-11.36 (m, 1H).

¹⁹F NMR (377 MHz, DMSO-d6) δ ppm −184.63 (s, 1F), −111.63 (s, 2F).

### Example S13. Synthesis of P-40

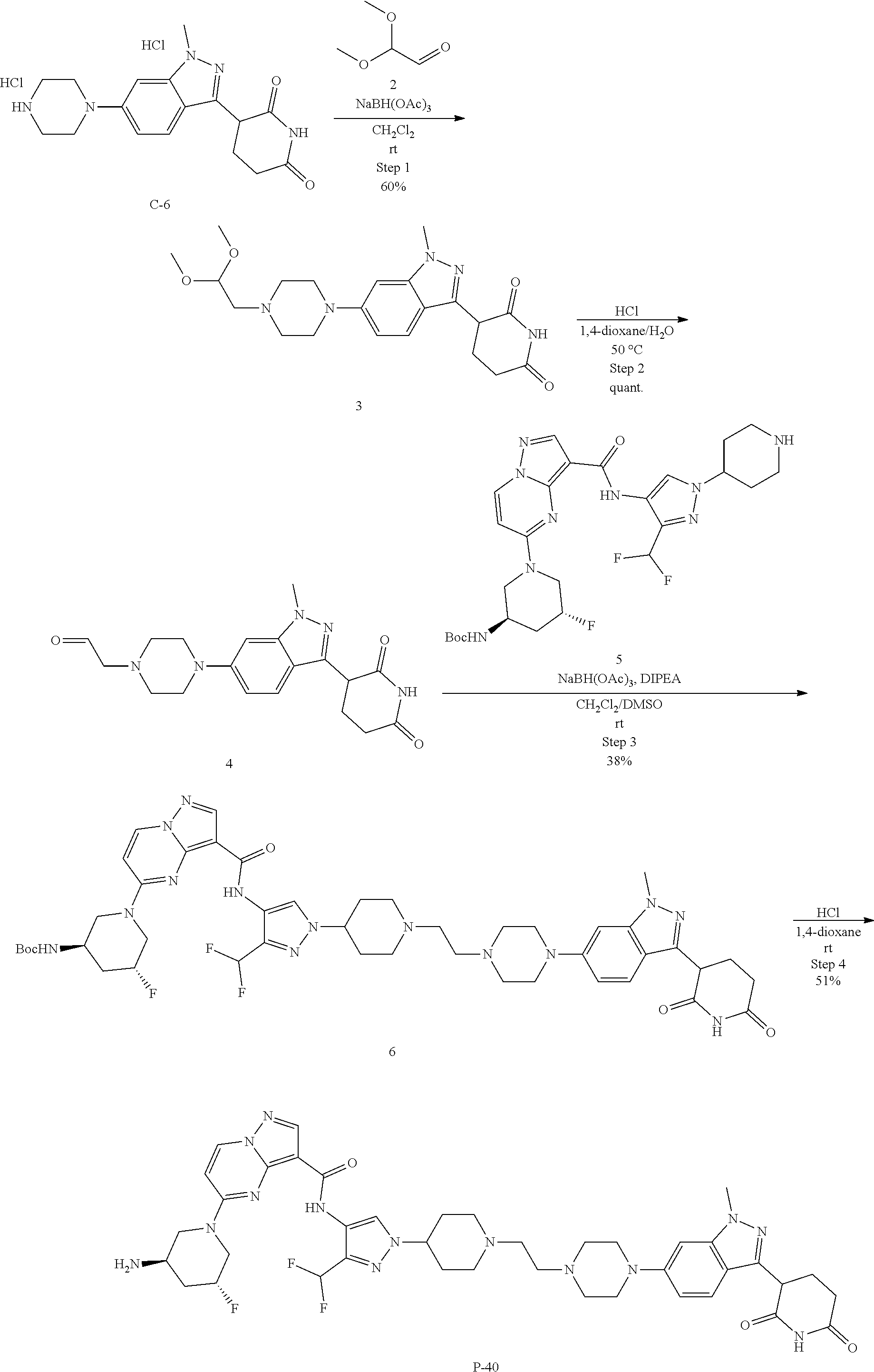

Step 1. Preparation of 3-[6-[4-(2,2-Dimethoxyethyl)piperazin-1-yl]-1-methyl-indazol-3-yl]piperidine-2,6-dione (3). To a solution of 3-(1-methyl-6-piperazin-1-yl-indazol-3-yl)piperidine-2,6-dione dihydrochloride C-6 (180 mg, 0.45 mmol, 1 eq.) and DIPEA (0.31 mL, 1.8 mmol, 4 eq.) in CH₂Cl₂ (4.5 mL) were added 2,2-dimethoxyacetaldehyde 2 (0.14 mL, 0.90 mmol, 2 eq.) and NaBH(OAc)₃ (285.9 mg, 1.35 mmol, 3 eq.). Then the mixture was stirred at room temperature. After 2 h, LCMS showed full conversion. The mixture was then concentrated to remove CH₂Cl₂, and the residue was purified by reverse phase FC (30 g C18 RediSep Rf Gold column, liquid deposit (DMSO+formic acid), elution: 5% MeCN/0.1% HCOOH over 5 CV, then 5 to 100% MeCN/0.1% HCOOH over 30 CV, then 100% MeCN/0.1% HCOOH over 5 CV). Fractions were combined and concentrated to give 3 (121 mg, 60% yield) as a tan solid.

LCMS method 1: 92.3% purity at 215 nm, [M+H]⁺=416.2.

¹H NMR (400 MHz, DMSO-d6) δ ppm 2.10-2.21 (m, 1H), 2.23-2.36 (m, 2H), 2.52-2.74 (m, 6H), 3.26 (br s, 6H), 3.28-3.30 (m, 6H), 4.26 (dd, J=9.3, 5.1 Hz, 1H), 4.51-4.60 (m, 1H), 6.83-6.88 (m, 1H), 6.91 (br d, J=8.8 Hz, 1H), 7.50 (d, J=9.0 Hz, 1H), 10.84 (s, 1H).

Step 2. Preparation of 2-[4-[3-(2,6-Dioxo-3-piperidyl)-1-methyl-indazol-6-yl]piperazin-1-yl]acetaldehyde (4). To a round-bottom flask containing 3-[6-[4-(2,2-dimethoxyethyl)piperazin-1-yl]-1-methyl-indazol-3-yl]piperidine-2,6-dione 3 (121 mg, 0.27 mmol, 1 eq.) were added water (0.10 mL) and 4 M HCl in 1,4-dioxane (1.01 mL, 4.03 mmol, 15 eq.) at room temperature, which was then stirred at 50° C. After 3 h, LCMS showed full conversion. Solvents were removed under reduced pressure, and excess HCl was chased with MeCN (3×) to give 4 (143 mg, quantitative yield) as a yellow solid, which was used without purification in the next step.

LCMS method 1: 81.40% purity at 215 nm, [M+H₂O+H]⁺=388.2.

Step 3. tert-Butyl N-[(3R,5R)-1-[3-[[3-(difluoromethyl)-1-[1-[2-[4-[3-(2,6-dioxo-3-piperidyl)-1-methyl-indazol-6-yl]piperazin-1-yl]ethyl]-4-piperidyl]pyrazol-4-yl]carbamoyl]pyrazolo[1,5-a]pyrimidin-5-yl]-5-fluoro-3-piperidyl]carbamate (6). To a solution of 2-[4-[3-(2,6-dioxo-3-piperidyl)-1-methyl-indazol-6-yl]piperazin-1-yl]acetaldehyde 4 (140 mg, 0.27 mmol, 1.4 eq.), tert-butyl N-[(3R,5R)-1-[3-[[3-(difluoromethyl)-1-(4-piperidyl)pyrazol-4-yl]carbamoyl]pyrazolo[1,5-a]pyrimidin-5-yl]-5-fluoro-3-piperidyl]carbamate 5—intermediate 8 from synthesis of P-37 —(140 mg, 0.22 mmol, 1 eq.) and DIPEA (0.17 mL, 1.11 mmol, 5 eq.) in DMSO (0.5 mL) and CH₂Cl₂ (2 mL) was added NaBH(OAc)₃ (142 mg, 0.67 mmol, 3 eq.) at room temperature. Then the mixture was stirred at room temperature. After 18 h, LCMS showed full conversion with impurities. CH₂Cl₂ was removed under reduced pressure and the residue in DMSO was purified by reverse phase FC (50 g C18 RediSep Rf Gold column, liquid deposit (DMSO+formic acid), elution: 5% MeCN/0.1% HCOOH over 5 CV, then 5 to 100% MeCN/0.1% HCOOH over 20 CV, then 100% MeCN/0.1% HCOOH over 5 CV). Fractions were combined, concentrated and lyophilised to give 6 (78 mg, 38% yield) as a light yellow solid.

LCMS method 1: 88.0% purity at 254 nm, [M+H]⁺=931.2.

¹H NMR (400 MHz, DMSO-d6) δ ppm 1.25-1.45 (m, 11H), 1.76-2.03 (m, 6H), 2.06-2.22 (m, 4H), 2.57-2.64 (m, 5H), 2.97-3.06 (m, 2H), 3.18-3.24 (m, 4H), 3.44-3.72 (m, 4H), 3.89 (s, 3H), 4.15 (s, 2H), 4.94-5.13 (m, 1H), 6.83-6.95 (m, 3H), 7.06-7.16 (m, 1H), 7.49 (d, J=8.8 Hz, 1H), 8.17 (s, 1H), 8.29 (s, 1H), 8.33-8.38 (m, 1H), 8.82 (d, J=7.6 Hz, 1H), 9.29-9.39 (m, 1H), 10.79-10.92 (m, 1H).

Step 4. N-[3-(Difluoromethyl)-1-[1-[2-[4-[3-(2,6-dioxo-3-piperidyl)-1-methyl-indazol-6-yl]piperazin-1-yl]ethyl]-4-piperidyl]pyrazol-4-yl]-5-[(3R,5R)-3-amino-5-fluoro-1-piperidyl]pyrazolo[1,5-a]pyrimidine-3-carboxamide (P-40). To tert-butyl N-[(3R,5R)-1-[3-[[3-(difluoromethyl)-1-[1-[2-[4-[3-(2,6-dioxo-3-piperidyl)-1-methyl-indazol-6-yl]piperazin-1-yl]ethyl]-4-piperidyl]pyrazol-4-yl]carbamoyl]pyrazolo[1,5-a]pyrimidin-5-yl]-5-fluoro-3-piperidyl]carbamate 6 (46 mg, 0.05 mmol, 1 eq.) was added 4 M HCl in 1,4-dioxane (0.62 mL, 2.47 mmol, 50 eq.). The reaction mixture was stirred at room temperature. After 16 h, LCMS showed full conversion. Volatiles were removed under reduced pressure and excess HCl was chased by co-evaporation with MeCN (3×). The residue was then purified by reverse phase flash chromatography (30 g C18 RediSep Rf Gold column, liquid deposit (water), elution: 5% MeCN/0.1% HCOOH over 5 CV, then 5 to 100% MeCN/0.1% HCOOH over 20 CV, then 100% MeCN/0.1% HCOOH over 5 CV). Fractions were combined, concentrated and lyophilised to give P-40 (21.4 mg, 51% yield) as a light pink solid.

LCMS method 2: 96.1% purity at 215 nm, [M+H]⁺=831.4, [M+2H]²⁺=416.2, [M+3H]3+=277.9.

¹H NMR (400 MHz, DMSO-d6) δ ppm 1.06-1.11 (m, 1H), 1.58-1.78 (m, 1H), 1.90-2.04 (m, 4H), 2.11-2.21 (m, 4H), 2.26-2.34 (m, 1H), 2.52-2.54 (m, 3H), 2.59-2.66 (m, 6H), 2.92-3.12 (m, 4H), 3.17-3.26 (m, 4H), 3.29-3.43 (m, 2H), 3.55-3.61 (m, 1H), 3.67-3.74 (m, 1H), 3.89 (s, 3H), 4.17-4.29 (m, 3H), 4.95-5.09 (m, 1H), 6.84 (s, 1H), 6.92 (d, J=8.1 Hz, 2H), 7.02-7.30 (m, 1H), 7.49 (d, J=9.0 Hz, 1H), 8.29 (s, 1H), 8.40 (s, 1H), 8.82 (d, J=7.8 Hz, 1H), 9.34 (s, 1H), 10.85 (s, 1H).

¹⁹F NMR (377 MHz, DMSO-d6) δ −183.57 (s, 1F), −111.18 (d, J=49.0 Hz, 2F).

The following compounds were synthesized via the same general routes with modifications to CBM (C—X) in step 6 (Table 8).

TABLE 8

| Com- | | | |
|---|---|---|---|
| pound | | | |
| No. | CBM | Structure | Characterization Data |
| P-36 | C-18 | 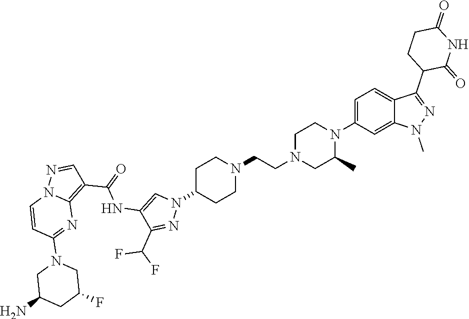 | 59% yield as a tan solid. LCMS method 2: retention time: 1.706 min, 98% purity at 215 nm, [M − 3HCl + H]⁺ = 845.4, [M − 3HCl + 2H]²⁺ = 423.2, [M − 3HCl + 3H]³⁺ = 282.6. ¹H NMR (400 MHz, DMSO-d6) δ ppm 0.93- 1.38 (m, 3 H), 2.00 (t, J = 11.4 Hz, 1 H), 2.08- 2.23 (m, 2 H), 2.32-2.45 (m, 5 H), 2.56-2.74 (m, 2 H), 3.14-3.31 (m, 3 H), 3.32-3.42 (m, 4 H), 3.43-3.52 (m, 2 H), 3.63-3.75 (m, 4 H), 3.76-3.90 (m, 4 H), 3.94 (br s, 3 H), 4.20-4.42 (m, 1 H), 4.42-4.57 (m, 1 H), 4.59-4.76 (m, 2 H), 4.79-4.91 (m, 1 H), 5.15 (d, J = 41.7 Hz, 1 H), 6.80-7.05 (m, 3 H), 7.21 (t, J = 53.9 Hz, 1 H), 7.41-7.85 (m, 1 H), 8.33 (s, 1 H), 8.47 (br s, 1 H), 8.55 (br s, 3 H), 8.93 (d, J = 7.8 Hz, 1 H), 9.37 (s, 1 H), 10.67-10.99 (m, 2 H), 11.00-11.55 (m, 1 H). ¹⁹F NMR (377 MHz, DMSO-d6) δ ppm −184.59 (s, 1 F), −111.65 (s, 2 F). |
| P-37 | C-19 | 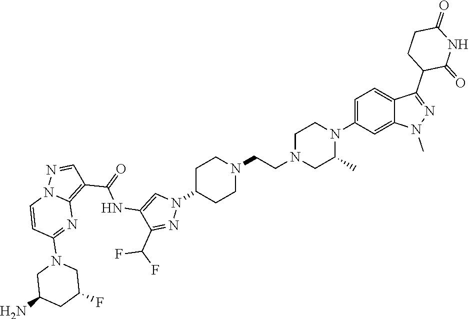 | 40% yield as a white solid. LCMS method 2: retention time: 1.705 min, 98% purity at 215 nm, [M − 3HCl + H]⁺ = 845.5, [M − 3HCl + 2H]²⁺ = 423.2, [M − 3HCl + 3H]3+ = 282.6. ¹H NMR (400 MHz, DMSO-d6) δ 0.97-1.30 (m, 4 H), 1.98 (t, J = 12.8 Hz, 1 H), 2.08 (t, J = 11.9 Hz, 1 H), 2.12-2.23 (m, 1 H), 2.31- 2.43 (m, 5 H), 2.57-2.72 (m, 2 H), 3.12-3.28 (m, 3 H), 3.32-3.45 (m, 5 H), 3.65-3.77 (m, 4 H), 3.78-3.88 (m, 3 H), 3.89-4.04 (m, 4 H), 4.23-4.42 (m, 1 H), 4.42-4.57 (m, 1 H), 4.58- 4.74 (m, 2 H), 4.75-5.01 (m, 1 H), 5.10 (d, J = 46.6 Hz, 1 H), 6.85-7.06 (m, 3 H), 7.21 (t, J = 53.7 Hz, 1 H), 7.51-7.77 (m, 1 H), 8.33 (s, 1 H), 8.42 (br s, 3 H), 8.47 (br s, 1 H), 8.94 (d, J = 7.8 Hz, 1 H), 9.37 (s, 1 H), 10.55-10.80 (m, 1 H), 10.87 (br s, 1 H), 10.97-11.36 (m, 1 H). ¹⁹F NMR (377 MHz, DMSO-d6) δ ppm −184.63 (s, 1 F), −111.63 (s, 2 F). |
| P-40 | C-6 | 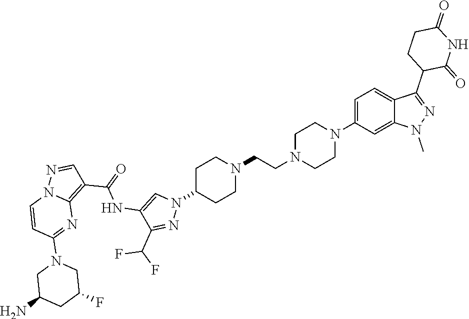 | 50% yield as a light pink solid. LCMS method 2: retention time: 1.691 min, 96% purity at 215 nm, [M + H]⁺ = 831.4, [M + 2H]²⁺ = 416.2, [M + 3H]3+ = 277.9. ¹H NMR (400 MHz, DMSO-d6) δ ppm 1.06- 1.11 (m, 1 H), 1.58-1.78 (m, 1 H), 1.90-2.04 (m, 4 H), 2.11-2.21 (m, 4 H), 2.26-2.34 (m, 1 H), 2.52-2.54 (m, 3 H), 2.59-2.66 (m, 6 H), 2.92-3.12 (m, 4 H), 3.17-3.26 (m, 4 H), 3.29-3.43 (m, 2 H), 3.55-3.61 (m, 1 H), 3.67-3.74 (m, 1 H), 3.89 (s, 3 H), 4.17-4.29 (m, 3 H), 4.95-5.09 (m, 1 H), 6.84 (s, 1 H), 6.92 (d, J = 8.1 Hz, 2 H), 7.02-7.30 (m, 1 H), 7.49 (d, J = 9.0 Hz, 1 H), 8.29 (s, 1 H), 8.40 (s, 1 H), 8.82 (d, J = 7.8 Hz, 1 H), 9.34 (s, 1 H), 10.85 (s, 1 H). ¹⁹F NMR (377 MHz, DMSO-d6) δ −183.57 (s, 1 F), −111.18 (d, J = 49.0 Hz, 2 F). |

### BIOLOGICAL EXAMPLES

### Example B1. IRAK4 PhosphoSens Biochemical Assay (BIOCHEM) Procedure

The PhosphoSens® biochemical assay was performed as described by the vendor (AssayQuant Technologies Inc. Marlborough, MA). A 1.25× stock solution of IRAK4 (PV4002, ThermoFisher Scientific, Waltham, MA) and a 5× stock solution of ATP and Sox conjugated peptide substrate, AQT0326 (CSKS-AQT0326B, AssayQuant Technologies), were prepared in 1× kinase reaction buffer consisting of 50 mM HEPES, pH 7.5, 0.01% Brij-35, 0.5 mM EGTA, 20 mM MgCl₂ and 1 mM DTT. 10 μL of the ATP and substrate solution mix, for a final concentration of 200 μM ATP and 10 μM peptide substrate, was added to a Coming 3574 384-well, white, non-binding surface microtiter plate containing 0.5 μL of serially diluted test compounds prepared in DMSO. The reactions were started with the addition of 40 μL of the enzyme solution, for a final IRAK4 concentration of 1 nM, and monitored every 71 seconds for 240 minutes at λ_{EX} 360/λ_{EM} 485 in a BioTek Synergy H4 plate reader (Agilent Technologies, Santa Clara, CA) at room temperature. The initial linear portions of the progress curves were fit according to a linear equation to yield the slopes and converted to % inhibition based on a value of 100% activity for the no inhibitor treated control. IC₅₀ values of each compound were obtained by fitting the % inhibition-compound concentration curves using Dotmatics software (Dotmatics, Bishops Stortford, Hertfordshire, England).

### Example B2. Reagent Preparations

Cell culture media was prepared in a tissue culture hood in a sterile environment by adding 10% FBS and 1% Penicillin Streptomycin to 500 mL no phenol red RPMI 1640 media. The media was filtered through a Nalgene Bottle Top Filter and stored at 4° C.

The Cell titer Glo (CTG) buffer and substrate (CellTiter-Glo Luminescent Cell Viability Assay, Promega Ref. #G7573) were stored in −20° C. The CTG buffer (100 mL) was warmed in a bead bath and added to the CTG substrate bottle in a tissue culture hood. The solution was mixed with a pipette until it became homogenous. CTG reagent were aliquoted into 15 mL falcon tubes and stored at −20° C.

For Homogenous Time Resolved Fluorescence (HTRF) assays, a Cisbio HTRF kit was used, which included: Lysis Buffer #1 4×, Blocking Reagent #3 100×, 20× Antibody 1 (Anti-IRAK4 d2), 20× Antibody 2 (Anti-IRAK4 k), and Detection Buffer.

4× Lysis Buffer was stored at 4° C. For use as 1× Lysis buffer, the 4× solution was diluted with de-ionized water (distilled water, Gibco Cat. #15230279) and 100× Blocking Reagent in a 1:3:0.04 volume ratio.

20× Antibody Solution aliquots were stored in −80° C. and the Detection Buffer was stored in 4° C. For use as a 1× Antibody Solution, the 20× Antibody Solution aliquot was diluted with Detection Buffer in a 1:19 volume ratio.

### Example B3. Advanced Lipoxidation End Product THP1 Homogeneous Time Resolved Fluorescence (ALE THP1 HTRF) Procedure

Cells were lysed at room temperature for 45 min with shaking. A BCA protein assay was performed and normalization was conducted to the desired total protein concentration with 1× lysis buffer. Next, 1× Antibody Solutions were prepared by adding 380 μL Detection Buffer to 20 μL 20× Antibody Solution aliquots and mixing well. 1× Antibody Solutions were combined 1:1 and briefly vortexed. For control wells, 20 μL 1× anti IRAK4-k Antibody Solution was saved. The 384-well plate (ProxiPlate-384 Plus, Perkin Elmer Cat. #6008289) was loaded by adding 4 μL of the mixed Antibody Solution to empty wells using a single channel repeater. Using a multi-channel repeater, 16 μL lysate was added per well, and any bubbles that were formed were popped with 20 μL pipette tips and Kimwipe edges. In column 10, triplicates of each control was prepared. In wells A10, B10, and C10, buffer control was prepared by adding 16 μL Lysis Buffer and 4 μL Detection Buffer. In wells D10, E10, and F10, cryptate control was prepared by adding 16 μL Lysis Buffer, 2 μL Detection Buffer, and 2 μL 1× anti IRAK4-k Antibody Solution. In wells G10, H10, and 110, a negative control was prepared by adding 16 μL Lysis Buffer and 4 μL mixed Antibody Solution. The plate was sealed with a clear seal and covered with an aluminum lid. The plate was spun down at 800 g for 5 min and incubated in the dark at room temperature overnight. The next day, the plate was spun down at 800 g for 5 min. The samples were analyzed by a plate reader (Envision, PerkinElmer) using the Desnor 384 HTRF program.

A summary of the ALE THP1 HTRF data for the tested compounds is provided in Table 9 below.

TABLE 9
BIOCHEM and ALE THP1 HTRF Results of the Compounds.

| | | ALE THP1 | ALE THP1 |
|---|---|---|---|
| Compound | BIOCHEM | HTRF | HTRF |
| No. | IC₅₀ (nM) | EC₅₀ (μM) | Y_{MIN} (%) |
| P-1 | 0.5 | >10 | 58 |
| P-2 | <0.5 | 0.004 | 12 |
| P-3 | <0.5 | 0.008 | 14 |
| P-4 | <0.5 | 0.0056 | 23 |
| P-5 | <0.5 | 0.005 | 14 |
| P-6 | <0.5 | 0.005 | 9 |
| P-7 | <0.5 | 0.012 | 13 |
| P-8 | <0.5 | 0.007 | 12 |
| P-9 | <0.5 | 0.004 | 12 |
| P-10 | <0.5 | 0.014 | 17 |
| P-11 | <0.5 | 0.017 | 11 |
| P-12 | <0.5 | 0.006 | 16 |
| P-13 | <0.5 | 0.001 | 56 |
| P-14 | <0.5 | >10 | 63 |
| P-15 | <0.5 | 0.010 | 9 |
| P-16 | <0.5 | 0.005 | 14 |
| P-17 | <0.5 | 0.001 | 53 |
| P-18 | <0.5 | 0.001 | 29 |
| P-19 | <0.5 | 0.005 | 13 |
| P-20 | <0.5 | 0.005 | 41 |
| P-21 | <0.5 | >10 | 48 |
| P-22 | <0.5 | 0.005 | 16 |
| P-23 | <0.5 | 0.004 | 17 |
| P-24 | <0.5 | 0.004 | 17 |
| P-25 | <0.5 | 0.008 | 23 |
| P-26 | <0.5 | 0.011 | 20 |
| P-27 | <0.5 | 0.001 | 68 |
| P-28 | <0.5 | 0.005 | 13 |
| P-29 | <0.5 | >10 | 73 |
| P-30 | <0.5 | 0.014 | 6 |
| P-31 | <0.5 | 0.003 | 18 |
| P-32 | <0.5 | 0.001 | 42 |
| P-33 | <0.5 | 0.010 | 14 |
| P-34 | 0.8 | 0.005 | 37 |
| P-35 | 0.8 | 0.004 | 24 |
| P-36 | <0.5 | 0.005 | 12 |
| P-37 | <0.5 | 0.005 | 11 |
| P-38 | <0.5 | >10 | 54 |
| P-39 | <0.5 | >10 | 63 |
| P-40 | <0.5 | 0.006 | 14 |
| P-41 | <0.5 | >10 | 63 |
| P-42 | <0.5 | >10 | 73 |
| P-43 | <0.5 | >10 | 87 |
| P-44 | 7.3 | >10 | 66 |
| P-45 | <0.5 | 0.004 | 16 |
| P-46 | 0.8 | >3 | 65 |
| P-47 | 2.4 | 3.75 | 30 |
| P-48 | >1 | 3.51 | 19 |
| P-49 | 0.8 | >10 | 92 |
| P-50 | 0.8 | >10 | 52 |

Although the present invention has been described in some detail by way of illustration and example for purposes of clarity of understanding, the descriptions and examples should not be construed as limiting the scope of the invention. The disclosures of all patent and scientific literature cited herein are expressly incorporated herein in their entirety by reference.

1. A compound of Formula (I): 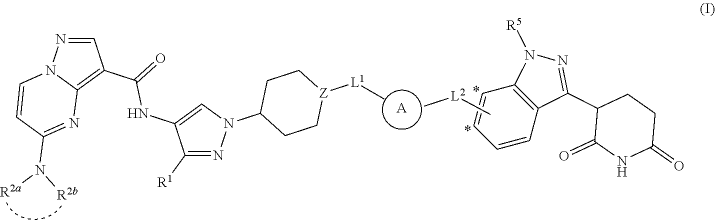
or a pharmaceutically acceptable salt thereof, wherein:
R¹ is C₁-C₆ haloalkyl;
R^{a} is H or C₁-C₆ alkyl;
R^{b} is C₁-C₆ alkyl, 5- to 6-membered heteroaryl, —(C₁-C₆ alkylene)(5- to 6-membered heteroaryl), or —(C₁-C₆ alkylene)NH₂, wherein the heteroaryl contains 1-2 nitrogen atoms and is optionally substituted by 1-5 R² groups;
or the dashed line between R^{a} and R^{b} represents a ring structure where R^{a} and R^{b} are taken together with the nitrogen atom to which they are attached to form a 5- to 10-membered monocyclic or bicyclic heterocyclyl optionally containing 1-2 additional heteroatoms selected from N and O, and is optionally substituted by 1-5 R² groups;
each R² is independently —NH₂, halo, C₁-C₆ alkyl, C₁-C₆ haloalkyl, —CN, or 5- to 6-membered heteroaryl, wherein the heteroaryl contains 1-2 nitrogen atoms and is optionally substituted by 1-5 groups selected from C₁-C₆ alkyl, halo, and C₁-C₆ haloalkyl;
L¹ is —C(O)N(H)—, —C(O)—, —(C₁-C₆ alkylene)N(R³)—, or C₁-C₆ alkylene;
L² is a bond, —C(O)—, —N(R³)—, or O;
each R³ is independently H or C₁-C₆ alkyl;
Ring A is a monocyclic 4- to 6-membered heterocyclylene or a bicyclic 6- to 9-membered spiro heterocyclylene, wherein the heterocyclylene contains 1-2 nitrogen atoms, and wherein the heterocyclylene is substituted by m R⁴ groups;
each R⁴ is independently halo, C₁-C₆ alkyl, or C₁-C₆ haloalkyl;
m is 0-5;
R⁵ is H or C₁-C₆ alkyl;
Z is CH or N; and
* indicates an attachment point for the L²-containing moiety.

2. The compound of claim 1, or a pharmaceutically acceptable salt thereof, wherein:
R¹ is C₁-C₃ haloalkyl.

3. The compound of claim 2, or a pharmaceutically acceptable salt thereof, wherein:
R¹ is —CHF₂.

4. The compound of any one of claims 1-3, or a pharmaceutically acceptable salt thereof, wherein:
R^{a} is H or C₁-C₃ alkyl;
R^{b} is C₁-C₃ alkyl, 6-membered heteroaryl, —(C₁-C₃ alkylene)(6-membered heteroaryl), or —(C₁-C₃ alkylene)NH₂, wherein the heteroaryl contains 1-2 nitrogen atoms and is optionally substituted by 1-2 R² groups; and
each R² is independently —NH₂, halo, C₁-C₃ alkyl, C₁-C₃ haloalkyl, or —CN.

5. The compound of claim 4, or a pharmaceutically acceptable salt thereof, wherein:

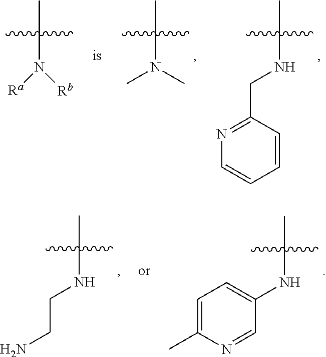

6. The compound of any one of claims 1-3, or a pharmaceutically acceptable salt thereof, wherein:
R^{a} and R^{b} are taken together with the nitrogen atom to which they are attached to form a 5- to 8-membered monocyclic or bicyclic heterocyclyl optionally containing 1-2 additional heteroatoms selected from N and O, and optionally substituted by 1-3 R² groups; and
each R² is independently —NH₂, halo, C₁-C₃ alkyl, C₁-C₃ haloalkyl, —CN, or 5-membered heteroaryl, wherein the heteroaryl contains 1-2 nitrogen atoms and is optionally substituted by 1-3 groups selected from C₁-C₃ alkyl, halo, and C₁-C₃ haloalkyl.

7. The compound of claim 6, or a pharmaceutically acceptable salt thereof, wherein:

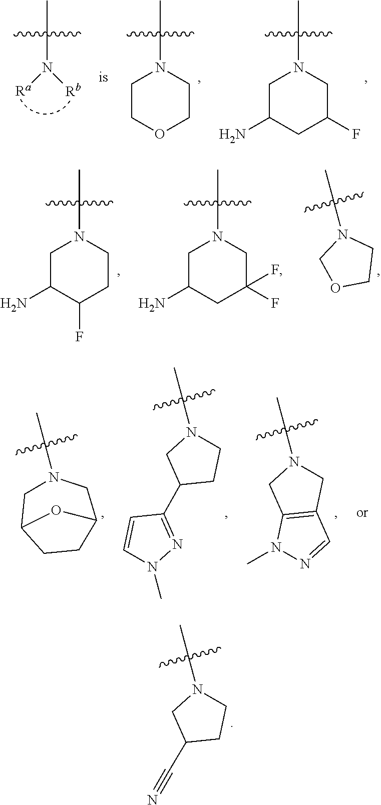

8. The compound of any one of claims 1-7, or a pharmaceutically acceptable salt thereof, wherein:
L¹ is —C(O)N(H)—, —C(O)—, —(C₁-C₃ alkylene)N(R³)—, or C₁-C₃ alkylene;
L² is a bond, —C(O)—, —N(R³)—, or O; and
each R³ is independently H or C₁-C₃ alkyl.

9. The compound of any one of claims 1-8, or a pharmaceutically acceptable salt thereof, wherein:
Ring A is: 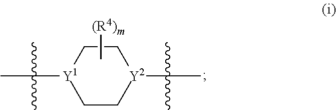
and
Y¹ and Y² are independently CH or N, provided that at least one of Y¹ and Y² is N;

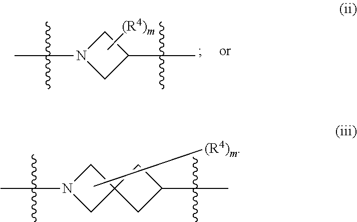

10. The compound of any one of claims 1-9, or a pharmaceutically acceptable salt thereof, wherein:
wherein each R⁴ is independently halo, C₁-C₃ alkyl, or C₁-C₃ haloalkyl.

11. The compound of any one of claims 1-10, or a pharmaceutically acceptable salt thereof, wherein:
m is 0, 1, or 2.

12. The compound of any one of claims 1-11, or a pharmaceutically acceptable salt thereof, wherein: 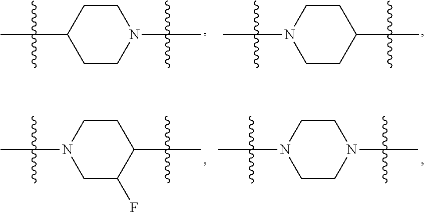
Ring A is

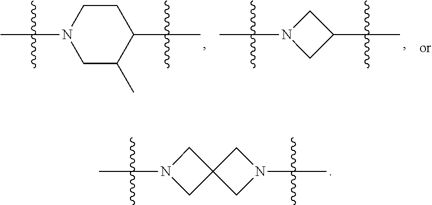

13. The compound of any one of claims 1-12, or a pharmaceutically acceptable salt thereof, wherein:
R⁵ is H or C₁-C₃ alkyl.

14. The compound of claim 13, or a pharmaceutically acceptable salt thereof, wherein:
R⁵ is —CH₃.

15. The compound of any one of claims 1-14, or a pharmaceutically acceptable salt thereof, wherein:

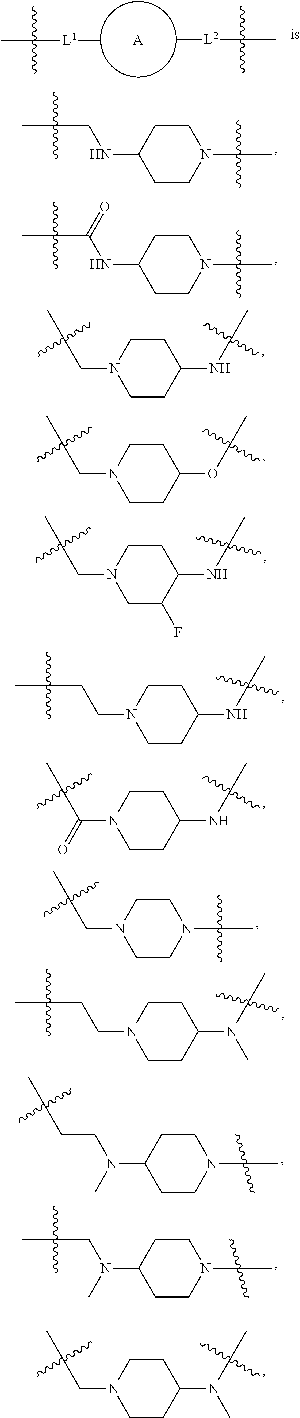

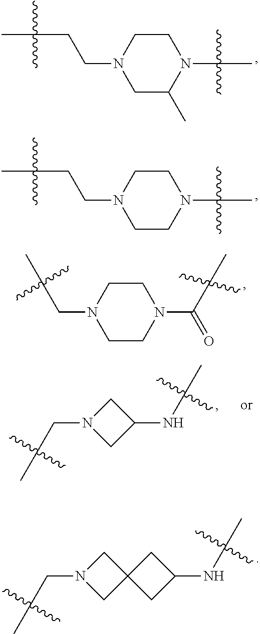 [SMILES: *CCN1CCN(*)C(C)C1 ; *CCN1CCN(*)CC1 ; *CN1CCN(C(*)=O)CC1 ; *CN1CC(N*)C1 ; *CN1CC2(CC(N*)C2)C1]

16. The compound of any one of claims 1-15, or a pharmaceutically acceptable salt thereof, wherein the compound is of Formula (IA), (IB), (IIa), (IIb), (IIIa), or (IVa): 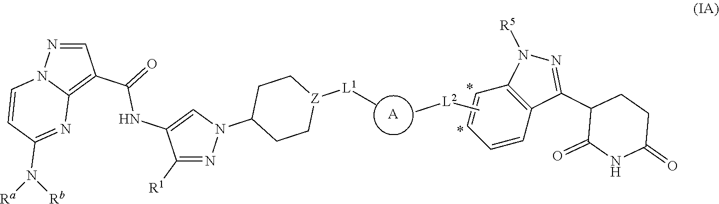
wherein:
R^{a} is H or C₁-C₆ alkyl; and
R^{b} is C₁-C₆ alkyl, 5- to 6-membered heteroaryl, —(C₁-C₆ alkylene)(5- to 6-membered heteroaryl), or —(C₁-C₆ alkylene)NH₂, wherein the heteroaryl contains 1-2 nitrogen atoms and is optionally substituted by 1-5 R² groups; 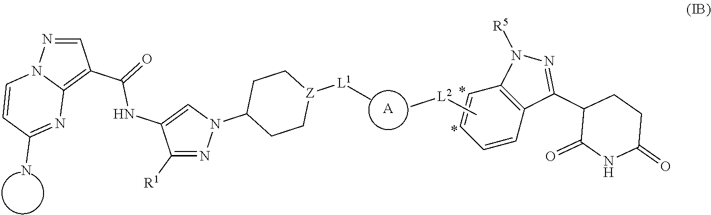
wherein: 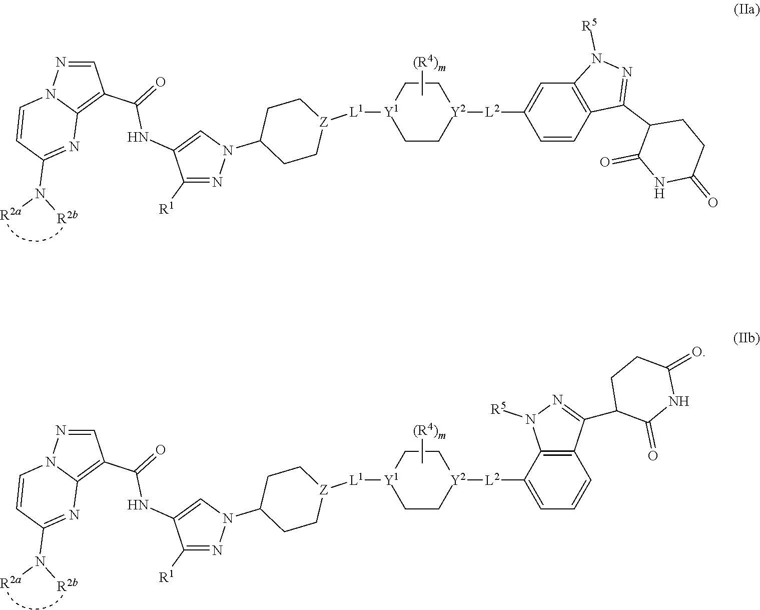
is a 5- to 10-membered monocyclic or bicyclic heterocyclyl optionally containing 1-2 additional heteroatoms selected from N and O, and is optionally substituted by 1-5 R² groups;

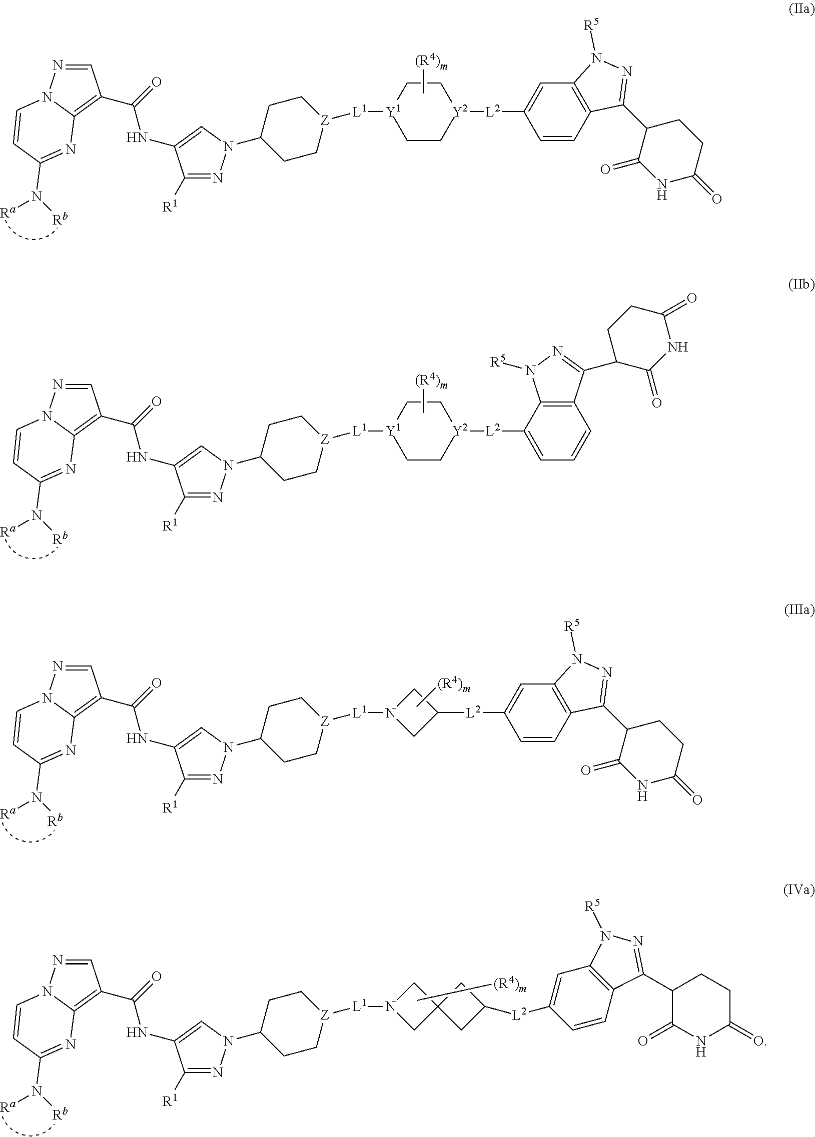

17. A compound selected from the compounds of Table 1 and pharmaceutically acceptable salts thereof.

18. A pharmaceutical composition comprising the compound of any one of claims 1-17, or a pharmaceutically acceptable salt thereof, and a pharmaceutically acceptable excipient.

19. A method of modulating interleukin-1 (IL1) receptor-associated kinase 4 (IRAK4) comprising contacting IRAK4 with an effective amount of the compound of any one of claims 1-17, or a pharmaceutically acceptable salt thereof, or the pharmaceutical composition of claim 18.

20. A method of treating an inflammatory or autoimmune disease in a subject in need thereof, comprising administering to the subject an effective amount of the compound of any one of claims 1-17, or a pharmaceutically acceptable salt thereof, or the pharmaceutical composition of claim 18, optionally wherein the inflammatory or autoimmune disease is atopic dermatitis, asthma, lupus, rheumatoid arthritis, familial mediterranean fever, psoriasis, generalized pustular psoriasis, cryoprin-associated periodic syndrome, hidradenitis suppurativa, Bechet's syndrome, or familial cold autoinflammatory syndrome.
